(19) United States
(12) Patent Application Publication
(10) Pub. No.: US 2026/0234112 A1
(43) Pub. Date: Aug. 13, 2026

(54) COMPOUNDS, COMPOSITIONS, AND METHODS OF USE THEREOF

(71) Applicant: Type6 Therapeutics, Inc., Nicasio, CA (US)

(72) Inventors: Gert Kiss, Nicasio, CA (US); Alexander J. Bridges, Saline, MI (US)

(21) Appl. No.: 19/153,883

(22) PCT Filed: Feb. 5, 2024
(86) PCT No.: PCT/US2024/014466

§ 371 Date: Aug. 5, 2025

Related U.S. Application Data
(60) Provisional application No. 63/483,467, filed on Feb. 6, 2023.

(51) Int. Cl. C07D 239/42; A61K 31/506; A61K 47/64; C07D 401/04; C07D 401/12; C07D 401/14; C07D 405/12; C07D 405/14; C07D 409/14

(52) U.S. Cl. CPC C07D 239/42; A61K 31/506; A61K 47/64; C07D 401/04; C07D 401/12; C07D 401/14; C07D 405/12; C07D 405/14; C07D 409/14; C12Y 207/11022

(57) ABSTRACT

The present disclosure provides, in part, compounds of formula: (I) and pharmaceutically acceptable salts thereof, and compositions comprising said compounds or salts thereof and a pharmaceutically acceptable excipient. In some embodiments, the compounds or salts thereof and compositions disclosed herein are effective at inhibiting CDK2 activity. In some embodiments, the compounds or salts thereof and compositions disclosed herein are selective at inhibiting CDK2 over other CDKs. Thus, also provided herein are uses of the compounds or salts thereof and compositions in methods of treating, preventing, or reducing the risk or severity of certain diseases or disorders mediated by CDK2, such as cancer.

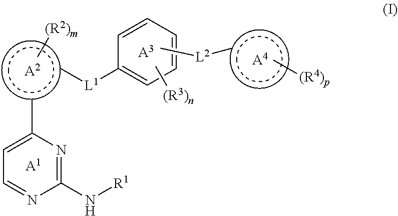

### CROSS REFERENCE

This application claims priority to and the benefit of U.S. Provisional Patent Application No. 63/483,467 filed Feb. 6, 2023, which is incorporated herein by reference in its entirety.

### SUMMARY

Disclosed herein, in some embodiments, are compounds of Formula (I), (II), (III), (IV), (V), (VI), and (VII), and pharmaceutically acceptable salts thereof, compositions comprising said compounds or pharmaceutically acceptable salts thereof and a pharmaceutically acceptable excipient, and uses thereof such as in treating, preventing, or reducing the risk or severity of certain diseases or disorders (e.g., cancer).

In some embodiments, provided herein are compounds of Formula I:

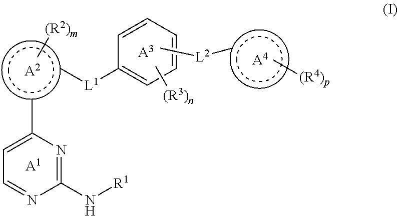

or a pharmaceutically acceptable salt thereof, wherein the variables are as defined herein.

In some embodiments, provided herein are compounds of Formula II:

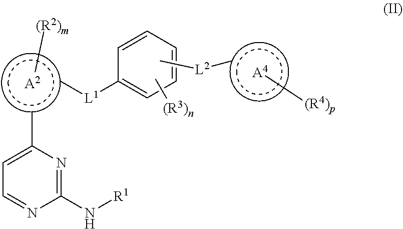

or a pharmaceutically acceptable salt thereof, wherein the variables are as defined herein.

In some embodiments, provided herein are compounds of Formula III:

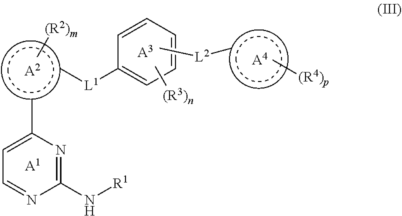

or a pharmaceutically acceptable salt thereof, wherein the variables are as defined herein.

In some embodiments, provided herein are compounds of Formula IV:

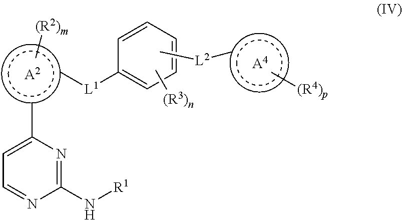

or a pharmaceutically acceptable salt thereof, wherein the variables are as defined herein.

In some embodiments, provided herein are compounds of Formula V:

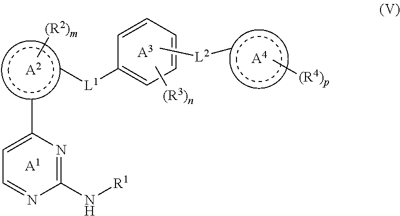

or a pharmaceutically acceptable salt thereof, wherein the variables are as defined herein.

In some embodiments, provided herein are compounds of Formula VI:

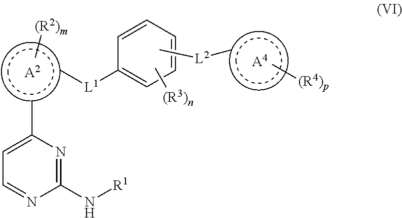

or a pharmaceutically acceptable salt thereof, wherein the variables are as defined herein.

In some embodiments, provided herein are compounds of Formula VII:

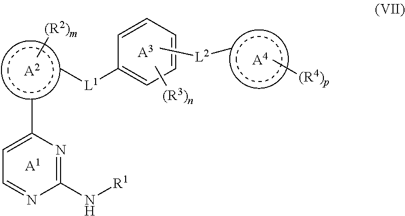

or a pharmaceutically acceptable salt thereof, wherein the variables are as defined herein.

In some embodiments, a compound of the present disclosure is provided in Table 1 as described herein.

In some embodiments, provided herein are pharmaceutical compositions, comprising: a) a compound described herein, or a pharmaceutically acceptable salt thereof; and b) a pharmaceutically acceptable excipient.

In some embodiments, provided herein are methods of inhibiting CDK2 in a subject in need thereof, the methods comprising administering an effective amount of a compound described herein, or a pharmaceutically acceptable salt thereof, or a pharmaceutical composition described herein.

In some embodiments, provided herein are methods of treating a disease or disorder characterized by overexpression or amplification of cyclin E in a subject in need thereof, the methods comprising administering an effective amount of a compound described herein, or a pharmaceutically acceptable salt thereof, or a pharmaceutical composition described herein. In some embodiments, the cyclin E is CCNE1 or CCNE2.

In some embodiments, provided herein are methods of treating a disease or disorder characterized by overexpression or amplification of cyclin A in a subject in need thereof, the methods comprising administering an effective amount of a compound described herein, or a pharmaceutically acceptable salt thereof, or a pharmaceutical composition described herein.

In some embodiments, provided herein are methods of treating cancer in a subject in need thereof, the methods comprising administering an effective amount of a compound described herein, or a pharmaceutically acceptable salt thereof, or a pharmaceutical composition described herein.

In some embodiments, the cancer is associated with amplification or overexpression of cyclin E or cyclin A. In some embodiments, the cancer is breast cancer, ovarian cancer, uterine cancer, endometrial cancer, cervical cancer, melanoma, lung cancer, pancreatic cancer, stomach cancer, esophageal cancer, bladder cancer, colon cancer, rectal cancer, testicular cancer, prostate cancer, renal cancer, hepatic cancer, head and neck cancer, thyroid cancer, glioblastoma, sarcoma, lymphoma, or leukemia.

In some embodiments, provided herein are methods of treating an autoimmune disorder or disease in a subject in need thereof, the methods comprising administering an effective amount of a compound described herein, or a pharmaceutically acceptable salt thereof, or a pharmaceutical composition described herein. In some embodiments, the autoimmune disorder or disease is associated with amplification or overexpression of cyclin E or cyclin A. In some embodiments, the autoimmune disorder or disease is rheumatoid arthritis, Lupus, Crohn's Disease, Addison disease, celiac disease, dermatomyositis, Graves' disease, thyroiditis, multiple sclerosis, pernicious anemia, reactive arthritis, or pemphigus vulgaris.

In some embodiments, provided herein are methods of treating an inflammatory disease or disorder in a subject in need thereof, the methods comprising administering an effective amount of a compound described herein, or a pharmaceutically acceptable salt thereof, or a pharmaceutical composition described herein. In some embodiments, the inflammatory disease or disorder is associated with amplification or overexpression of cyclin E or cyclin A. In some embodiments, the inflammatory disease or disorder is asthma, chronic peptic ulcers, psoriasis, inflammatory bowel disease, tuberculosis, rheumatoid arthritis, periodontitis, ulcerative colitis, or hepatitis.

In some embodiments, provided herein are methods of treating a neurodegenerative disease or disorder in a subject in need thereof, the methods comprising administering an effective amount of a compound described herein, or a pharmaceutically acceptable salt thereof, or a pharmaceutical composition described herein. In some embodiments, the neurodegenerative disease or disorder is associated with amplification or overexpression of cyclin E or cyclin A. In some embodiments, the neurodegenerative disease or disorder is Alzheimer's disease, AIDS-related dementia, Parkinson's disease, amyotrophic lateral sclerosis, retinitis pigmentosa, spinal muscular atrophy, or cerebellar degeneration.

In some embodiments, provided herein are conjugates, comprising a compound described herein, or a pharmaceutically acceptable salt thereof, bound to CDK2.

Other objects and advantages will become apparent to those skilled in the art from a consideration of the ensuing Detailed Description, Examples, and Claims.

### DETAILED DESCRIPTION

The present disclosure provides, in some embodiments, compounds of Formula (I), (II), (III), (IV), (V), (VI), and (VII), and pharmaceutically acceptable salts thereof, and compositions comprising said compounds or pharmaceutically acceptable salts thereof and a pharmaceutically acceptable excipient. In some embodiments, the compounds or pharmaceutically acceptable salts thereof and compositions disclosed herein are effective at inhibiting or reducing the activity of cyclin-dependent kinase 2 (CDK2). In some embodiments, the compounds or pharmaceutically acceptable salts thereof and compositions disclosed herein are selective at inhibiting CDK2 over other cyclin-dependent kinases (CDKs), such as CDK1. In some embodiments, also provided herein are uses of the compounds or pharmaceutically acceptable salts thereof and compositions disclosed herein in methods of treating, preventing, or reducing the risk or severity of certain diseases or disorders mediated by unwanted activity of CDK2 or over-expression of cyclin E or cyclin A2, such as but not limited to, cancer.

### Definitions

The following are definitions of terms used in the present specification. The initial definition provided for a group or term herein applies to that group or term throughout the present specification individually or as part of another group, unless otherwise indicated. Unless otherwise defined, all technical and scientific terms used herein have the same meaning as commonly understood by one of ordinary skill in the art.

As used herein the specification, “a” or “an” may mean one or more. As used herein, when used in conjunction with the word “comprising”, the words “a” or “an” may mean one or more than one. As used herein “another” may mean at least a second or more. Still further, the terms “having”, “including”, “containing” and “comprising” are interchangeable and one of skill in the art is cognizant that these terms are open ended terms. Some embodiments of the disclosure may consist of or consist essentially of one or more elements, method steps, and/or methods of the disclosure. It is contemplated that any method, compound, or composition described herein can be implemented with respect to any other method, compound, or composition described herein.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The following terms are intended to have the meanings presented therewith below and are useful in understanding the description and intended scope of the present disclosure. When describing the disclosure, which may include compounds and pharmaceutically acceptable salts thereof, pharmaceutical compositions containing such compounds, and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

As used herein, “alkyl” refers to a radical of a straight-chain or branched saturated hydrocarbon group, e.g., having 1 to 20 carbon atoms (“C₁₋₂₀ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). Examples of C₁₋₆ alkyl groups include methyl, ethyl, propyl, isopropyl, butyl, isobutyl, pentyl, hexyl, and the like. In some embodiments, “alkyl” is unsubstituted or substituted with one or more substituents as described herein.

As used herein, “alkenyl” refers to a radical of a straight-chain or branched hydrocarbon group having from 2 to 20 carbon atoms, one or more carbon-carbon double bonds (e.g., 1, 2, 3, or 4 carbon-carbon double bonds), and optionally one or more carbon-carbon triple bonds (e.g., 1, 2, 3, or 4 carbon-carbon triple bonds) (“C₂₋₂₀ alkenyl”). In some embodiments, alkenyl does not contain any triple bonds. In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. In some embodiments, “alkenyl” is unsubstituted or substituted with one or more substituents as described herein.

As used herein, “alkynyl” refers to a radical of a straight-chain or branched hydrocarbon group having from 2 to 20 carbon atoms, one or more carbon-carbon triple bonds (e.g., 1, 2, 3, or 4 carbon-carbon triple bonds), and optionally one or more carbon-carbon double bonds (e.g., 1, 2, 3, or 4 carbon-carbon double bonds) (“C₂₋₂₀ alkynyl”). In some embodiments, alkynyl does not contain any double bonds. In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. In some embodiments, “alkynyl” is unsubstituted or substituted with one or more substituents as described herein.

As used herein, “alkylene,” “alkenylene,” and “alkynylene,” refer to a divalent radical of an alkyl, alkenyl, and alkynyl group respectively. When a range or number of carbons is provided for a particular “alkylene,” “alkenylene,” or “alkynylene,” group, it is understood that the range or number refers to the range or number of carbons in the linear carbon divalent chain. “Alkylene,” “alkenylene,” and “alkynylene,” groups may be unsubstituted or substituted with one or more substituents as described herein.

As used herein, “aryl” refers to a radical of a monocyclic or polycyclic (e.g., bicyclic, tricyclic, or tetracyclic) 4n+2 aromatic ring system (e.g., having 6, 10, or 14π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene. Particularly aryl groups include phenyl, naphthyl, indenyl, and tetrahydronaphthyl. In some embodiments, “aryl” is unsubstituted or substituted with one or more substituents as described herein.

As used herein, “heteroaryl” refers to a radical of an aromatic ring system (e.g., having 6 or 10 electrons shared in a cyclic array) having ring carbon atoms and 1-4 ring heteroatoms provided in the aromatic ring system, wherein each heteroatom is independently selected from nitrogen, oxygen and sulfur. In heteroaryl groups that contain one or more nitrogen atoms, the point of attachment can be a carbon or nitrogen atom, as valency permits. Heteroaryl can be monocyclic or polycyclic (e.g., bicyclic, tricyclic, tetracyclic). Heteroaryl bicyclic ring systems can include one or more heteroatoms in one or both rings. Heteroaryl tricyclic ring systems can include one or more heteroatoms in one or two or all three rings. Heteroaryl tetracyclic ring systems can include one or more heteroatoms in one or two or three or all four rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). In some embodiments, “heteroaryl” is unsubstituted or substituted with one or more substituents as described herein.

In some embodiments, a heteroaryl group is a 5-12 membered aromatic ring system having ring carbon atoms and 1-4 ring heteroatoms provided in the aromatic ring system, wherein each heteroatom is independently selected from nitrogen, oxygen, and sulfur (“5-12 membered heteroaryl”). In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

Examples of representative heteroaryls include the following:

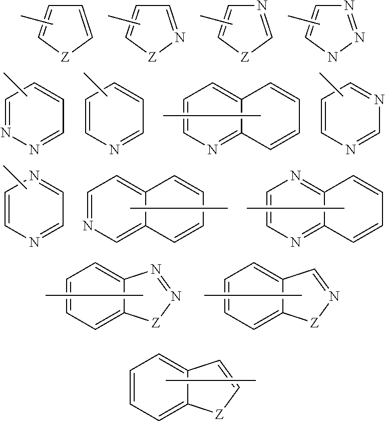

wherein each Z is selected from carbonyl, N, NR^{NN}, O, and S; and R^{NN} is independently hydrogen, C₁₋₈ alkyl, C₃₋₁₀ carbocyclyl, 4-10 membered heterocyclyl, C₆₋₁₀ aryl, and 5-10 membered heteroaryl.

As used herein, “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 12 ring carbon atoms (“C₃₋₁₂ carbocyclyl”). In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in some embodiments, the carbocyclyl group is either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. In some embodiments, “carbocyclyl” is unsubstituted or substituted with one or more substituents as described herein.

The term “cycloalkyl” refers to a monovalent saturated cyclic, bicyclic, or bridged cyclic (e.g., adamantyl) hydrocarbon group of 3-12, 3-10, 3-8, 4-8, or 4-6 carbons, referred to herein, e.g., as “C₃₋₁₂cycloalkyl,” “C₃₋₁₀cycloalkyl,” “C₃₋₈cycloalkyl,” “C₄₋₈cycloalkyl,” or “C₄₋₆cycloalkyl,” respectively, derived from a cycloalkane. Exemplary cycloalkyl groups include, but are not limited to, cyclohexanes, cyclopentanes, cyclobutanes and cyclopropanes. In some embodiments, “cycloalkyl” is unsubstituted or substituted with one or more substituents as described herein.

The term “cycloalkenyl” refers to a monovalent cyclic, bicyclic, or bridged cyclic (e.g., norbornenyl) hydrocarbon group containing at least one C═C of 3-12, 3-10, 3-8, 4-8, or 4-6 carbons, referred to herein, e.g., as “C₃₋₁₂cycloalkenyl,” “C₃₋₁₀cycloalkenyl,” “C₃₋₈cycloalkenyl,” “C₄₋₈cycloalkenyl,” or “C₄₋₆cycloalkenyl,” respectively, derived from a cycloalkene. In some embodiments, “cycloalkenyl” is unsubstituted or substituted with one or more substituents as described herein.

As used herein, “heterocyclyl” or “heterocyclic” refers to a radical of a 3- to 12-membered non-aromatic ring system having ring carbon atoms and 1 to 4 ring heteroatoms, wherein each heteroatom is independently selected from nitrogen, oxygen, sulfur, boron, phosphorus, and silicon (“3-12 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. The terms “heterocycle,” “heterocyclyl,” “heterocyclyl ring,” “heterocyclic group,” “heterocyclic moiety,” “heterocyclic radical,” and “heterocycloalkyl” may be used interchangeably. In some embodiments, “heterocyclyl” is unsubstituted or substituted with one or more substituents as described herein.

In some embodiments, a heterocyclyl group is a 4-12 membered non-aromatic ring system having ring carbon atoms and 1-4 ring heteroatoms, wherein each heteroatom is independently selected from nitrogen, oxygen, and sulfur (“4-12 membered heterocyclyl”). In some embodiments, a heterocyclyl group is a 4-10 membered non-aromatic ring system having ring carbon atoms and 1-4 ring heteroatoms, wherein each heteroatom is independently selected from nitrogen, oxygen, and sulfur (“4-10 membered heterocyclyl”). In some embodiments, a heterocyclyl group is a 4-7 membered non-aromatic ring system having ring carbon atoms and 1-4 ring heteroatoms, wherein each heteroatom is independently selected from nitrogen, oxygen, and sulfur (“4-7 membered heterocyclyl”). In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, and thiiranyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

Examples of saturated or partially unsaturated heterocyclic radicals include, without limitation, tetrahydrofuranyl, tetrahydrothienyl, tetrahydropyranyl, pyrrolidinyl, pyridinonyl, pyrrolidonyl, piperidinyl, oxazolidinyl, piperazinyl, dioxanyl, dioxolanyl, morpholinyl, dihydrofuranyl, dihydropyranyl, dihydropyridinyl, tetrahydropyridinyl, dihydropyrimidinyl, oxetanyl, azetidinyl and tetrahydropyrimidinyl. Where specified as being optionally substituted or substituted, substituents on a heterocyclyl (e.g., in the case of an optionally substituted heterocyclyl) may be present on any substitutable position and, include, e.g., the position at which the heterocyclyl group is attached.

“Hetero” when used to describe a compound or a group present on a compound means that one or more carbon atoms in the compound or group have been replaced by a nitrogen, oxygen, or sulfur heteroatom. Hetero may be applied to any of the hydrocarbyl groups described above such as alkyl, e.g., heteroalkyl; carbocyclyl, e.g., heterocyclyl; aryl, e.g., heteroaryl; and the like having from 1 to 5, and particularly from 1 to 3 heteroatoms.

The terms “halo” and “halogen” as used herein refer to an atom selected from fluorine (fluoro, —F), chlorine (chloro, —Cl), bromine (bromo, —Br), and iodine (iodo, —I). In some embodiments, the halo group is either fluoro or chloro.

The term “haloalkyl” includes mono, poly, and perhaloalkyl groups substituted with one or more halogen atoms where the halogens are independently selected from fluorine, chlorine, bromine, and iodine. For the group C₁₋₄haloalkyl-O—C₁₋₄alkyl, the point of attachment occurs on the alkyl moiety which is halogenated.

The term “alkoxy,” as used herein, refers to an alkyl group which is attached to another moiety via an oxygen atom (—O(alkyl)). Non-limiting examples include e.g., methoxy, ethoxy, propoxy, and butoxy.

The term “alkoxyalkyl” refers to an alkyl group substituted with one or more (e.g., one or two) alkoxy groups. Exemplary alkoxyalkyls include, for example, C₁₋₂-alkylene-O—(C₁₋₄alkyl) and C₁₋₂-alkylene-O—(C₁₋₂alkylene)-O—(C₁₋₄alkyl).

The term “alkenoxy,” as used herein, refers to an alkenyl group which is attached to another moiety via an oxygen atom (—O(alkenyl)).

The term “alkynoxy,” as used herein, refers to an alkynyl group which is attached to another moiety via an oxygen atom (—O(alkynyl)).

“Haloalkoxy” is a haloalkyl group which is attached to another moiety via an oxygen atom such as, e.g., but are not limited to —OCHCF₂ or —OCF₃.

As used herein, “amino” refers to —NH₂.

As used herein, “alkylamino” refers to —NHR^{aa}, wherein R^{aa} is an alkyl group. For example, “C₁₋₆alkylamino” refers to —NHR^{aa}, wherein R^{a} is an unsubstituted or substituted C₁₋₆alkyl group.

As used herein, “dialkylamino” refers to —NR^{aa}R^{bb}, wherein R^{aa} and R^{bb} are each independently an alkyl group. For example, “C₁₋₆dialkylamino” refers to —NR^{aa}Rb^{bb}, wherein R^{aa} and R^{bb} are each independently unsubstituted or substituted C₁₋₆alkyl group.

As used herein, “alkylthio” refers to an alkyl group which is attached to another moiety via a sulfur atom (—S(alkyl)).

As used herein, “alkenylthio” refers to an alkenyl group which is attached to another moiety via a sulfur atom (—S(alkenyl)).

As used herein, “alkylsulfonyl” refers to an alkyl group which is attached to another moiety via a sulfonyl (—SO₂(alkyl)).

As used herein, “alkenylsulfonyl” refers to an alkenyl group which is attached to another moiety via a sulfonyl (—SO₂(alkenyl)).

As used herein, “acyl” refers to —C(═O)R^{cc}, wherein R^{cc} is an alkyl, alkenyl, alkynyl, cycloalkyl, cycloalkenyl, or cycloalkynyl group. For example, “C₁₋₆ acyl” refers to —C(═O)R^{cc} wherein R^{cc} is an unsubstituted or substituted C₁₋₆alkyl, C₂₋₆ alkenyl, or C₂₋₆alkynyl group.

As used herein, “aroyl” refers to —C(═O)R^{dd}, wherein R^{dd} is an aryl group as defined herein. For example, “C₆ aroyl” refers to —C(═O)R^{dd} wherein R^{dd} is an unsubstituted or substituted phenyl ring.

As used herein, “heteroaroyl” refers to —C(═O)R^{ee}, wherein R^{ee} is a heteroaryl group as defined above. For example, “monocyclic heteroaroyl” refers to —C(═O)R^{ee} wherein R^{ee} is an unsubstituted or substituted 5- or 6-membered heteroaryl group.

As used herein, “heterocycloacyl” refers to —C(═O)R^{ff}, wherein R^{ff} is a heterocyclyl group as defined above. For example, “C₃₋₆ monocyclic heterocycloacyl” refers to —C(═O)R^{ff} wherein R^{ff} is an unsubstituted or substituted 3-6 membered heterocyclyl group.

As used herein, “cyano” refers to —CN.

As used herein, “nitro” refers to —NO₂.

As used herein, “azido” refers to —N═N═N.

As used herein, “oxo” refers to refers to an oxygen atom doubly bonded to a carbon atom (═O).

As used herein, “oxa” refers to refers to an oxygen atom singly bonded to two different atoms in a chain or ring.

As used herein, “aza” refers to refers to a nitrogen atom bonded to at least two other atoms in a chain or ring.

As used herein, “thiono” refers to refers to an sulfur atom doubly bonded to a carbon atom (═S).

As used herein, “thia” refers to refers to an sulfur atom of formal oxidation state zero singly bonded to two different atoms in a chain or ring.

As used herein, “sulfinyl” refers to refers to an S═O group singly bonded to two different atoms in a chain or ring.

As used herein, “sulfonyl” refers to refers to an S(═O)₂ group singly bonded to two different atoms in a chain or ring.

As used herein, “sulfonamido” and “aminosulfonyl” refer to refers to an —S(═O)₂—NR^{gg}— group singly bonded to two different atoms in a chain or ring wherein R^{gg} is a hydrocarbyl group such as alkyl, alkenyl, alkynyl, heteroalkyl, carbocyclyl, heterocyclyl, aryl, or heteroaryl.

As used herein, the term “hydroxy” or “hydroxyl” refers to —OH group.

As used herein, the term “hydroxyalkyl” refers to an alkyl group substituted with a hydroxy group.

It will be appreciated that the compounds, as described herein, may be substituted with any number of substituents or functional moieties. In general, the term “substituted” whether preceded by the term “optionally” or not, and substituents contained in formulas of this disclosure, refer to the replacement of hydrogen radicals in a given structure with the radical of a specified substituent. When more than one position in any given structure may be substituted with more than one substituent selected from a specified group, the substituent may be either the same or different at every position.

Unless otherwise indicated, any heteroatom with unsatisfied valences is assumed to have hydrogen atoms sufficient to satisfy the valences.

Certain compounds of the present disclosure may exist in particular geometric or stereoisomeric forms. The present disclosure contemplates all such compounds, including cis- and trans-isomers, R- and S-enantiomers, diastereomers, (d)-isomers, (l)-isomers, the racemic mixtures thereof, and other mixtures thereof, as falling within the scope of the disclosure. Additional asymmetric carbon atoms may be present in a substituent such as an alkyl group. All such isomers, as well as mixtures thereof, are intended to be included in this disclosure.

All stereoisomers of the present compounds (for example, those which may exist due to asymmetric carbons on various substituents), including enantiomeric forms and diastereomeric forms, are contemplated within the scope of this disclosure. Individual stereoisomers of the compounds of the disclosure may, for example, be substantially free of other isomers (e.g., as a pure or substantially pure optical isomer having a specified activity), or may be admixed, for example, as racemates or with all other, or other selected, stereoisomers. The chiral centers of the present disclosure may have the S or R configuration as defined by the International Union of Pure and Applied Chemistry (IUPAC) 1974 Recommendations. The racemic forms can be resolved by physical methods, such as, for example, fractional crystallization, separation or crystallization of diastereomeric derivatives, or separation by chiral column chromatography. The individual optical isomers can be obtained from the racemates by any suitable method, including without limitation, conventional methods, such as, for example, salt formation with an optically active acid followed by crystallization.

All configurational isomers of the compounds of the present disclosure are contemplated, either in admixture or in pure or substantially pure form. The definition of compounds of the present disclosure embraces both cis (Z) and trans (E) alkene isomers, as well as cis and trans isomers of cyclic hydrocarbon or heterocyclic rings.

Isomeric mixtures containing any of a variety of isomer ratios may be utilized in accordance with the present disclosure. For example, where only two isomers are combined, mixtures containing 50:50, 60:40, 70:30, 80:20, 90:10, 95:5, 96:4, 97:3, 98:2, 99:1, or 100:0 isomer ratios are all contemplated by the present disclosure.

The present disclosure also includes isotopically-labeled compounds, which are identical to the compounds disclosed herein, but for the fact that one or more atoms are replaced by an atom having an atomic mass or mass number different from the atomic mass or mass number usually found in nature. Examples of isotopes that can be incorporated into compounds of the present disclosure include isotopes of hydrogen, carbon, nitrogen, oxygen, phosphorous, sulfur, fluorine, and chlorine, such as ²H, ³H, ¹³C, ¹¹C, ¹⁴C, ¹⁵N, ¹⁸O, ¹⁷O, ³¹P, ³²P, ³⁵S, ¹⁸F, and ³⁶Cl, respectively. Compounds of the present disclosure, or a stereoisomer, tautomer, or pharmaceutically acceptable salt or solvate thereof, which contain the aforementioned isotopes and/or other isotopes of other atoms are within the scope of this disclosure.

As used herein, “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As used herein, “pharmaceutically acceptable excipient” refers to any substance in a pharmaceutical formulation other than the active pharmaceutical ingredient(s). Exemplary pharmaceutical excipients include those that aid the manufacturing process; protect, support or enhance stability; increase bioavailability; or increase patient acceptability. They may also assist in product identification or enhance the overall safety or function of the product during storage or use.

As used herein, a “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In some embodiments, the subject is a human. In some embodiments, the subject is a non-human animal. The terms “human,” “patient,” “subject,” and “individual” are used interchangeably herein. None of these terms require the active supervision of medical personnel.

Disease, disorder, and condition are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or reverses or slows the progression of the disease, disorder or condition (also “therapeutic treatment”).

In general, the “effective amount” of a compound refers to an amount sufficient to elicit the desired biological response. As will be appreciated by those of ordinary skill in this art, the effective amount of a compound of the disclosure may vary depending on such factors as the desired biological endpoint, the pharmacokinetics of the compound, the disease being treated, the mode of administration, and the age, weight, health, and condition of the subject. A “therapeutically effective amount” of a compound is an amount sufficient to provide a therapeutic benefit (e.g., treating, preventing, and/or ameliorating cancer in a subject, or inhibiting protein-protein interactions mediated by CDK2 in a subject, at a reasonable benefit/risk ratio applicable to any medical treatment) in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent. A “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent. A “prophylactic treatment” contemplates an action that occurs before a subject begins to suffer from the specified disease, disorder or condition.

An “anti-cancer agent” is a chemical compound useful in the treatment of cancer, regardless of mechanism of action. Classes of anti-cancer agents include, but are not limited to: alkylating agents, antimetabolites, anti-hormone therapies, endocrine therapies, immunomodulatory agents, spindle poison plant alkaloids, cytotoxic/antitumor antibiotics, topoisomerase inhibitors, antibodies, photosensitizers, and kinase inhibitors. Anti-cancer agents include compounds used in targeted therapy and conventional chemotherapy. Exemplary anti-cancer agents include proteasome inhibitors such as bortezomib (e.g., VELCADE®), carfilzomib (e.g., KYPROLIS®) and ixazomib (e.g., NINLARO®). Other examples include immunomodulatory agents such as lenalidomide (e.g., REVLIMID®) and pomalidomide (e.g., POMALYST®). Other exemplary anti-cancer agents include inhibitors of B-cell receptor targets such as BTK, Bcl-2 and JAK inhibitors and include, for example, venetoclax (e.g., VENCLEXTA®) and ibrutinib (e.g., IMBRUVICA®). Additional anti-cancer agents include, for example, abemaciclib (e.g., VERZENIO®); abiraterone (e.g., ZYTIGA®, YONSA®); aclarubicin; acivicin; acodazole; acronine; actinomycin; acylfulvene; adecypenol; adozelesin; adriamycin; aldesleukin; altretamine; ambamustine; ambomycin; ametantrone; amidox; amifostine; aminoglutethimide; aminolevulinic acid; amrubicin; amsacrine; anagrelide; anastrozole; andrographolide; antarelix; anthramycin; aphidicolin glycinate; apurinic acid; ARRY-300; arabinoside; asperlin; asulacrine; atamestane; atrimustine; azasetron; azatoxin; azatyrosine; azacitidine; selumetinib (AZD6244); AZD8330 (2-[(2-fluoro-4-iodophenyl)amino]-N-(2-hydroxyethoxy)-1,5-dimethyl-6-oxo-1,6-dihydropyridine-3-carboxamide); azetepa; azotomycin; balanol; batimastat; bendamustine; benzochlorins; benzodopa; benzoylstaurosporine; beta-alethine; betaclamycin B; betulinic acid; bicalutamide; binimetinib; bisantrene; bisaziridinylspermine; bisnafide; bistratene; bleomycin; busulfan; bizelesin; breflate; bortezomib; brequinar; bropirimine; budotitane; buthionine; bryostatin; cactinomycin; calusterone; calcipotriol; calphostin C; camptothecin; capecitabine (e.g., XELODA®); caracemide; carbetimer; carboplatin; carboquone; carmustine; carubicin; carzelesin; castanospermine; celecoxib; cetrorelix; cetuximab (e.g., ERBITUX®); chloroquinoxaline; cicaprost; chlorambucil; chlorofusin; cisplatin; cladribine; clomifene; clotrimazole; crisnatol; crisnatol; cypemycin; cyclophosphamide; cytarabine; cytostatin; dacarbazine; dactinomycin; daratumamab; daunorubicin; decarbazine; dacliximab; dasatinib; decitabine; deslorelin; dexamethasone; dexifosfamide; dexrazoxane; dexverapamil; dexormaplatin; dezaguanine; diaziquone; dihydrotaxol; docosanol; dolasetron; docetaxel; doxorubicin; doxifluridine; droloxifene; dromostanolone; dronabinol; duazomycin; ebselen; ecomustine; edelfosine; edrecolomab; edatrexate; elfomithine; elemene; emitefur; elsamitrucin; enloplatin; enpromate; epipropidine; epirubicin; epristeride; erbulozole; erlotinib (e.g., TARCEVA®); esorubicin; estramustine; etanidazole; etoposide; etoprine; exemestane; fadrozole; fazarabine; fenretinide; filgrastim; finasteride; flavopiridol; flezelastine; fluasterone; floxuridine; fludarabine; fludarabine; fluorodaunorunicin; forfenimex; formestane; fluorouracil; floxouridine; flurocitabine; fosquidone; fostriecin; fotemustine; fulvestrant (e.g., FASLODEX®); gadolinium; gallium; galocitabine; ganirelix; gemcitabine; geldanamycin; gefitinib; gossyphol; hydroxyurea; hepsulfam; heregulin; ibandronate; ibrutinib; idarubicin; idelalisib (e.g., ZYDELIG®), ifosfamide; canfosfamide; ilmofosine; iproplatin; idoxifene; idramantone; ilmofosine; ilomastat; imatinib mesylate (e.g., GLEEVEC®); imiquimod; iobenguane; iododoxorubicin; ipomeanol; irinotecan; itasetron; ilmofosine; lanreotide; lapatinib (e.g., TYKERB®); leinamycin; lenograstim; lentinan; leptolstatin; letrozole; leuprorelin; levamisole; liarozole; lobaplatin; lombricine; lometrexol; lonidamine; lonafamib (e.g., SARASAR®); losoxantrone; lovastatin; loxoribine; lurtotecan; lapatinib; leucovorin; lometrexol; lomustine; maitansine; marimastat; masoprocol; maspin; menogaril; merbarone; meterelin; methioninase; metoclopramide; mifepristone; miltefosine; mirimostim; mitoguazone; mitolactol; mitonafide; mitoxantrone; mofarotene; molgramostim; mopidamol; maytansine; megestrol acetate; melengestrol acetate; melphalan; mercaptopurine; methotrexate; methotrexate sodium; metoprine; meturedepa; mitinmitomycin; mitosper; mitotane; mitoxantrone; mycophenolic acid; nafarelin; nagrestip; napavin; nedaplatin; nemorubicin; neridronic acid; nilutamide; nisamycin; oblimersen (e.g., GENASENSE®); octreotide; okicenone; onapristone; ondansetron; ormaplatin; oxisuran; oxaloplatin; osaterone; oxaliplatin; oxaunomycin; palauamine; palbociclib (e.g., IBRANCE®); panitumumab (e.g., VECTIBIX®); panomifene; pegaspargase; picibanil; pirarubicin; piritrexim; prednisone; prednisolone, paclitaxel; nab-paclitaxel (e.g., ABRAXANE®); prednimustine; procarbazine; puromycin; raltitrexed; ramosetron; rapamycin (e.g., RAPAMUNE®); rhizoxin; ribociclib (e.g., KISQALI®), rituximab; rogletimide; rohitukine; romurtide; roquinimex; romidepsin; safingol; saintopin; sargramostim; semustine; sizofiran; sobuzoxane; sorafenib (e.g., NEXAVAR®); sunitinib; spiromustine; squalamine; suradista; suramin; swainsonine; spiroplatin; streptonigrin; streptozocin; sulofenur; tallimustine; tamoxifen; tauromustine; tazarotene; tellurapyrylium; temoporfin; temozolomide; teniposide; tetrachlorodecaoxide; tetrazomine; thrombopoietin; thymalfasin; thymotrinan; tirapazamine; toremifene; tretinoin; trimetrexate; triptorelin; tropisetron; talisomycin; taxotere; teroxirone; testolactone; thiamiprine; thiotepa; tirapazamine; toremifene; trastuzumab; trastuzumab emtansine; trestolone acetate; triciribine phosphate; trimetrexate; uracil mustard; vandetanib (e.g., CAPRELSA®); variolin B; velaresol; veramine; verteporfin; vemurafenib; vinorelbine; vinxaltine; vitaxin; vinblastine; vincristine; vindesine; vinepidine; vinglycinate; vinleurosine; vinorelbine; vinrosidine; vinzolidine; vorozole; wortmannin; zanoterone; zeniplatin; zilascorb; zinostatin stimalamer; zinostatin; and zorubicin. In some embodiments, the anti-cancer agent includes, for example, idelalisib (e.g., ZYDELIG®), docetaxel, fluorouracil, gemcitabine (e.g., GEMZAR®), cisplatin, cis-diamine, carboplatin, paclitaxel, nab-paclitaxel, trastuzumab (e.g., HERCEPTIN®), temozolomide, tamoxifen, 4-hydroxytamoxifen, and doxorubicin.

Also included in the definition of anti-cancer agent are: (i) anti-estrogens and selective estrogen receptor modulators (SERMs), including, for example, tamoxifen, raloxifene, droloxifene, 4-hydroxytamoxifen, trioxifene, keoxifene, LY117018 ([6-hydroxy-2-(4-hydroxyphenyl)-1-benzothiophen-3-yl]-[4-(2-pyrrolidin-1-ylethoxy)phenyl]methanone), onapristone, and toremifine citrate; (ii) selective estrogen receptor modulators (SERDs) such as brilanestrant, GDC-0927 ((2S)-2-[4-[2-[3-(fluoromethyl)azetidinedin-1-yl]ethoxy]phenyl]-3-(3-hydroxyphenyl)-4-methyl-2H-chromen-6-ol), giredestrant (GDC-9545), AZ9496 ((E)-3-[3,5-difluoro-4-[(1S,3R)-2-(2-fluoro-2-methylpropyl)-3-methyl-1,3,4,9-tetrahydropyrido[3,4-b]indol-1-yl]phenyl]prop-2-enoic acid), camizestrant (AZ9833), GNE-274 ((S)-3-(3-hydroxyphenyl)-4-methyl-2-(4-((1-propylazetidin-3-yl)methoxy)phenyl)-2H-chromen-6-ol), and fulvestrant (e.g., FASLODEX®); (iii) aromatase inhibitors such as, for example, 4(5)-imidazoles, aminoglutethimide, megestrol acetate, exemestane, formestanie, fadrozole, vorozole, letrozole, and anastrozole; (iv) anti-androgens such as apalutamide, abiraterone, enzalutamide, flutamide, nilutamide, bicalutamide, leuprolide, and goserelin. Further included in the definition of anti-cancer agents are: (v) MEK inhibitors such as cobimetinib; (vi) lipid kinase inhibitors, such as taselisib; (vii) antisense oligonucleotides such as oblimersen; (viii) ribozymes such as VEGF expression inhibitors such as angiozyme; (ix) vaccines such as gene therapy vaccines, for example, velimogene aliplasmid (allovectin), plasmid DNA/lipid complex comprised of a plasmid DNA expression vector encoding human interleukin (IL)-2 complexed in a 5:1 mass ratio with DMRIE/DOPE lipid (leuvectin), and anti-idiotype naked DNA vaccine (vaxid); (x) topoisomerase 1 inhibitors such as lurtotecan; abarelix; and (xi) anti-angiogenic agents such as bevacizumab. In some embodiments herein, the anti-cancer agents is a therapeutic antibody such as atezolizumab, nivolumab, daratumumab, pembrolizumab, alemtuzumab, bevacizumab; cetuximab; panitumumab, rituximab, pertuzumab, trastuzumab, trastuzumab emtansine, or tositumomab.

The term “package insert” is used to refer to instructions customarily included in commercial packages of therapeutic products, that contain information about the indications, usage, dosage, administration, contraindications and/or warnings concerning the use of such therapeutic products.

### CDK2 Inhibition

Cyclin-dependent kinases (CDKs) are important cellular enzymes that perform essential functions in regulating cell division and proliferation. The CDK catalytic units are activated by binding to regulatory subunits, known as cyclins, followed by phosphorylation. The cyclins are divided into four general classes, G₁, G₂/S, S and M cyclins, whose expression levels vary at different points in the cell cycle.

In particular, CDK2 becomes active when cyclin E or cyclin A binds at the active site of the kinase. The cyclin E/CDK2 complex plays an important role in regulation of the G₁/S transition, histone biosynthesis, and centrosome duplication. Progressive phosphorylation of retinoblastoma (Rb) by cyclin D/Cdk4/6 and cyclin E/CDK2 releases the G₁ transcription factor, E2F, and promotes S-phase entry. Activation of cyclin A/CDK2 during early S-phase promotes phosphorylation of endogenous substrates that permit DNA replication and inactivation of E2F, for S-phase completion.

CDK2 is critical to the abnormal growth processes of certain disease or disorders such as cancer. The CCNE1 gene which produces cyclin E is frequently overexpressed in various cancers, causing the cells to become dependent on Cdk2 and cyclin E. In addition, abnormal expression of cyclin A2 is associated with chromosomal instability and tumor proliferation. However, Cyclin A also binds CDK1 during the late S/G2 phase. CDK1 is an essential cyclin dependent kinase in the cell cycle, and its inhibition could lead to undesired toxicity. Thus, there is a need for CDK2 inhibitors that are selective for CDK2 over CDK1.

In addition, CDK2 has been implicated in regulatory events in autoimmune diseases, such as pemphigus vulgaris, inflammation, and neurodegenerative diseases or disorders. Thus, inhibitors of CDK2 are viable candidates for the methods of treating, preventing, or reducing the risk or severity of certain autoimmune diseases or disorders, inflammatory diseases or disorders, and neurodegenerative diseases or disorders.

The present disclosure provides, in some embodiments, compounds of Formula (I), (II), (III), (IV), (V), (VI), and (VII), and pharmaceutically acceptable salts thereof, compositions comprising a compound of Formula (I), (II), (III), (IV), (V), (VI), and (VII), or a pharmaceutically acceptable salt thereof, and a pharmaceutically acceptable excipient, which inhibit the activity of CDK2, thereby effecting biological processes mediated by unwanted activity of CDK2. In some embodiments, the compounds and compositions disclosed herein are selective against CDK2 (e.g., have a lower inhibitory constant, K_{I} or IC₅₀ for CDK2) relative to other enzymatic targets of CDK such as CDK1. Compounds that are selective against CDK2 may provide improved safety profile, improved dosing schedule, and/or enhanced overall efficacy. Similarly selective inhibitors of CDK2 may have reduced risk of certain toxicities linked to inhibition of other CDKs. In some embodiments, the compounds and compositions disclosed herein are selective against CDK2 relative to CDK1.

### Compounds

In some embodiments, provided here are compounds of Formula I:

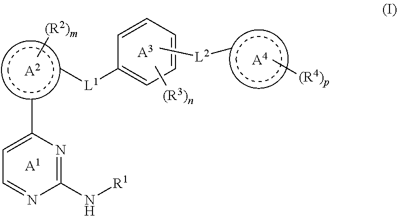

or pharmaceutically acceptable salts thereof, wherein:
A² is phenyl, 2,3-naphthyl, or 5 or 6-membered heteroaryl;
A⁴ is aryl, heteroaryl, C₃₋₁₂ carbocyclyl, or 4-12 membered heterocyclyl;
L¹ is —O—, —S—, —NH—, S(═O)₁₋₂—, —S(═O)(═NR⁵)—, —C(R⁶)₂—, or —C(═O)—;
L² is —NR⁷SO₂—, —C(R⁸)₂SO₂—, —NR⁷SO₂—C(R⁸)₂—, —C(R⁸)₂C(═O)—, —C(═O)C(R⁸)₂—, —C(═O)O—, —OC(═O)—, —C(R⁸)₂O—, —OC(R⁸)₂—, —C(═O)NR⁷—, —NR⁷C(═O)—, —C(R⁸)₂NR⁷—, —NR⁷C(R⁸)₂—, —C(R⁸)₂S(═O)₀₋₂—, —S(═O)₀₋₂C(R⁸)₂—, —C(R⁸)₂S(═O)NR⁷—, S(═O)NR⁷C(R⁸)₂—, or —C(R⁸)₂C(R⁸)₂—;
R¹ is C₃₋₁₂ carbocyclyl, 4-12 membered heterocyclyl, phenyl, 5-12 membered heteroaryl, —(C₁₋₆ alkylene)-(C₃₋₁₀ carbocyclyl), —(C₁₋₆ alkylene)-(4-12 membered heterocyclyl), —(C₁₋₆alkylene)-(phenyl), —(C₁₋₆alkylene)-(5-12 membered heteroaryl), —(C₁₋₆alkylene)-OR⁹, or —(C₁₋₆alkylene)-NR⁷R¹⁰, wherein R¹ is optionally substituted by 0-5 R¹¹;
R² is each independently H, halogen, cyano, nitro, hydroxy, —NR⁷R¹⁰, —NHCOR⁹, C₁₋₆ alkyl, C₂₋₆ alkenyl, C₂₋₆ alkynyl, C₁₋₆ alkoxy, C₂₋₆ alkenoxy, C₂₋₆ alkynoxy, COR⁹, CON(R⁹)₂, CF₃, CF₂H, CF₃CF₂, CF₃CH₂, C₁₋₆haloalkyl, CF₃O, CF₂HO, CF₃CF₂O, CF₃CH₂O, C₁₋₆ haloalkoxy, C₃₋₈ cycloalkyl, C₃₋₈ cycloalkenyl, C₁₋₆ alkylthio, C₂₋₆ alkenylthio, C₁₋₆ alkylsulfonyl, aryl, or heteroaryl, wherein R² is each independently optionally substituted by 0-4 R¹¹;
R³ is each independently H, halogen, cyano, nitro, hydroxy, —NR⁷R¹⁰, —NHCOR⁹, C₁₋₆ alkyl, C₂₋₆ alkenyl, C₂₋₆ alkynyl, C₁₋₆ alkoxy, C₂₋₆ alkenoxy, C₂₋₆ alkynoxy, COR⁹, CON(R⁹)₂, CF₃, CF₂H, CF₃CF₂, CF₃CH₂, C₁₋₆haloalkyl, CF₃O, CF₂HO, CF₃CF₂O, CF₃CH₂O, C₁₋₆ haloalkoxy, C₃₋₈ cycloalkyl, C₃₋₈ cycloalkenyl, C₁₋₆ alkylthio, C₂₋₆ alkenylthio, or C₁₋₆ alkylsulfonyl, wherein R³ is each independently optionally substituted by 0-4 R¹¹;
R⁴ is each independently H, halogen, cyano, nitro, hydroxy, NR⁷R¹⁰, NHCOR⁹, NR⁷CONR⁷, C₁₋₆ alkyl, C₂₋₆ alkenyl, C₂₋₆ alkynyl, COR⁹, CO₂R⁹, CONR⁷R¹⁰, C₁₋₆ alkoxy, C₂₋₆ alkenoxy, C₂₋₆ alkynoxy, CF₃, CF₂H, CF₃CF₂, CF₃CH₂, C₁₋₆ haloalkyl, CF₃O, CF₂HO, CF₃CF₂O, CF₃CH₂O, C₁₋₆ haloalkoxy, C₃₋₈ cycloalkyl, C₃₋₈ cycloalkenyl, C₁₋₆ alkylthio, C₂₋₆ alkenylthio, C₁₋₆ alkylsulfonyl, SO₂N(R⁷)₂, or phenyl, or any two occurrences of R⁴ on contiguous atoms, taken together with the contiguous atoms to which the two occurrences of R⁴ are attached, form a saturated or unsaturated 5-7 membered ring comprising 0-2 heteroatoms each independently selected from O, S(═O)₀₋₂, P(═O)R¹², N, and NR¹⁰, wherein R⁴ is each independently optionally substituted by 0-4 R¹¹;
R⁵ is each independently H or C₁₋₄ alkyl;
R⁶ is each independently H, F, OH, cyano, or C₁₋₆ alkyl;
R⁷ is each independently H, C₁₋₆ alkyl, C₁₋₆ haloalkyl, C₂₋₆ alkenyl, C₂₋₆ alkynyl, C₃₋₆ cycloalkyl, C₄₋₆ heterocycloalkyl, aryl, 5-10 membered heteroaryl, C₁₋₃ alkylene-C₃₋₆ cycloalkyl, or C₁₋₃ alkylene-C₄₋₆ heterocycloalkyl, or two R⁷s along with the N atom to which they are attached form a 4-7 membered ring comprising an additional 0-1 heteroatoms selected from NR⁵, O and S(═O)₀₋₂;
R⁸ is each independently H, halogen, cyano, C₁₋₆ alkyl, C₁₋₆ haloalkyl, C₂₋₆ alkenyl, C₂₋₆ alkynyl, C₂₋₆ cycloalkyl, C₄₋₆heterocycloalkyl, C₁₋₃ alkylene-C₃_cycloalkyl, C₁₋₃ alkylene-C₄₋₆ heterocycloalkyl, or two R⁸s taken together with the C atom to which they are attached form a 4-7 membered ring comprising 0-1 heteroatom selected from O, S(═O)₀₋₂, P(═O)R¹², and NR⁵, wherein R⁸ is optionally substituted by 1-3 R¹¹;
each R⁹ is H, C₁₋₆ alkyl, C₁₋₆ haloalkyl, C₂₋₆ alkenyl, C₂₋₆ alkynyl, C₃₋₁₀ cycloalkyl, C₄₋₁₀ heterocycloalkyl, C₁₋₃ alkylene-C₃₋₆ cycloalkyl, C₁₋₃ alkylene-C₄₋₆ heterocycloalkyl, C₁₋₆ acyl, phenyl, or 5-12 membered heteroaryl, wherein R⁹ is optionally substituted by 1-3 R¹¹;
   each R¹⁰ is independently H, C₁₋₆ alkyl, C₁₋₆ haloalkyl, C₂₋₆ alkenyl, C₂₋₆ alkynyl, C₃₋₁₀ cycloalkyl, C₄₋₁₀ heterocycloalkyl, C₁₋₃ alkylene-C₃₋₆ cycloalkyl, C₁₋₃ alkylene-C₄₋₆ heterocycloalkyl, C₁₋₆ acyl, C₁₋₆ alkylsulfonyl, phenyl, or 5-12 membered heteroaryl, wherein R¹⁰ is optionally substituted by 1-3 R¹¹,
or R⁷ and R¹⁰ on the same N atom may be taken together with the N atom to which they are attached form a 4-7 membered ring comprising an additional 0-1 heteroatoms selected from NR⁵, O, S(═O)₀₋₂, P(═O)R¹², and NR¹³;
R¹¹ is each independently R⁶, oxo, nitro, C₂₋₆ alkenyl, C₂₋₆ alkynyl, C₁₋₆ alkoxy, C₁₋₆ alkoxyalkyl, C₁₋₆hydroxyalkyl, C₁₋₆ haloalkyl, C₁₋₆ haloalkoxy, C₃₋₆ cycloalkyl, 4-6 membered heterocycloalkyl, C₁₋₃ alkylene-C₃₋₆ cycloalkyl, C₁₋₃ alkylene-C₄₋₆ heterocycloalkyl, C₆₋₁₀ aryl, C₅₋₁₀ heteroaryl, SO₂R⁷, S(═O)NR⁵, SO₂N(R⁷)₂, CON(R⁷)₂, or N(R⁷)₂;
R¹² is each independently H, OH, C₁₋₆ alkyl, C₁₋₆ alkoxy, or —N(R⁷)₂;
R¹³ is each independently H, C₁₋₆ alkyl, C₁₋₆ haloalkyl, C₂₋₆ alkenyl, C₂₋₆ alkynyl, C₂₋₆ cycloalkyl, C₄₋₆ heterocycloalkyl, C₁₋₃ alkylene-C₃₋₆ cycloalkyl, or C₁₋₃ alkylene-C₄₋₆ heterocycloalkyl, C₁₋₆ acyl, aroyl, or heteroaroyl;
m is an integer 0, 1 or 2;
n is an integer 0, 1, 2, or 3; and
p is an integer 0, 1, 2, or 3,
provided that
the A¹ ring and the L¹ linker are on contiguous C atoms of the A² ring, whereas L¹ and L² are not situated on contiguous atoms of the A³ ring;
and when A² is

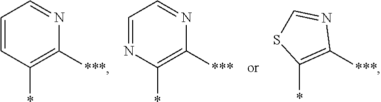

L¹ is not —O—, wherein * is the site covalently linked to the A¹ ring, and *** is the site covalently linked to L¹.

In some embodiments, A⁴ is phenyl, naphthyl, 5-12 membered heteroaryl, C₃₋₁₀ carbocyclyl, or 4-12 membered heterocyclyl. In some embodiments, A⁴ is phenyl, naphthyl, 5-10 membered heteroaryl, C₃₋₁₀ carbocyclyl, or 5-10 membered heterocyclyl. In some embodiments, A⁴ is phenyl, naphthyl, 5-10 membered heteroaryl, C₃₋₆ carbocyclyl, or 5-10 membered heterocyclyl. In some embodiments, A⁴ is phenyl, naphthyl, or 5-12 membered heteroaryl. In some embodiments, A⁴ is phenyl, naphthyl, or 5-10 membered heteroaryl. In some embodiments, A⁴ is phenyl, naphthyl, 5-6 membered monocyclic heteroaryl, or 8-12 membered bicyclic heteroaryl. In some embodiments, A⁴ is phenyl, pyridyl, pyridazinyl, pyrimidyl, pyrazinyl, triazinyl, thienyl, thiazolyl, thiadiazolyl, oxazolyl, oxadiazolyl, pyrazolyl, imidazolyl, triazolyl, or tetrazolyl. In some embodiments, A⁴ is phenyl, naphthyl, thiophenyl, cyclopropyl, cyclobutyl, cyclopentyl, cyclohexyl,

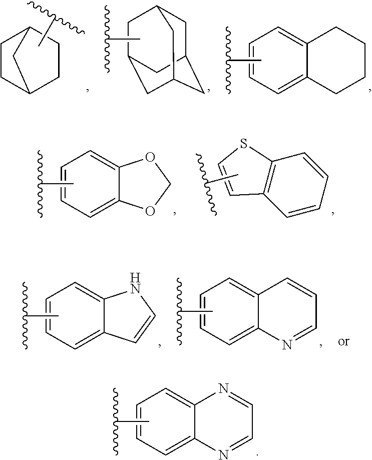

In some embodiments, A⁴ is phenyl.

In some embodiments, A² is phenyl, 2,3-naphthyl, pyridinyl, or pyrimidinyl. In some embodiments, A² is phenyl. In some embodiments, A² is 2,3-naphthyl. In some embodiments, A² is 5-6 membered heteroaryl. In some embodiments, A² is 1,2-linked phenylene, 2,3-linked-naphthylene, 2-L¹-3-biaryl linked pyridylene, 4-L¹-3-biaryl linked pyridylene or 4-L¹-5-biaryl linked pyrimidylene. In some embodiments, A² is 1,2-linked phenylene, 2-L¹-3-biaryl linked pyridylene or 4-L¹-3-biaryl linked pyridylene. In some embodiments, A² is phenyl, 2,3-naphthyl,

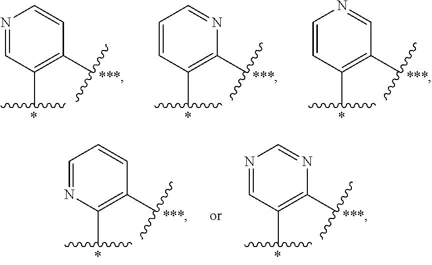 [SMILES: *c1ccncc1* ; *c1cccnc1* ; *c1ccncc1* ; *c1cccnc1* ; *c1cncnc1*]

wherein * is the site covalently linked to the A¹ ring, and *** is the site covalently linked to L¹.

In some embodiments, R¹ is C₃₋₁₀carbocyclyl, 4-12 membered heterocyclyl, phenyl, 5-12 membered heteroaryl, —(C₁₋₆alkylene)-(C₃₋₁₀carbocyclyl), —(C₁₋₆alkylene)-(4-12 membered heterocyclyl), —(C₁₋₆alkylene)-(phenyl), —(C₁₋₆alkylene)-(5-12 membered heteroaryl), —(C₁₋₆alkylene)-OR⁹, or —(C₁₋₆alkylene)-NR⁷R¹⁰, wherein R¹ is optionally substituted by 1-2 R¹¹. In some embodiments, R¹ is C₆₋₁₀carbocyclyl, 4-8 membered heterocyclyl, phenyl, 5-6 membered heteroaryl, —(C₁₋₆alkylene)-(C₃₋₁₀carbocyclyl), —(C₁₋₆alkylene)-(4-8 membered heterocyclyl), —(C₁₋₆alkylene)-(5-6 membered heteroaryl), —(C₁₋₆alkylene)-OR⁹, or —(C₁₋₆alkylene)-NR⁷R¹⁰, wherein R¹ is optionally substituted by 1-2 R¹¹. In some embodiments, R¹ is C₆₋₁₀carbocyclyl, 4-8 membered heterocyclyl, phenyl, 5-6 membered heteroaryl, —(C₁₋₆alkylene)-(C₃₋₁₀carbocyclyl), —(C₁₋₆alkylene)-(4-8 membered heterocyclyl), —(C₁₋₆alkylene)-(5-6 membered heteroaryl), —(C₁₋₆alkylene)-OR⁹, or —(C₁₋₆alkylene)-NR⁷R¹⁰, wherein R¹ is optionally substituted by 1 R¹¹. In some embodiments, R¹ is C₆₋₁₀carbocyclyl, 4-8 membered heterocyclyl containing 1-2 heteroatoms selected from the group consisting of O, S, and N, —(C₁₋₆alkylene)-(C₃₋₁₀carbocyclyl), —(C₁₋₆alkylene)-(4-8 membered heterocyclyl containing 1-2 heteroatoms selected from the group consisting of O, S, and N), —(C₁₋₆alkylene)-(5-6 membered heteroaryl containing 1-2 heteroatoms selected from the group consisting of O, S, and N), —(C₁₋₆alkylene)-OR⁹, or —(C₁₋₆alkylene)-NR⁷R¹⁰, wherein R¹ is optionally substituted by 1 R¹¹. In some embodiments, R¹ is C₆₋₁₀carbocyclyl, 4-8 membered heterocyclyl containing 1-2 heteroatoms selected from the group consisting of O, S, and N, —(C₁₋₃alkylene)-(4-6 membered heterocyclyl containing 1-2 heteroatoms selected from the group consisting of O, S, and N), —(C₁₋₃alkylene)-(5-6 membered heteroaryl containing 1-2 heteroatoms selected from the group consisting of O, S, and N), —(C₁₋₆alkylene)-OR⁹, or —(C₁₋₆alkylene)-NR⁷R¹⁰, wherein R¹ is optionally substituted by 1 R¹¹.

In some embodiments, R¹ is cyclohexyl, piperidinyl, oxetanyl, azetidinyl, pyrrolidinyl, or azaspiroheptanyl, wherein R¹ is optionally substituted with 1 R¹¹.

In some embodiments, R¹ is cyclohexyl optionally substituted with 1 R¹¹.

In some embodiments, R¹ is piperidinyl, oxetanyl, azetidinyl, pyrrolidinyl, or azaspiroheptanyl, wherein R¹ is optionally substituted with 1 R¹¹. In some embodiments, R¹ is piperidinyl optionally substituted with 1 R¹¹.

In some embodiments, R¹ is —(C₁₋₃alkylene)-OR⁹ wherein R⁹ is C₁₋₆alkyl substituted by 1-2 R¹¹. In some embodiments, R¹ is —(C₁₋₃alkylene)-OR⁹ wherein R⁹ is C₁₋₆alkyl substituted by 1 R¹¹. In some embodiments, R¹¹ is hydroxy or C₁₋₄alkoxy. In some embodiments, R¹¹ is hydroxy or C₁₋₂alkoxy.

In some embodiments, R¹ is —(C₁₋₃alkylene)-NR⁷R¹⁰ wherein R⁹ is H or C₁₋₆alkyl and R¹⁰ is H or C₁₋₆alkyl, wherein the R¹⁰ is optionally substituted by 1-2 R¹¹. In some embodiments, R¹ is —(C₁₋₃alkylene)-NR⁷R¹⁰ wherein R⁹ is H or C₁₋₆alkyl and R¹⁰ is H or C₁₋₆alkyl, wherein the R¹⁰ is optionally substituted by 1 R¹¹. In some embodiments, R¹¹ is hydroxy or C₁₋₄alkoxy. In some embodiments, R¹¹ is hydroxy or C₁₋₂alkoxy.

In some embodiments, R¹ is —(C₁₋₃alkylene)-(4-6 membered heterocyclyl containing 1-2 heteroatoms selected from the group consisting of O, S, and N) optionally substituted by 1 R¹¹.

In some embodiments, R¹ is —(C₁₋₃alkylene)-(5-6 membered heteroaryl containing 1-2 heteroatoms selected from the group consisting of O, S, and N) optionally substituted by 1 R¹¹.

In some embodiments, L¹ is —O—, —S—, —NH—, —S(═O)₁₋₂—, —S(═O)(═NR⁵)—, —CHR⁶—, or —CF₂—. In some embodiments, L¹ is —O—, —S—, —NH—, —S(═O)₁₋₂—, or —C(R⁶)₂—. In some embodiments, L¹ is —O— or —S—. In some embodiments, L¹ is —O—. In some embodiments L¹ is —S—.

In some embodiments, L² is —NR⁷SO₂—, —C(R⁸)₂SO₂—, —NR⁷SO₂—C(R⁸)₂—, —C(R⁸)₂C(═O)—, —C(═O)C(R⁸)₂—, —C(═O)O—, —OC(═O)—, —C(R⁸)₂O—, —OC(R⁸)₂—, or —C(═O)NR⁷—. In some embodiments, L² is —NR⁷SO₂—. In some embodiments, L² is —NHSO₂—, —NHSO₂—CHR⁸—, —C(═O)NH—, or —NHC(═O)—. In some embodiments, L² —NHSO₂—, —NHSO₂—CH₂—, —C(═O)NH—, or —NHC(═O)—. In some embodiments L² is —NHSO₂—.

In some embodiments, R⁵ is each independently H or C₁₋₂alkyl. In some embodiments, R⁵ is H or methyl.

In some embodiments, R⁶ is each independently H, methyl, ethyl, fluoro, chloro, cyano, CF₃, CHF₂, or CH₂F.

In some embodiments, R⁷ is each independently H, C₁₋₆ alkyl, C₁₋₆ haloalkyl, C₂₋₆ alkenyl, C₂₋₆ alkynyl, C₂₋₆ cycloalkyl, C₄₋₆heterocycloalkyl, aryl, 5-10 membered heteroaryl, C₁₋₃ alkylene-C₃₋₆ cycloalkyl, or C₁₋₃ alkylene-C₄₋₆ heterocycloalkyl. In some embodiments, R⁷ is each independently H, C₁₋₆ alkyl, C₃₋₆ cycloalkyl, C₄₋₆ heterocycloalkyl, aryl, or 5-10 membered heteroaryl. In some embodiments, R⁷ is H or C₁₋₆alkyl. In some embodiments, two R⁷s, along with the N atom to which they are attached form a 4-7 membered ring comprising an additional 0-1 heteroatoms selected from NR⁵, O and S(═O)₀₋₂.

In some embodiments, R⁸ is each independently H, halogen, cyano, C₁₋₆ alkyl, C₁₋₆ haloalkyl, C₂₋₆ alkenyl, C₂₋₆ alkynyl, C₃₋₆cycloalkyl, C₄₋₆heterocycloalkyl, C₁₋₃ alkylene-C₃₋₆ cycloalkyl, C₁₋₃ alkylene-C₄₋₆ heterocycloalkyl. In some embodiments, two R⁸s taken together with the C atom to which they are attached form a 4-7 membered ring comprising 0-1 heteroatom selected from O, S(═O)₀₋₂, P(═O)R¹², and NR⁵. In some embodiments, R⁸ is each independently H, methyl, ethyl, fluoro, chloro, cyano, CF₃, CHF₂, CH₂F, methoxy, or ethoxy.

In some embodiments, R² is each independently H, halogen, cyano, nitro, hydroxy, —NR⁷R¹⁰, —NHCOR⁹, C₁₋₆ alkyl, C₂₋₆ alkenyl, C₂₋₆ alkynyl, C₁₋₆ alkoxy, C₂₋₆ alkenoxy, C₂₋₆ alkynoxy, COR⁹, CON(R⁹)₂, CF₃, CF₂H, CF₃CF₂, CF₃CH₂, C₁₋₆haloalkyl, CF₃O, CF₂HO, CF₃CF₂O, CF₃CH₂O, C₁₋₆haloalkoxy, C₃₋₈ cycloalkyl, C₃₋₈ cycloalkenyl, C₁₋₆ alkylthio, C₂₋₆ alkenylthio, C₁₋₆ alkylsulfonyl, aryl, or heteroaryl, wherein R² is each independently optionally substituted by 0-2 R¹¹. In some embodiments, R² is each independently H, halogen, cyano, C₁₋₆ alkyl, C₂₋₆ alkenyl, C₂₋₆ alkynyl, C₁₋₆haloalkyl, or C₃₋₆cycloalkyl. In some embodiments, R² is each independently H, halogen, cyano, C₁₋₆ alkyl, C₂₋₄alkenyl, C₂₋₄alkynyl, C₁₋₄haloalkyl, or C₃₋₆cycloalkyl.

In some embodiments, R³ is each independently H, halogen, cyano, nitro, hydroxy, —NR⁷R¹⁰, —NHCOR⁹, C₁₋₆ alkyl, C₂₋₆ alkenyl, C₂₋₆ alkynyl, C₁₋₆ alkoxy, C₂₋₆ alkenoxy, C₂₋₆ alkynoxy, COR⁹, CON(R⁹)₂, CF₃, CF₂H, CF₃CF₂, CF₃CH₂, C₁₋₆haloalkyl, CF₃O, CF₂HO, CF₃CF₂O, CF₃CH₂O, C₁₋₆haloalkoxy, C₃₋₈ cycloalkyl, C₃₋₈ cycloalkenyl, C₁₋₆ alkylthio, C₂₋₆ alkenylthio, or C₁₋₆ alkylsulfonyl, wherein R³ is each independently optionally substituted by 0-2 R¹¹. In some embodiments, R³ is each independently H, halogen, cyano, C₁₋₆alkyl, or C₁₋₆haloalkyl. In some embodiments, R³ is each independently H, halogen, cyano, C₁₋₄alkyl, or C₁₋₄haloalkyl. In some embodiments, R³ is each independently H, halogen, cyano, or C₁₋₂haloalkyl. In some embodiments, R³ is each independently H or halogen.

In some embodiments, R⁴ is each independently H, halogen, cyano, nitro, hydroxy, NR⁷R¹⁰, NHCOR⁹, NR⁷CONR⁷, C₁₋₆ alkyl, C₂₋₆ alkenyl, C₂₋₆ alkynyl, COR⁹, CO₂R⁹, CONR⁷R¹⁰, C₁₋₆ alkoxy, C₂₋₆ alkenoxy, C₂₋₆ alkynoxy, CF₃, CF₂H, CF₃CF₂, CF₃CH₂, C₁₋₆ haloalkyl, CF₃O, CF₂HO, CF₃CF₂O, CF₃CH₂O, C₁₋₆haloalkoxy, C₃₋₈ cycloalkyl, C₃₋₈ cycloalkenyl, C₁₋₆ alkylthio, C₂₋₆ alkenylthio, C₁₋₆ alkylsulfonyl, SO₂N(R⁷)₂, or phenyl, or any two occurrences of R⁴ on contiguous atoms, taken together with the contiguous atoms to which the two occurrences of R⁴ are attached, form a saturated or unsaturated 5-7 membered ring comprising 0-2 heteroatoms each independently selected from O, S(═O)₀₋₂, P(═O)R¹², N, and NR¹⁰, wherein R⁴ is each independently optionally substituted by 0-2 R¹¹. In some embodiments, R⁴ is each independently H, halogen, cyano, nitro, hydroxy, NR⁷R¹⁰, C₁₋₆alkyl, C₂₋₆alkenyl, C₂₋₆alkynyl, C₁₋₆alkoxy, C₂₋₆alkenoxy, C₂₋₆alkynoxy, C₁₋₆haloalkyl, C₁₋₆haloalkoxy, C₃₋₈cycloalkyl, C₃₋₈cycloalkenyl, C₁₋₆alkylsulfonyl, SO₂N(R⁷)₂, or phenyl, wherein R⁴ is each independently optionally substituted by 0-2 R¹¹. In some embodiments, R⁴ is each independently H, halogen, cyano, nitro, hydroxy, NR⁷R¹⁰, NHCOR⁹, NR⁷CONR⁷, C₁₋₆alkyl, C₂₋₆alkenyl, C₂₋₆alkynyl, COR⁹, CO₂R⁹, CONR⁷R¹⁰, C₁₋₆alkoxy, C₁₋₆haloalkyl, C₁₋₆haloalkoxy, or C₃₋₈cycloalkyl, wherein R⁴ is each independently optionally substituted by 0-1 R¹¹. In some embodiments, R⁴ is each independently H, halogen, cyano, nitro, C₁₋₆ alkyl optionally substituted with R¹¹, C₂₋₆alkenyl, C₂₋₆alkynyl, C₁₋₆haloalkyl, C₁₋₆alkoxy, C₃₋₆cycloalkyl, or CONR⁷R¹⁰.

In some embodiments, R⁹ is H, C₁₋₆ alkyl, C₁₋₆ haloalkyl, C₂₋₆ alkenyl, C₂₋₆ alkynyl, C₃₋₁₀ cycloalkyl, C₄₋₁₀ heterocycloalkyl, C₁₋₃ alkylene-C₃₋₆ cycloalkyl, C₁₋₃ alkylene-C₄₋₆ heterocycloalkyl, C₁₋₆ acyl, phenyl, or 5-12 membered heteroaryl, wherein R⁹ is optionally substituted by 1-2 R¹¹. In some embodiments, R⁹ is H, C₃₋₁₀ cycloalkyl, C₄₋₁₀ heterocycloalkyl, phenyl, or 5-12 membered heteroaryl, wherein R⁹ is optionally substituted by 1-2 R¹¹. In some embodiments, R⁹ is C₁₋₆alkyl optionally substituted by 1 R¹¹.

In some embodiments, R¹⁰ is H, C₁₋₆ alkyl, C₁₋₆ haloalkyl, C₂₋₆ alkenyl, C₂₋₆ alkynyl, C₃₋₁₀ cycloalkyl, C₄₋₁₀ heterocycloalkyl, C₁₋₃ alkylene-C₃₋₆ cycloalkyl, C₁₋₃ alkylene-C₄₋₆ heterocycloalkyl, C₁₋₆ acyl, phenyl, or 5-12 membered heteroaryl, wherein R¹⁰ is optionally substituted by 1-2 R¹¹. In some embodiments, R¹⁰ is H or C₁₋₆alkyl, wherein the R¹⁰ is optionally substituted by 1-2 R¹¹. In some embodiments, R¹⁰ is H or C₁₋₆alkyl, wherein the R¹⁰ is optionally substituted by 1 R¹¹. In some embodiments, R¹⁰ is H or C₁₋₆alkyl.

In some embodiments, R⁷ and R¹⁰ on the same N atom may be taken together with the N atom to which they are attached form a 4-7 membered ring comprising an additional 0-1 heteroatoms selected from NR⁵, O, S(═O)₀₋₂, P(═O)R¹², and NR¹³, wherein R¹⁰ is optionally substituted by 1-2 R¹¹. In some embodiments, R⁷ and R¹⁰ on the same N atom may be taken together with the N atom to which they are attached form a 4-7 membered ring comprising an additional 0-1 heteroatoms selected from NR⁵, O, S(═O)₀₋₂, P(═O)R¹², and NR¹³.

In some embodiments, R¹¹ is each independently R⁶, oxo, nitro, C₂₋₆ alkenyl, C₂₋₆ alkynyl, C₁₋₆ alkoxy, C₁₋₆ haloalkyl, C₁₋₆haloalkoxy, C₃₋₆ cycloalkyl, 4-6 membered heterocycloalkyl, C₁₋₃ alkylene-C₃₋₆ cycloalkyl, C₁₋₃ alkylene-C₄₋₆ heterocycloalkyl, C₆₋₁₀ aryl, C₅₋₆ heteroaryl, SO₂R⁷, S(═O)NR⁵, SO₂N(R⁷)₂, CON(R⁷)₂, or N(R⁷)₂. In some embodiments, R¹¹ is each independently R⁶, nitro, C₂₋₆ alkenyl, C₂₋₆ alkynyl, C₁₋₆ alkoxy, C₁₋₆haloalkyl, C₁₋₆ haloalkoxy, C₃₋₆ cycloalkyl, 4-6 membered heterocycloalkyl, C₁₋₃ alkylene-C₃₋₆ cycloalkyl, C₁₋₃ alkylene-C₄₋₆ heterocycloalkyl, C₆₋₁₀ aryl, C₅₋₆heteroaryl, SO₂R⁷, S(═O)NR⁵, SO₂N(R⁷)₂, CON(R⁷)₂, or N(R⁷)₂. In some embodiments, wherein R¹¹ is each independently R⁶, C₁₋₆ alkoxy, C₁₋₆haloalkyl, C₃₋₆ cycloalkyl, 4-6 membered heterocycloalkyl, C₆₋₁₀ aryl, C₅₋₆ heteroaryl, SO₂R⁷, S(═O)NR⁵, SO₂N(R⁷)₂, CON(R⁷)₂, or N(R⁷)₂. In some embodiments, R¹¹ is each independently N(R⁷)₂.

In some embodiments, R¹² is each independently H, hydroxy, C₁₋₆alkyl, or C₁₋₆alkoxy. In some embodiments, In some embodiments, R¹² is each independently H or C₁₋₆alkyl.

In some embodiments, R¹³ is each independently H, C₁₋₆ alkyl, C₁₋₆ haloalkyl, C₂₋₆ alkenyl, C₂₋₆ alkynyl, C₂₋₆ cycloalkyl, C₄₋₆ heterocycloalkyl, C₁₋₃ alkylene-C₃₋₆ cycloalkyl, or C₁₋₃ alkylene-C₄₋₆ heterocycloalkyl, C₁₋₆ acyl, C₆₋₁₀ aroyl, or C₅₋₆ heteroaroyl.

In some embodiments, m is an integer 0 or 1. In some embodiments, m is 0. In some embodiments, m is 1.

In some embodiments, n is an integer 0, 1, or 2. In some embodiments, n is an integer 0 or 1. In some embodiments, n is an integer 1 or 2. In In some embodiments, n is 0. In some embodiments, n is 1. In some embodiments, n is 2.

In some embodiments, p is an integer 0, 1, or 2. In some embodiments, p is an integer 1, 2, or 3. In some embodiments, p is an integer 0 or 1. In some embodiments, p is 0. In some embodiments, p is 1. In some embodiments, p is 2. In some embodiments, p is 3.

In some embodiments, the compound is a compound of Formula I-aa:

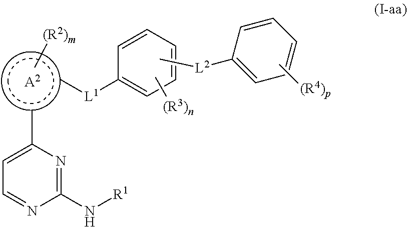

or a pharmaceutically acceptable salt thereof, wherein the variables are as defined for Formula I.

In some embodiments, the compound is a compound of Formula I-ba:

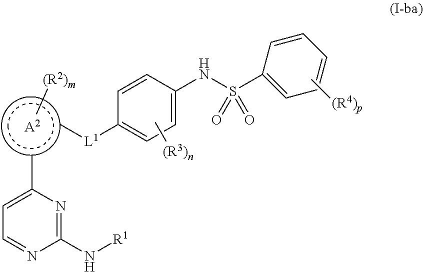

or a pharmaceutically acceptable salt thereof, wherein the variables are as defined for Formula I.

In some embodiments, the compound is a compound of Formula I-ab:

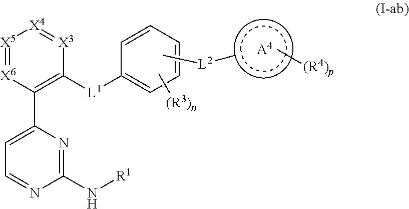

or a pharmaceutically acceptable salt thereof, wherein X³, X⁴, X⁵, and X⁶ are each independently N or CH, wherein the hydrogen of each CH is independently optionally substituted by R², wherein the variables are as defined for Formula I.

In some embodiments, the compound is a compound of Formula I-bb:

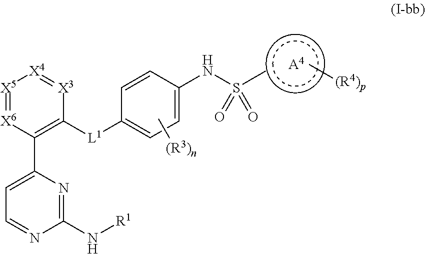

or a pharmaceutically acceptable salt thereof, wherein X³, X⁴, X⁵, and X⁶ are each independently N or CH, wherein the hydrogen of each CH is independently optionally substituted by R², wherein the variables are as defined for Formula I.

In some embodiments, the compound is a compound of Formula I-ac:

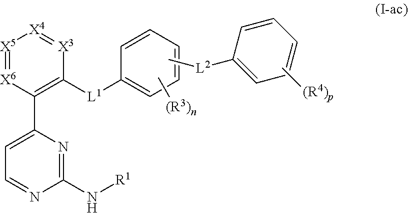

or a pharmaceutically acceptable salt thereof, wherein X³, X⁴, X⁵, and X⁶ are each independently N or CH, wherein the hydrogen of each CH is independently optionally substituted by R², wherein the variables are as defined for Formula I.

In some embodiments, the compound is a compound of Formula I-bc:

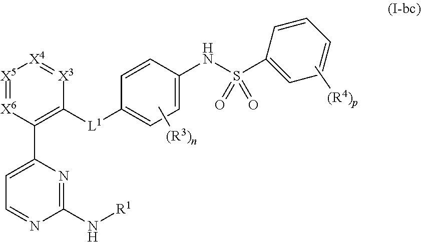

or a pharmaceutically acceptable salt thereof, wherein X³, X⁴, X⁵, and X⁶ are each independently N or CH, wherein the hydrogen of each CH is independently optionally substituted by R², wherein the variables are as defined for Formula I.

In some embodiments, the compound is a compound of Formula I-ad, I-ae, I-af, or I-ag:

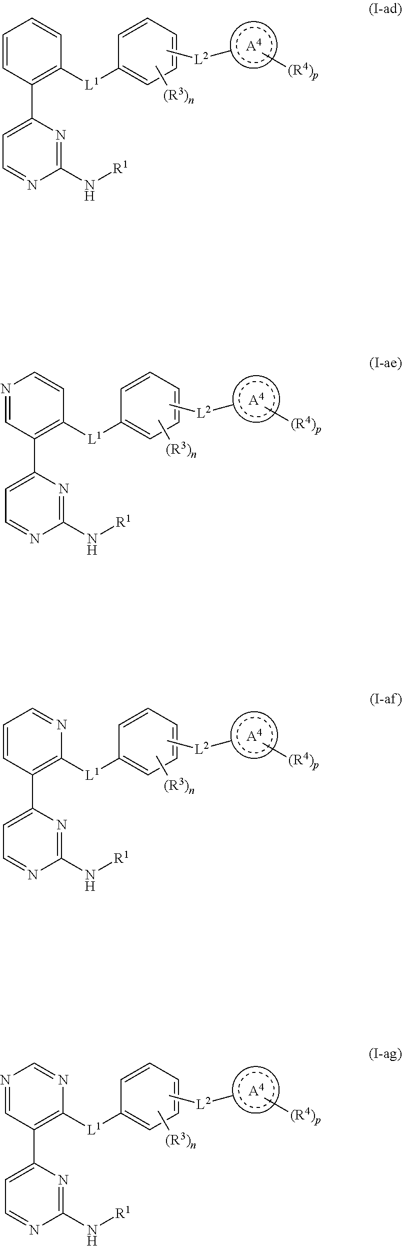

or a pharmaceutically acceptable salt thereof, wherein the variables are as defined for Formula I.

In some embodiments, the compound is a compound of Formula I-bd, I-be, I-bf, or I-bg:

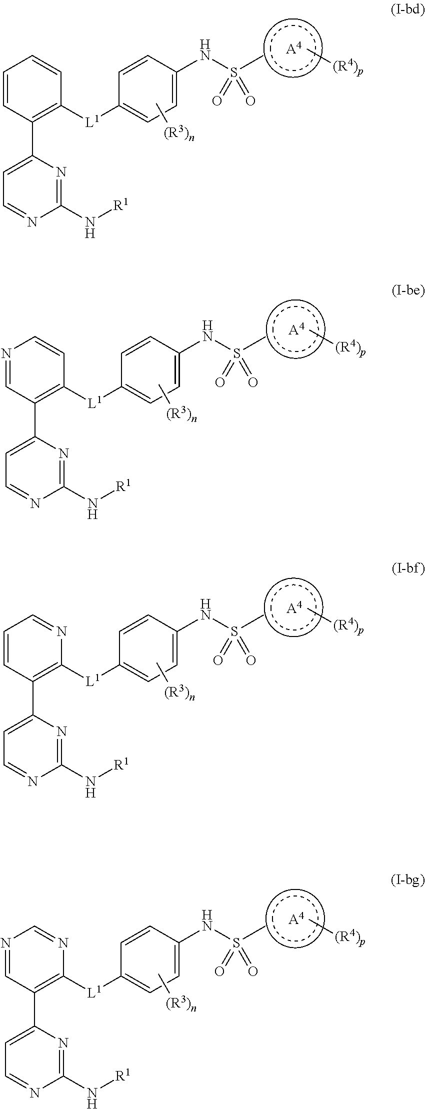

or a pharmaceutically acceptable salt thereof, wherein the variables are as defined for Formula I.

In some embodiments, the compound is a compound of Formula I-ah, I-ai, I-aj, or I-ak:

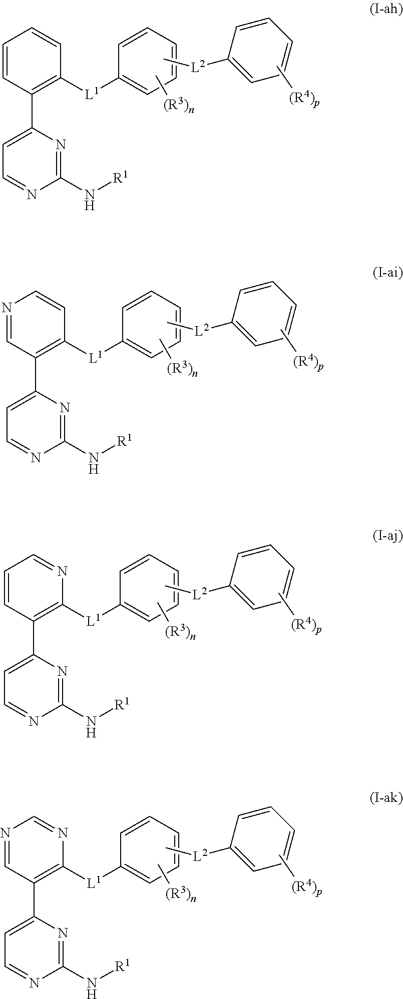

or a pharmaceutically acceptable salt thereof, wherein the variables are as defined for Formula I.

In some embodiments, the compound is a compound of Formula I-bh, I-bi, I-bj, or I-bk:

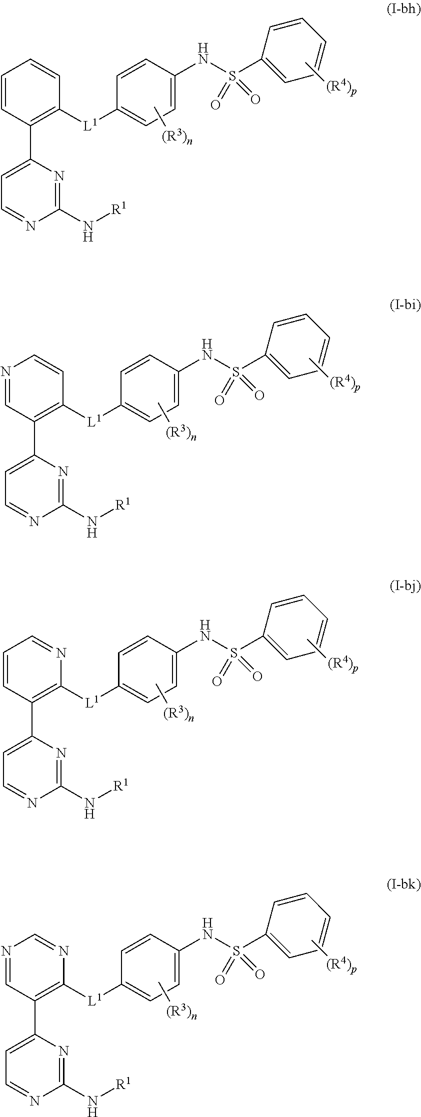

or a pharmaceutically acceptable salt thereof, wherein the variables are as defined for Formula I.

In some embodiments, provided herein are compounds of Formula II:

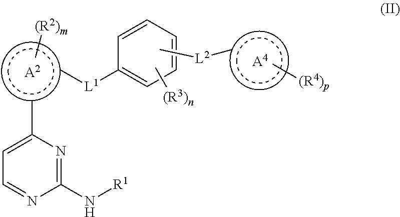

or a pharmaceutically acceptable salt thereof, wherein:
A² is aryl or heteroaryl;
A⁴ is aryl, heteroaryl, or C₃₋₁₂carbocyclyl;
L¹ is —O—, —NR^{N1}—, —S(═O)₀₋₂—, —S(═O)(═NR^{N1})—, —C(R⁶)₂—, or —C(═O)—;
L² is —NR^{N1}SO₂—, —SO₂NR^{N1}—, —C(R⁷)₂C(═O)—, —C(═O)C(R⁷)₂—, —C(═O)O—, —OC(═O)—, —C(R⁶)₂O—, —OC(R⁶)₂—, —C(═O)NR^{N1}—, —NR^{N1}C(═O)—, —C(R⁶)₂NR^{N1}—, —NR^{N1}C(R⁶)₂—, —C(R⁶)₂S(═O)₀₋₂—, —S(═O)₀₋₂C(R⁶)₂—, —C(R⁶)₂S(═O)NR^{N1}—, —S(═O)NR^{N1}C(R⁶)₂—, —C(R⁶)₂C(R⁷)₂—, or —C(R⁷)₂C(R⁶)₂—;
R¹ is C₃₋₁₀carbocyclyl, 4-12 membered heterocyclyl, phenyl, 5-12 membered heteroaryl, —(C₁₋₆alkylene)-(C₃₋₁₀carbocyclyl), —(C₁₋₆alkylene)-(4-12 membered heterocyclyl), —(C₁₋₆alkylene)-(phenyl), —(C₁₋₆alkylene)-(5-12 membered heteroaryl), —(C₁₋₆alkylene)-OR⁸, or —(C₁₋₆alkylene)-NR⁹R¹⁰, wherein R¹ is optionally substituted by 1-3 R¹²;
R², R³, and R⁴ are each independently halogen, cyano, nitro, oxo, hydroxy, amino, C₁₋₆alkyl, C₁₋₆alkoxy, C₁₋₆alkylamino, C₁₋₆dialkylamino, C₁₋₆haloalkyl, C₁₋₆haloalkoxy, C₃₋₁₀carbocyclyl, C₁₋₆alkylthio, or C₁₋₆alkylsulfonyl, or any two occurrences of R², R³, or R⁴ on contiguous atoms taken together with the contiguous atoms to which the two occurrences of R², R³, or R⁴ are attached form a 5-7 membered ring comprising 0-2 heteroatoms selected from O, S(═O)₀₋₂, P(═O)R^{P1}, N, and NR^{N1}, wherein R², R³, or R⁴ are each independently optionally substituted by 1-3 R¹²;
R⁶ is each independently H, cyano, C₁₋₆alkyl, or C₁₋₆haloalkyl, wherein R⁶ is optionally substituted by 1-3 R¹²;
R⁷ is each independently H, halogen, cyano, C₁₋₆alkyl, C₁₋₆haloalkyl, C₁₋₆alkoxy, or C₁₋₆haloalkoxy, or two occurrences of R⁷ taken together with the C atom to which they are attached form a 4-7 membered ring comprising 0-1 heteroatom selected from O, S(═O)₀₋₂, P(═O)R^{P1}, and NR^{N1}, wherein R⁷ is optionally substituted by 1-3 R¹²;
R⁸ is C₃₋₁₀carbocyclyl, 4-12 membered heterocyclyl, phenyl, or 5-12 membered heteroaryl, wherein R⁸ is optionally substituted by 1-3 R¹²;
R⁹ is H, C₁₋₆alkyl, C₃₋₁₀carbocyclyl, 4-12 membered heterocyclyl, phenyl, or 5-12 membered heteroaryl, wherein R⁹ is optionally substituted by 1-3 R¹²;
R¹⁰ is H or C₁₋₆alkyl;
or R⁹ and R¹⁰ taken together with the N atom to which they are attached form a 4-12 membered heterocyclyl optionally substituted by 1-3 R¹²;
R¹¹ is each independently C₁₋₆alkyl, C₁₋₆haloalkyl, C₃₋₁₀carbocyclyl, or 4-12 membered heterocyclyl;
R¹² is each independently halogen, cyano, oxo, hydroxy, C₁₋₆alkyl, C₂₋₆alkenyl, C₂₋₆alkynyl, C₁₋₆alkoxy, C₁₋₆haloalkyl, C₁₋₆haloalkoxy, SO₂R¹¹, S(═O)NR^{N1}R¹¹, or NR^{N1}R^{N2},
R^{P1} is each independently H or C₁₋₆alkyl;
R^{N1} is each independently H or C₁₋₆alkyl;
R^{N2} is each independently H, C₁₋₆alkyl, SO₂R¹¹, or S(═O)NR^{N1}R¹¹;
m is an integer 0, 1, 2, or 3;
n is an integer 0, 1, 2, or 3; and
p is an integer 0, 1, 2, or 3,
provided that
L¹ and L² are not situated on contiguous atoms of the phenyl;
when A² is

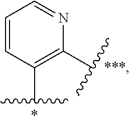 [SMILES: *c1cccnc1*]

L¹ is not —O—, wherein * is the site covalently linked to the pyrimidyl, and *** is the site covalently linked to L¹; or
when A² is

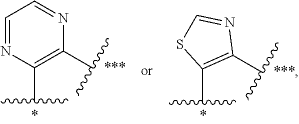 [SMILES: *c1nccnc1* ; *c1ncsc1*]

L¹ is not —O—, wherein * is the site covalently linked to the pyrimidyl, and *** is the site covalently linked to L¹.

In some embodiments, A⁴ is phenyl, naphthyl, or 5-12 membered heteroaryl. In some embodiments, A⁴ is phenyl, naphthyl, 5-6 membered monocyclic heteroaryl, or 8-12 membered bicyclic heteroaryl. In some embodiments, A⁴ is phenyl, pyridyl, pyridazinyl, pyrimidyl, pyrazinyl, triazinyl, thienyl, thiazolyl, thiadiazolyl, oxazolyl, oxadiazolyl, pyrazolyl, imidazolyl, triazolyl, or tetrazolyl.

In some embodiments, A² is phenyl. In some embodiments, A² is 5-6 membered heteroaryl. In some embodiments, A² is 1,2-linked phenylene, 2-L¹-3-biaryl linked pyridylene or 4-L¹-3-biaryl linked pyridylene.

In some embodiments, R¹ is C₃₋₁₀carbocyclyl, 4-12 membered heterocyclyl, phenyl, or 5-12 membered heteroaryl, wherein R¹ is optionally substituted by 1-3 R¹². In some embodiments, R¹ is C₃₋₁₀carbocyclyl or 4-12 membered heterocyclyl, wherein R¹ is optionally substituted by 1-3 R¹².

In some embodiments, L¹ is —O—, —NR^{N1}—, S(═O)₀₋₂, —S(═O)(═NR^{N1})—, —CHR⁶—, or —CF₂—. In some embodiments, L¹ is —O—, —S(═O)₀₋₂—, or —C(R⁶)₂—. In some embodiments, L¹ is —O—. In some embodiments L¹ is —S—. In some embodiments L¹ is —NH—.

In some embodiments, L² is NHSO₂—, —SO₂NH—, —C(R⁷)₂C(═O)—, —C(═O)C(R⁷)₂—, —C(═O)O—, —OC(═O)—, —C(R⁶)₂O—, —OC(R⁶)₂—, —C(═O)NH—, —NHC(═O)—, —C(R⁶)₂NH—, —NHC(R⁶)₂—, —C(R⁶)₂S(═O)₀₋₂—, —S(═O)₀₋₂C(R⁶)₂—, —C(R⁶)₂S(═O)NH—, —S(═O)NHC(R⁶)₂—, —C(R⁶)₂C(R⁷)₂—, or —C(R⁷)₂C(R⁶)₂—. In some embodiments, L² is —NR^{N1}SO₂—.

In some embodiments, R⁶ is each independently H, methyl, ethyl, fluoro, chloro, cyano, CF₃, CHF₂, or CH₂F.

In some embodiments, R⁷ is each independently H, methyl, ethyl, fluoro, chloro, cyano, CF₃, CHF₂, CH₂F, methoxy, or ethoxy.

In some embodiments, m is an integer 0, 1, or 2.

In some embodiments, n is an integer 0, 1, or 2.

In some embodiments, p is an integer 0, 1, or 2.

Also provided, in some embodiments, are compounds of Formula III:

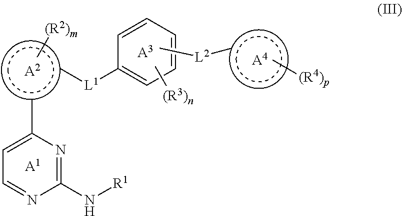

or pharmaceutically acceptable salts thereof, wherein:
A² is

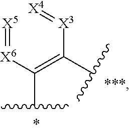

wherein * is the site covalently linked to the A¹ ring, and *** is the site covalently linked to L¹;
A⁴ is aryl, heteroaryl, C₃₋₁₂ carbocyclyl, or 4-12 membered heterocyclyl;
L¹ is —O—, —S—, —NH—, S(═O)₁₋₂—, —S(═O)(═NR⁵)—, —C(R⁶)₂—, or —C(═O)—;
L² is —NR⁷SO₂—, —C(R⁸)₂SO₂—, —NR⁷SO₂—C(R⁸)₂—, —C(R⁸)₂C(═O)—, —C(═O)C(R⁸)₂—, —C(═O)O—, —OC(═O)—, —C(R⁸)₂O—, —OC(R⁸)₂—, —C(═O)NR⁷—, —NR⁷C(═O)—, —C(R⁸)₂NR⁷—, —NR⁷C(R⁸)₂—, —C(R⁸)₂S(═O)₀₋₂—, —S(═O)₀₋₂C(R⁸)₂—, —C(R⁸)₂S(═O)NR⁷—, S(═O)NR⁷C(R⁸)₂—, or —C(R⁸)₂C(R⁸)₂—;
R¹ is C₃₋₁₂ carbocyclyl, 4-12 membered heterocyclyl, phenyl, 5-12 membered heteroaryl, —(C₁₋₆ alkylene)-(C₃₋₁₀carbocyclyl), —(C₁₋₆ alkylene)-(4-12 membered heterocyclyl), —(C₁₋₆alkylene)-(phenyl), —(C₁₋₆alkylene)-(5-12 membered heteroaryl), —(C₁₋₆alkylene)-OR⁹, or —(C₁₋₆alkylene)-NR⁷R¹⁰, wherein R¹ is optionally substituted by 0-5 R¹¹;
R² is each independently H, halogen, cyano, nitro, hydroxy, —NR⁷R¹⁰, —NHCOR⁹, C₁₋₆alkyl, C₂₋₆ alkenyl, C₂₋₆ alkynyl, C₁₋₆ alkoxy, C₂₋₆ alkenoxy, C₂₋₆ alkynoxy, COR⁹, CON(R⁹)₂, CF₃, CF₂H, CF₃CF₂, CF₃CH₂, C₁₋₆haloalkyl, CF₃O, CF₂HO, CF₃CF₂O, CF₃CH₂O, C₁₋₆ haloalkoxy, C₃₋₈ cycloalkyl, C₃₋₈ cycloalkenyl, C₁₋₆ alkylthio, C₂₋₆ alkenylthio, C₁₋₆ alkylsulfonyl, aryl, or heteroaryl, wherein R² is each independently optionally substituted by 0-4 R¹¹;
R³ is each independently H, halogen, cyano, nitro, hydroxy, —NR⁷R¹⁰, —NHCOR⁹, C₁₋₆ alkyl, C₂₋₆ alkenyl, C₂₋₆ alkynyl, C₁₋₆ alkoxy, C₂₋₆ alkenoxy, C₂₋₆ alkynoxy, COR⁹, CON(R⁹)₂, CF₃, CF₂H, CF₃CF₂, CF₃CH₂, C₁₋₆haloalkyl, CF₃O, CF₂HO, CF₃CF₂O, CF₃CH₂O, C₁₋₆ haloalkoxy, C₃₋₈ cycloalkyl, C₃₋₈ cycloalkenyl, C₁₋₆ alkylthio, C₂₋₆ alkenylthio, or C₁₋₆ alkylsulfonyl, wherein R³ is each independently optionally substituted by 0-4 R¹¹;
R⁴ is each independently H, halogen, cyano, nitro, hydroxy, NR⁷R¹⁰, NHCOR⁹, NR⁷CONR⁷, C₁₋₆ alkyl, C₂₋₆ alkenyl, C₂₋₆ alkynyl, COR⁹, CO₂R⁹, CONR⁷R¹⁰, C₁₋₆ alkoxy, C₂₋₆ alkenoxy, C₂₋₆ alkynoxy, CF₃, CF₂H, CF₃CF₂, CF₃CH₂, C₁₋₆ haloalkyl, CF₃O, CF₂HO, CF₃CF₂O, CF₃CH₂O, C₁₋₆ haloalkoxy, C₃₋₈ cycloalkyl, C₃₋₈ cycloalkenyl, C₁₋₆ alkylthio, C₂₋₆ alkenylthio, C₁₋₆ alkylsulfonyl, SO₂N(R⁷)₂, or phenyl, or any two occurrences of R⁴ on contiguous atoms, taken together with the contiguous atoms to which the two occurrences of R⁴ are attached, form a saturated or unsaturated 5-7 membered ring comprising 0-2 heteroatoms each independently selected from O, S(═O)₀₋₂, P(═O)R¹², N, and NR¹⁰, wherein R⁴ is each independently optionally substituted by 0-4 R¹¹;
R⁵ is each independently H or C₁₋₄ alkyl;
R⁶ is each independently H, F, OH, cyano, or C₁₋₆ alkyl;
R⁷ is each independently H, C₁₋₆ alkyl, C₁₋₆ haloalkyl, C₂₋₆ alkenyl, C₂₋₆ alkynyl, C₃₋₆ cycloalkyl, C₄₋₆ heterocycloalkyl, aryl, 5-10 membered heteroaryl, C₁₋₃ alkylene-C₃₋₆ cycloalkyl, or C₁₋₃ alkylene-C₄₋₆ heterocycloalkyl, or two R⁷s along with the N atom to which they are attached form a 4-7 membered ring comprising an additional 0-1 heteroatoms selected from NR⁵, O and S(═O)₀₋₂;
R⁸ is each independently H, halogen, cyano, C₁₋₆ alkyl, C₁₋₆ haloalkyl, C₂₋₆ alkenyl, C₂₋₆ alkynyl, C₂₋₆ cycloalkyl, C₄₋₆ heterocycloalkyl, C₁₋₃ alkylene-C₃₋₆ cycloalkyl, C₁₋₃ alkylene-C₄₋₆ heterocycloalkyl, or two R⁸s taken together with the C atom to which they are attached form a 4-7 membered ring comprising 0-1 heteroatom selected from O, S(═O)₀₋₂, P(═O)R¹², and NR⁵, wherein R⁸ is optionally substituted by 1-3 R¹¹;
each R⁹ is H, C₁₋₆ alkyl, C₁₋₆ haloalkyl, C₂₋₆ alkenyl, C₂₋₆ alkynyl, C₃₋₁₀ cycloalkyl, C₄₋₁₀ heterocycloalkyl, C₁₋₃ alkylene-C₃₋₆ cycloalkyl, C₁₋₃ alkylene-C₄₋₆ heterocycloalkyl, C₁₋₆ acyl, phenyl, or 5-12 membered heteroaryl, wherein R⁹ is optionally substituted by 1-3 R¹¹;
each R¹⁰ is independently H, C₁₋₆ alkyl, C₁₋₆ haloalkyl, C₂₋₆ alkenyl, C₂₋₆ alkynyl, C₃₋₁₀ cycloalkyl, C₄₋₁₀heterocycloalkyl, C₁₋₃ alkylene-C₃₋₆ cycloalkyl, C₁₋₃ alkylene-C₄₋₆ heterocycloalkyl, C₁₋₆ acyl, C₁₋₆ alkylsulfonyl, phenyl, or 5-12 membered heteroaryl, wherein R¹⁰ is optionally substituted by 1-3 R¹¹,
or R⁷ and R¹⁰ on the same N atom may be taken together with the N atom to which they are attached form a 4-7 membered ring comprising an additional 0-1 heteroatoms selected from NR⁵, O, S(═O)₀₋₂, P(═O)R¹², and NR¹³;
R¹¹ is each independently R⁶, oxo, nitro, C₂₋₆ alkenyl, C₂₋₆ alkynyl, C₁₋₆ alkoxy, C₁₋₆ alkoxyalkyl, C₁₋₆hydroxyalkyl, C₁₋₆ haloalkyl, C₁₋₆ haloalkoxy, C₃₋₆ cycloalkyl, 4-6 membered heterocycloalkyl, C₁₋₃ alkylene-C₃₋₆ cycloalkyl, C₁₋₃ alkylene-C₄₋₆ heterocycloalkyl, C₆₋₁₀ aryl, C₅₋₁₀ heteroaryl, SO₂R⁷, S(═O)NR⁵, SO₂N(R⁷)₂, CON(R⁷)₂, or N(R⁷)₂;
R¹² is each independently H, OH, C₁₋₆ alkyl, C₁₋₆ alkoxy, or —N(R⁷)₂;
R¹³ is each independently H, C₁₋₆ alkyl, C₁₋₆ haloalkyl, C₂₋₆ alkenyl, C₂₋₆ alkynyl, C₂₋₆ cycloalkyl, C₄₋₆ heterocycloalkyl, C₁₋₃ alkylene-C₃₋₆ cycloalkyl, or C₁₋₃ alkylene-C₄₋₆ heterocycloalkyl, C₁₋₆ acyl, aroyl, or heteroaroyl;
m is an integer 0, 1 or 2;
n is an integer 0, 1, 2, or 3; and
p is an integer 0, 1, 2, or 3,
provided that
L¹ and L² are not situated on contiguous atoms of the A³ ring;
and when A² is

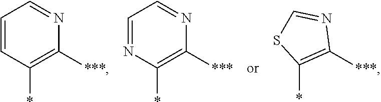

L¹ is not —O—, wherein * is the site covalently linked to the A¹ ring, and *** is the site covalently linked to L¹.

In some embodiments, when X⁴ and X⁵ are both CH wherein the hydrogen of CH is each optionally and independently substituted by R² and X⁶ is N or CH, X³ is not N. In some embodiments, when X⁵ and X⁶ are taken together as S and X⁴ is CH wherein the hydrogen of CH is optionally substituted by R², X³ is not N.

In some embodiments, A⁴ is phenyl, naphthyl, 5-12 membered heteroaryl, C₃₋₁₀ carbocyclyl, or 4-12 membered heterocyclyl. In some embodiments, A⁴ is phenyl, naphthyl, 5-10 membered heteroaryl, C₃₋₁₀ carbocyclyl, or 5-10 membered heterocyclyl. In some embodiments, A⁴ is phenyl, naphthyl, 5-10 membered heteroaryl, C₃₋₆ carbocyclyl, or 5-10 membered heterocyclyl. In some embodiments, A⁴ is phenyl, naphthyl, or 5-12 membered heteroaryl. In some embodiments, A⁴ is phenyl, naphthyl, or 5-10 membered heteroaryl. In some embodiments, A⁴ is phenyl, naphthyl, 5-6 membered monocyclic heteroaryl, or 8-12 membered bicyclic heteroaryl. In some embodiments, A⁴ is phenyl, pyridyl, pyridazinyl, pyrimidyl, pyrazinyl, triazinyl, thienyl, thiazolyl, thiadiazolyl, oxazolyl, oxadiazolyl, pyrazolyl, imidazolyl, triazolyl, or tetrazolyl. In some embodiments, A⁴ is phenyl, naphthyl, thiophenyl, cyclopropyl, cyclobutyl, cyclopentyl, cyclohexyl,

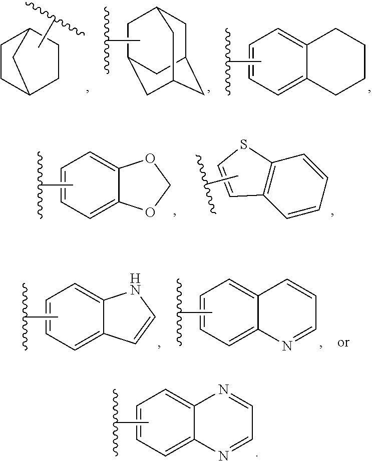

In some embodiments, A⁴ is phenyl.

In some embodiments, A² is phenyl, 2,3-naphthyl, pyridinyl, or pyrimidinyl. In some embodiments, A² is phenyl. In some embodiments, A² is 2,3-naphthyl. In some embodiments, A² is 5-6 membered heteroaryl. In some embodiments, A² is 1,2-linked phenylene, 2,3-linked-naphthylene, 2-L¹-3-biaryl linked pyridylene, 4-L¹-3-biaryl linked pyridylene or 4-L¹-5-biaryl linked pyrimidylene. In some embodiments, A² is 1,2-linked phenylene, 2-L¹-3-biaryl linked pyridylene or 4-L¹-3-biaryl linked pyridylene. In some embodiments, A² is phenyl, 2,3-naphthyl,

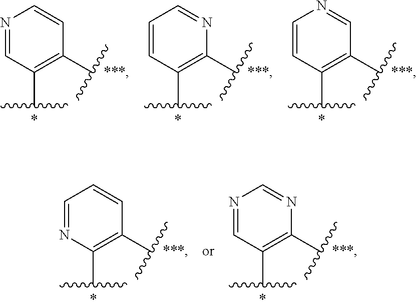 [SMILES: *c1ccncc1* ; *c1cccnc1* ; *c1ccncc1* ; *c1cccnc1* ; *c1cncnc1*]

wherein * is the site covalently linked to the A¹ ring, and *** is the site covalently linked to L¹.

In some embodiments, R¹ is C₃₋₁₀carbocyclyl, 4-12 membered heterocyclyl, phenyl, 5-12 membered heteroaryl, —(C₁₋₆alkylene)-(C₃₋₁₀carbocyclyl), —(C₁₋₆alkylene)-(4-12 membered heterocyclyl), —(C₁₋₆alkylene)-(phenyl), —(C₁₋₆alkylene)-(5-12 membered heteroaryl), —(C₁₋₆alkylene)-OR⁹, or —(C₁₋₆alkylene)-NR⁷R¹⁰, wherein R¹ is optionally substituted by 1-2 R¹¹. In some embodiments, R¹ is C₆₋₁₀carbocyclyl, 4-8 membered heterocyclyl, phenyl, 5-6 membered heteroaryl, —(C₁₋₆alkylene)-(C₃₋₁₀carbocyclyl), —(C₁₋₆alkylene)-(4-8 membered heterocyclyl), —(C₁₋₆alkylene)-(5-6 membered heteroaryl), —(C₁₋₆alkylene)-OR⁹, or —(C₁₋₆alkylene)-NR⁷R¹⁰, wherein R¹ is optionally substituted by 1-2 R¹¹. In some embodiments, R¹ is C₆₋₁₀carbocyclyl, 4-8 membered heterocyclyl, phenyl, 5-6 membered heteroaryl, —(C₁₋₆alkylene)-(C₃₋₁₀carbocyclyl), —(C₁₋₆alkylene)-(4-8 membered heterocyclyl), —(C₁₋₆alkylene)-(5-6 membered heteroaryl), —(C₁₋₆alkylene)-OR⁹, or —(C₁₋₆alkylene)-NR⁷R¹⁰, wherein R¹ is optionally substituted by 1 R¹¹. In some embodiments, R¹ is C₆₋₁₀carbocyclyl, 4-8 membered heterocyclyl containing 1-2 heteroatoms selected from the group consisting of O, S, and N, —(C₁₋₆alkylene)-(C₃₋₁₀carbocyclyl), —(C₁₋₆alkylene)-(4-8 membered heterocyclyl containing 1-2 heteroatoms selected from the group consisting of O, S, and N), —(C₁₋₆alkylene)-(5-6 membered heteroaryl containing 1-2 heteroatoms selected from the group consisting of O, S, and N), —(C₁₋₆alkylene)-OR⁹, or —(C₁₋₆alkylene)-NR⁷R¹⁰, wherein R¹ is optionally substituted by 1 R¹¹. In some embodiments, R¹ is C₆₋₁₀carbocyclyl, 4-8 membered heterocyclyl containing 1-2 heteroatoms selected from the group consisting of O, S, and N, —(C₁₋₃alkylene)-(4-6 membered heterocyclyl containing 1-2 heteroatoms selected from the group consisting of O, S, and N), —(C₁₋₃alkylene)-(5-6 membered heteroaryl containing 1-2 heteroatoms selected from the group consisting of O, S, and N), —(C₁₋₆alkylene)-OR⁹, or —(C₁₋₆alkylene)-NR⁷R¹⁰, wherein R¹ is optionally substituted by 1 R¹¹.

In some embodiments, R¹ is cyclohexyl, piperidinyl, oxetanyl, azetidinyl, pyrrolidinyl, or azaspiroheptanyl, wherein R¹ is optionally substituted with 1 R¹¹.

In some embodiments, R¹ is cyclohexyl optionally substituted with 1 R¹¹.

In some embodiments, R¹ is piperidinyl, oxetanyl, azetidinyl, pyrrolidinyl, or azaspiroheptanyl, wherein R¹ is optionally substituted with 1 R¹¹. In some embodiments, R¹ is piperidinyl optionally substituted with 1 R¹¹.

In some embodiments, R¹ is —(C₁₋₃alkylene)-OR⁹ wherein R⁹ is C₁₋₆alkyl substituted by 1-2 R¹¹. In some embodiments, R¹ is —(C₁₋₃alkylene)-OR⁹ wherein R⁹ is C₁₋₆alkyl substituted by 1 R¹¹. In some embodiments, R¹¹ is hydroxy or C₁₋₄alkoxy. In some embodiments, R¹¹ is hydroxy or C₁₋₂alkoxy.

In some embodiments, R¹ is —(C₁₋₃alkylene)-NR⁷R¹⁰ wherein R⁹ is H or C₁₋₆alkyl and R¹⁰ is H or C₁₋₆alkyl, wherein the R¹⁰ is optionally substituted by 1-2 R¹¹. In some embodiments, R¹ is —(C₁₋₃alkylene)-NR⁷R¹⁰ wherein R⁹ is H or C₁₋₆alkyl and R¹⁰ is H or C₁₋₆alkyl, wherein the R¹⁰ is optionally substituted by 1 R¹¹. In some embodiments, R¹¹ is hydroxy or C₁₋₄alkoxy. In some embodiments, R¹¹ is hydroxy or C₁₋₂alkoxy.

In some embodiments, R¹ is —(C₁₋₃alkylene)-(4-6 membered heterocyclyl containing 1-2 heteroatoms selected from the group consisting of O, S, and N) optionally substituted by 1 R¹¹.

In some embodiments, R¹ is —(C₁₋₃alkylene)-(5-6 membered heteroaryl containing 1-2 heteroatoms selected from the group consisting of O, S, and N) optionally substituted by 1 R¹¹.

In some embodiments, L¹ is —O—, —S—, —NH—, —S(═O)₁₋₂—, —S(═O)(═NR⁵)—, —CHR⁶—, or —CF₂—. In some embodiments, L¹ is —O—, —S—, —NH—, —S(═O)₁₋₂—, or —C(R⁶)₂—. In some embodiments, L¹ is —O— or —S—. In some embodiments, L¹ is —O—. In some embodiments L¹ is —S—.

In some embodiments, L² is —NR⁷SO₂—, —C(R⁸)₂SO₂—, —NR⁷SO₂—C(R⁸)₂—, —C(R⁸)₂C(═O)—, —C(═O)C(R⁸)₂—, —C(═O)O—, —OC(═O)—, —C(R⁸)₂O—, —OC(R⁸)₂—, or —C(═O)NR⁷—. In some embodiments, L² is —NR⁷SO₂—. In some embodiments, L² is —NHSO₂—, —NHSO₂—CHR⁸—, —C(═O)NH—, or —NHC(═O)—. In some embodiments, L² —NHSO₂—, —NHSO₂—CH₂—, —C(═O)NH—, or —NHC(═O)—. In some embodiments L² is —NHSO₂—.

In some embodiments, R⁵ is each independently H or C₁₋₂alkyl. In some embodiments, R⁵ is H or methyl.

In some embodiments, R⁶ is each independently H, methyl, ethyl, fluoro, chloro, cyano, CF₃, CHF₂, or CH₂F.

In some embodiments, R⁷ is each independently H, C₁₋₆ alkyl, C₁₋₆ haloalkyl, C₂₋₆ alkenyl, C₂₋₆ alkynyl, C₂₋₆ cycloalkyl, C₄₋₆heterocycloalkyl, aryl, 5-10 membered heteroaryl, C₁₋₃ alkylene-C₃₋₆ cycloalkyl, or C₁₋₃ alkylene-C₄₋₆ heterocycloalkyl. In some embodiments, R⁷ is each independently H, C₁₋₆ alkyl, C₃₋₆ cycloalkyl, C₄₋₆ heterocycloalkyl, aryl, or 5-10 membered heteroaryl. In some embodiments, R⁷ is H or C₁₋₆alkyl. In some embodiments, two R⁷s, along with the N atom to which they are attached form a 4-7 membered ring comprising an additional 0-1 heteroatoms selected from NR⁵, O and S(═O)₀₋₂.

In some embodiments, R⁸ is each independently H, halogen, cyano, C₁₋₆ alkyl, C₁₋₆ haloalkyl, C₂₋₆ alkenyl, C₂₋₆ alkynyl, C₃₋₆ cycloalkyl, C₄₋₆ heterocycloalkyl, C₁₋₃ alkylene-C₃₋₆ cycloalkyl, C₁₋₃ alkylene-C₄₋₆ heterocycloalkyl. In some embodiments, two R⁸s taken together with the C atom to which they are attached form a 4-7 membered ring comprising 0-1 heteroatom selected from O, S(═O)₀₋₂, P(═O)R¹², and NR⁵. In some embodiments, R⁸ is each independently H, methyl, ethyl, fluoro, chloro, cyano, CF₃, CHF₂, CH₂F, methoxy, or ethoxy.

In some embodiments, R² is each independently H, halogen, cyano, nitro, hydroxy, —NR⁷R¹⁰, —NHCOR⁹, C₁₋₆ alkyl, C₂₋₆ alkenyl, C₂₋₆ alkynyl, C₁₋₆ alkoxy, C₂₋₆ alkenoxy, C₂₋₆ alkynoxy, COR⁹, CON(R⁹)₂, CF₃, CF₂H, CF₃CF₂, CF₃CH₂, C₁₋₆haloalkyl, CF₃O, CF₂HO, CF₃CF₂O, CF₃CH₂O, C₁₋₆ haloalkoxy, C₃₋₈ cycloalkyl, C₃₋₈ cycloalkenyl, C₁₋₆ alkylthio, C₂₋₆ alkenylthio, C₁₋₆ alkylsulfonyl, aryl, or heteroaryl, wherein R² is each independently optionally substituted by 0-2 R¹¹. In some embodiments, R² is each independently H, halogen, cyano, C₁₋₆ alkyl, C₂₋₆ alkenyl, C₂₋₆ alkynyl, C₁₋₆haloalkyl, or C₃₋₆cycloalkyl. In some embodiments, R² is each independently H, halogen, cyano, C₁₋₆ alkyl, C₂₋₄alkenyl, C₂₋₄alkynyl, C₁₋₄haloalkyl, or C₃₋₆cycloalkyl.

In some embodiments, R³ is each independently H, halogen, cyano, nitro, hydroxy, —NR⁷R¹⁰, —NHCOR⁹, C₁₋₆ alkyl, C₂₋₆ alkenyl, C₂₋₆ alkynyl, C₁₋₆ alkoxy, C₂₋₆ alkenoxy, C₂₋₆ alkynoxy, COR⁹, CON(R⁹)₂, CF₃, CF₂H, CF₃CF₂, CF₃CH₂, C₁₋₆haloalkyl, CF₃O, CF₂HO, CF₃CF₂O, CF₃CH₂O, C₁₋₆ haloalkoxy, C₃₋₈ cycloalkyl, C₃₋₈ cycloalkenyl, C₁₋₆ alkylthio, C₂₋₆ alkenylthio, or C₁₋₆ alkylsulfonyl, wherein R³ is each independently optionally substituted by 0-2 R¹¹. In some embodiments, R³ is each independently H, halogen, cyano, C₁₋₆alkyl, or C₁₋₆haloalkyl. In some embodiments, R³ is each independently H, halogen, cyano, C₁₋₄alkyl, or C₁₋₄haloalkyl. In some embodiments, R³ is each independently H, halogen, cyano, or C₁₋₂haloalkyl. In some embodiments, R³ is each independently H or halogen.

In some embodiments, R⁴ is each independently H, halogen, cyano, nitro, hydroxy, NR⁷R¹⁰, NHCOR⁹, NR⁷CONR⁷, C₁₋₆ alkyl, C₂₋₆ alkenyl, C₂₋₆ alkynyl, COR⁹, CO₂R⁹, CONR⁷R¹⁰, C₁₋₆ alkoxy, C₂₋₆ alkenoxy, C₂₋₆ alkynoxy, CF₃, CF₂H, CF₃CF₂, CF₃CH₂, C₁₋₆ haloalkyl, CF₃O, CF₂HO, CF₃CF₂O, CF₃CH₂O, C₁₋₆haloalkoxy, C₃₋₈ cycloalkyl, C₃₋₈ cycloalkenyl, C₁₋₆ alkylthio, C₂₋₆ alkenylthio, C₁₋₆ alkylsulfonyl, SO₂N(R⁷)₂, or phenyl, or any two occurrences of R⁴ on contiguous atoms, taken together with the contiguous atoms to which the two occurrences of R⁴ are attached, form a saturated or unsaturated 5-7 membered ring comprising 0-2 heteroatoms each independently selected from O, S(═O)₀₋₂, P(═O)R¹², N, and NR¹⁰, wherein R⁴ is each independently optionally substituted by 0-2 R¹¹. In some embodiments, R⁴ is each independently H, halogen, cyano, nitro, hydroxy, NR⁷R¹⁰, C₁₋₆alkyl, C₂₋₆alkenyl, C₂₋₆alkynyl, C₁₋₆alkoxy, C₂₋₆alkenoxy, C₂₋₆alkynoxy, C₁₋₆haloalkyl, C₁₋₆haloalkoxy, C₃₋₈cycloalkyl, C₃₋₈cycloalkenyl, C₁₋₆alkylsulfonyl, SO₂N(R⁷)₂, or phenyl, wherein R⁴ is each independently optionally substituted by 0-2 R¹¹. In some embodiments, R⁴ is each independently H, halogen, cyano, nitro, hydroxy, NR⁷R¹⁰, NHCOR⁹, NR⁷CONR⁷, C₁₋₆alkyl, C₂₋₆alkenyl, C₂₋₆alkynyl, COR⁹, CO₂R⁹, CONR⁷R¹⁰, C₁₋₆alkoxy, C₁₋₆haloalkyl, C₁₋₆haloalkoxy, or C₃₋₈cycloalkyl, wherein R⁴ is each independently optionally substituted by 0-1 R¹¹. In some embodiments, R⁴ is each independently H, halogen, cyano, nitro, C₁₋₆ alkyl optionally substituted with R¹¹, C₂₋₆alkenyl, C₂₋₆alkynyl, C₁₋₆haloalkyl, C₁₋₆alkoxy, C₃₋₆cycloalkyl, or CONR⁷R¹⁰.

In some embodiments, R⁹ is H, C₁₋₆ alkyl, C₁₋₆ haloalkyl, C₂₋₆ alkenyl, C₂₋₆ alkynyl, C₃₋₁₀ cycloalkyl, C₄₋₁₀ heterocycloalkyl, C₁₋₃ alkylene-C₃₋₆ cycloalkyl, C₁₋₃ alkylene-C₄₋₆ heterocycloalkyl, C₁₋₆ acyl, phenyl, or 5-12 membered heteroaryl, wherein R⁹ is optionally substituted by 1-2 R¹¹. In some embodiments, R⁹ is H, C₃₋₁₀ cycloalkyl, C₄₋₁₀ heterocycloalkyl, phenyl, or 5-12 membered heteroaryl, wherein R⁹ is optionally substituted by 1-2 R¹¹. In some embodiments, R⁹ is C₁₋₆alkyl optionally substituted by 1 R¹¹.

In some embodiments, R¹⁰ is H, C₁₋₆ alkyl, C₁₋₆ haloalkyl, C₂₋₆ alkenyl, C₂₋₆ alkynyl, C₃₋₁₀ cycloalkyl, C₄₋₁₀ heterocycloalkyl, C₁₋₃ alkylene-C₃₋₆ cycloalkyl, C₁₋₃ alkylene-C₄₋₆ heterocycloalkyl, C₁₋₆ acyl, phenyl, or 5-12 membered heteroaryl, wherein R¹⁰ is optionally substituted by 1-2 R¹¹. In some embodiments, R¹⁰ is H or C₁₋₆alkyl, wherein the R¹⁰ is optionally substituted by 1-2 R¹¹. In some embodiments, R¹⁰ is H or C₁₋₆alkyl, wherein the R¹⁰ is optionally substituted by 1 R¹¹. In some embodiments, R¹⁰ is H or C₁₋₆alkyl.

In some embodiments, R⁷ and R¹⁰ on the same N atom may be taken together with the N atom to which they are attached form a 4-7 membered ring comprising an additional 0-1 heteroatoms selected from NR⁵, O, S(═O)₀₋₂, P(═O)R¹², and NR¹³, wherein R¹⁰ is optionally substituted by 1-2 R¹¹. In some embodiments, R⁷ and R¹⁰ on the same N atom may be taken together with the N atom to which they are attached form a 4-7 membered ring comprising an additional 0-1 heteroatoms selected from NR⁵, O, S(═O)₀₋₂, P(═O)R¹², and NR¹³.

In some embodiments, R¹¹ is each independently R⁶, oxo, nitro, C₂₋₆ alkenyl, C₂₋₆ alkynyl, C₁₋₆ alkoxy, C₁₋₆ haloalkyl, C₁₋₆ haloalkoxy, C₃₋₆ cycloalkyl, 4-6 membered heterocycloalkyl, C₁₋₃ alkylene-C₃₋₆ cycloalkyl, C₁₋₃ alkylene-C₄₋₆ heterocycloalkyl, C₆₋₁₀ aryl, C₅₋₆ heteroaryl, SO₂R⁷, S(═O)NR⁵, SO₂N(R⁷)₂, CON(R⁷)₂, or N(R⁷)₂. In some embodiments, R¹¹ is each independently R⁶, nitro, C₂₋₆ alkenyl, C₂₋₆ alkynyl, C₁₋₆ alkoxy, C₁₋₆ haloalkyl, C₁₋₆ haloalkoxy, C₃₋₆ cycloalkyl, 4-6 membered heterocycloalkyl, C₁₋₃ alkylene-C₃₋₆ cycloalkyl, C₁₋₃ alkylene-C₄₋₆ heterocycloalkyl, C₆₋₁₀ aryl, C₅₋₆ heteroaryl, SO₂R⁷, S(═O)NR⁵, SO₂N(R⁷)₂, CON(R⁷)₂, or N(R⁷)₂. In some embodiments, wherein R¹¹ is each independently R⁶, C₁₋₆ alkoxy, C₁₋₆haloalkyl, C₃₋₆ cycloalkyl, 4-6 membered heterocycloalkyl, C₆₋₁₀ aryl, C₅₋₆ heteroaryl, SO₂R⁷, S(═O)NR⁵, SO₂N(R⁷)₂, CON(R⁷)₂, or N(R⁷)₂. In some embodiments, R¹¹ is each independently N(R⁷)₂.

In some embodiments, R¹² is each independently H, hydroxy, C₁₋₆alkyl, or C₁₋₆alkoxy. In some embodiments, In some embodiments, R¹² is each independently H or C₁₋₆alkyl.

In some embodiments, R¹³ is each independently H, C₁₋₆ alkyl, C₁₋₆ haloalkyl, C₂₋₆ alkenyl, C₂₋₆ alkynyl, C₂₋₆ cycloalkyl, C₄₋₆heterocycloalkyl, C₁₋₃ alkylene-C₃₋₆ cycloalkyl, or C₁₋₃ alkylene-C₄₋₆ heterocycloalkyl, C₁₋₆ acyl, C₆₋₁₀ aroyl, or C₅₋₆ heteroaroyl.

In some embodiments, m is an integer 0 or 1. In some embodiments, m is 0. In some embodiments, m is 1.

In some embodiments, n is an integer 0, 1, or 2. In some embodiments, n is an integer 0 or 1. In some embodiments, n is an integer 1 or 2. In In some embodiments, n is 0. In some embodiments, n is 1. In some embodiments, n is 2.

In some embodiments, p is an integer 0, 1, or 2. In some embodiments, p is an integer 1, 2, or 3. In some embodiments, p is an integer 0 or 1. In some embodiments, p is 0. In some embodiments, p is 1. In some embodiments, p is 2. In some embodiments, p is 3.

In some embodiments, the compound is a compound of Formula III-aa:

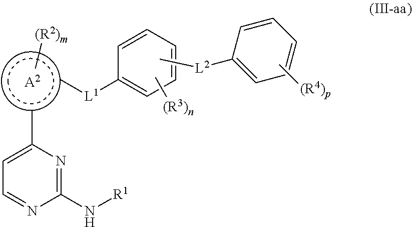

or a pharmaceutically acceptable salt thereof, wherein the variables are as defined for Formula III.

In some embodiments, the compound is a compound of Formula III-ba:

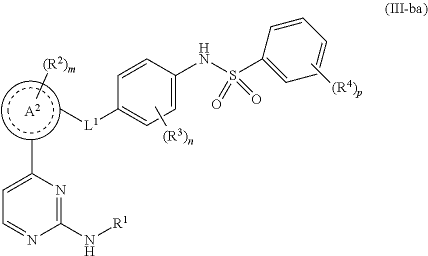

or a pharmaceutically acceptable salt thereof, wherein the variables are as defined for Formula III.

In some embodiments, the compound is a compound of Formula III-ab:

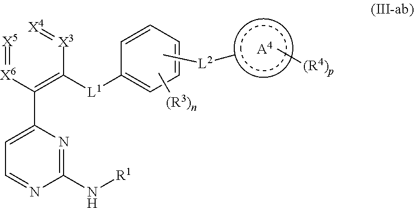

or a pharmaceutically acceptable salt thereof, wherein the variables are as defined for Formula III.

In some embodiments, the compound is a compound of Formula III-bb:

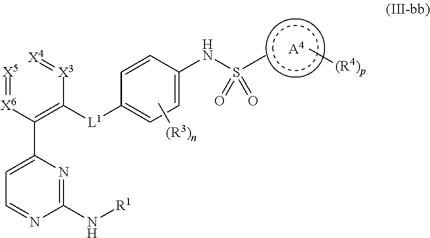

or a pharmaceutically acceptable salt thereof, wherein the variables are as defined for Formula III.

In some embodiments, the compound is a compound of Formula III-ac:

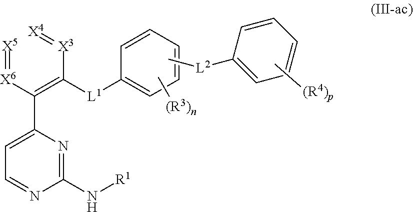

or a pharmaceutically acceptable salt thereof, wherein the variables are as defined for Formula III.

In some embodiments, the compound is a compound of Formula III-bc:

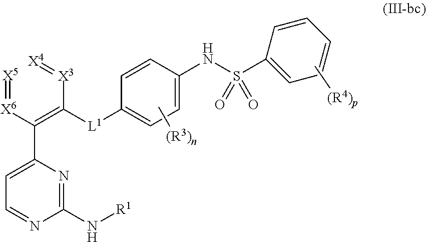

or a pharmaceutically acceptable salt thereof, wherein the variables are as defined for Formula III.

In some embodiments, the compound is a compound of Formula III-ad, III-ae, III-af, or III-ag:

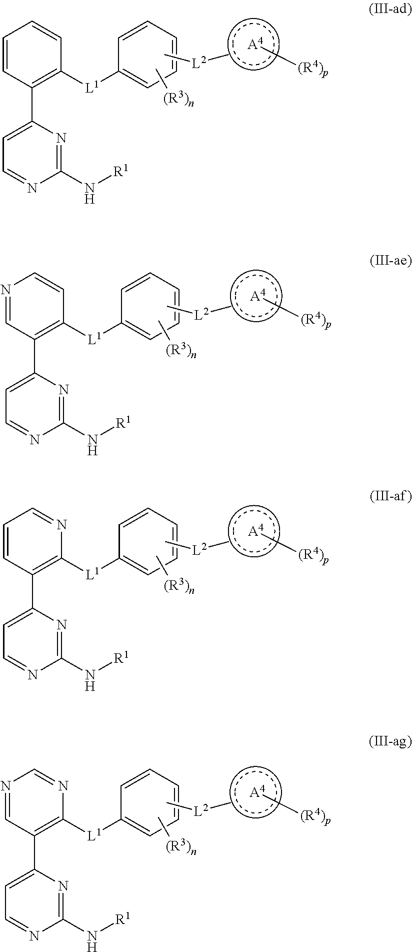

or a pharmaceutically acceptable salt thereof, wherein the variables are as defined for Formula III.

In some embodiments, the compound is a compound of Formula III-bd, III-be, III-bf, or III-bg:

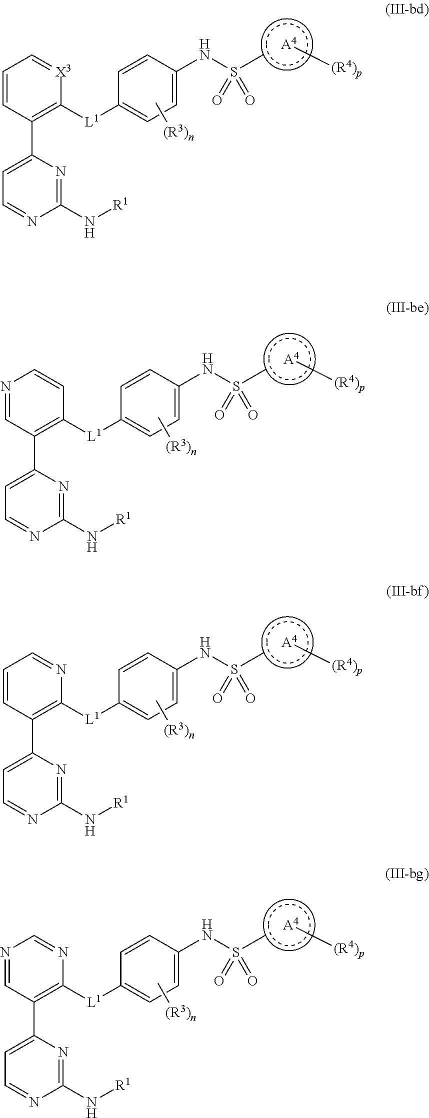

or a pharmaceutically acceptable salt thereof, wherein the variables are as defined for Formula III.

In some embodiments, the compound is a compound of Formula III-ah, III-ai, III-aj, or III-ak:

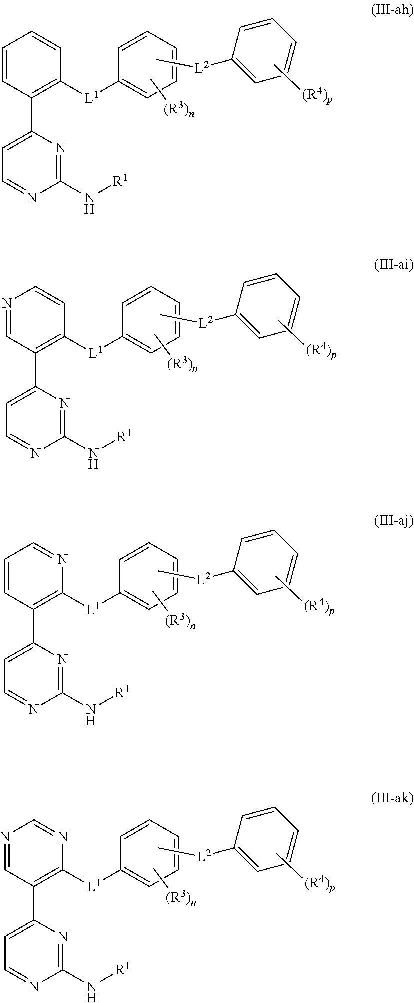

or a pharmaceutically acceptable salt thereof, wherein the variables are as defined for Formula III.

In some embodiments, the compound is a compound of Formula III-bh, III-bi, III-bj, or III-bk:

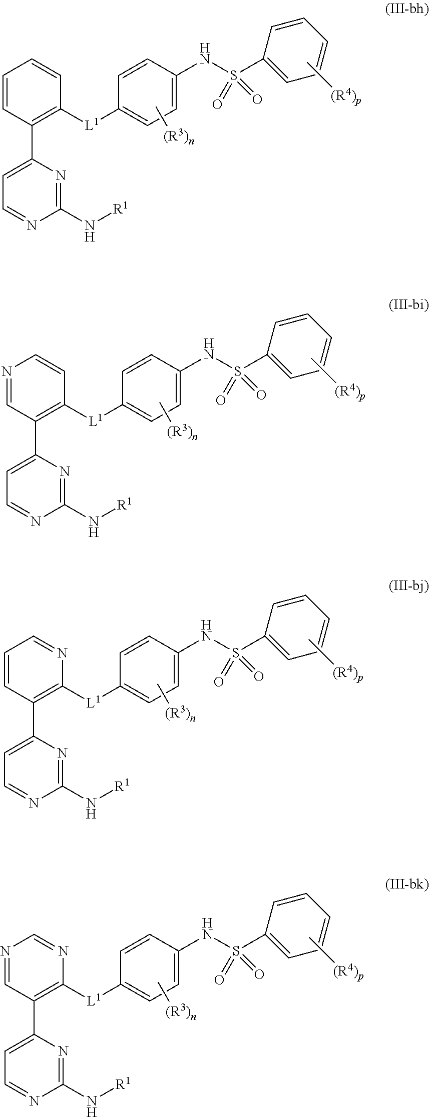

or a pharmaceutically acceptable salt thereof, wherein the variables are as defined for Formula III.

Also provided, in some embodiments, are compounds of Formula IV:

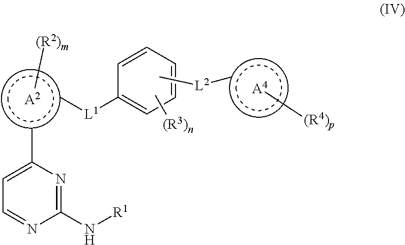

or a pharmaceutically acceptable salt thereof, wherein:
A² is

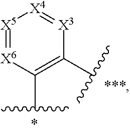

naphthyl, or 8-12-membered heteroaryl, wherein * is the site covalently linked to the pyrimidyl, and *** is the site covalently linked to L¹;
A⁴ is aryl, heteroaryl, or C₃₋₁₂carbocyclyl;
X³, X⁴, X⁵, and X⁶ are each independently N or CH, wherein the hydrogen of each CH is independently optionally substituted by R², or any two contiguous of X³, X⁴, X⁵, and X⁶ can be taken together as O, S, or NR^{N1},
provided that when X³ is N, X⁴ and X⁵ are both CH wherein the hydrogen of CH is each optionally and independently substituted by R², and X⁶ is N or CH, L¹ is not —O—; and when X³ is N, X⁵ and X⁶ are taken together as S, and X⁴ is CH wherein the hydrogen of CH is optionally substituted by R², L¹ is not —O;
L¹ is —O—, —NR^{N1}—, —S(═O)₀₋₂—, —S(═O)(═NR^{N1})—, —C(R⁶)₂—, or —C(═O)—;
L² is —NR^{N1}SO₂—, —SO₂NR^{N1}—, —C(R⁷)₂C(═O)—, —C(═O)C(R⁷)₂—, —C(═O)O—, —OC(═O)—, —C(R⁶)₂O—, —OC(R⁶)₂—, —C(═O)NR^{N1}—, —NR^{N1}C(═O)—, —C(R⁶)₂NR^{N1}—, —NR^{N1}C(R⁶)₂—, —C(R⁶)₂S(═O)₀₋₂—, —S(═O)₀₋₂C(R⁶)₂—, —C(R⁶)₂S(═O)NR^{N1}—, —S(═O)NR^{N1}C(R⁶)₂—, —C(R⁶)₂C(R⁷)₂—, or —C(R⁷)₂C(R⁶)₂—;
R¹ is C₃₋₁₀carbocyclyl, 4-12 membered heterocyclyl, phenyl, 5-12 membered heteroaryl, —(C₁₋₆alkylene)-(C₃₋₁₀carbocyclyl), —(C₁₋₆alkylene)-(4-12 membered heterocyclyl), —(C₁₋₆alkylene)-(phenyl), —(C₁₋₆alkylene)-(5-12 membered heteroaryl), —(C₁₋₆alkylene)-OR⁸, or —(C₁₋₆alkylene)-NR⁹R¹⁰, wherein R¹ is optionally substituted by 1-3 R¹²;
R², R³, and R⁴ are each independently halogen, cyano, nitro, oxo, hydroxy, amino, C₁₋₆alkyl, C₁₋₆alkoxy, C₁₋₆alkylamino, C₁₋₆dialkylamino, C₁₋₆haloalkyl, C₁₋₆haloalkoxy, C₃₋₁₀carbocyclyl, C₁₋₆alkylthio, or C₁₋₆alkylsulfonyl, or any two occurrences of R², R³, or R⁴ on contiguous atoms taken together with the contiguous atoms to which the two occurrences of R², R³, or R⁴ are attached form a 5-7 membered ring comprising 0-2 heteroatoms selected from O, S(═O)₀₋₂, P(═O)R^{P1}, N, and NR^{N1}, wherein R², R³, or R⁴ are each independently optionally substituted by 1-3 R¹²;
R⁶ is each independently H, cyano, C₁₋₆alkyl, or C₁₋₆haloalkyl, wherein R⁶ is optionally substituted by 1-3 R¹²;
R⁷ is each independently H, halogen, cyano, C₁₋₆alkyl, C₁₋₆haloalkyl, C₁₋₆alkoxy, or C₁₋₆haloalkoxy, or two occurrences of R⁷ taken together with the C atom to which they are attached form a 4-7 membered ring comprising 0-1 heteroatom selected from O, S(═O)₀₋₂, P(═O)R^{P1}, and NR^{N1}, wherein R⁷ is optionally substituted by 1-3 R¹²;
R⁸ is C₃₋₁₀carbocyclyl, 4-12 membered heterocyclyl, phenyl, or 5-12 membered heteroaryl, wherein R⁸ is optionally substituted by 1-3 R¹²;
R⁹ is H, C₁₋₆alkyl, C₃₋₁₀carbocyclyl, 4-12 membered heterocyclyl, phenyl, or 5-12 membered heteroaryl, wherein R⁹ is optionally substituted by 1-3 R¹²;
R¹⁰ is H or C₁₋₆alkyl;
or R⁹ and R¹⁰ taken together with the N atom to which they are attached form a 4-12 membered heterocyclyl optionally substituted by 1-3 R¹²;
R¹¹ is each independently C₁₋₆alkyl, C₁₋₆haloalkyl, C₃₋₁₀carbocyclyl, or 4-12 membered heterocyclyl;
R¹² is each independently halogen, cyano, oxo, hydroxy, C₁₋₆alkyl, C₂₋₆alkenyl, C₂₋₆alkynyl, C₁₋₆alkoxy, C₁₋₆haloalkyl, C₁₋₆haloalkoxy, SO₂R¹¹, S(═O)NR^{N1}R¹¹, or NR^{N1}R^{N2};
R^{P1} is each independently H or C₁₋₆alkyl;
R^{N1} is each independently H or C₁₋₆alkyl;
R^{N2} is each independently H, C₁₋₆alkyl, SO₂R¹¹, or S(═O)NR^{N1}R¹¹;
m is an integer 0, 1, 2, or 3;
n is an integer 0, 1, 2, or 3; and
p is an integer 0, 1, 2, or 3,
provided that L¹ and L² are not situated on contiguous atoms of the phenyl.

In some embodiments, A⁴ is phenyl, naphthyl, or 5-12 membered heteroaryl. In some embodiments, A⁴ is phenyl, naphthyl, 5-6 membered monocyclic heteroaryl, or 8-12 membered bicyclic heteroaryl. In some embodiments, A⁴ is phenyl, pyridyl, pyridazinyl, pyrimidyl, pyrazinyl, triazinyl, thienyl, thiazolyl, thiadiazolyl, oxazolyl, oxadiazolyl, pyrazolyl, imidazolyl, triazolyl, or tetrazolyl.

In some embodiments, A² is phenyl.

In some embodiments, A² is

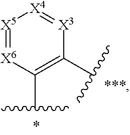

and at least one of X³, X⁴, X⁵, and X⁶ is N.

In some embodiments, when X⁴ and X⁵ are both CH wherein the hydrogen of CH is each optionally and independently substituted by R² and X⁶ is N or CH, X³ is not N. In some embodiments, when X⁵ and X⁶ are taken together as S and X⁴ is CH wherein the hydrogen of CH is optionally substituted by R², X³ is not N.

In some embodiments, A² is 1,2-linked phenylene, 2-L¹-3-biaryl linked pyridylene or 4-L¹-3-biaryl linked pyridylene.

In some embodiments, R¹ is C₃₋₁₀carbocyclyl, 4-12 membered heterocyclyl, phenyl, or 5-12 membered heteroaryl, wherein R¹ is optionally substituted by 1-3 R¹². In some embodiments, R¹ is C₃₋₁₀carbocyclyl or 4-12 membered heterocyclyl, wherein R¹ is optionally substituted by 1-3 R¹².

In some embodiments, L¹ is —O—, —NR^{N1}—, S(═O)₀₋₂, —S(═O)(═NR^{N1})—, —CHR⁶—, or —CF₂—. In some embodiments, L¹ is —O—, —S(═O)₀₋₂—, or —C(R⁶)₂—. In some embodiments, L¹ is —O—. In some embodiments, L¹ is —S—. In some embodiments, L¹ is —NH—.

In some embodiments, L² is NHSO₂—, —SO₂NH—, —C(R⁷)₂C(═O)—, —C(═O)C(R⁷)₂—, —C(═O)O—, —OC(═O)—, —C(R⁶)₂O—, —OC(R⁶)₂—, —C(═O)NH—, —NHC(═O)—, —C(R⁶)₂NH—, —NHC(R⁶)₂—, —C(R⁶)₂S(═O)₀₋₂—, —S(═O)₀₋₂C(R⁶)₂—, —C(R⁶)₂S(═O)NH—, —S(═O)NHC(R⁶)₂—, —C(R⁶)₂C(R⁷)₂—, or —C(R⁷)₂C(R⁶)₂—. In some embodiments, L² is —NR^{N1}SO₂—.

In some embodiments, R⁶ is each independently H, methyl, ethyl, fluoro, chloro, cyano, CF₃, CHF₂, or CH₂F.

In some embodiments, R⁷ is each independently H, methyl, ethyl, fluoro, chloro, cyano, CF₃, CHF₂, CH₂F, methoxy, or ethoxy.

In some embodiments, m is an integer 0, 1, or 2.

In some embodiments, n is an integer 0, 1, or 2.

In some embodiments, p is an integer 0, 1, or 2.

Also provided herein, in some embodiments, are compounds of Formula V:

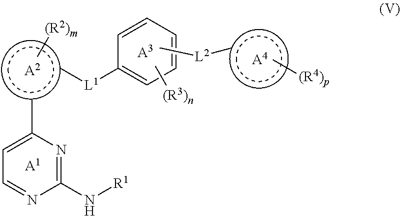

or a pharmaceutically acceptable salt thereof, wherein:
A² is phenyl, 2,3-naphthyl, or monocyclic heteroaryl;
A⁴ is aryl, heteroaryl, or C₃₋₁₂carbocyclyl;
L¹ is —S—, —NH—, —S(═O)₁₋₂—, —S(═O)(═NR⁵)—, —C(R⁶)₂—, or —C(═O)—;
L² is —NR⁷SO₂—, —C(R⁸)₂SO₂—, —NR⁷SO₂—C(R⁸)₂—, —C(R⁸)₂C(═O)—, —C(═O)C(R⁸)₂—, —C(═O)O—, —OC(═O)—, —C(R⁸)₂O—, —OC(R⁸)₂—, —C(═O)NR⁷—, —NR⁷C(═O)—, —C(R⁸)₂NR⁷—, —NR⁷C(R⁸)₂—, —C(R⁸)₂S(═O)₀₋₂—, —S(═O)₀₋₂C(R⁸)₂—, —C(R⁸)₂S(═O)NR⁷—, S(═O)NR⁷C(R⁸)₂—, or —C(R⁸)₂C(R⁸)₂—;
R¹ is C₃₋₁₂ carbocyclyl, 4-12 membered heterocyclyl, phenyl, 5-12 membered heteroaryl, —(C₁₋₆ alkylene)-(C₃₋₁₀ carbocyclyl), —(C₁₋₆ alkylene)-(4-12 membered heterocyclyl), —(C₁₋₆alkylene)-(phenyl), —(C₁₋₆alkylene)-(5-12 membered heteroaryl), —(C₁₋₆alkylene)-OR⁹, or —(C₁₋₆alkylene)-NR⁷R¹⁰, wherein R¹ is optionally substituted by 0-5 R¹¹;
R² is each independently H, halogen, cyano, nitro, hydroxy, —NR⁷R¹⁰, —NHCOR⁹, C₁₋₆ alkyl, C₂₋₆ alkenyl, C₂₋₆ alkynyl, C₁₋₆ alkoxy, C₂₋₆ alkenoxy, C₂₋₆ alkynoxy, COR⁹, CON(R⁹)₂, CF₃, CF₂H, CF₃CF₂, CF₃CH₂, C₁₋₆haloalkyl, CF₃O, CF₂HO, CF₃CF₂O, CF₃CH₂O, C₁₋₆ haloalkoxy, C₃₋₈ cycloalkyl, C₃₋₈ cycloalkenyl, C₁₋₆ alkylthio, C₂₋₆ alkenylthio, C₁₋₆ alkylsulfonyl, aryl, or heteroaryl, wherein R² is each independently optionally substituted by 0-4 R¹¹;
R³ is each independently H, halogen, cyano, nitro, hydroxy, —NR⁷R¹⁰, —NHCOR⁹, C₁₋₆ alkyl, C₂₋₆ alkenyl, C₂₋₆ alkynyl, C₁₋₆ alkoxy, C₂₋₆ alkenoxy, C₂₋₆ alkynoxy, COR⁹, CON(R⁹)₂, CF₃, CF₂H, CF₃CF₂, CF₃CH₂, C₁₋₆ haloalkyl, CF₃O, CF₂HO, CF₃CF₂O, CF₃CH₂O, C₁₋₆ haloalkoxy, C₃₋₈ cycloalkyl, C₃₋₈ cycloalkenyl, C₁₋₆ alkylthio, C₂₋₆ alkenylthio, or C₁₋₆ alkylsulfonyl, wherein R³ is each independently optionally substituted by 0-4 R¹¹;
R⁴ is each independently H, halogen, cyano, nitro, hydroxy, NR⁷R¹⁰, NHCOR⁹, NR⁷CONR⁷, C₁₋₆ alkyl, C₂₋₆ alkenyl, C₂₋₆ alkynyl, COR⁹, CO₂R⁹, CONR⁷R¹⁰, C₁₋₆ alkoxy, C₂₋₆ alkenoxy, C₂₋₆ alkynoxy, CF₃, CF₂H, CF₃CF₂, CF₃CH₂, C₁₋₆ haloalkyl, CF₃O, CF₂HO, CF₃CF₂O, CF₃CH₂O, C₁₋₆ haloalkoxy, C₃₋₈ cycloalkyl, C₃₋₈ cycloalkenyl, C₁₋₆ alkylthio, C₂₋₆ alkenylthio, C₁₋₆ alkylsulfonyl, SO₂N(R⁷)₂, or phenyl, or any two occurrences of R⁴ on contiguous atoms, taken together with the contiguous atoms to which the two occurrences of R⁴ are attached, form a saturated or unsaturated 5-7 membered ring comprising 0-2 heteroatoms each independently selected from O, S(═O)₀₋₂, P(═O)R¹², N, and NR¹⁰, wherein R⁴ is each independently optionally substituted by 0-4 R¹¹;
R⁵ is each independently H or C₁₋₄ alkyl;
R⁶ is each independently H, F, OH, cyano, or C₁₋₆ alkyl;
R⁷ is each independently H, C₁₋₆ alkyl, C₁₋₆ haloalkyl, C₂₋₆ alkenyl, C₂₋₆ alkynyl, C₃₋₆ cycloalkyl, C₄₋₆ heterocycloalkyl, aryl, 5-10 membered heteroaryl, C₁₋₃ alkylene-C₃₋₆ cycloalkyl, or C₁₋₃ alkylene-C₄₋₆ heterocycloalkyl, or two R⁷s along with the N atom to which they are attached form a 4-7 membered ring comprising an additional 0-1 heteroatoms selected from NR⁵, O and S(═O)₀₋₂;
R⁸ is each independently H, halogen, cyano, C₁₋₆ alkyl, C₁₋₆ haloalkyl, C₂₋₆ alkenyl, C₂₋₆ alkynyl, C₂₋₆ cycloalkyl, C₄₋₆ heterocycloalkyl, C₁₋₃ alkylene-C₃₋₆ cycloalkyl, C₁₋₃ alkylene-C₄₋₆ heterocycloalkyl, or two R⁸s taken together with the C atom to which they are attached form a 4-7 membered ring comprising 0-1 heteroatom selected from O, S(═O)₀₋₂, P(═O)R¹², and NR⁵, wherein R⁸ is optionally substituted by 1-3 R¹¹;
each R⁹ is H, C₁₋₆ alkyl, C₁₋₆ haloalkyl, C₂₋₆ alkenyl, C₂₋₆ alkynyl, C₃₋₁₀ cycloalkyl, C₄₋₁₀ heterocycloalkyl, C₁₋₃ alkylene-C₃₋₆ cycloalkyl, C₁₋₃ alkylene-C₄₋₆ heterocycloalkyl, C₁₋₆ acyl, phenyl, or 5-12 membered heteroaryl, wherein R⁹ is optionally substituted by 1-3 R¹¹;
each R¹⁰ is independently H, C₁₋₆ alkyl, C₁₋₆ haloalkyl, C₂₋₆ alkenyl, C₂₋₆ alkynyl, C₃₋₁₀ cycloalkyl, C₄₋₁₀ heterocycloalkyl, C₁₋₃ alkylene-C₃₋₆ cycloalkyl, C₁₋₃ alkylene-C₄₋₆ heterocycloalkyl, C₁₋₆ acyl, C₁₋₆ alkylsulfonyl, phenyl, or 5-12 membered heteroaryl, wherein R¹⁰ is optionally substituted by 1-3 R¹¹,
or R⁷ and R¹⁰ on the same N atom may be taken together with the N atom to which they are attached form a 4-7 membered ring comprising an additional 0-1 heteroatoms selected from NR⁵, O, S(═O)₀₋₂, P(═O)R¹², and NR¹³;
R¹¹ is each independently R⁶, oxo, nitro, C₂₋₆ alkenyl, C₂₋₆ alkynyl, C₁₋₆ alkoxy, C₁₋₆ alkoxyalkyl, C₁₋₆hydroxyalkyl, C₁₋₆ haloalkyl, C₁₋₆ haloalkoxy, C₃₋₆ cycloalkyl, 4-6 membered heterocycloalkyl, C₁₋₃ alkylene-C₃₋₆ cycloalkyl, C₁₋₃ alkylene-C₄₋₆ heterocycloalkyl, C₆₋₁₀ aryl, C₅₋₁₀ heteroaryl, SO₂R⁷, S(═O)NR⁵, SO₂N(R⁷)₂, CON(R⁷)₂, or N(R⁷)₂;
R¹² is each independently H, OH, C₁₋₆ alkyl, C₁₋₆ alkoxy, or —N(R⁷)₂;
R¹³ is each independently H, C₁₋₆ alkyl, C₁₋₆ haloalkyl, C₂₋₆ alkenyl, C₂₋₆ alkynyl, C₂₋₆ cycloalkyl, C₄₋₆ heterocycloalkyl, C₁₋₃ alkylene-C₃₋₆ cycloalkyl, or C₁₋₃ alkylene-C₄₋₆ heterocycloalkyl, C₁₋₆ acyl, aroyl, or heteroaroyl;
m is an integer 0, 1 or 2;
n is an integer 0, 1, 2, or 3; and
p is an integer 0, 1, 2, or 3,
provided that the A¹ ring and the L¹ linker are on contiguous C atoms of A², whereas L¹ and L² are not situated on contiguous atoms of the A³ ring.

In some embodiments, A⁴ is phenyl, naphthyl, 5-12 membered heteroaryl, or C₃₋₁₀ carbocyclyl. In some embodiments, A⁴ is phenyl, naphthyl, 5-10 membered heteroaryl, or C₃₋₁₀ carbocyclyl. In some embodiments, A⁴ is phenyl, naphthyl, 5-10 membered heteroaryl, or C₃₋₆ carbocyclyl. In some embodiments, A⁴ is phenyl, naphthyl, or 5-12 membered heteroaryl. In some embodiments, A⁴ is phenyl, naphthyl, or 5-10 membered heteroaryl. In some embodiments, A⁴ is phenyl, naphthyl, 5-6 membered monocyclic heteroaryl, or 8-12 membered bicyclic heteroaryl. In some embodiments, A⁴ is phenyl, pyridyl, pyridazinyl, pyrimidyl, pyrazinyl, triazinyl, thienyl, thiazolyl, thiadiazolyl, oxazolyl, oxadiazolyl, pyrazolyl, imidazolyl, triazolyl, or tetrazolyl. In some embodiments, A⁴ is phenyl, naphthyl, thiophenyl, cyclopropyl, cyclobutyl, cyclopentyl, cyclohexyl,

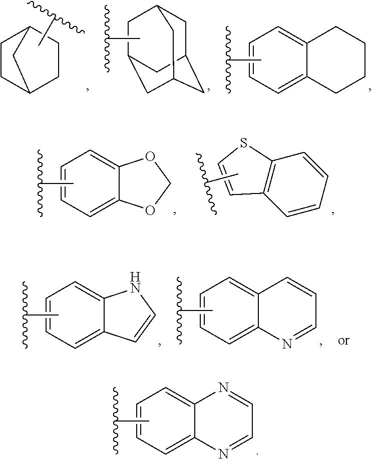

In some embodiments, A⁴ is phenyl.

In some embodiments, A² is phenyl, 2,3-naphthyl, pyridinyl, or pyrimidinyl. In some embodiments, A² is phenyl. In some embodiments, A² is 2,3-naphthyl. In some embodiments, A² is 5-6 membered heteroaryl. In some embodiments, A² is 1,2-linked phenylene, 2,3-linked-naphthylene, 2-L¹-3-biaryl linked pyridylene, 4-L¹-3-biaryl linked pyridylene or 4-L¹-5-biaryl linked pyrimidylene. In some embodiments, A² is 1,2-linked phenylene, 2-L¹-3-biaryl linked pyridylene or 4-L¹-3-biaryl linked pyridylene. In some embodiments, A² is phenyl, 2,3-naphthyl,

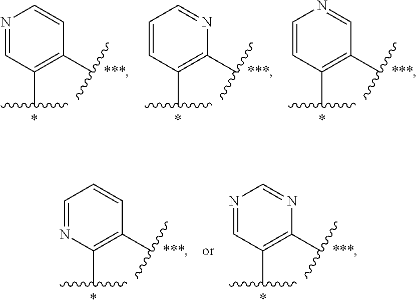 [SMILES: *c1ccncc1* ; *c1cccnc1* ; *c1ccncc1* ; *c1cccnc1* ; *c1cncnc1*]

wherein * is the site covalently linked to the A¹ ring, and *** is the site covalently linked to L¹.

In some embodiments, R¹ is C₃₋₁₀carbocyclyl, 4-12 membered heterocyclyl, phenyl, 5-12 membered heteroaryl, —(C₁₋₆alkylene)-(C₃₋₁₀carbocyclyl), —(C₁₋₆alkylene)-(4-12 membered heterocyclyl), —(C₁₋₆alkylene)-(phenyl), —(C₁₋₆alkylene)-(5-12 membered heteroaryl), —(C₁₋₆alkylene)-OR⁹, or —(C₁₋₆alkylene)-NR⁷R¹⁰, wherein R¹ is optionally substituted by 1-2 R¹¹. In some embodiments, R¹ is C₆₋₁₀carbocyclyl, 4-8 membered heterocyclyl, phenyl, 5-6 membered heteroaryl, —(C₁₋₆alkylene)-(C₃₋₁₀carbocyclyl), —(C₁₋₆ alkylene)-(4-8 membered heterocyclyl), —(C₁₋₆alkylene)-(5-6 membered heteroaryl), —(C₁₋₆alkylene)-OR⁹, or —(C₁₋₆alkylene)-NR⁷R¹⁰, wherein R¹ is optionally substituted by 1-2 R¹¹. In some embodiments, R¹ is C₆₋₁₀carbocyclyl, 4-8 membered heterocyclyl, phenyl, 5-6 membered heteroaryl, —(C₁₋₆alkylene)-(C₃₋₁₀carbocyclyl), —(C₁₋₆alkylene)-(4-8 membered heterocyclyl), —(C₁₋₆alkylene)-(5-6 membered heteroaryl), —(C₁₋₆alkylene)-OR⁹, or —(C₁₋₆alkylene)-NR⁷R¹⁰, wherein R¹ is optionally substituted by 1 R¹¹. In some embodiments, R¹ is C₆₋₁₀carbocyclyl, 4-8 membered heterocyclyl containing 1-2 heteroatoms selected from the group consisting of O, S, and N, —(C₁₋₆alkylene)-(C₃₋₁₀carbocyclyl), —(C₁₋₆alkylene)-(4-8 membered heterocyclyl containing 1-2 heteroatoms selected from the group consisting of O, S, and N), —(C₁₋₆alkylene)-(5-6 membered heteroaryl containing 1-2 heteroatoms selected from the group consisting of O, S, and N), —(C₁₋₆alkylene)-OR⁹, or —(C₁₋₆alkylene)-NR⁷R¹⁰, wherein R¹ is optionally substituted by 1 R¹¹. In some embodiments, R¹ is C₆₋₁₀carbocyclyl, 4-8 membered heterocyclyl containing 1-2 heteroatoms selected from the group consisting of O, S, and N, —(C₁₋₃alkylene)-(4-6 membered heterocyclyl containing 1-2 heteroatoms selected from the group consisting of O, S, and N), —(C₁₋₃alkylene)-(5-6 membered heteroaryl containing 1-2 heteroatoms selected from the group consisting of O, S, and N), —(C₁₋₆alkylene)-OR⁹, or —(C₁₋₆alkylene)-NR⁷R¹⁰, wherein R¹ is optionally substituted by 1 R¹¹.

In some embodiments, R¹ is cyclohexyl, piperidinyl, oxetanyl, azetidinyl, pyrrolidinyl, or azaspiroheptanyl, wherein R¹ is optionally substituted with 1 R¹¹.

In some embodiments, R¹ is cyclohexyl optionally substituted with 1 R¹¹.

In some embodiments, R¹ is piperidinyl, oxetanyl, azetidinyl, pyrrolidinyl, or azaspiroheptanyl, wherein R¹ is optionally substituted with 1 R¹¹. In some embodiments, R¹ is piperidinyl optionally substituted with 1 R¹¹.

In some embodiments, R¹ is —(C₁₋₃alkylene)-OR⁹ wherein R⁹ is C₁₋₆alkyl substituted by 1-2 R¹¹. In some embodiments, R¹ is —(C₁₋₃alkylene)-OR⁹ wherein R⁹ is C₁₋₆ alkyl substituted by 1 R¹¹. In some embodiments, R¹¹ is hydroxy or C₁₋₄alkoxy. In some embodiments, R¹¹ is hydroxy or C₁₋₂alkoxy.

In some embodiments, R¹ is —(C₁₋₃alkylene)-NR⁷R¹⁰ wherein R⁹ is H or C₁₋₆alkyl and R¹⁰ is H or C₁₋₆alkyl, wherein the R¹⁰ is optionally substituted by 1-2 R¹¹. In some embodiments, R¹ is —(C₁₋₃alkylene)-NR⁷R¹⁰ wherein R⁹ is H or C₁₋₆alkyl and R¹⁰ is H or C₁₋₆alkyl, wherein the R¹⁰ is optionally substituted by 1 R¹¹. In some embodiments, R¹¹ is hydroxy or C₁₋₄alkoxy. In some embodiments, R¹¹ is hydroxy or C₁₋₂alkoxy.

In some embodiments, R¹ is —(C₁₋₃alkylene)-(4-6 membered heterocyclyl containing 1-2 heteroatoms selected from the group consisting of O, S, and N) optionally substituted by 1 R¹¹.

In some embodiments, R¹ is —(C₁₋₃alkylene)-(5-6 membered heteroaryl containing 1-2 heteroatoms selected from the group consisting of O, S, and N) optionally substituted by 1 R¹¹.

In some embodiments, L¹ is —S—, —NH—, —S(═O)₁₋₂—, —S(═O)(═NR⁵)—, —CHR⁶—, or —CF₂—. In some embodiments, L¹ is —S—, —NH—, —S(═O)₁₋₂—, or —C(R⁶)₂—. In some embodiments L¹ is —S—.

In some embodiments, L² is —NR⁷SO₂—, —C(R⁸)₂SO₂—, —NR⁷SO₂—C(R⁸)₂—, —C(R⁸)₂C(═O)—, —C(═O)C(R⁸)₂—, —C(═O)O—, —OC(═O)—, —C(R⁸)₂O—, —OC(R⁸)₂—, or —C(═O)NR⁷—. In some embodiments, L² is —NR⁷SO₂—. In some embodiments, L² is —NHSO₂—, —NHSO₂—CHR⁸—, —C(═O)NH—, or —NHC(═O)—. In some embodiments, L² is —NHSO₂—, —NHSO₂—CH₂—, —C(═O)NH—, or —NHC(═O)—. In some embodiments L² is —NHSO₂—.

In some embodiments, R⁵ is each independently H or C₁₋₂alkyl. In some embodiments, R⁵ is H or methyl.

In some embodiments, R⁶ is each independently H, methyl, ethyl, fluoro, chloro, cyano, CF₃, CHF₂, or CH₂F.

In some embodiments, R⁷ is each independently H, C₁₋₆ alkyl, C₁₋₆ haloalkyl, C₂₋₆ alkenyl, C₂₋₆ alkynyl, C₂₋₆ cycloalkyl, C₄₋₆heterocycloalkyl, aryl, 5-10 membered heteroaryl, C₁₋₃ alkylene-C₃₋₆ cycloalkyl, or C₁₋₃ alkylene-C₄₋₆ heterocycloalkyl. In some embodiments, R⁷ is each independently H, C₁₋₆ alkyl, C₃₋₆ cycloalkyl, C₄₋₆ heterocycloalkyl, aryl, or 5-10 membered heteroaryl. In some embodiments, R⁷ is H or C₁₋₆alkyl. In some embodiments, two R⁷s, along with the N atom to which they are attached form a 4-7 membered ring comprising an additional 0-1 heteroatoms selected from NR⁵, O and S(═O)₀₋₂.

In some embodiments, R⁸ is each independently H, halogen, cyano, C₁₋₆ alkyl, C₁₋₆ haloalkyl, C₂₋₆ alkenyl, C₂₋₆ alkynyl, C₃₋₆ cycloalkyl, C₄₋₆ heterocycloalkyl, C₁₋₃ alkylene-C₃₋₆ cycloalkyl, C₁₋₃ alkylene-C₄₋₆ heterocycloalkyl. In some embodiments, two R⁸s taken together with the C atom to which they are attached form a 4-7 membered ring comprising 0-1 heteroatom selected from O, S(═O)₀₋₂, P(═O)R¹², and NR⁵. In some embodiments, R⁸ is each independently H, methyl, ethyl, fluoro, chloro, cyano, CF₃, CHF₂, CH₂F, methoxy, or ethoxy.

In some embodiments, R² is each independently H, halogen, cyano, nitro, hydroxy, —NR⁷R¹⁰, —NHCOR⁹, C₁₋₆ alkyl, C₂₋₆ alkenyl, C₂₋₆ alkynyl, C₁₋₆ alkoxy, C₂₋₆ alkenoxy, C₂₋₆ alkynoxy, COR⁹, CON(R⁹)₂, CF₃, CF₂H, CF₃CF₂, CF₃CH₂, C₁₋₆haloalkyl, CF₃O, CF₂HO, CF₃CF₂O, CF₃CH₂O, C₁₋₆haloalkoxy, C₃₋₈ cycloalkyl, C₃₋₈ cycloalkenyl, C₁₋₆ alkylthio, C₂₋₆ alkenylthio, C₁₋₆ alkylsulfonyl, aryl, or heteroaryl, wherein R² is each independently optionally substituted by 0-2 R¹¹. In some embodiments, R² is each independently H, halogen, cyano, C₁₋₆ alkyl, C₂₋₆ alkenyl, C₂₋₆ alkynyl, C₁₋₆haloalkyl, or C₃₋₆cycloalkyl. In some embodiments, R² is each independently H, halogen, cyano, C₁₋₆ alkyl, C₂₋₄alkenyl, C₂₋₄alkynyl, C₁₋₄haloalkyl, or C₃₋₆cycloalkyl.

In some embodiments, R³ is each independently H, halogen, cyano, nitro, hydroxy, —NR⁷R¹⁰, —NHCOR⁹, C₁₋₆ alkyl, C₂₋₆ alkenyl, C₂₋₆ alkynyl, C₁₋₆ alkoxy, C₂₋₆ alkenoxy, C₂₋₆ alkynoxy, COR⁹, CON(R⁹)₂, CF₃, CF₂H, CF₃CF₂, CF₃CH₂, C₁₋₆haloalkyl, CF₃O, CF₂HO, CF₃CF₂O, CF₃CH₂O, C₁₋₆ haloalkoxy, C₃₋₈ cycloalkyl, C₃₋₈ cycloalkenyl, C₁₋₆ alkylthio, C₂₋₆ alkenylthio, or C₁₋₆ alkylsulfonyl, wherein R³ is each independently optionally substituted by 0-2 R¹¹. In some embodiments, R³ is each independently H, halogen, cyano, C₁₋₆alkyl, or C₁₋₆haloalkyl. In some embodiments, R³ is each independently H, halogen, cyano, C₁₋₄alkyl, or C₁₋₄haloalkyl. In some embodiments, R³ is each independently H, halogen, cyano, or C₁₋₂haloalkyl. In some embodiments, R³ is each independently H or halogen.

In some embodiments, R⁴ is each independently H, halogen, cyano, nitro, hydroxy, NR⁷R¹⁰, NHCOR⁹, NR⁷CONR⁷, C₁₋₆ alkyl, C₂₋₆ alkenyl, C₂₋₆ alkynyl, COR⁹, CO₂R⁹, CONR⁷R¹⁰, C₁₋₆ alkoxy, C₂₋₆ alkenoxy, C₂₋₆ alkynoxy, CF₃, CF₂H, CF₃CF₂, CF₃CH₂, C₁₋₆ haloalkyl, CF₃O, CF₂HO, CF₃CF₂O, CF₃CH₂O, C₁₋₆haloalkoxy, C₃₋₈ cycloalkyl, C₃₋₈ cycloalkenyl, C₁₋₆ alkylthio, C₂₋₆ alkenylthio, C₁₋₆ alkylsulfonyl, SO₂N(R⁷)₂, or phenyl, or any two occurrences of R⁴ on contiguous atoms, taken together with the contiguous atoms to which the two occurrences of R⁴ are attached, form a saturated or unsaturated 5-7 membered ring comprising 0-2 heteroatoms each independently selected from O, S(═O)₀₋₂, P(═O)R¹², N, and NR¹⁰, wherein R⁴ is each independently optionally substituted by 0-2 R¹¹. In some embodiments, R⁴ is each independently H, halogen, cyano, nitro, hydroxy, NR⁷R¹⁰, C₁₋₆alkyl, C₂₋₆alkenyl, C₂₋₆alkynyl, C₁₋₆alkoxy, C₂₋₆alkenoxy, C₂₋₆alkynoxy, C₁₋₆haloalkyl, C₁₋₆haloalkoxy, C₃₋₈cycloalkyl, C₃₋₈cycloalkenyl, C₁₋₆alkylsulfonyl, SO₂N(R⁷)₂, or phenyl, wherein R⁴ is each independently optionally substituted by 0-2 R¹¹. In some embodiments, R⁴ is each independently H, halogen, cyano, nitro, hydroxy, NR⁷R¹⁰, NHCOR⁹, NR⁷CONR⁷, C₁₋₆alkyl, C₂₋₆alkenyl, C₂₋₆alkynyl, COR⁹, CO₂R⁹, CONR⁷R¹⁰, C₁₋₆alkoxy, C₁₋₆haloalkyl, C₁₋₆haloalkoxy, or C₃₋₈cycloalkyl, wherein R⁴ is each independently optionally substituted by 0-1 R¹¹. In some embodiments, R⁴ is each independently H, halogen, cyano, nitro, C₁₋₆ alkyl optionally substituted with R¹¹, C₂₋₆alkenyl, C₂₋₆alkynyl, C₁₋₆haloalkyl, C₁₋₆alkoxy, C₃₋₆cycloalkyl, or CONR⁷R¹⁰.

In some embodiments, R⁹ is H, C₁₋₆ alkyl, C₁₋₆ haloalkyl, C₂₋₆ alkenyl, C₂₋₆ alkynyl, C₃₋₁₀ cycloalkyl, C₄₋₁₀ heterocycloalkyl, C₁₋₃ alkylene-C₃₋₆ cycloalkyl, C₁₋₃ alkylene-C₄₋₆ heterocycloalkyl, C₁₋₆ acyl, phenyl, or 5-12 membered heteroaryl, wherein R⁹ is optionally substituted by 1-2 R¹¹. In some embodiments, R⁹ is H, C₃₋₁₀ cycloalkyl, C₄₋₁₀ heterocycloalkyl, phenyl, or 5-12 membered heteroaryl, wherein R⁹ is optionally substituted by 1-2 R¹¹. In some embodiments, R⁹ is C₁₋₆alkyl optionally substituted by 1 R¹¹.

In some embodiments, R¹⁰ is H, C₁₋₆ alkyl, C₁₋₆ haloalkyl, C₂₋₆ alkenyl, C₂₋₆ alkynyl, C₃₋₁₀ cycloalkyl, C₄₋₁₀ heterocycloalkyl, C₁₋₃ alkylene-C₃₋₆ cycloalkyl, C₁₋₃ alkylene-C₄₋₆ heterocycloalkyl, C₁₋₆ acyl, phenyl, or 5-12 membered heteroaryl, wherein R¹⁰ is optionally substituted by 1-2 R¹¹. In some embodiments, R¹⁰ is H or C₁₋₆alkyl, wherein the R¹⁰ is optionally substituted by 1-2 R¹¹. In some embodiments, R¹⁰ is H or C₁₋₆alkyl, wherein the R¹⁰ is optionally substituted by 1 R¹¹. In some embodiments, R¹⁰ is H or C₁₋₆alkyl.

In some embodiments, R⁷ and R¹⁰ on the same N atom may be taken together with the N atom to which they are attached form a 4-7 membered ring comprising an additional 0-1 heteroatoms selected from NR⁵, O, S(═O)₀₋₂, P(═O)R¹², and NR¹³, wherein R¹⁰ is optionally substituted by 1-2 R¹¹. In some embodiments, R⁷ and R¹⁰ on the same N atom may be taken together with the N atom to which they are attached form a 4-7 membered ring comprising an additional 0-1 heteroatoms selected from NR⁵, O, S(═O)₀₋₂, P(═O)R¹², and NR¹³.

In some embodiments, R¹¹ is each independently R⁶, oxo, nitro, C₂₋₆ alkenyl, C₂₋₆ alkynyl, C₁₋₆ alkoxy, C₁₋₆ haloalkyl, C₁₋₆ haloalkoxy, C₃₋₆ cycloalkyl, 4-6 membered heterocycloalkyl, C₁₋₃ alkylene-C₃₋₆ cycloalkyl, C₁₋₃ alkylene-C₄₋₆ heterocycloalkyl, C₆₋₁₀ aryl, C₅₋₆ heteroaryl, SO₂R⁷, S(═O)NR⁵, SO₂N(R⁷)₂, CON(R⁷)₂, or N(R⁷)₂. In some embodiments, R¹¹ is each independently R⁶, nitro, C₂₋₆ alkenyl, C₂₋₆ alkynyl, C₁₋₆ alkoxy, C₁₋₆ haloalkyl, C₁₋₆ haloalkoxy, C₃₋₆ cycloalkyl, 4-6 membered heterocycloalkyl, C₁₋₃ alkylene-C₃₋₆ cycloalkyl, C₁₋₃ alkylene-C₄₋₆ heterocycloalkyl, C₆₋₁₀ aryl, C₅₋₆ heteroaryl, SO₂R⁷, S(═O)NR⁵, SO₂N(R⁷)₂, CON(R⁷)₂, or N(R⁷)₂. In some embodiments, wherein R¹¹ is each independently R⁶, C₁₋₆ alkoxy, C₁₋₆ haloalkyl, C₃₋₆ cycloalkyl, 4-6 membered heterocycloalkyl, C₆₋₁₀ aryl, C₅₋₆ heteroaryl, SO₂R⁷, S(═O)NR⁵, SO₂N(R⁷)₂, CON(R⁷)₂, or N(R⁷)₂. In some embodiments, R¹¹ is each independently N(R⁷)₂.

In some embodiments, R¹² is each independently H, hydroxy, C₁₋₆alkyl, or C₁₋₆alkoxy. In some embodiments, In some embodiments, R¹² is each independently H or C₁₋₆alkyl.

In some embodiments, R¹³ is each independently H, C₁₋₆ alkyl, C₁₋₆ haloalkyl, C₂₋₆ alkenyl, C₂₋₆ alkynyl, C₂₋₆ cycloalkyl, C₄₋₆ heterocycloalkyl, C₁₋₃ alkylene-C₃₋₆ cycloalkyl, or C₁₋₃ alkylene-C₄₋₆ heterocycloalkyl, C₁₋₆ acyl, C₆₋₁₀ aroyl, or C₅₋₆ heteroaroyl.

In some embodiments, m is an integer 0 or 1. In some embodiments, m is 0. In some embodiments, m is 1.

In some embodiments, n is an integer 0, 1, or 2. In some embodiments, n is an integer 0 or 1. In some embodiments, n is an integer 1 or 2. In In some embodiments, n is 0. In some embodiments, n is 1. In some embodiments, n is 2.

In some embodiments, p is an integer 0, 1, or 2. In some embodiments, p is an integer 1, 2, or 3. In some embodiments, p is an integer 0 or 1. In some embodiments, p is 0. In some embodiments, p is 1. In some embodiments, p is 2. In some embodiments, p is 3.

Also provided herein, in some embodiments, are compounds of Formula VI:

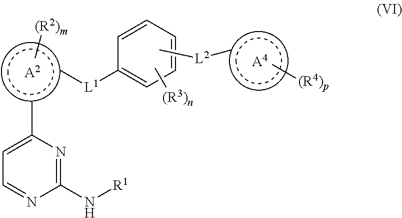

or a pharmaceutically acceptable salt thereof, wherein:
A² is aryl or heteroaryl;
A⁴ is aryl, heteroaryl, or C₃₋₁₂carbocyclyl;
L¹ is —NR^{N1}—, —S(═O)₀₋₂—, —S(═O)(═NR^{N1})—, —C(R⁶)₂—, or —C(═O)—;
L² is —NR^{N1}SO₂—, —SO₂NR^{N1}—, —C(R⁷)₂C(═O)—, —C(═O)C(R⁷)₂—, —C(═O)O—, —OC(═O)—, —C(R⁶)₂O—, —OC(R⁶)₂—, —C(═O)NR^{N1}—, —NRN C(═O)—, —C(R⁶)₂NR^{N1}—, —NR^{N1}C(R⁶)₂—, —C(R⁶)₂S(═O)₀₋₂—, —S(═O)₀₋₂C(R⁶)₂—, —C(R⁶)₂S(═O)NR^{N1}—, —S(═O)NR^{N1}C(R⁶)₂—, —C(R⁶)₂C(R⁷)₂—, or —C(R⁷)₂C(R⁶)₂—;
R¹ is C₃₋₁₀carbocyclyl, 4-12 membered heterocyclyl, phenyl, 5-12 membered heteroaryl, —(C₁₋₆alkylene)-(C₃₋₁₀carbocyclyl), —(C₁₋₆alkylene)-(4-12 membered heterocyclyl), —(C₁₋₆alkylene)-(phenyl), —(C₁₋₆alkylene)-(5-12 membered heteroaryl), —(C₁₋₆alkylene)-OR⁸, or —(C₁₋₆alkylene)-NR⁹R¹⁰, wherein R¹ is optionally substituted by 1-3 R¹²;
R², R³, and R⁴ are each independently halogen, cyano, nitro, oxo, hydroxy, amino, C₁₋₆alkyl, C₁₋₆alkoxy, C₁₋₆alkylamino, C₁₋₆dialkylamino, C₁₋₆haloalkyl, C₁₋₆haloalkoxy, C₃₋₁₀carbocyclyl, C₁₋₆alkylthio, or C₁₋₆alkylsulfonyl, or any two occurrences of R², R³, or R⁴ on contiguous atoms taken together with the contiguous atoms to which the two occurrences of R², R³, or R⁴ are attached form a 5-7 membered ring comprising 0-2 heteroatoms selected from O, S(═O)₀₋₂, P(═O)R^{P1}, N, and NR^{N1}, wherein R², R³, or R⁴ are each independently optionally substituted by 1-3 R¹²;
R⁶ is each independently H, cyano, C₁₋₆alkyl, or C₁₋₆haloalkyl, wherein R⁶ is optionally substituted by 1-3 R¹²;
R⁷ is each independently H, halogen, cyano, C₁₋₆alkyl, C₁₋₆haloalkyl, C₁₋₆alkoxy, or C₁₋₆haloalkoxy, or two occurrences of R⁷ taken together with the C atom to which they are attached form a 4-7 membered ring comprising 0-1 heteroatom selected from O, S(═O)₀₋₂, P(═O)R^{P1}, and NR^{N1}, wherein R⁷ is optionally substituted by 1-3 R¹²;
R⁸ is C₃₋₁₀carbocyclyl, 4-12 membered heterocyclyl, phenyl, or 5-12 membered heteroaryl, wherein R⁸ is optionally substituted by 1-3 R¹²;
R⁹ is H, C₁₋₆alkyl, C₃₋₁₀carbocyclyl, 4-12 membered heterocyclyl, phenyl, or 5-12 membered heteroaryl, wherein R⁹ is optionally substituted by 1-3 R¹²;
R¹⁰ is H or C₁₋₆alkyl;
or R⁹ and R¹⁰ taken together with the N atom to which they are attached form a 4-12 membered heterocyclyl optionally substituted by 1-3 R¹²;
R¹¹ is each independently C₁₋₆alkyl, C₁₋₆haloalkyl, C₃₋₁₀carbocyclyl, or 4-12 membered heterocyclyl;
R¹² is each independently halogen, cyano, oxo, hydroxy, C₁₋₆alkyl, C₂₋₆alkenyl, C₂₋₆alkynyl, C₁₋₆alkoxy, C₁₋₆haloalkyl, C₁₋₆haloalkoxy, SO₂R¹¹, S(═O)NR^{N1}R¹¹, or NR^{N1}R^{N2};
R^{P1} is each independently H or C₁₋₆alkyl;
R^{N1} is each independently H or C₁₋₆alkyl;
R^{N2} is each independently H, C₁₋₆alkyl, SO₂R¹¹, or S(═O)NR^{N1}R¹¹;
m is an integer 0, 1, 2, or 3;
n is an integer 0, 1, 2, or 3; and
p is an integer 0, 1, 2, or 3,
provided that L¹ and L² are not situated on contiguous atoms of the phenyl.

In some embodiments, A⁴ is phenyl, naphthyl, or 5-12 membered heteroaryl. In some embodiments, A⁴ is phenyl, naphthyl, 5-6 membered monocyclic heteroaryl, or 8-12 membered bicyclic heteroaryl. In some embodiments, A⁴ is phenyl, naphthyl, or 5-6 membered monocyclic heteroaryl. In some embodiments, A⁴ is phenyl, pyridyl, pyridazinyl, pyrimidyl, pyrazinyl, triazinyl, thienyl, thiazolyl, thiadiazolyl, oxazolyl, oxadiazolyl, pyrazolyl, imidazolyl, triazolyl, or tetrazolyl.

In some embodiments, A² is phenyl. In some embodiments, A² is 5-6 membered heteroaryl. In some embodiments, A² is 1,2-linked phenylene, 2,3-linked naphthalene, 2-L¹-3-biaryl linked pyridylene or 4-L¹-3-biaryl linked pyridylene.

In some embodiments, A² is

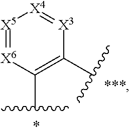

2,3-naphthyl, or 5-6-membered heteroaryl, wherein * is the site covalently linked to the pyrimidyl, and *** is the site covalently linked to L¹; and X³, X⁴, X⁵, and X⁶ are each independently N or CH, wherein the hydrogen of each CH is independently optionally substituted by R², or any two contiguous of X³, X⁴, X⁵, and X⁶ can be taken together as O, S, or NR^{N1}. In some embodiments, at least one of X³, X⁴, X⁵, and X⁶ is N.

In some embodiments, R¹ is C₃₋₁₀carbocyclyl, 4-12 membered heterocyclyl, phenyl, or 5-12 membered heteroaryl, wherein R¹ is optionally substituted by 1-3 R¹². In some embodiments, R¹ is C₃₋₁₀carbocyclyl or 4-12 membered heterocyclyl, wherein R¹ is optionally substituted by 1-3 R¹².

In some embodiments, L¹ is —NR^{N1}—, S(═O)₀₋₂, —S(═O)(═NR^{N1})—, —CHR⁶—, or —CF₂—. In some embodiments, L¹ is —S(═O)₀₋₂— or —C(R⁶)₂—. In some embodiments, L¹ is —S—. In some embodiments, L¹ is —NH—.

In some embodiments, L² is NHSO₂—, —SO₂NH—, —C(R⁷)₂C(═O)—, —C(═O)C(R⁷)₂—, —C(═O)O—, —OC(═O)—, —C(R⁶)₂O—, —OC(R⁶)₂—, —C(═O)NH—, —NHC(═O)—, —C(R⁶)₂NH—, —NHC(R⁶)₂—, —C(R⁶)₂S(═O)₀₋₂—, —S(═O)₀₋₂C(R⁶)₂—, —C(R⁶)₂S(═O)NH—, —S(═O)NHC(R⁶)₂—, —C(R⁶)₂C(R⁷)₂—, or —C(R⁷)₂C(R⁶)₂—. In some embodiments, L² is —NR^{N1}SO₂—.

In some embodiments, R⁶ is each independently H, methyl, ethyl, fluoro, chloro, cyano, CF₃, CHF₂, or CH₂F.

In some embodiments, R⁷ is each independently H, methyl, ethyl, fluoro, chloro, cyano, CF₃, CHF₂, CH₂F, methoxy, or ethoxy.

In some embodiments, m is an integer 0, 1, or 2.

In some embodiments, n is an integer 0, 1, or 2.

In some embodiments, p is an integer 0, 1, or 2.

In some embodiments, provided herein are compounds of Formula VII:

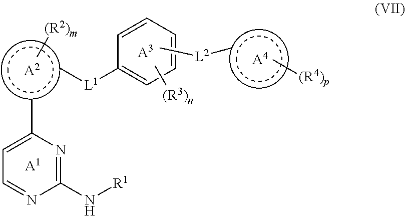

or a pharmaceutically acceptable salt thereof, wherein:
A² is phenyl, 2,3-naphthyl, or 5 or 6-membered heteroaryl;
A⁴ is aryl, heteroaryl, C₃₋₁₂ carbocyclyl, 4-12 membered heterocyclyl, or C₁₋₆alkyl;
L¹ is —O—, —S—, —NH—, —S(═O)₁₋₂—, —S(═O)(═NR⁵)—, —C(R⁶)₂—, or —C(═O)—;
L² is —NR⁷SO₂—, —C(R⁸)₂SO₂—, —NR⁷SO₂—C(R⁸)₂—, —C(R⁸)₂C(═O)—, —C(═O)C(R⁸)₂—, —C(═O)O—, —OC(═O)—, —C(R⁸)₂O—, —OC(R⁸)₂—, —C(═O)NR⁷—, —NR⁷C(═O)—, —C(R⁸)₂NR⁷—, —NR⁷C(R⁸)₂—, —C(R⁸)₂S(═O)₀₋₂—, —S(═O)₀₋₂C(R⁸)₂—, —C(R⁸)₂S(═O)NR⁷—, S(═O)NR⁷C(R⁸)₂—, or —C(R⁸)₂C(R⁸)₂—;
R¹ is C₃₋₁₂ carbocyclyl, 4-12 membered heterocyclyl, phenyl, 5-12 membered heteroaryl, —(C₁₋₆ alkylene)-(C₃₋₁₀ carbocyclyl), —(C₁₋₆ alkylene)-(4-12 membered heterocyclyl), —(C₁₋₆alkylene)-(phenyl), —(C₁₋₆alkylene)-(5-12 membered heteroaryl), —(C₁₋₆alkylene)-OR⁹, or —(C₁₋₆alkylene)-NR⁷R¹⁰, wherein R¹ is optionally substituted by 0-5 R¹¹;
R² is each independently H, halogen, cyano, nitro, hydroxy, —NR⁷R¹⁰, —NHCOR⁹, C₁₋₆ alkyl, C₂₋₆ alkenyl, C₂₋₆ alkynyl, C₁₋₆ alkoxy, C₂₋₆ alkenoxy, C₂₋₆ alkynoxy, COR⁹, CON(R⁹)₂, CF₃, CF₂H, CF₃CF₂, CF₃CH₂, C₁₋₆haloalkyl, CF₃O, CF₂HO, CF₃CF₂O, CF₃CH₂O, C₁₋₆ haloalkoxy, C₃₋₈ cycloalkyl, C₃₋₈ cycloalkenyl, C₁₋₆ alkylthio, C₂₋₆ alkenylthio, C₁₋₆ alkylsulfonyl, aryl, or heteroaryl, wherein R² is each independently optionally substituted by 0-4 R¹¹;
R³ is each independently H, halogen, cyano, nitro, hydroxy, —NR⁷R¹⁰, —NHCOR⁹, C₁₋₆ alkyl, C₂₋₆ alkenyl, C₂₋₆ alkynyl, C₁₋₆ alkoxy, C₂₋₆ alkenoxy, C₂₋₆ alkynoxy, COR⁹, CON(R⁹)₂, CF₃, CF₂H, CF₃CF₂, CF₃CH₂, C₁₋₆ haloalkyl, CF₃O, CF₂HO, CF₃CF₂O, CF₃CH₂O, C₁₋₆ haloalkoxy, C₃₋₈ cycloalkyl, C₃₋₈ cycloalkenyl, C₁₋₆ alkylthio, C₂₋₆ alkenylthio, or C₁₋₆ alkylsulfonyl, wherein R³ is each independently optionally substituted by 0-4 R¹¹;
R⁴ is each independently H, halogen, cyano, nitro, hydroxy, NR⁷R¹⁰, NHCOR⁹, NR⁷CONR⁷, C₁₋₆ alkyl, C₂₋₆ alkenyl, C₂₋₆ alkynyl, COR⁹, CO₂R⁹, CONR⁷R¹⁰, C₁₋₆ alkoxy, C₂₋₆ alkenoxy, C₂₋₆ alkynoxy, CF₃, CF₂H, CF₃CF₂, CF₃CH₂, C₁₋₆ haloalkyl, CF₃O, CF₂HO, CF₃CF₂O, CF₃CH₂O, C₁₋₆ haloalkoxy, C₃₋₈ cycloalkyl, C₃₋₈ cycloalkenyl, C₁₋₆ alkylthio, C₂₋₆ alkenylthio, C₁₋₆ alkylsulfonyl, SO₂N(R⁷)₂, or phenyl, or any two occurrences of R⁴ on contiguous atoms, taken together with the contiguous atoms to which the two occurrences of R⁴ are attached, form a saturated or unsaturated 5-7 membered ring comprising 0-2 heteroatoms each independently selected from O, S(═O)₀₋₂, P(═O)R¹², N, and NR¹⁰, wherein R⁴ is each independently optionally substituted by 0-4 R¹¹;
R⁵ is each independently H or C₁₋₄ alkyl;
R⁶ is each independently H, F, OH, cyano, or C₁₋₆ alkyl;
R⁷ is each independently H, C₁₋₆ alkyl, C₁₋₆ haloalkyl, C₂₋₆ alkenyl, C₂₋₆ alkynyl, C₃₋₆ cycloalkyl, C₄₋₆ heterocycloalkyl, aryl, 5-10 membered heteroaryl, C₁₋₃ alkylene-C₃₋₆ cycloalkyl, or C₁₋₃ alkylene-C₄₋₆ heterocycloalkyl, or two R⁷s along with the N atom to which they are attached form a 4-7 membered ring comprising an additional 0-1 heteroatoms selected from NR⁵, O and S(═O)₀₋₂;
R⁸ is each independently H, halogen, cyano, C₁₋₆ alkyl, C₁₋₆ haloalkyl, C₂₋₆ alkenyl, C₂₋₆ alkynyl, C₂₋₆ cycloalkyl, C₄₋₆ heterocycloalkyl, C₁₋₃ alkylene-C₃₋₆ cycloalkyl, C₁₋₃ alkylene-C₄₋₆ heterocycloalkyl, or two R⁸s taken together with the C atom to which they are attached form a 4-7 membered ring comprising 0-1 heteroatom selected from O, S(═O)₀₋₂, P(═O)R¹², and NR⁵, wherein R⁸ is optionally substituted by 1-3 R¹¹;
each R⁹ is H, C₁₋₆ alkyl, C₁₋₆ haloalkyl, C₂₋₆ alkenyl, C₂₋₆ alkynyl, C₃₋₁₀ cycloalkyl, C₄₋₁₀ heterocycloalkyl, C₁₋₃ alkylene-C₃₋₆ cycloalkyl, C₁₋₃ alkylene-C₄₋₆ heterocycloalkyl, C₁₋₆ acyl, phenyl, or 5-12 membered heteroaryl, wherein R⁹ is optionally substituted by 1-3 R¹¹;
each R¹⁰ is independently H, C₁₋₆ alkyl, C₁₋₆ haloalkyl, C₂₋₆ alkenyl, C₂₋₆ alkynyl, C₃₋₁₀ cycloalkyl, C₄₋₁₀ heterocycloalkyl, C₁₋₃ alkylene-C₃₋₆ cycloalkyl, C₁₋₃ alkylene-C₄₋₆ heterocycloalkyl, C₁₋₆ acyl, C₁₋₆ alkylsulfonyl, phenyl, or 5-12 membered heteroaryl, wherein R¹⁰ is optionally substituted by 1-3 R¹¹,
or R⁷ and R¹⁰ on the same N atom may be taken together with the N atom to which they are attached form a 4-7 membered ring comprising an additional 0-1 heteroatoms selected from NR⁵, O, S(═O)₀₋₂, P(═O)R¹², and NR¹³;
R¹¹ is each independently R⁶, oxo, nitro, C₂₋₆ alkenyl, C₂₋₆ alkynyl, C₁₋₆ alkoxy, C₁₋₆ alkoxyalkyl, C₁₋₆hydroxyalkyl, C₁₋₆ haloalkyl, C₁₋₆ haloalkoxy, C₃₋₆cycloalkyl, 4-6 membered heterocycloalkyl, C₁₋₃ alkylene-C₃₋₆ cycloalkyl, C₁₋₃ alkylene-C₄₋₆ heterocycloalkyl, C₆₋₁₀ aryl, C₅₋₁₀ heteroaryl, SO₂R⁷, S(═O)NR⁵, SO₂N(R⁷)₂, CON(R⁷)₂, or N(R⁷)₂;
R¹² is each independently H, OH, C₁₋₆ alkyl, C₁₋₆ alkoxy, or —N(R⁷)₂;
R¹³ is each independently H, C₁₋₆ alkyl, C₁₋₆ haloalkyl, C₂₋₆ alkenyl, C₂₋₆ alkynyl, C₂₋₆ cycloalkyl, C₄₋₆ heterocycloalkyl, C₁₋₃ alkylene-C₃₋₆ cycloalkyl, or C₁₋₃ alkylene-C₄₋₆ heterocycloalkyl, C₁₋₆ acyl, aroyl, or heteroaroyl;
m is an integer 0, 1 or 2;
n is an integer 0, 1, 2, or 3; and
p is an integer 0, 1, 2, or 3,
provided that
the A¹ ring and the L¹ linker are on contiguous C atoms of the A² ring, whereas L¹ and L² are not situated on contiguous atoms of the A³ ring;
and when A² is

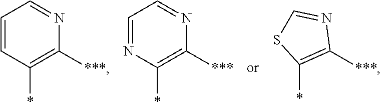

L¹ is not —O—, wherein * is the site covalently linked to the A¹ ring, and *** is the site covalently linked to L¹.

In some embodiments, A⁴ is phenyl, naphthyl, 5-12 membered heteroaryl, C₃₋₁₀ carbocyclyl, or 4-12 membered heterocyclyl. In some embodiments, A⁴ is phenyl, naphthyl, 5-10 membered heteroaryl, C₃₋₁₀ carbocyclyl, or 5-10 membered heterocyclyl. In some embodiments, A⁴ is phenyl, naphthyl, 5-10 membered heteroaryl, C₃₋₆ carbocyclyl, or 5-10 membered heterocyclyl. In some embodiments, A⁴ is phenyl, naphthyl, or 5-12 membered heteroaryl. In some embodiments, A⁴ is phenyl, naphthyl, or 5-10 membered heteroaryl. In some embodiments, A⁴ is phenyl, naphthyl, 5-6 membered monocyclic heteroaryl, or 8-12 membered bicyclic heteroaryl. In some embodiments, A⁴ is phenyl, pyridyl, pyridazinyl, pyrimidyl, pyrazinyl, triazinyl, thienyl, thiazolyl, thiadiazolyl, oxazolyl, oxadiazolyl, pyrazolyl, imidazolyl, triazolyl, or tetrazolyl. In some embodiments, A⁴ is phenyl, naphthyl, thiophenyl, cyclopropyl, cyclobutyl, cyclopentyl, cyclohexyl,

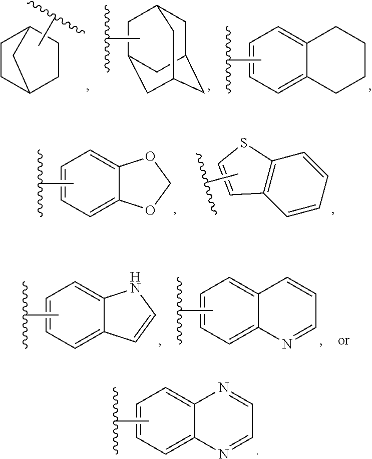

In some embodiments, A⁴ is phenyl.

In some embodiments, A² is phenyl, 2,3-naphthyl, pyridinyl, or pyrimidinyl. In some embodiments, A² is phenyl. In some embodiments, A² is 2,3-naphthyl. In some embodiments, A² is 5-6 membered heteroaryl. In some embodiments, A² is 1,2-linked phenylene, 2,3-linked-naphthylene, 2-L¹-3-biaryl linked pyridylene, 4-L¹-3-biaryl linked pyridylene or 4-L¹-5-biaryl linked pyrimidylene. In some embodiments, A² is 1,2-linked phenylene, 2-L¹-3-biaryl linked pyridylene or 4-L¹-3-biaryl linked pyridylene. In some embodiments, A² is phenyl, 2,3-naphthyl,

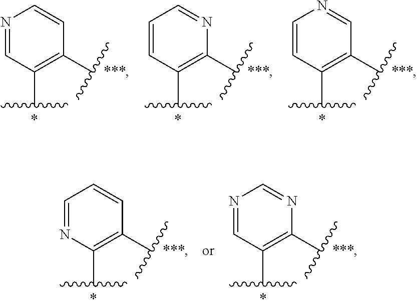 [SMILES: *c1ccncc1* ; *c1cccnc1* ; *c1ccncc1* ; *c1cccnc1* ; *c1cncnc1*]

wherein * is the site covalently linked to the A¹ ring, and *** is the site covalently linked to L¹.

In some embodiments, R¹ is C₃₋₁₀carbocyclyl, 4-12 membered heterocyclyl, phenyl, 5-12 membered heteroaryl, —(C₁₋₆alkylene)-(C₃₋₁₀carbocyclyl), —(C₁₋₆alkylene)-(4-12 membered heterocyclyl), —(C₁₋₆alkylene)-(phenyl), —(C₁₋₆alkylene)-(5-12 membered heteroaryl), —(C₁₋₆alkylene)-OR⁹, or —(C₁₋₆alkylene)-NR⁷R¹⁰, wherein R¹ is optionally substituted by 1-2 R¹¹. In some embodiments, R¹ is C₆₋₁₀carbocyclyl, 4-8 membered heterocyclyl, phenyl, 5-6 membered heteroaryl, —(C₁₋₆alkylene)-(C₃₋₁₀carbocyclyl), —(C₁₋₆alkylene)-(4-8 membered heterocyclyl), —(C₁₋₆alkylene)-(5-6 membered heteroaryl), —(C₁₋₆alkylene)-OR⁹, or —(C₁₋₆alkylene)-NR⁷R¹⁰, wherein R¹ is optionally substituted by 1-2 R¹¹. In some embodiments, R¹ is C₆₋₁₀carbocyclyl, 4-8 membered heterocyclyl, phenyl, 5-6 membered heteroaryl, —(C₁₋₆alkylene)-(C₃₋₁₀carbocyclyl), —(C₁₋₆alkylene)-(4-8 membered heterocyclyl), —(C₁₋₆alkylene)-(5-6 membered heteroaryl), —(C₁₋₆alkylene)-OR⁹, or —(C₁₋₆alkylene)-NR⁷R¹⁰, wherein R¹ is optionally substituted by 1 R¹¹. In some embodiments, R¹ is C₆₋₁₀carbocyclyl, 4-8 membered heterocyclyl containing 1-2 heteroatoms selected from the group consisting of O, S, and N, —(C₁₋₆alkylene)-(C₃₋₁₀carbocyclyl), —(C₁₋₆alkylene)-(4-8 membered heterocyclyl containing 1-2 heteroatoms selected from the group consisting of O, S, and N), —(C₁₋₆alkylene)-(5-6 membered heteroaryl containing 1-2 heteroatoms selected from the group consisting of O, S, and N), —(C₁₋₆alkylene)-OR⁹, or —(C₁₋₆alkylene)-NR⁷R¹⁰, wherein R¹ is optionally substituted by 1 R¹¹. In some embodiments, R¹ is C₆₋₁₀carbocyclyl, 4-8 membered heterocyclyl containing 1-2 heteroatoms selected from the group consisting of O, S, and N, —(C₁₋₃alkylene)-(4-6 membered heterocyclyl containing 1-2 heteroatoms selected from the group consisting of O, S, and N), —(C₁₋₃alkylene)-(5-6 membered heteroaryl containing 1-2 heteroatoms selected from the group consisting of O, S, and N), —(C₁₋₆alkylene)-OR⁹, or —(C₁₋₆alkylene)-NR⁷R¹⁰, wherein R¹ is optionally substituted by 1 R¹¹.

In some embodiments, R¹ is cyclohexyl, piperidinyl, oxetanyl, azetidinyl, pyrrolidinyl, or azaspiroheptanyl, wherein R¹ is optionally substituted with 1 R¹¹.

In some embodiments, R¹ is cyclohexyl optionally substituted with 1 R¹¹.

In some embodiments, R¹ is piperidinyl, oxetanyl, azetidinyl, pyrrolidinyl, or azaspiroheptanyl, wherein R¹ is optionally substituted with 1 R¹¹. In some embodiments, R¹ is piperidinyl optionally substituted with 1 R¹¹.

In some embodiments, R¹ is —(C₁₋₃alkylene)-OR⁹ wherein R⁹ is C₁₋₆alkyl substituted by 1-2 R¹¹. In some embodiments, R¹ is —(C₁₋₃alkylene)-OR⁹ wherein R⁹ is C₁₋₆alkyl substituted by 1 R¹¹. In some embodiments, R¹¹ is hydroxy or C₁₋₄alkoxy. In some embodiments, R¹¹ is hydroxy or C₁₋₂alkoxy.

In some embodiments, R¹ is —(C₁₋₃alkylene)-NR⁷R¹⁰ wherein R⁹ is H or C₁₋₆alkyl and R¹⁰ is H or C₁₋₆alkyl, wherein the R¹⁰ is optionally substituted by 1-2 R¹¹. In some embodiments, R¹ is —(C₁₋₃alkylene)-NR⁷R¹⁰ wherein R⁹ is H or C₁₋₆alkyl and R¹⁰ is H or C₁₋₆alkyl, wherein the R¹⁰ is optionally substituted by 1 R¹¹. In some embodiments, R¹¹ is hydroxy or C₁₋₄alkoxy. In some embodiments, R¹¹ is hydroxy or C₁₋₂alkoxy.

In some embodiments, R¹ is —(C₁₋₃alkylene)-(4-6 membered heterocyclyl containing 1-2 heteroatoms selected from the group consisting of O, S, and N) optionally substituted by 1 R¹¹.

In some embodiments, R¹ is —(C₁₋₃alkylene)-(5-6 membered heteroaryl containing 1-2 heteroatoms selected from the group consisting of O, S, and N) optionally substituted by 1 R¹¹.

In some embodiments, L¹ is —O—, —S—, —NH—, —S(═O)₁₋₂—, —S(═O)(═NR⁵)—, —CHR⁶—, or —CF₂—. In some embodiments, L¹ is —O—, —S—, —NH—, —S(═O)₁₋₂—, or —C(R⁶)₂—. In some embodiments, L¹ is —O— or —S—. In some embodiments, L¹ is —O—. In some embodiments L¹ is —S—.

In some embodiments, L² is —NR⁷SO₂—, —C(R⁸)₂SO₂—, —NR⁷SO₂—C(R⁸)₂—, —C(R⁸)₂C(═O)—, —C(═O)C(R⁸)₂—, —C(═O)O—, —OC(═O)—, —C(R⁸)₂O—, —OC(R⁸)₂—, or —C(═O)NR⁷—. In some embodiments, L² is —NR⁷SO₂—. In some embodiments, L² is —NHSO₂—, —NHSO₂—CHR⁸—, —C(═O)NH—, or —NHC(═O)—. In some embodiments, L² is —NHSO₂—, —NHSO₂—CH₂—, —C(═O)NH—, or —NHC(═O)—. In some embodiments L² is —NHSO₂—.

In some embodiments, R⁵ is each independent H or C₁₋₂alkyl. In some embodiments, R⁵ is H or methyl.

In some embodiments, R⁶ is each independently H, methyl, ethyl, fluoro, chloro, cyano, CF₃, CHF₂, or CH₂F.

In some embodiments, R⁷ is each independently H, C₁₋₆ alkyl, C₁₋₆ haloalkyl, C₂₋₆ alkenyl, C₂₋₆ alkynyl, C₂₋₆ cycloalkyl, C₄₋₆heterocycloalkyl, aryl, 5-10 membered heteroaryl, C₁₋₃ alkylene-C₃₋₆ cycloalkyl, or C₁₋₃ alkylene-C₄₋₆ heterocycloalkyl. In some embodiments, R⁷ is each independently H, C₁₋₆ alkyl, C₃₋₆ cycloalkyl, C₄₋₆ heterocycloalkyl, aryl, or 5-10 membered heteroaryl. In some embodiments, R⁷ is H or C₁₋₆alkyl. In some embodiments, two R⁷s, along with the N atom to which they are attached form a 4-7 membered ring comprising an additional 0-1 heteroatoms selected from NR⁵, O and S(═O)₀₋₂.

In some embodiments, R⁸ is each independently H, halogen, cyano, C₁₋₆ alkyl, C₁₋₆ haloalkyl, C₂₋₆ alkenyl, C₂₋₆ alkynyl, C₃₋₆ cycloalkyl, C₄₋₆ heterocycloalkyl, C₁₋₃ alkylene-C₃₋₆ cycloalkyl, C₁₋₃ alkylene-C₄₋₆ heterocycloalkyl. In some embodiments, two R⁸s taken together with the C atom to which they are attached form a 4-7 membered ring comprising 0-1 heteroatom selected from O, S(═O)₀₋₂, P(═O)R¹²—, and NR⁵. In some embodiments, R⁸ is each independently H, methyl, ethyl, fluoro, chloro, cyano, CF₃, CHF₂, CH₂F, methoxy, or ethoxy.

In some embodiments, R² is each independently H, halogen, cyano, nitro, hydroxy, —NR⁷R¹⁰, —NHCOR⁹, C₁₋₆ alkyl, C₂₋₆ alkenyl, C₂₋₆ alkynyl, C₁₋₆ alkoxy, C₂₋₆ alkenoxy, C₂₋₆ alkynoxy, COR⁹, CON(R⁹)₂, CF₃, CF₂H, CF₃CF₂, CF₃CH₂, C₁₋₆haloalkyl, CF₃O, CF₂HO, CF₃CF₂O, CF₃CH₂O, C₁₋₆ haloalkoxy, C₃₋₈ cycloalkyl, C₃₋₈ cycloalkenyl, C₁₋₆ alkylthio, C₂₋₆ alkenylthio, C₁₋₆ alkylsulfonyl, aryl or heteroaryl, wherein R² is each independently optionally substituted by 0-2 R¹¹. In some embodiments, R² is each independently H, halogen, cyano, C₁₋₆ alkyl, C₂₋₆ alkenyl, C₂₋₆ alkynyl, C₁₋₆haloalkyl, or C₃₋₆cycloalkyl. In some embodiments, R² is each independently H, halogen, cyano, C₁₋₆ alkyl, C₂₋₄alkenyl, C₂₋₄alkynyl, C₁₋₄haloalkyl, or C₃₋₆cycloalkyl.

In some embodiments, R³ is each independently H, halogen, cyano, nitro, hydroxy, —NR⁷R¹⁰, —NHCOR⁹, C₁₋₆ alkyl, C₂₋₆ alkenyl, C₂₋₆ alkynyl, C₁₋₆ alkoxy, C₂₋₆ alkenoxy, C₂₋₆ alkynoxy, COR⁹, CON(R⁹)₂, CF₃, CF₂H, CF₃CF₂, CF₃CH₂, C₁₋₆haloalkyl, CF₃O, CF₂HO, CF₃CF₂O, CF₃CH₂O, C₁₋₆ haloalkoxy, C₃₋₈ cycloalkyl, C₃₋₈ cycloalkenyl, C₁₋₆ alkylthio, C₂₋₆ alkenylthio, or C₁₋₆ alkylsulfonyl, wherein R³ is each independently optionally substituted by 0-2 R¹¹. In some embodiments, R³ is each independently H, halogen, cyano, C₁₋₆alkyl, or C₁₋₆haloalkyl. In some embodiments, R³ is each independently H, halogen, cyano, C₁₋₄alkyl, or C₁₋₄haloalkyl. In some embodiments, R³ is each independently H, halogen, cyano, or C₁₋₂haloalkyl. In some embodiments, R³ is each independently H or halogen.

In some embodiments, R⁴ is each independently H, halogen, cyano, nitro, hydroxy, NR⁷R¹⁰, NHCOR⁹, NR⁷CONR⁷, C₁₋₆ alkyl, C₂₋₆ alkenyl, C₂₋₆ alkynyl, COR⁹, CO₂R⁹, CONR⁷R¹⁰, C₁₋₆ alkoxy, C₂₋₆ alkenoxy, C₂₋₆ alkynoxy, CF₃, CF₂H, CF₃CF₂, CF₃CH₂, C₁₋₆ haloalkyl, CF₃O, CF₂HO, CF₃CF₂O, CF₃CH₂O, C₁₋₆haloalkoxy, C₃₋₈ cycloalkyl, C₃₋₈ cycloalkenyl, C₁₋₆ alkylthio, C₂₋₆ alkenylthio, C₁₋₆ alkylsulfonyl, SO₂N(R⁷)₂, or phenyl, or any two occurrences of R⁴ on contiguous atoms, taken together with the contiguous atoms to which the two occurrences of R⁴ are attached, form a saturated or unsaturated 5-7 membered ring comprising 0-2 heteroatoms each independently selected from O, S(═O)₀₋₂, P(═O)R¹², N, and NR¹⁰, wherein R⁴ is each independently optionally substituted by 0-2 R¹¹. In some embodiments, R⁴ is each independently H, halogen, cyano, nitro, hydroxy, NR⁷R¹⁰, C₁₋₆alkyl, C₂₋₆alkenyl, C₂₋₆alkynyl, C₁₋₆alkoxy, C₂₋₆alkenoxy, C₂₋₆alkynoxy, C₁₋₆haloalkyl, C₁₋₆haloalkoxy, C₃₋₈cycloalkyl, C₃₋₈cycloalkenyl, C₁₋₆alkylsulfonyl, SO₂N(R⁷)₂, or phenyl, wherein R⁴ is each independently optionally substituted by 0-2 R¹¹. In some embodiments, R⁴ is each independently H, halogen, cyano, nitro, hydroxy, NR⁷R¹⁰, NHCOR⁹, NR⁷CONR⁷, C₁₋₆alkyl, C₂₋₆alkenyl, C₂₋₆alkynyl, COR⁹, CO₂R⁹, CONR⁷R¹⁰, C₁₋₆alkoxy, C₁₋₆haloalkyl, C₁₋₆haloalkoxy, or C₃₋₈cycloalkyl, wherein R⁴ is each independently optionally substituted by 0-1 R¹¹. In some embodiments, R⁴ is each independently H, halogen, cyano, nitro, C₁₋₆ alkyl optionally substituted with R¹¹, C₂₋₆alkenyl, C₂₋₆alkynyl, C₁₋₆haloalkyl, C₁₋₆alkoxy, C₃₋₆cycloalkyl, or CONR⁷R¹⁰.

In some embodiments, R⁹ is H, C₁₋₆ alkyl, C₁₋₆ haloalkyl, C₂₋₆ alkenyl, C₂₋₆ alkynyl, C₃₋₁₀ cycloalkyl, C₄₋₁₀ heterocycloalkyl, C₁₋₃ alkylene-C₃₋₆ cycloalkyl, C₁₋₃ alkylene-C₄₋₆ heterocycloalkyl, C₁₋₆ acyl, phenyl, or 5-12 membered heteroaryl, wherein R⁹ is optionally substituted by 1-2 R¹¹. In some embodiments, R⁹ is H, C₃₋₁₀ cycloalkyl, C₄₋₁₀ heterocycloalkyl, phenyl, or 5-12 membered heteroaryl, wherein R⁹ is optionally substituted by 1-2 R¹¹. In some embodiments, R⁹ is C₁₋₆alkyl optionally substituted by 1 R¹¹.

In some embodiments, R¹⁰ is H, C₁₋₆ alkyl, C₁₋₆ haloalkyl, C₂₋₆ alkenyl, C₂₋₆ alkynyl, C₃₋₁₀ cycloalkyl, C₄₋₁₀ heterocycloalkyl, C₁₋₃ alkylene-C₃₋₆ cycloalkyl, C₁₋₃ alkylene-C₄₋₆ heterocycloalkyl, C₁₋₆ acyl, phenyl, or 5-12 membered heteroaryl, wherein R¹⁰ is optionally substituted by 1-2 R¹¹. In some embodiments, R¹⁰ is H or C₁₋₆alkyl, wherein the R¹⁰ is optionally substituted by 1-2 R¹¹. In some embodiments, R¹⁰ is H or C₁₋₆alkyl, wherein the R¹⁰ is optionally substituted by 1 R¹¹. In some embodiments, R¹⁰ is H or C₁₋₆alkyl.

In some embodiments, R⁷ and R¹⁰ on the same N atom may be taken together with the N atom to which they are attached form a 4-7 membered ring comprising an additional 0-1 heteroatoms selected from NR⁵, O, S(═O)₀₋₂, P(═O)R¹², and NR¹³, wherein R¹⁰ is optionally substituted by 1-2 R¹¹. In some embodiments, R⁷ and R¹⁰ on the same N atom may be taken together with the N atom to which they are attached form a 4-7 membered ring comprising an additional 0-1 heteroatoms selected from NR⁵, O, S(═O)₀₋₂, P(═O)R¹², and NR¹³.

In some embodiments, R¹¹ is each independently R⁶, oxo, nitro, C₂₋₆ alkenyl, C₂₋₆alkynyl, C₁₋₆ alkoxy, C₁₋₆haloalkyl, C₁₋₆haloalkoxy, C₃₋₆cycloalkyl, 4-6 membered heterocycloalkyl, C₁₋₃ alkylene-C₃₋₆ cycloalkyl, C₁₋₃ alkylene-C₄₋₆ heterocycloalkyl, C₆₋₁₀ aryl, C₅₋₆ heteroaryl, SO₂R⁷, S(═O)NR⁵, SO₂N(R⁷)₂, CON(R⁷)₂, or N(R⁷)₂. In some embodiments, R¹¹ is each independently R⁶, nitro, C₂₋₆ alkenyl, C₂₋₆ alkynyl, C₁₋₆ alkoxy, C₁₋₆ haloalkyl, C₁₋₆ haloalkoxy, C₃₋₆ cycloalkyl, 4-6 membered heterocycloalkyl, C₁₋₃ alkylene-C₃₋₆ cycloalkyl, C₁₋₃ alkylene-C₄₋₆ heterocycloalkyl, C₆₋₁₀ aryl, C₅₋₆ heteroaryl, SO₂R⁷, S(═O)NR⁵, SO₂N(R⁷)₂, CON(R⁷)₂, or N(R⁷)₂. In some embodiments, wherein R¹¹ is each independently R⁶, C₁₋₆ alkoxy, C₁₋₆haloalkyl, C₃₋₆ cycloalkyl, 4-6 membered heterocycloalkyl, C₆₋₁₀ aryl, C₅₋₆ heteroaryl, SO₂R⁷, S(═O)NR⁵, SO₂N(R⁷)₂, CON(R⁷)₂, or N(R⁷)₂. In some embodiments, R¹¹ is each independently N(R⁷)₂.

In some embodiments, R¹² is each independently H, hydroxy, C₁₋₆alkyl, or C₁₋₆alkoxy. In some embodiments, In some embodiments, R¹² is each independently H or C₁₋₆alkyl.

In some embodiments, R¹³ is each independently H, C₁₋₆ alkyl, C₁₋₆ haloalkyl, C₂₋₆ alkenyl, C₂₋₆ alkynyl, C₂₋₆ cycloalkyl, C₄₋₆ heterocycloalkyl, C₁₋₃ alkylene-C₃₋₆ cycloalkyl, or C₁₋₃ alkylene-C₄₋₆ heterocycloalkyl, C₁₋₆ acyl, C₆₋₁₀ aroyl, or C₅₋₆ heteroaroyl.

In some embodiments, m is an integer 0 or 1. In some embodiments, m is 0. In some embodiments, m is 1.

In some embodiments, n is an integer 0, 1, or 2. In some embodiments, n is an integer 0 or 1. In some embodiments, n is an integer 1 or 2. In In some embodiments, n is 0. In some embodiments, n is 1. In some embodiments, n is 2.

In some embodiments, p is an integer 0, 1, or 2. In some embodiments, p is an integer 1, 2, or 3. In some embodiments, p is an integer 0 or 1. In some embodiments, p is 0. In some embodiments, p is 1. In some embodiments, p is 2. In some embodiments, p is 3.

In some embodiments, a compound described herein is a compound depicted in Table 1.

TABLE 1

| Compound | |
|---|---|
| Number | Structure |
| 1 | 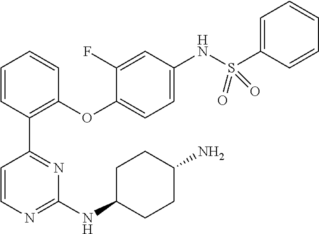 [SMILES: N[C@H]1CC[C@H](Nc2nccc(-c3ccccc3Oc3ccc(NS(=O)(=O)c4ccccc4)cc3F)n2)CC1] |
| 2 | 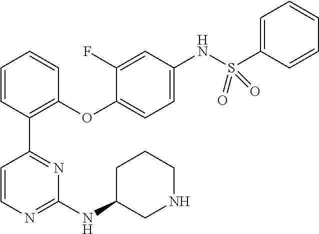 [SMILES: O=S(=O)(Nc1ccc(Oc2ccccc2-c2ccnc(N[C@H]3CCCNC3)n2)c(F)c1)c1ccccc1] |
| 3 | 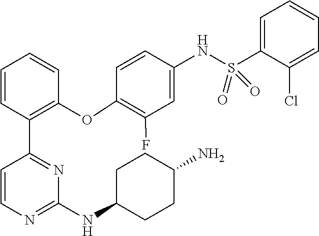 [SMILES: N[C@H]1CC[C@H](Nc2nccc(-c3ccccc3Oc3ccc(NS(=O)(=O)c4ccccc4Cl)cc3F)n2)CC1] |
| 4 | 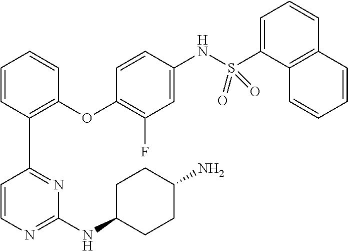 [SMILES: N[C@H]1CC[C@H](Nc2nccc(-c3ccccc3Oc3ccc(NS(=O)(=O)c4cccc5ccccc45)cc3F)n2)CC1] |
| 5 | 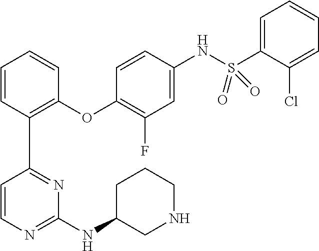 [SMILES: O=S(=O)(Nc1ccc(Oc2ccccc2-c2ccnc(N[C@H]3CCCNC3)n2)c(F)c1)c1ccccc1Cl] |
| 6 | 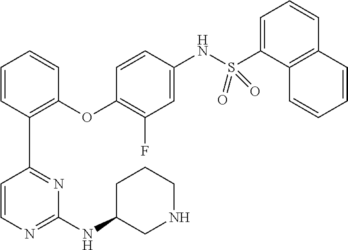 [SMILES: O=S(=O)(Nc1ccc(Oc2ccccc2-c2ccnc(N[C@H]3CCCNC3)n2)c(F)c1)c1cccc2ccccc12] |
| 7 | 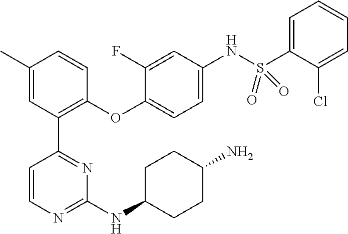 [SMILES: Cc1ccc(Oc2ccc(NS(=O)(=O)c3ccccc3Cl)cc2F)c(-c2ccnc(N[C@H]3CC[C@H](N)CC3)n2)c1] |
| 8 | 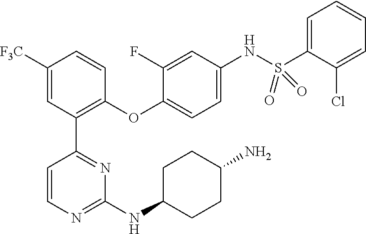 [SMILES: N[C@H]1CC[C@H](Nc2nccc(-c3cc(C(F)(F)F)ccc3Oc3ccc(NS(=O)(=O)c4ccccc4Cl)cc3F)n2)CC1] |
| 9 | 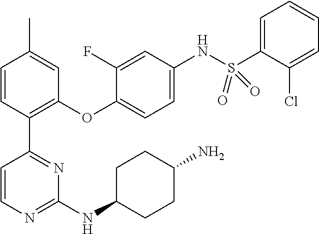 [SMILES: Cc1ccc(-c2ccnc(N[C@H]3CC[C@H](N)CC3)n2)c(Oc2ccc(NS(=O)(=O)c3ccccc3Cl)cc2F)c1] |
| 10 | 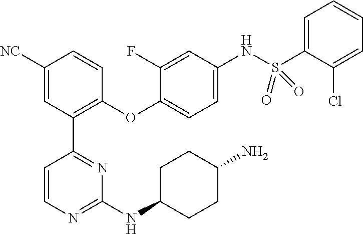 [SMILES: N#Cc1ccc(Oc2ccc(NS(=O)(=O)c3ccccc3Cl)cc2F)c(-c2ccnc(N[C@H]3CC[C@H](N)CC3)n2)c1] |
| 11 | 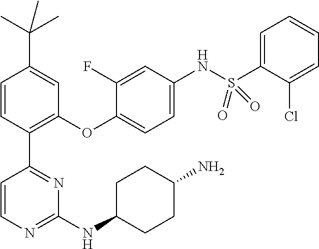 [SMILES: CC(C)(C)c1ccc(-c2ccnc(N[C@H]3CC[C@H](N)CC3)n2)c(Oc2ccc(NS(=O)(=O)c3ccccc3Cl)cc2F)c1] |
| 12 | 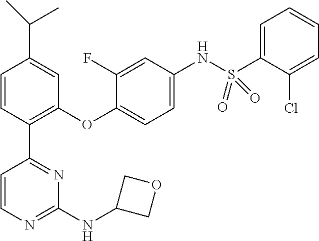 [SMILES: CC(C)c1ccc(-c2ccnc(NC3COC3)n2)c(Oc2ccc(NS(=O)(=O)c3ccccc3Cl)cc2F)c1] |
| 13 | 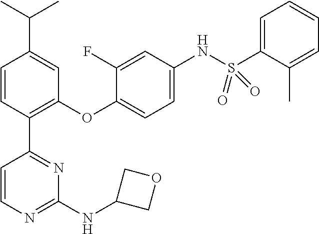 [SMILES: Cc1ccccc1S(=O)(=O)Nc1ccc(Oc2cc(C(C)C)ccc2-c2ccnc(NC3COC3)n2)c(F)c1] |
| 14 | 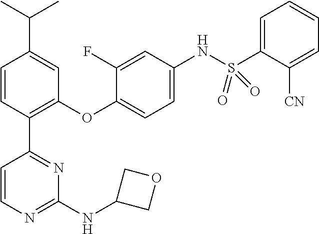 [SMILES: CC(C)c1ccc(-c2ccnc(NC3COC3)n2)c(Oc2ccc(NS(=O)(=O)c3ccccc3C#N)cc2F)c1] |
| 15 | 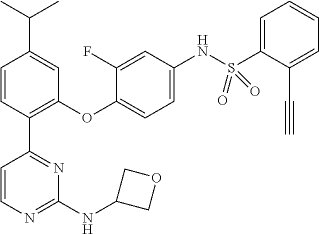 [SMILES: C#Cc1ccccc1S(=O)(=O)Nc1ccc(Oc2cc(C(C)C)ccc2-c2ccnc(NC3COC3)n2)c(F)c1] |
| 16 | 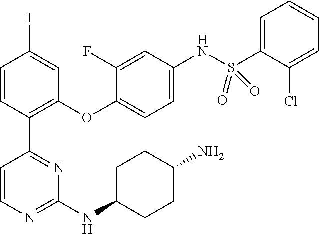 [SMILES: N[C@H]1CC[C@H](Nc2nccc(-c3ccc(I)cc3Oc3ccc(NS(=O)(=O)c4ccccc4Cl)cc3F)n2)CC1] |
| 17 | 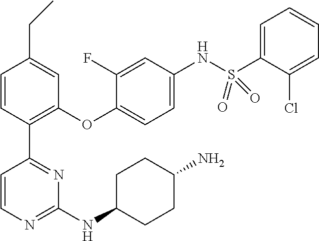 [SMILES: CCc1ccc(-c2ccnc(N[C@H]3CC[C@H](N)CC3)n2)c(Oc2ccc(NS(=O)(=O)c3ccccc3Cl)cc2F)c1] |
| 18 | 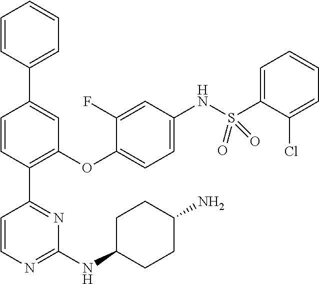 [SMILES: N[C@H]1CC[C@H](Nc2nccc(-c3ccc(-c4ccccc4)cc3Oc3ccc(NS(=O)(=O)c4ccccc4Cl)cc3F)n2)CC1] |
| 19 | 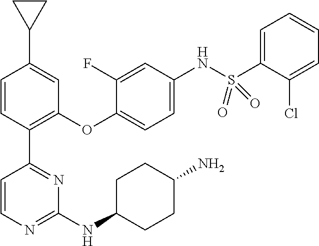 [SMILES: N[C@H]1CC[C@H](Nc2nccc(-c3ccc(C4CC4)cc3Oc3ccc(NS(=O)(=O)c4ccccc4Cl)cc3F)n2)CC1] |
| 20 | 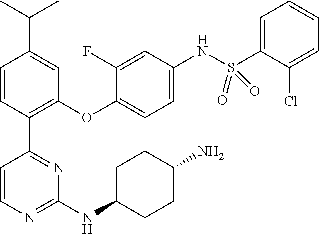 [SMILES: CC(C)c1ccc(-c2ccnc(N[C@H]3CC[C@H](N)CC3)n2)c(Oc2ccc(NS(=O)(=O)c3ccccc3Cl)cc2F)c1] |
| 21 | 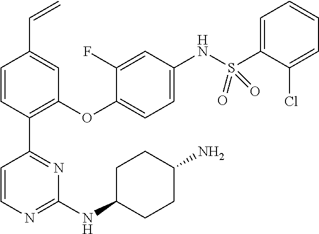 [SMILES: C=Cc1ccc(-c2ccnc(N[C@H]3CC[C@H](N)CC3)n2)c(Oc2ccc(NS(=O)(=O)c3ccccc3Cl)cc2F)c1] |
| 22 | 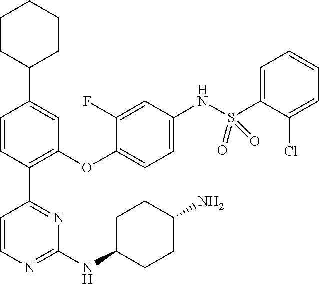 [SMILES: N[C@H]1CC[C@H](Nc2nccc(-c3ccc(C4CCCCC4)cc3Oc3ccc(NS(=O)(=O)c4ccccc4Cl)cc3F)n2)CC1] |
| 23 | 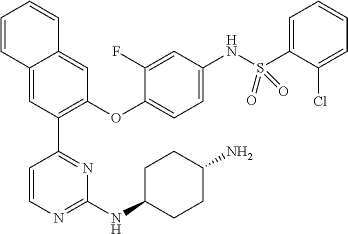 [SMILES: N[C@H]1CC[C@H](Nc2nccc(-c3cc4ccccc4cc3Oc3ccc(NS(=O)(=O)c4ccccc4Cl)cc3F)n2)CC1] |
| 24 | 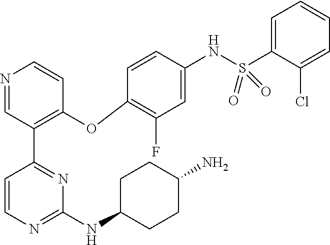 [SMILES: N[C@H]1CC[C@H](Nc2nccc(-c3cnccc3Oc3ccc(NS(=O)(=O)c4ccccc4Cl)cc3F)n2)CC1] |
| 25 | 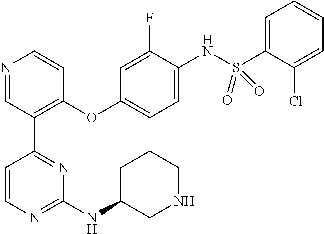 [SMILES: O=S(=O)(Nc1ccc(Oc2ccncc2-c2ccnc(N[C@H]3CCCNC3)n2)cc1F)c1ccccc1Cl] |
| 26 | 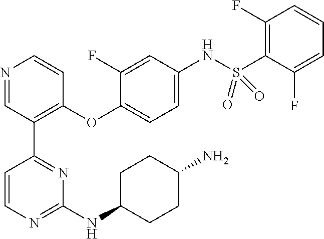 [SMILES: N[C@H]1CC[C@H](Nc2nccc(-c3cnccc3Oc3ccc(NS(=O)(=O)c4c(F)cccc4F)cc3F)n2)CC1] |
| 27 | 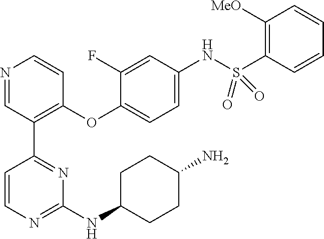 [SMILES: COc1ccccc1S(=O)(=O)Nc1ccc(Oc2ccncc2-c2ccnc(N[C@H]3CC[C@H](N)CC3)n2)c(F)c1] |
| 28 | 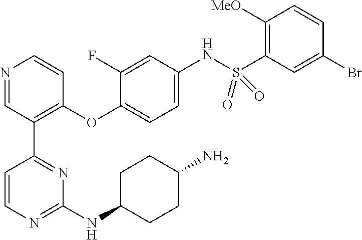 [SMILES: COc1ccc(Br)cc1S(=O)(=O)Nc1ccc(Oc2ccncc2-c2ccnc(N[C@H]3CC[C@H](N)CC3)n2)c(F)c1] |
| 29 | 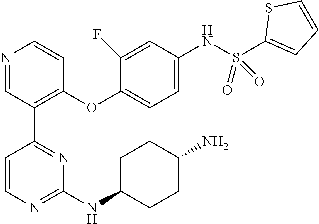 [SMILES: N[C@H]1CC[C@H](Nc2nccc(-c3cnccc3Oc3ccc(NS(=O)(=O)c4cccs4)cc3F)n2)CC1] |
| 30 | 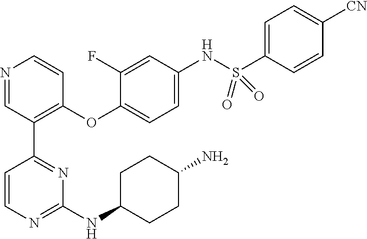 [SMILES: N#Cc1ccc(S(=O)(=O)Nc2ccc(Oc3ccncc3-c3ccnc(N[C@H]4CC[C@H](N)CC4)n3)c(F)c2)cc1] |
| 31 | 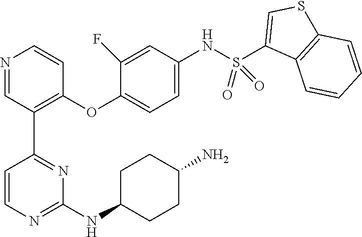 [SMILES: N[C@H]1CC[C@H](Nc2nccc(-c3cnccc3Oc3ccc(NS(=O)(=O)c4csc5ccccc45)cc3F)n2)CC1] |
| 32 | 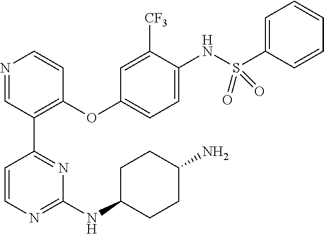 [SMILES: N[C@H]1CC[C@H](Nc2nccc(-c3cnccc3Oc3ccc(NS(=O)(=O)c4ccccc4)c(C(F)(F)F)c3)n2)CC1] |
| 33 | 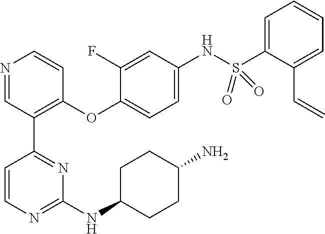 [SMILES: C=Cc1ccccc1S(=O)(=O)Nc1ccc(Oc2ccncc2-c2ccnc(N[C@H]3CC[C@H](N)CC3)n2)c(F)c1] |
| 34 | 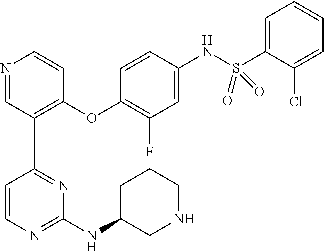 [SMILES: O=S(=O)(Nc1ccc(Oc2ccncc2-c2ccnc(N[C@H]3CCCNC3)n2)c(F)c1)c1ccccc1Cl] |
| 35 | 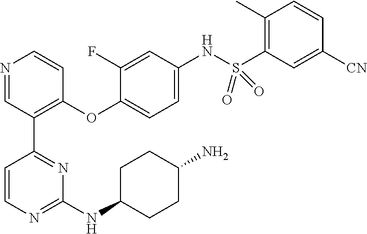 [SMILES: Cc1ccc(C#N)cc1S(=O)(=O)Nc1ccc(Oc2ccncc2-c2ccnc(N[C@H]3CC[C@H](N)CC3)n2)c(F)c1] |
| 36 | 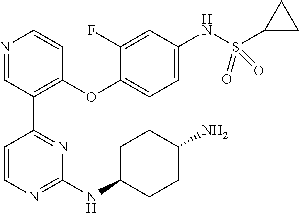 [SMILES: N[C@H]1CC[C@H](Nc2nccc(-c3cnccc3Oc3ccc(NS(=O)(=O)C4CC4)cc3F)n2)CC1] |
| 37 | 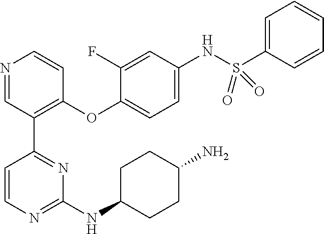 [SMILES: N[C@H]1CC[C@H](Nc2nccc(-c3cnccc3Oc3ccc(NS(=O)(=O)c4ccccc4)cc3F)n2)CC1] |
| 38 | 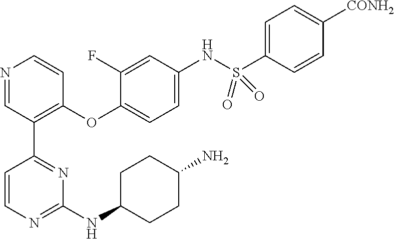 [SMILES: NC(=O)c1ccc(S(=O)(=O)Nc2ccc(Oc3ccncc3-c3ccnc(N[C@H]4CC[C@H](N)CC4)n3)c(F)c2)cc1] |
| 39 | 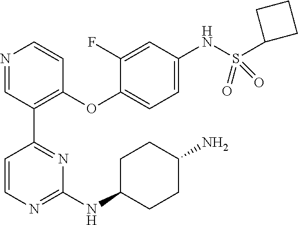 [SMILES: N[C@H]1CC[C@H](Nc2nccc(-c3cnccc3Oc3ccc(NS(=O)(=O)C4CCC4)cc3F)n2)CC1] |
| 40 | 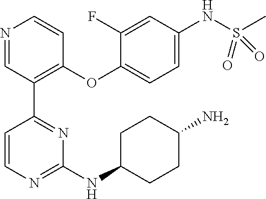 [SMILES: CS(=O)(=O)Nc1ccc(Oc2ccncc2-c2ccnc(N[C@H]3CC[C@H](N)CC3)n2)c(F)c1] |
| 41 | 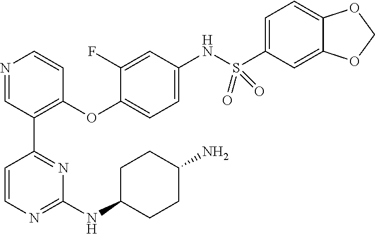 [SMILES: N[C@H]1CC[C@H](Nc2nccc(-c3cnccc3Oc3ccc(NS(=O)(=O)c4ccc5c(c4)OCO5)cc3F)n2)CC1] |
| 42 | 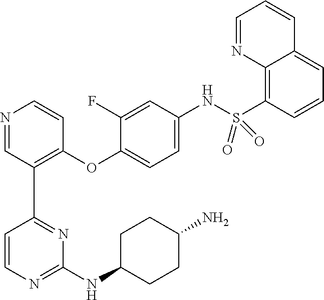 [SMILES: N[C@H]1CC[C@H](Nc2nccc(-c3cnccc3Oc3ccc(NS(=O)(=O)c4cccc5cccnc45)cc3F)n2)CC1] |
| 43 | 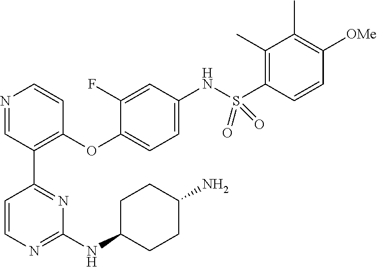 [SMILES: COc1ccc(S(=O)(=O)Nc2ccc(Oc3ccncc3-c3ccnc(N[C@H]4CC[C@H](N)CC4)n3)c(F)c2)c(C)c1C] |
| 44 | 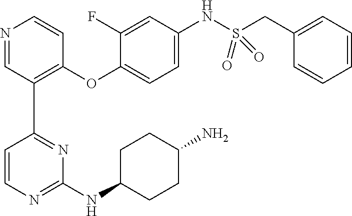 [SMILES: N[C@H]1CC[C@H](Nc2nccc(-c3cnccc3Oc3ccc(NS(=O)(=O)Cc4ccccc4)cc3F)n2)CC1] |
| 45 | 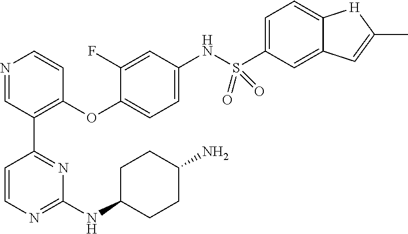 |
| 46 | 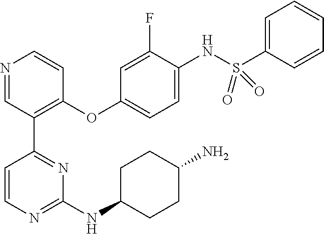 [SMILES: N[C@H]1CC[C@H](Nc2nccc(-c3cnccc3Oc3ccc(NS(=O)(=O)c4ccccc4)c(F)c3)n2)CC1] |
| 47 | 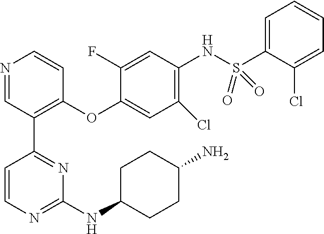 [SMILES: N[C@H]1CC[C@H](Nc2nccc(-c3cnccc3Oc3cc(Cl)c(NS(=O)(=O)c4ccccc4Cl)cc3F)n2)CC1] |
| 48 | 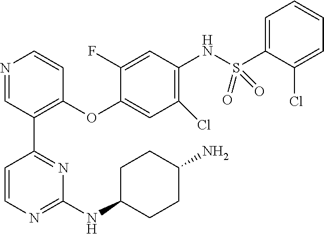 [SMILES: N[C@H]1CC[C@H](Nc2nccc(-c3cnccc3Oc3cc(Cl)c(NS(=O)(=O)c4ccccc4Cl)cc3F)n2)CC1] |
| 49 | 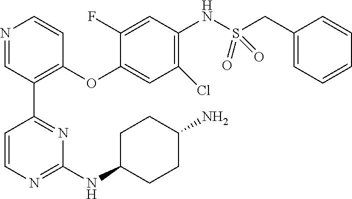 [SMILES: N[C@H]1CC[C@H](Nc2nccc(-c3cnccc3Oc3cc(Cl)c(NS(=O)(=O)Cc4ccccc4)cc3F)n2)CC1] |
| 50 | 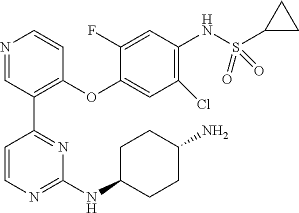 [SMILES: N[C@H]1CC[C@H](Nc2nccc(-c3cnccc3Oc3cc(Cl)c(NS(=O)(=O)C4CC4)cc3F)n2)CC1] |
| 51 | 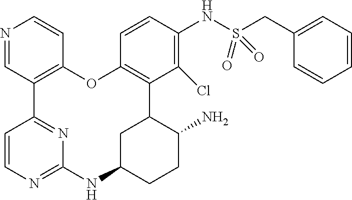 [SMILES: Cc1c(Oc2ccncc2-c2ccnc(N[C@H]3CC[C@H](N)CC3)n2)ccc(NS(=O)(=O)Cc2ccccc2)c1Cl] |
| 52 | 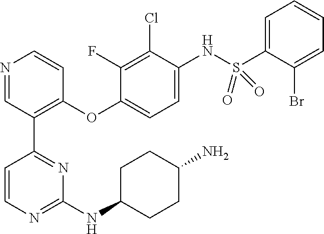 [SMILES: N[C@H]1CC[C@H](Nc2nccc(-c3cnccc3Oc3ccc(NS(=O)(=O)c4ccccc4Br)c(Cl)c3F)n2)CC1] |
| 53 | 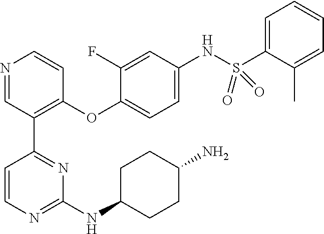 [SMILES: Cc1ccccc1S(=O)(=O)Nc1ccc(Oc2ccncc2-c2ccnc(N[C@H]3CC[C@H](N)CC3)n2)c(F)c1] |
| 54 | 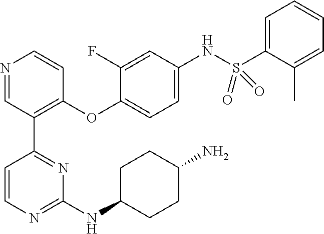 [SMILES: Cc1ccccc1S(=O)(=O)Nc1ccc(Oc2ccncc2-c2ccnc(N[C@H]3CC[C@H](N)CC3)n2)c(F)c1] |
| 55 | 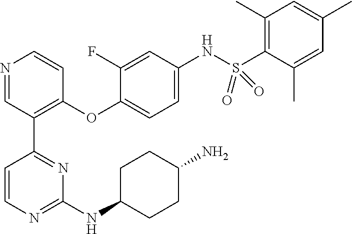 [SMILES: Cc1cc(C)c(S(=O)(=O)Nc2ccc(Oc3ccncc3-c3ccnc(N[C@H]4CC[C@H](N)CC4)n3)c(F)c2)c(C)c1] |
| 56 | 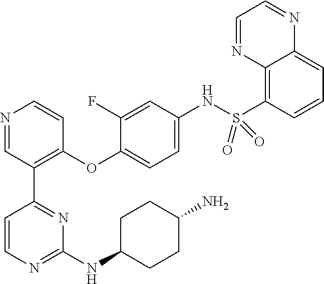 [SMILES: N[C@H]1CC[C@H](Nc2nccc(-c3cnccc3Oc3ccc(NS(=O)(=O)c4cccc5nccnc45)cc3F)n2)CC1] |
| 57 | 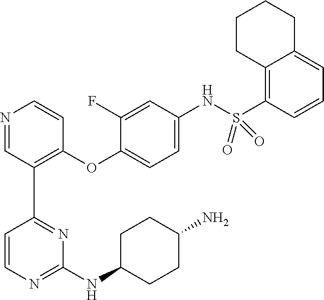 [SMILES: N[C@H]1CC[C@H](Nc2nccc(-c3cnccc3Oc3ccc(NS(=O)(=O)c4cccc5c4CCCC5)cc3F)n2)CC1] |
| 58 | 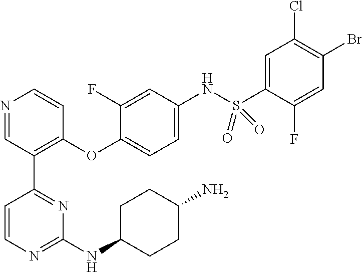 [SMILES: N[C@H]1CC[C@H](Nc2nccc(-c3cnccc3Oc3ccc(NS(=O)(=O)c4cc(Cl)c(Br)cc4F)cc3F)n2)CC1] |
| 59 | 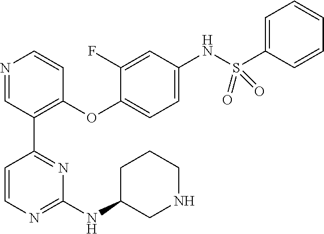 [SMILES: O=S(=O)(Nc1ccc(Oc2ccncc2-c2ccnc(N[C@H]3CCCNC3)n2)c(F)c1)c1ccccc1] |
| 60 | 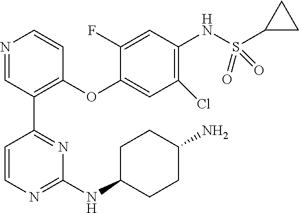 [SMILES: N[C@H]1CC[C@H](Nc2nccc(-c3cnccc3Oc3cc(Cl)c(NS(=O)(=O)C4CC4)cc3F)n2)CC1] |
| 61 | 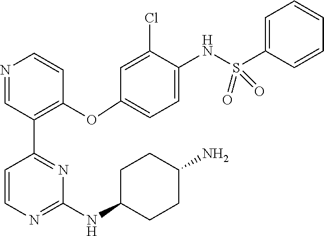 [SMILES: N[C@H]1CC[C@H](Nc2nccc(-c3cnccc3Oc3ccc(NS(=O)(=O)c4ccccc4)c(Cl)c3)n2)CC1] |
| 62 | 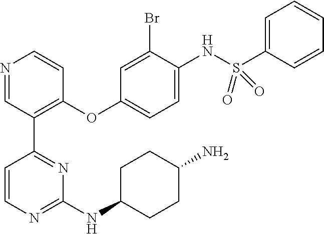 [SMILES: N[C@H]1CC[C@H](Nc2nccc(-c3cnccc3Oc3ccc(NS(=O)(=O)c4ccccc4)c(Br)c3)n2)CC1] |
| 63 | 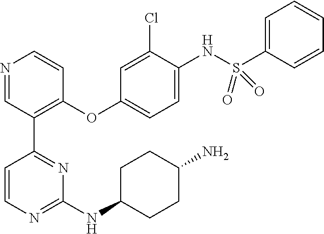 [SMILES: N[C@H]1CC[C@H](Nc2nccc(-c3cnccc3Oc3ccc(NS(=O)(=O)c4ccccc4)c(Cl)c3)n2)CC1] |
| 64 | 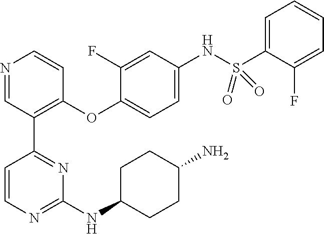 [SMILES: N[C@H]1CC[C@H](Nc2nccc(-c3cnccc3Oc3ccc(NS(=O)(=O)c4ccccc4F)cc3F)n2)CC1] |
| 65 | 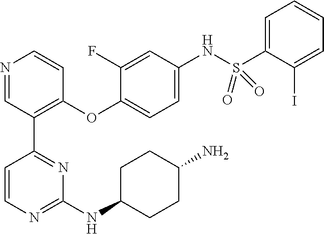 [SMILES: N[C@H]1CC[C@H](Nc2nccc(-c3cnccc3Oc3ccc(NS(=O)(=O)c4ccccc4I)cc3F)n2)CC1] |
| 66 | 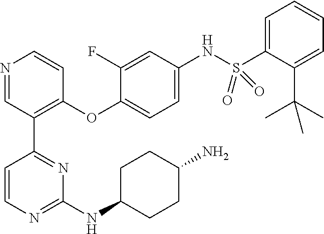 [SMILES: CC(C)(C)c1ccccc1S(=O)(=O)Nc1ccc(Oc2ccncc2-c2ccnc(N[C@H]3CC[C@H](N)CC3)n2)c(F)c1] |
| 67 | 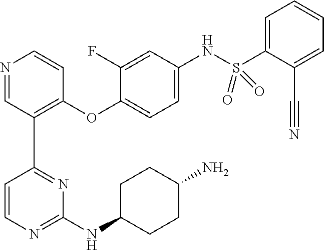 [SMILES: N#Cc1ccccc1S(=O)(=O)Nc1ccc(Oc2ccncc2-c2ccnc(N[C@H]3CC[C@H](N)CC3)n2)c(F)c1] |
| 68 | 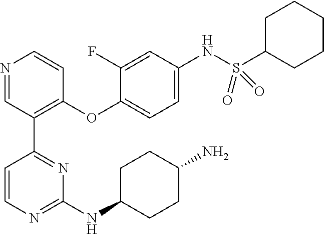 [SMILES: N[C@H]1CC[C@H](Nc2nccc(-c3cnccc3Oc3ccc(NS(=O)(=O)C4CCCCC4)cc3F)n2)CC1] |
| 69 | 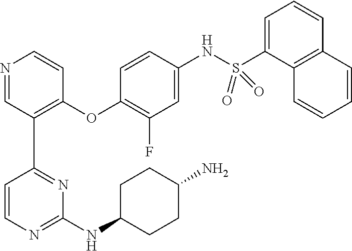 [SMILES: N[C@H]1CC[C@H](Nc2nccc(-c3cnccc3Oc3ccc(NS(=O)(=O)c4cccc5ccccc45)cc3F)n2)CC1] |
| 70 | 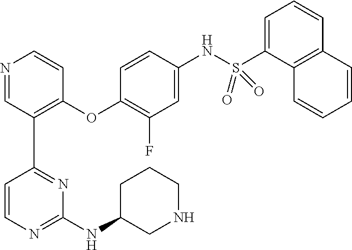 [SMILES: O=S(=O)(Nc1ccc(Oc2ccncc2-c2ccnc(N[C@H]3CCCNC3)n2)c(F)c1)c1cccc2ccccc12] |
| 71 | 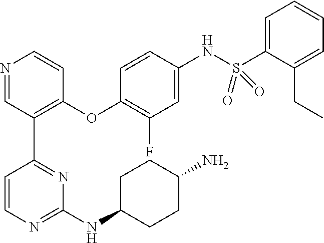 [SMILES: CCc1ccccc1S(=O)(=O)Nc1ccc(Oc2ccncc2-c2ccnc(N[C@H]3CC[C@H](N)CC3)n2)c(F)c1] |
| 72 | 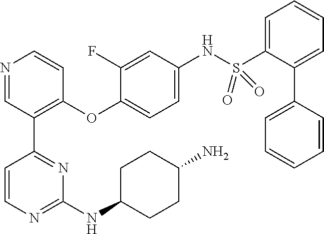 [SMILES: N[C@H]1CC[C@H](Nc2nccc(-c3cnccc3Oc3ccc(NS(=O)(=O)c4ccccc4-c4ccccc4)cc3F)n2)CC1] |
| 73 | 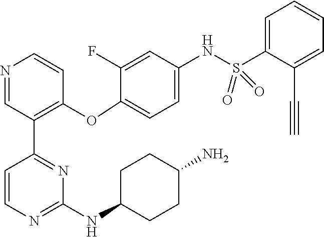 [SMILES: C#Cc1ccccc1S(=O)(=O)Nc1ccc(Oc2ccncc2-c2ccnc(N[C@H]3CC[C@H](N)CC3)n2)c(F)c1] |
| 74 | 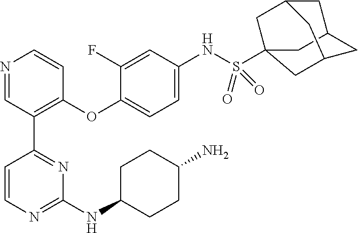 [SMILES: N[C@H]1CC[C@H](Nc2nccc(-c3cnccc3Oc3ccc(NS(=O)(=O)C45CC6CC(CC(C6)C4)C5)cc3F)n2)CC1] |
| 75 | 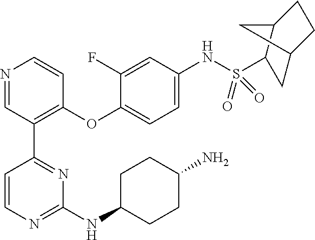 [SMILES: N[C@H]1CC[C@H](Nc2nccc(-c3cnccc3Oc3ccc(NS(=O)(=O)C4CC5CCC4C5)cc3F)n2)CC1] |
| 76 | 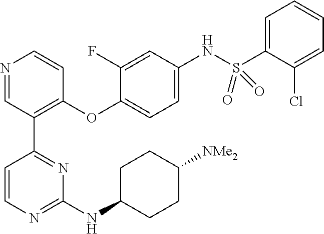 [SMILES: CN(C)[C@H]1CC[C@H](Nc2nccc(-c3cnccc3Oc3ccc(NS(=O)(=O)c4ccccc4Cl)cc3F)n2)CC1] |
| 77 | 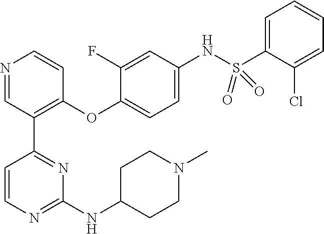 [SMILES: CN1CCC(Nc2nccc(-c3cnccc3Oc3ccc(NS(=O)(=O)c4ccccc4Cl)cc3F)n2)CC1] |
| 78 | 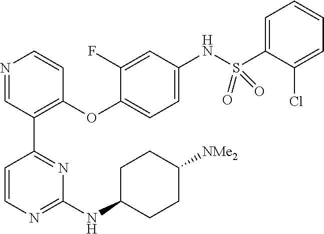 [SMILES: CN(C)[C@H]1CC[C@H](Nc2nccc(-c3cnccc3Oc3ccc(NS(=O)(=O)c4ccccc4Cl)cc3F)n2)CC1] |
| 79 | 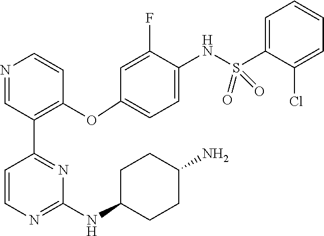 [SMILES: N[C@H]1CC[C@H](Nc2nccc(-c3cnccc3Oc3ccc(NS(=O)(=O)c4ccccc4Cl)c(F)c3)n2)CC1] |
| 80 | 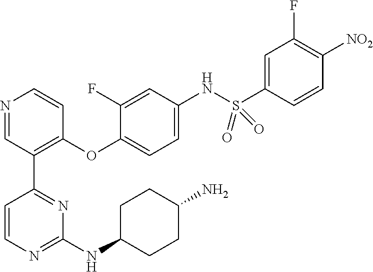 [SMILES: N[C@H]1CC[C@H](Nc2nccc(-c3cnccc3Oc3ccc(NS(=O)(=O)c4ccc([N+](=O)[O-])c(F)c4)cc3F)n2)CC1] |
| 81 | 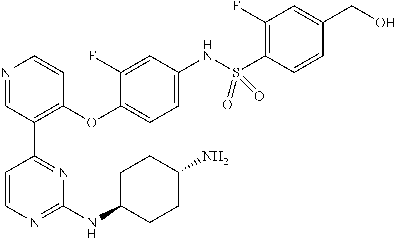 [SMILES: N[C@H]1CC[C@H](Nc2nccc(-c3cnccc3Oc3ccc(NS(=O)(=O)c4ccc(CO)cc4F)cc3F)n2)CC1] |
| 82 | 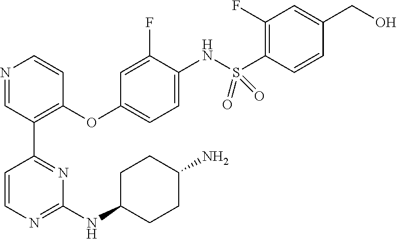 [SMILES: N[C@H]1CC[C@H](Nc2nccc(-c3cnccc3Oc3ccc(NS(=O)(=O)c4ccc(CO)cc4F)c(F)c3)n2)CC1] |
| 83 | 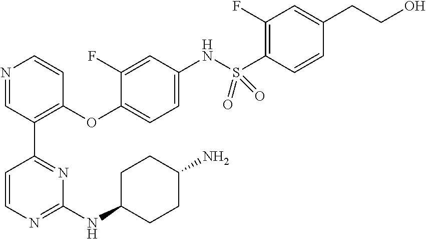 [SMILES: N[C@H]1CC[C@H](Nc2nccc(-c3cnccc3Oc3ccc(NS(=O)(=O)c4ccc(CCO)cc4F)cc3F)n2)CC1] |
| 84 | 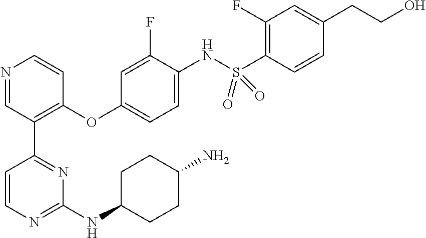 [SMILES: N[C@H]1CC[C@H](Nc2nccc(-c3cnccc3Oc3ccc(NS(=O)(=O)c4ccc(CCO)cc4F)c(F)c3)n2)CC1] |
| 85 | 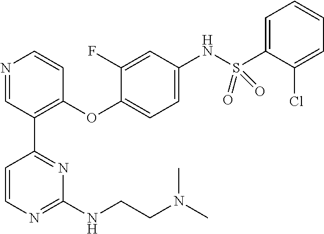 [SMILES: CN(C)CCNc1nccc(-c2cnccc2Oc2ccc(NS(=O)(=O)c3ccccc3Cl)cc2F)n1] |
| 86 | 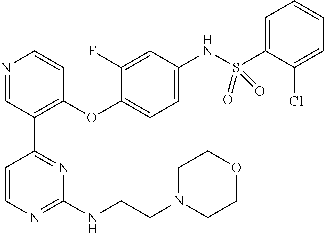 [SMILES: O=S(=O)(Nc1ccc(Oc2ccncc2-c2ccnc(NCCN3CCOCC3)n2)c(F)c1)c1ccccc1Cl] |
| 87 | 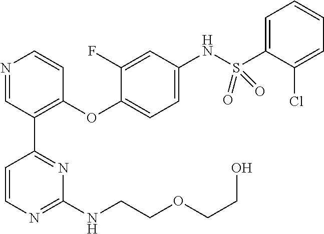 [SMILES: O=S(=O)(Nc1ccc(Oc2ccncc2-c2ccnc(NCCOCCO)n2)c(F)c1)c1ccccc1Cl] |
| 88 | 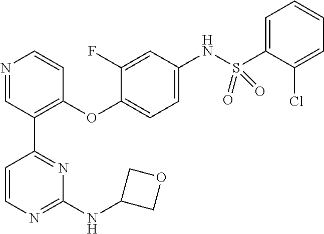 [SMILES: O=S(=O)(Nc1ccc(Oc2ccncc2-c2ccnc(NC3COC3)n2)c(F)c1)c1ccccc1Cl] |
| 89 | 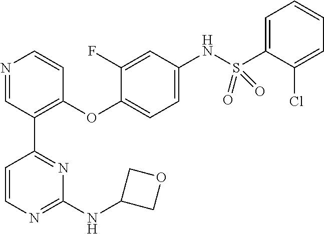 [SMILES: O=S(=O)(Nc1ccc(Oc2ccncc2-c2ccnc(NC3COC3)n2)c(F)c1)c1ccccc1Cl] |
| 90 | 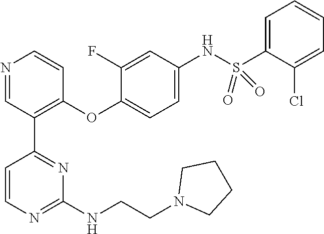 [SMILES: O=S(=O)(Nc1ccc(Oc2ccncc2-c2ccnc(NCCN3CCCC3)n2)c(F)c1)c1ccccc1Cl] |
| 91 | 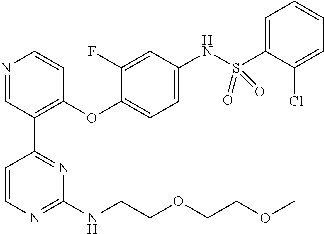 [SMILES: COCCOCCNc1nccc(-c2cnccc2Oc2ccc(NS(=O)(=O)c3ccccc3Cl)cc2F)n1] |
| 92 | 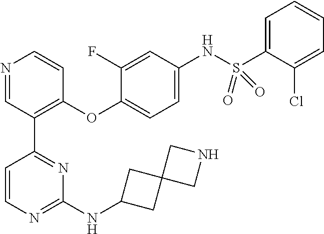 [SMILES: O=S(=O)(Nc1ccc(Oc2ccncc2-c2ccnc(NC3CC4(CNC4)C3)n2)c(F)c1)c1ccccc1Cl] |
| 93 | 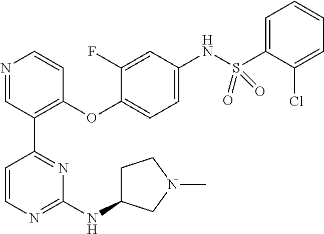 [SMILES: CN1CC[C@H](Nc2nccc(-c3cnccc3Oc3ccc(NS(=O)(=O)c4ccccc4Cl)cc3F)n2)C1] |
| 94 | 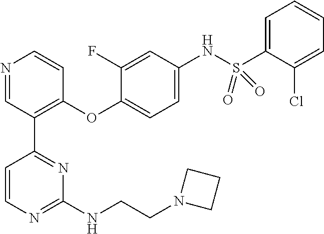 [SMILES: O=S(=O)(Nc1ccc(Oc2ccncc2-c2ccnc(NCCN3CCC3)n2)c(F)c1)c1ccccc1Cl] |
| 95 | 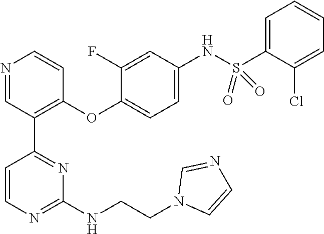 [SMILES: O=S(=O)(Nc1ccc(Oc2ccncc2-c2ccnc(NCCn3ccnc3)n2)c(F)c1)c1ccccc1Cl] |
| 96 | 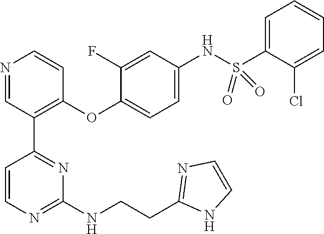 [SMILES: O=S(=O)(Nc1ccc(Oc2ccncc2-c2ccnc(NCCc3ncc[nH]3)n2)c(F)c1)c1ccccc1Cl] |
| 97 | 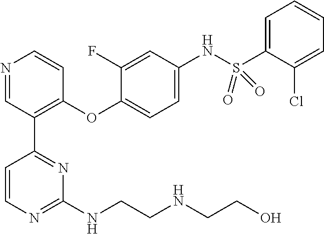 [SMILES: O=S(=O)(Nc1ccc(Oc2ccncc2-c2ccnc(NCCNCCO)n2)c(F)c1)c1ccccc1Cl] |
| 98 | 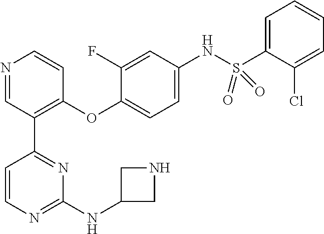 [SMILES: O=S(=O)(Nc1ccc(Oc2ccncc2-c2ccnc(NC3CNC3)n2)c(F)c1)c1ccccc1Cl] |
| 99 | 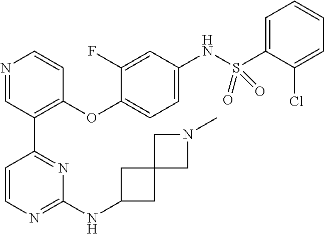 [SMILES: CN1CC2(CC(Nc3nccc(-c4cnccc4Oc4ccc(NS(=O)(=O)c5ccccc5Cl)cc4F)n3)C2)C1] |
| 100 | 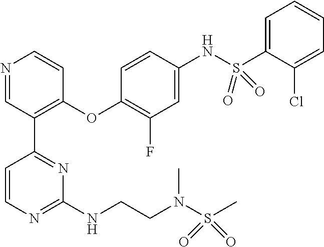 [SMILES: CN(CCNc1nccc(-c2cnccc2Oc2ccc(NS(=O)(=O)c3ccccc3Cl)cc2F)n1)S(C)(=O)=O] |
| 101 | 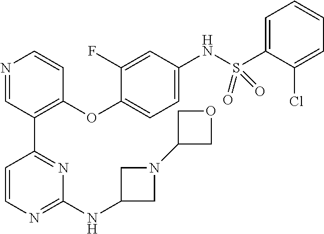 [SMILES: O=S(=O)(Nc1ccc(Oc2ccncc2-c2ccnc(NC3CN(C4COC4)C3)n2)c(F)c1)c1ccccc1Cl] |
| 102 | 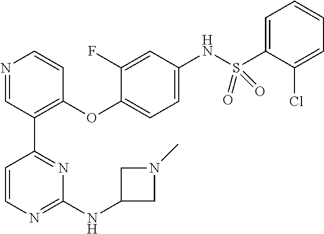 [SMILES: CN1CC(Nc2nccc(-c3cnccc3Oc3ccc(NS(=O)(=O)c4ccccc4Cl)cc3F)n2)C1] |
| 103 | 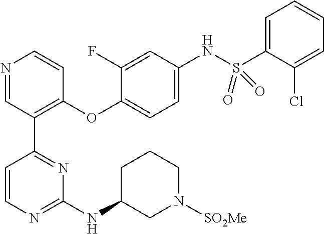 [SMILES: CS(=O)(=O)N1CCC[C@H](Nc2nccc(-c3cnccc3Oc3ccc(NS(=O)(=O)c4ccccc4Cl)cc3F)n2)C1] |
| 104 | 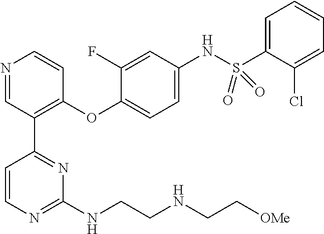 [SMILES: COCCNCCNc1nccc(-c2cnccc2Oc2ccc(NS(=O)(=O)c3ccccc3Cl)cc2F)n1] |
| 105 | 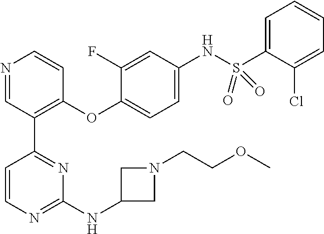 [SMILES: COCCN1CC(Nc2nccc(-c3cnccc3Oc3ccc(NS(=O)(=O)c4ccccc4Cl)cc3F)n2)C1] |
| 106 | 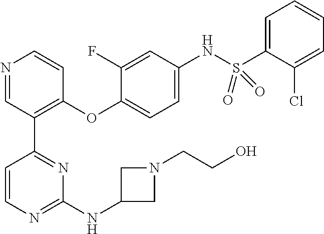 [SMILES: O=S(=O)(Nc1ccc(Oc2ccncc2-c2ccnc(NC3CN(CCO)C3)n2)c(F)c1)c1ccccc1Cl] |
| 107 | 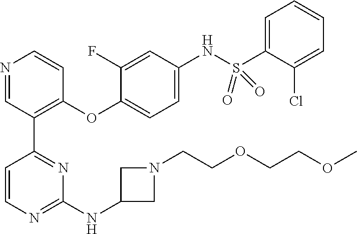 [SMILES: COCCOCCN1CC(Nc2nccc(-c3cnccc3Oc3ccc(NS(=O)(=O)c4ccccc4Cl)cc3F)n2)C1] |
| 108 | 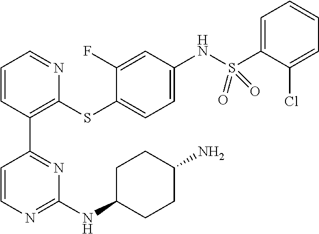 [SMILES: N[C@H]1CC[C@H](Nc2nccc(-c3cccnc3Sc3ccc(NS(=O)(=O)c4ccccc4Cl)cc3F)n2)CC1] |
| 109 | 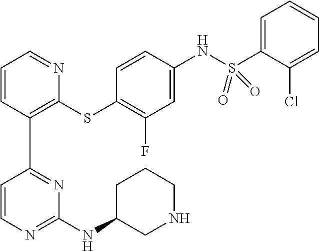 [SMILES: O=S(=O)(Nc1ccc(Sc2ncccc2-c2ccnc(N[C@H]3CCCNC3)n2)c(F)c1)c1ccccc1Cl] |
| 110 | 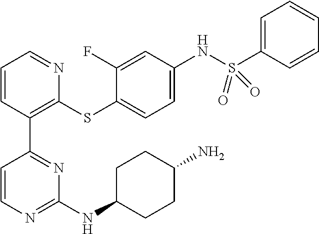 [SMILES: N[C@H]1CC[C@H](Nc2nccc(-c3cccnc3Sc3ccc(NS(=O)(=O)c4ccccc4)cc3F)n2)CC1] |
| 111 | 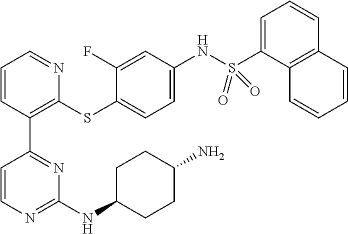 [SMILES: N[C@H]1CC[C@H](Nc2nccc(-c3cccnc3Sc3ccc(NS(=O)(=O)c4cccc5ccccc45)cc3F)n2)CC1] |
| 112 | 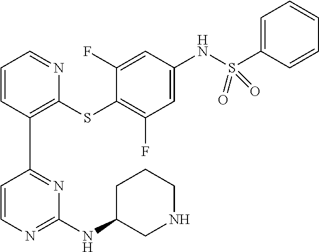 [SMILES: O=S(=O)(Nc1cc(F)c(Sc2ncccc2-c2ccnc(N[C@H]3CCCNC3)n2)c(F)c1)c1ccccc1] |
| 113 | 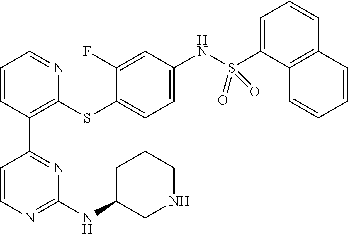 [SMILES: O=S(=O)(Nc1ccc(Sc2ncccc2-c2ccnc(N[C@H]3CCCNC3)n2)c(F)c1)c1cccc2ccccc12] |
| 114 | 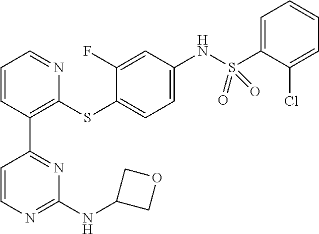 [SMILES: O=S(=O)(Nc1ccc(Sc2ncccc2-c2ccnc(NC3COC3)n2)c(F)c1)c1ccccc1Cl] |
| 115 | 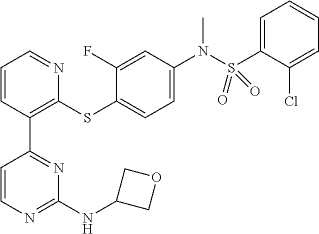 [SMILES: CN(c1ccc(Sc2ncccc2-c2ccnc(NC3COC3)n2)c(F)c1)S(=O)(=O)c1ccccc1Cl] |
| 116 | 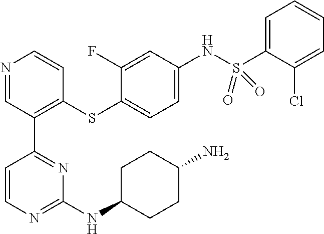 [SMILES: N[C@H]1CC[C@H](Nc2nccc(-c3cnccc3Sc3ccc(NS(=O)(=O)c4ccccc4Cl)cc3F)n2)CC1] |
| 117 | 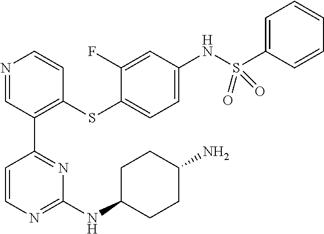 [SMILES: N[C@H]1CC[C@H](Nc2nccc(-c3cnccc3Sc3ccc(NS(=O)(=O)c4ccccc4)cc3F)n2)CC1] |
| 118 | 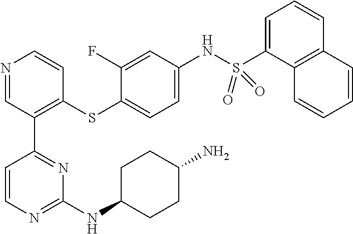 [SMILES: N[C@H]1CC[C@H](Nc2nccc(-c3cnccc3Sc3ccc(NS(=O)(=O)c4cccc5ccccc45)cc3F)n2)CC1] |
| 119 | 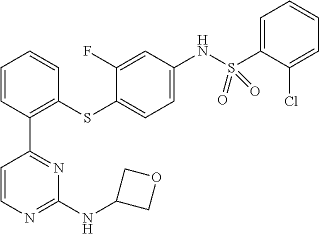 [SMILES: O=S(=O)(Nc1ccc(Sc2ccccc2-c2ccnc(NC3COC3)n2)c(F)c1)c1ccccc1Cl] |
| 120 | 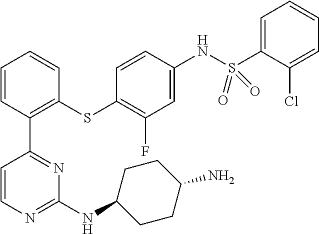 [SMILES: N[C@H]1CC[C@H](Nc2nccc(-c3ccccc3Sc3ccc(NS(=O)(=O)c4ccccc4Cl)cc3F)n2)CC1] |
| 121 | 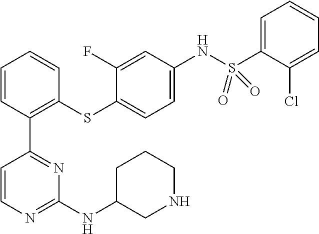 [SMILES: O=S(=O)(Nc1ccc(Sc2ccccc2-c2ccnc(NC3CCCNC3)n2)c(F)c1)c1ccccc1Cl] |
| 122 | 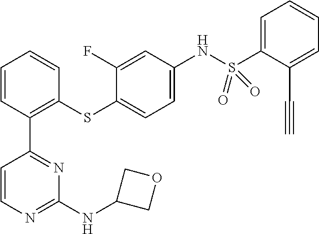 [SMILES: C#Cc1ccccc1S(=O)(=O)Nc1ccc(Sc2ccccc2-c2ccnc(NC3COC3)n2)c(F)c1] |
| 123 | 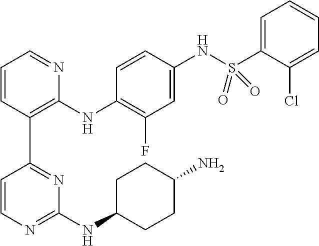 [SMILES: N[C@H]1CC[C@H](Nc2nccc(-c3cccnc3Nc3ccc(NS(=O)(=O)c4ccccc4Cl)cc3F)n2)CC1] |
| 124 | 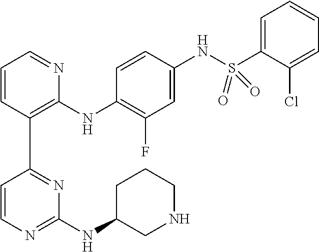 [SMILES: O=S(=O)(Nc1ccc(Nc2ncccc2-c2ccnc(N[C@H]3CCCNC3)n2)c(F)c1)c1ccccc1Cl] |
| 125 | 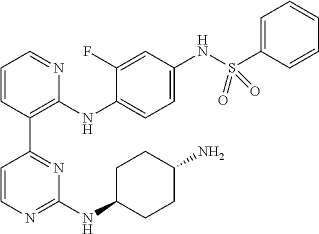 [SMILES: N[C@H]1CC[C@H](Nc2nccc(-c3cccnc3Nc3ccc(NS(=O)(=O)c4ccccc4)cc3F)n2)CC1] |
| 126 | 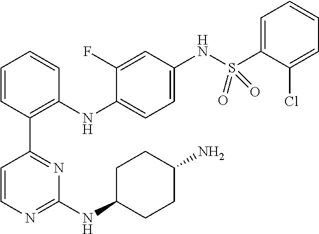 [SMILES: N[C@H]1CC[C@H](Nc2nccc(-c3ccccc3Nc3ccc(NS(=O)(=O)c4ccccc4Cl)cc3F)n2)CC1] |
| 127 | 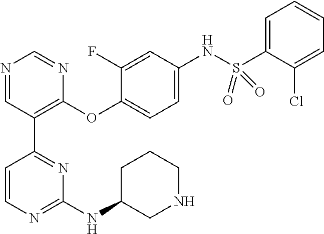 [SMILES: O=S(=O)(Nc1ccc(Oc2ncncc2-c2ccnc(N[C@H]3CCCNC3)n2)c(F)c1)c1ccccc1Cl] |
| 128 | 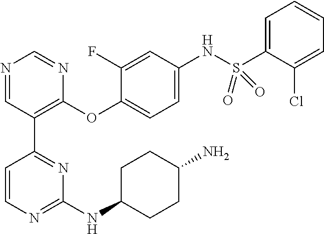 [SMILES: N[C@H]1CC[C@H](Nc2nccc(-c3cncnc3Oc3ccc(NS(=O)(=O)c4ccccc4Cl)cc3F)n2)CC1] |
| 129 | 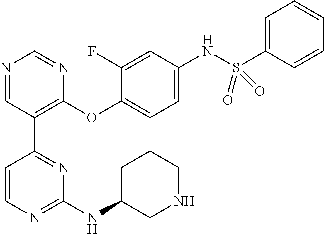 [SMILES: O=S(=O)(Nc1ccc(Oc2ncncc2-c2ccnc(N[C@H]3CCCNC3)n2)c(F)c1)c1ccccc1] |
| 130 | 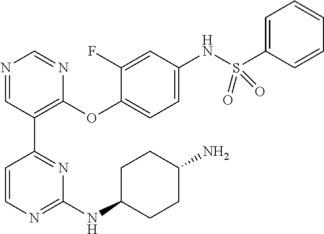 [SMILES: N[C@H]1CC[C@H](Nc2nccc(-c3cncnc3Oc3ccc(NS(=O)(=O)c4ccccc4)cc3F)n2)CC1] |
| 131 | 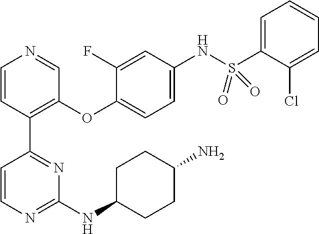 [SMILES: N[C@H]1CC[C@H](Nc2nccc(-c3ccncc3Oc3ccc(NS(=O)(=O)c4ccccc4Cl)cc3F)n2)CC1] |
| 132 | 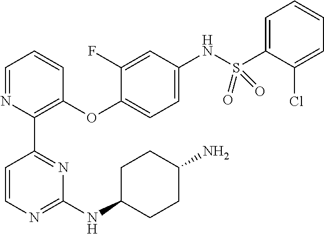 [SMILES: N[C@H]1CC[C@H](Nc2nccc(-c3ncccc3Oc3ccc(NS(=O)(=O)c4ccccc4Cl)cc3F)n2)CC1] |

or pharmaceutically acceptable salts thereof.

Also provided herein, in some embodiments, is a conjugate, comprising a compound disclosed herein, or a pharmaceutically acceptable salt thereof, bound to CDK2.

### Pharmaceutical Compositions

Compounds provided in accordance with the present disclosure, in some embodiments, are administered in the form of pharmaceutical compositions. This disclosure therefore provides pharmaceutical compositions that comprise, as the active ingredient, one or more of the compounds described, or a pharmaceutically acceptable salt thereof, and one or more pharmaceutically acceptable excipients, including inert solid diluents and fillers, diluents, including sterile aqueous solution and various organic solvents, permeation enhancers, solubilizers and adjuvants. In some embodiments, the pharmaceutical compositions are administered in combination with other therapeutic agents.

Provided herein, in some embodiments, are pharmaceutical compositions, comprising: a) a compound disclosed herein, or a pharmaceutically acceptable salt thereof; and b) a pharmaceutically acceptable excipient.

In some embodiments, the pharmaceutical compositions are administered in a single dose by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer. In some embodiments, the pharmaceutical compositions are administered in multiple doses.

One mode for administration is parenteral, for example by injection. Administration by injection comprises, in some embodiments, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline are also conventionally used for injection. In some embodiments, compositions suitable for injection comprise ethanol, glycerol, propylene glycol, liquid polyethylene glycol, or the like (and suitable mixtures thereof), cyclodextrin derivatives, or vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, or the like.

Sterile injectable solutions are prepared by incorporating a compound according to the present disclosure in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Oral administration is another route for administration of compounds in accordance with the disclosure. Oral administration includes, for example, capsule or enteric coated tablets, or the like. In making the pharmaceutical compositions that include at least one compound described herein, the active ingredient is, in some embodiments, diluted by an excipient and/or enclosed within such a carrier that can be in the form of a capsule, sachet, paper or other container. When the excipient serves as a diluent, it can be in the form of a solid, semi-solid, or liquid material (as above), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments comprising the active compound, soft and hard gelatin capsules, sterile injectable solutions, or sterile packaged powders.

The compositions of the disclosure can be formulated so as to provide quick, sustained or delayed release of the active ingredient after administration to the patient by employing procedures known in the art. Controlled release drug delivery systems for oral administration include osmotic pump systems and dissolutional systems containing polymer-coated reservoirs or drug-polymer matrix formulations. Another formulation for use in the methods of the present disclosure employs transdermal delivery devices (“patches”). Such transdermal patches may be used to provide continuous or discontinuous infusion of the compounds of the present disclosure in controlled amounts. Such patches may be constructed for continuous, pulsatile, or on demand delivery of pharmaceutical agents.

The compositions are preferably formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient (e.g., a tablet, capsule, ampoule). The compounds are generally administered in a pharmaceutically effective amount. It will be understood, however, that the amount of the compound actually administered usually will be determined by a physician, in the light of the relevant circumstances, including the condition to be treated, the chosen route of administration, the actual compound administered and its relative activity, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present disclosure. When referring to these preformulation compositions as homogeneous, it is meant that the active ingredient is dispersed evenly throughout the composition so that the composition may be readily subdivided into equally effective unit dosage forms such as tablets, pills and capsules.

In some embodiments, the tablets or pills of the present disclosure are coated or otherwise compounded to provide a dosage form affording the advantage of prolonged action, or to protect from the acid conditions of the stomach. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation include solutions and suspensions in pharmaceutically acceptable, aqueous or organic solvents, or mixtures thereof, and powders. In some embodiments, the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

### Methods of Use

Compounds and compositions described herein are utilized in methods for treating, preventing, or reducing the risk or severity of a disease or disorder mediated by unwanted CDK2 activity, a disease or disorder involving over-expression of cycle E, a disease or disorder involving over-expression of cyclin A2, or a disease or disorder that is otherwise treatable with a CDK2 inhibitor.

Provided herein, in some embodiments, are methods of inhibiting CDK2 activity in a subject in need thereof, comprising administering to the subject an effective amount of a compound disclosed herein or a pharmaceutically acceptable salt thereof, or a composition disclosed herein.

Also provided herein, in some embodiments, are methods of reducing CDK2 activity in a subject in need thereof, comprising administering to the subject an effective amount of a compound disclosed herein or a pharmaceutically acceptable salt thereof, or a composition disclosed herein.

Also provided herein, in some embodiments, are methods of treating a disease or disorder characterized by overexpression or amplification of cyclin E in a subject in need thereof, comprising administering to the subject an effective amount of a compound disclosed herein or a pharmaceutically acceptable salt thereof, or a composition disclosed herein.

In some embodiments, the cyclin E is CCNE1 or CCNE2.

In some embodiments, provided herein are methods of treating a disease or disorder characterized by overexpression or amplification of cyclin A in a subject in need thereof, comprising administering to the subject an effective amount of a compound disclosed herein or a pharmaceutically acceptable salt thereof, or a composition disclosed herein.

Also provided herein, in some embodiments, are methods of preventing a disease or disorder characterized by overexpression or amplification of cyclin E in a subject in need thereof, comprising administering to the subject an effective amount of a compound disclosed herein or a pharmaceutically acceptable salt thereof, or a composition disclosed herein.

In some embodiments, the cyclin E is CCNE1 or CCNE2.

In some embodiments, provided herein are methods of preventing a disease or disorder characterized by overexpression or amplification of cyclin A in a subject in need thereof, administering to the subject an effective amount of a compound disclosed herein or a pharmaceutically acceptable salt thereof, or a composition disclosed herein.

Also provided herein, in some embodiments, are methods of reducing the risk or severity a disease or disorder characterized by overexpression or amplification of cyclin E in a subject in need thereof, comprising administering to the subject an effective amount of a compound disclosed herein or a pharmaceutically acceptable salt thereof, or a composition disclosed herein.

In some embodiments, the cyclin E is CCNE1 or CCNE2.

In some embodiments, provided herein are methods of reducing the risk or severity of a disease or disorder characterized by overexpression or amplification of cyclin A in a subject in need thereof, comprising administering to the subject an effective amount of a compound disclosed herein or a pharmaceutically acceptable salt thereof, or a composition disclosed herein.

In some embodiments, provided herein are methods of treating cancer in a subject in need thereof, comprising administering to the subject an effective amount of a compound disclosed herein or a pharmaceutically acceptable salt thereof, or a composition disclosed herein.

In some embodiments, the cancer is associated with amplification or overexpression of cyclin E. In some embodiments, the cyclin E is CCNE1 or CCNE2. In some embodiments, the cancer is associated with amplification or overexpression of cyclin A.

In some embodiments, the cancer is breast cancer, ovary cancer, cervix cancer, prostate cancer, testis cancer, genitourinary tract cancer, esophagus cancer, larynx cancer, glioblastoma, neuroblastoma, stomach cancer, skin cancer, keratoacanthoma, lung cancer, epidermoid carcinoma, large cell cancer, non-small cell lung cancer (NSCLC), small cell carcinoma, lung adenocarcinoma, bone cancer, colon cancer, adenoma, pancreatic cancer, adenocarcinoma, thyroid cancer, follicular carcinoma, undifferentiated carcinoma, papillary carcinoma, seminoma, melanoma, sarcoma, bladder carcinoma, liver carcinoma and biliary passages, kidney carcinoma, buccal cavity cancer, naso-pharyngeal cancer, pharynx cancer, lip cancer, tongue cancer, mouth cancer, small intestine cancer, colon-rectum cancer, large intestine cancer, rectum cancer, bronchial cancer, hepatocellular cancer, gastric cancer, endometrial cancer, melanoma, renal cancer, urinary bladder cancer, uterine corpus cancer, or uterine cervix cancer.

In some embodiments, the cancer is squamous cell carcinoma, small-cell lung cancer, non-small cell lung cancer (NSCLC), lung adenocarcinoma, squamous cell lung cancer, peritoneum cancer, hepatocellular cancer, stomach cancer, gastrointestinal cancer, esophageal cancer, pancreatic cancer, glioblastoma, cervical cancer, ovarian cancer, liver cancer, bladder cancer, breast cancer, colon cancer, rectal cancer, colorectal cancer, endometrial cancer, uterine cancer, salivary gland carcinoma, renal cancer, prostate cancer, vulval cancer, thyroid cancer, hepatocellular carcinoma (HCC), anal carcinoma, penile carcinoma, or head and neck cancer.

In some embodiments, the cancer is breast cancer. The breast cancer can be Stage I, II, III, or IV as understood in the art. In some embodiments, the breast cancer is triple negative breast cancer (TNBC). In another embodiment, the breast cancer is Her2 negative breast cancer.

In some embodiments, the cancer is breast cancer, ovarian cancer, uterine cancer, endometrial cancer, cervical cancer, melanoma, lung cancer, pancreatic cancer, stomach cancer, esophageal cancer, bladder cancer, colon cancer, rectal cancer, testicular cancer, prostate cancer, renal cancer, hepatic cancer, head and neck cancer, thyroid cancer, glioblastoma, sarcoma, lymphoma, and leukemia.

In some embodiments, provided herein are methods of treating a hematological malignancy in a subject in need thereof, comprising administering to the subject an effective amount of a compound disclosed herein or a pharmaceutically acceptable salt thereof, or a composition disclosed herein.

In some embodiments, the hematological malignancy is associated with amplification or overexpression of cyclin E. In some embodiments, the cyclin E is CCNE1 or CCNE2. In some embodiments, the hematological malignancy is associated with amplification or overexpression of cyclin A.

In some embodiments, hematological malignancy is lymphoma, lymphocytic leukemia (acute (ALL) and chronic (CLL), multiple myeloma (MM), acute myelogenous leukemia (AML), chronic myelogenous leukemia (CML), myelodysplastic syndrome (MDS), myeloproliferative disease (MPD), or non-Hodgkin lymphoma. In some embodiments, the methods herein include treatment of lymphoma, lymphocytic leukemia, multiple myeloma (MM), acute myelogenous leukemia (AML), chronic myelogenous leukemia (CML), myelodysplastic syndrome (MDS), or myeloproliferative disease (MPD).

In some embodiments, the methods provided herein further comprises administering to the subject an additional therapeutic agent(s) selected from the group consisting of an anti-inflammatory agent, a corticosteroid, an immunomodulatory agent, anti-cancer agent as described herein, an apoptosis-enhancer, a neurotropic factor, an agent for treating cardiovascular disease, an agent for treating liver disease, an anti-viral agent, an agent for treating blood disorders, an agent for treating diabetes, an agent for treating metabolic disorders, an agent for treating autoimmune disorders, an agent for treating immunodeficiency disorders, and combinations thereof. In some embodiments, the additional therapeutic agent is a corticosteroid, a proteasome inhibitor, an IMiD, an antibody, or a combination thereof. In some embodiments, the additional therapeutic agent is a proteasome inhibitor (e.g. carfilzomib, bortezomib, or ixazomib). In some embodiments, the additional therapeutic agent is an IMiD (e.g. lenalidomide or pomalidomide). In some embodiments, the additional therapeutic agent is an antibody (e.g., an anti-CD38 antibody, an anti-VEGF-A antibody, an anti-PD-1 antibody, an anti-PD-L1 antibody or an anti-interleukin-6 antibody). In some embodiments, the additional therapeutic agent is a corticosteroid (e.g., dexamethasone). In some embodiments, the method further comprises radiotherapy.

Also provided herein are methods of treating an autoimmune disorder or disease in a subject in need thereof, administering to the subject an effective amount of a compound disclosed herein or a pharmaceutically acceptable salt thereof, or a composition disclosed herein.

In some embodiments, the autoimmune disorder or disease is associated with amplification or overexpression of cyclin E. In some embodiments, the cyclin E is CCNE1 or CCNE2. In some embodiments, the autoimmune disorder or disease is associated with amplification or overexpression of cyclin A.

Exemplary autoimmune disorder or disease contemplated in the methods provided herein includes, but not limited to, rheumatoid arthritis, Lupus, Crohn's Disease, Addison disease, celiac disease, dermatomyositis, Graves' disease, thyroiditis, multiple sclerosis, pernicious anemia, reactive arthritis, pemphigus vulgaris, diabetes mellitus type 1 (IDDM), systemic lupus erythematosus (SLE), Sjogren's syndrome, Churg-Strauss Syndrome, Hashimoto's thyroiditis, idiopathic thrombocytopenic purpura, or rheumatoid arthritis.

Also provided herein is are methods of treating an inflammatory disease or disorder in a subject in need thereof, comprising administering to the subject an effective amount of a compound disclosed herein or a pharmaceutically acceptable salt thereof, or a composition disclosed herein.

In some embodiments, the inflammatory disease or disorder is associated with amplification or overexpression of cyclin E. In some embodiments, the cyclin E is CCNE1 or CCNE2. In some embodiments, the inflammatory disease or disorder is associated with amplification or overexpression of cyclin A.

Exemplary inflammatory disease or disorder contemplated in the methods provided herein includes, but not limited to, asthma, chronic peptic ulcers, psoriasis, inflammatory bowel disease, tuberculosis, rheumatoid arthritis, periodontitis, ulcerative colitis, hepatitis chronic prostatitis, glomerulonephritis, hypersensitivities, pelvic inflammatory disease, reperfusion injury, transplant rejection, or vasculitis.

Also provided herein are methods of treating a neurodegenerative disease or disorder in a subject in need thereof, administering to the subject an effective amount of a compound disclosed herein or a pharmaceutically acceptable salt thereof, or a composition disclosed herein.

In some embodiments, the neurodegenerative disease or disorder is associated with amplification or overexpression of cyclin E. In some embodiments, the cyclin E is CCNE1 or CCNE2. In some embodiments, the neurodegenerative disease or disorder is associated with amplification or overexpression of cyclin A.

Exemplary neurodegenerative disease or disorder contemplated in the methods provided herein includes, but not limited to, Alzheimer's disease, AIDS-related dementia, Parkinson's disease, amyotrophic lateral sclerosis, retinitis pigmentosa, spinal muscular atrophy, cerebellar degeneration, Alexander's disease, Alper's disease, Ataxia telangiectasia, Batten disease (also known as Spielmeyer-Vogt-Sjogren-Batten disease), Bovine spongiform encephalopathy (BSF), Canavan disease, Cockayne syndrome, Corticobasal degeneration, Creutzfeldt-Jakob disease, Huntington's disease, HIV-associated dementia, Kennedy's disease, Krabbe's disease, Lewy body dementia, Machado-Joseph disease (Spinocerebellar ataxia type 3), Multiple sclerosis, Multiple System Atrophy, Narcolepsy, Neuroborreliosis, Pelizaeus-Merzbacher Disease, Pick's disease, Primary lateral sclerosis, Prion diseases, Refsum's disease, Sandhoffs disease, Schilder's disease, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, or Tabes dorsalis.

Also provided herein, in some embodiments, are methods of killing a cell over-expressing cyclin E in a subject in need thereof, comprising administering to the subject an effective amount of a compound of Formula (I), (II), (III), (IV), (V), (VI), and (VII), or a pharmaceutically acceptable salt thereof, or a pharmaceutical composition thereof. In some embodiments, also disclosed herein are methods of killing a cell over-expressing or amplifying cyclin E, comprising contacting the cell with an effective amount of a compound of Formula (I), (II), (III), (IV), (V), (VI), and (VII), or a pharmaceutically acceptable salt thereof, or a pharmaceutical composition thereof.

In some embodiments, the cyclin E is CCNE1 or CCNE2.

Also provided herein, in some embodiments, are methods of killing a cell over-expressing cyclin A in a subject in need thereof, comprising administering to the subject an effective amount of a compound of Formula (I), (II), (III), (IV), (V), (VI), and (VII), or a pharmaceutically acceptable salt thereof, or a pharmaceutical composition thereof. In some embodiments, also disclosed herein are methods of killing a cell over-expressing or amplifying cyclin A, comprising contacting the cell with an effective amount of a compound of Formula (I), (II), (III), (IV), (V), (VI), and (VII), or a pharmaceutically acceptable salt thereof, or a pharmaceutical composition thereof.

Also provided herein, in some embodiments, are methods of inhibiting CDK2 in a cell over-expressing cyclin E in a subject in need thereof, comprising administering to the subject an effective amount of a compound of Formula (I), (II), (III), (IV), (V), (VI), and (VII), or a pharmaceutically acceptable salt thereof, or a pharmaceutical composition thereof. In some embodiments, also disclosed herein are methods of inhibiting CDK2 in cell over-expressing or amplifying cyclin E, comprising contacting the cell with an effective amount of a compound of Formula (I), (II), (III), (IV), (V), (VI), and (VII), or a pharmaceutically acceptable salt thereof, or a pharmaceutical composition thereof.

In some embodiments, the cyclin E is CCNE1 or CCNE2.

Also provided herein, in some embodiments, are methods of inhibiting CDK2 in a cell over-expressing cyclin A in a subject in need thereof, comprising administering to the subject an effective amount of a compound of Formula (I), (II), (III), (IV), (V), (VI), and (VII), or a pharmaceutically acceptable salt thereof, or a pharmaceutical composition thereof. In some embodiments, also disclosed herein are methods of inhibiting CDK2 in a cell over-expressing or amplifying cyclin A, comprising contacting the cell with an effective amount of a compound of Formula (I), (II), (III), (IV), (V), (VI), and (VII), or a pharmaceutically acceptable salt thereof, or a pharmaceutical composition thereof.

Also provided herein, in some embodiments, are methods of killing a cell over-expressing or amplifying cyclin E, comprising contacting the cell with an effective amount of a compound of Formula (I), (II), (III), (IV), (V), (VI), and (VII), or a pharmaceutically acceptable salt thereof, or a pharmaceutical composition thereof.

Also provided herein, in some embodiments, are methods of killing a cell over-expressing or amplifying cyclin A, comprising contacting the cell with an effective amount of a compound of Formula (I), (II), (III), (IV), (V), (VI), and (VII), or a pharmaceutically acceptable salt thereof, or a pharmaceutical composition thereof.

### Dosage Regimens

Dosage regimens may be adjusted to provide the optimum desired response. The skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a patient in practicing the present disclosure.

It is to be noted that dosage values may vary with the type and severity of the condition to be alleviated and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present disclosure encompasses intra-patient dose-escalation as determined by the skilled artisan. Determining appropriate dosages and regimens for administration of the chemotherapeutic agent are well-known in the relevant art and would be understood to be encompassed by the skilled artisan once provided the teachings disclosed herein.

The amount of the compound of the disclosure administered will be dependent on the subject being treated, the severity of the disorder or condition, the rate of administration, the disposition of the compound and the discretion of the prescribing physician. However, an effective dosage is in the range of about 0.001 to about 100 mg per kg body weight per day. In some instances, dosage levels below the lower limit of the aforesaid range may be more than adequate, while in other cases still larger doses may be employed without causing any harmful side effect, provided that such larger doses are first divided into several small doses for administration throughout the day.

### Kits

In another aspect provided herein are kits, comprising materials useful for the treatment or prevention of the diseases and disorders described above. In some embodiments, the kit comprises a container comprising a compound of Formula (I), (II), (III), (IV), (V), (VI), and (VII), or a pharmaceutically acceptable salt thereof. In some embodiments, the kit further comprises a label or package insert on or associated with the container. Suitable containers include, for example, bottles, vials, syringes, blister pack, etc. The container can be formed from a variety of materials such as glass or plastic. The container can hold a compound of Formula (I), (II), (III), (IV), (V), (VI), and (VII), or a composition thereof which is effective for treating or preventing the condition and can have a sterile access port (for example, the container can be an intravenous solution bag or a vial having a stopper pierceable by a hypodermic injection needle). At least one active agent in the composition is a compound of Formula (I), (II), (III), (IV), (V), (VI), and (VII), or a pharmaceutically acceptable salt thereof. The label or package insert indicates that the composition is used for treating the condition of choice. The label or package insert can also indicate that the composition can be used to treat other disorders. Alternatively, or additionally, the article of manufacture can further comprise a second container comprising a pharmaceutically acceptable buffer, such as bacteriostatic water for injection (BWFI), phosphate-buffered saline, Ringer's solution and dextrose solution. It can further include other materials desirable from a commercial and user standpoint, including other buffers, diluents, filters, needles, and syringes.

The kit can further comprise directions for the administration of the compound of Formula (I), (II), (III), (IV), (V), (VI), and (VII), or a pharmaceutically acceptable salt thereof and, if present, the second pharmaceutical formulation. For example, if the kit comprises a first composition comprising a compound of Formula (I), (II), (III), (IV), (V), (VI), and (VII), or a pharmaceutically acceptable salt thereof and a second pharmaceutical formulation, the kit can further comprise directions for the simultaneous, sequential or separate administration of the first and second pharmaceutical compositions to a patient in need thereof.

In another embodiment, the kits are suitable for the delivery of solid oral forms of a compound of Formula (I), (II), (III), (IV), (V), (VI), and (VII), or a pharmaceutically acceptable salt thereof, such as tablets or capsules. Such a kit preferably includes a number of unit dosages. Such kits can include a card having the dosages oriented in the order of their intended use. An example of such a kit is a blister pack. Blister packs are well known in the packaging industry and are widely used for packaging pharmaceutical unit dosage forms. If desired, a memory aid can be provided, for example in the form of numbers, letters, or other markings or with a calendar insert, designating the days in the treatment schedule in which the dosages can be administered.

In some embodiments, a kit comprises (a) a first container with a compound of Formula (I), (II), (III), (IV), (V), (VI), and (VII), or a pharmaceutically acceptable salt thereof contained therein; and optionally (b) a second container with a second pharmaceutical formulation contained therein. Alternatively, or additionally, the kit can further comprise a third container comprising a pharmaceutically-acceptable buffer, such as bacteriostatic water for injection (BWFI), phosphate-buffered saline, Ringer's solution and dextrose solution. It can further include other materials desirable from a commercial and user standpoint, including other buffers, diluents, filters, needles, and syringes.

In some embodiments wherein the kit comprises a composition of Formula (I), (II), (III), (IV), (V), (VI), and (VII), or a pharmaceutically acceptable salt thereof and a second therapeutic agent, the kit can comprise a container for containing the separate compositions such as a divided bottle or a divided foil packet, however, the separate compositions can also be contained within a single, undivided container. Typically, the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

### EXAMPLES

Selected abbreviations: ACN: acetonitrile; DCM: dichloromethane; DMF: N,N-dimethylformamide; DMSO: dimethyl sulfoxide; EtOAc: ethyl acetate; PE: petroleum ether; THF: tetrahydrofuran; TLC: thin layer chromatography.

### Example 1. Synthetic Methods

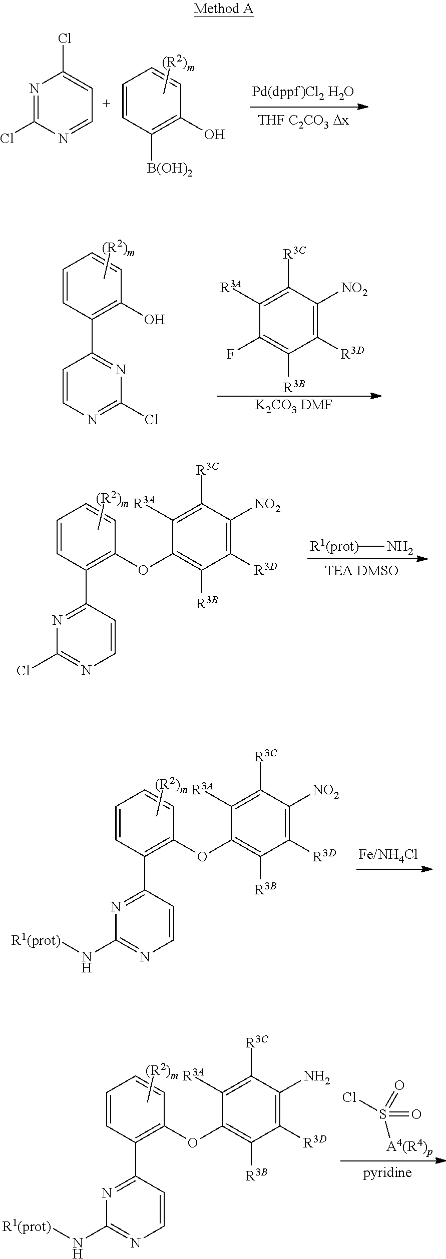

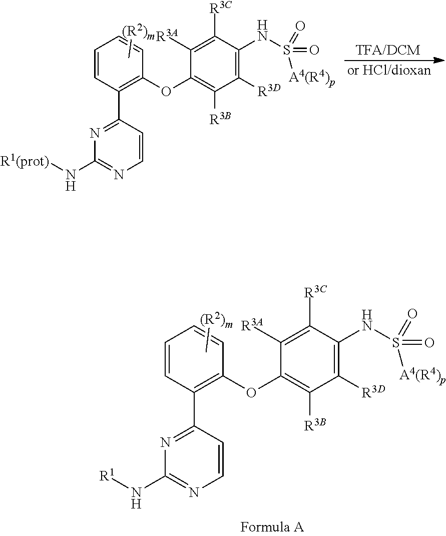

Compounds of general Formula A can be synthesized by routes familiar to one of skill in the art, such as that illustrated above. A Suzuki coupling between 2,4-dichloropyrimidine and a 2-hydroxyphenyl boronic acid forms a 2-chloro-4-(2-hydroxyphenyl)pyrimidine. Under basic conditions, the phenoxide derived from this can displace the 4-fluoro in a suitably substituted 4-fluoronitrobenzene, to form a 4-nitrophenyl ether. This compound can be reacted with the C2 amine, which may contain a suitably protected primary or secondary amine substituent. Reduction of the nitro group can be carried out in many ways, including dissolving metal reductions, as illustrated herein, and the aniline amino can be sulfonated with a suitable sulfonyl halide, such as the chloride, to give a 4-sulfonamidophenoxy ether product, which may require a final deprotection of a C2-side chain amine to give the final product. Timing of individual steps may also vary depending on the chemistry needed to incorporate various substituents. For example, a sulfonamide bond may be formed to link the A³ and A⁴ rings before the S_{N}Ar reaction to link the A² and A³ rings, or the S_{N}Ar reaction may be carried out to link the A² and A³ rings before the Suzuki-Miyauri biaryl coupling to link the A¹ and A² rings.

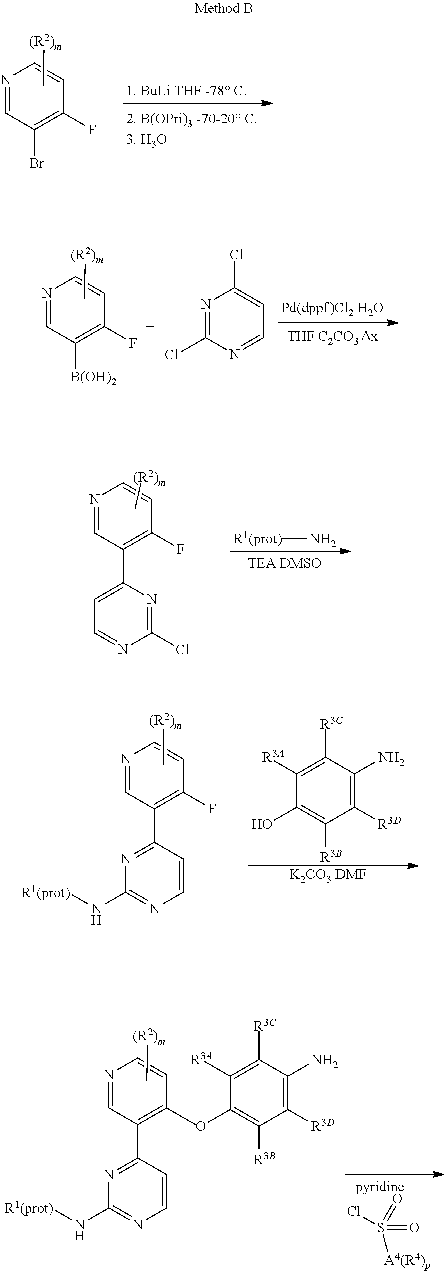

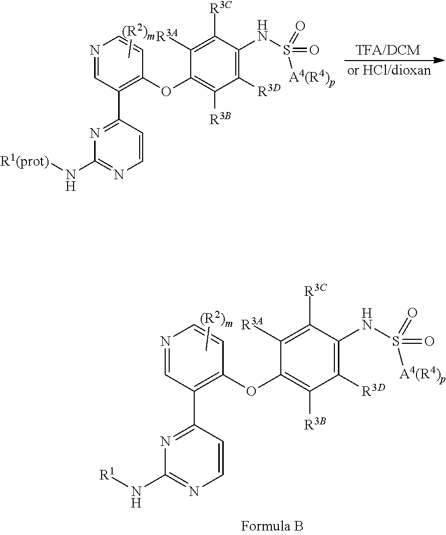

Compounds of general Formula B can be synthesized by routes familiar to one of skill in the art, such as that illustrated above. An optionally substituted 3-bromo-4-fluoropyridine is converted to the corresponding pyridine-3-boronic acid, by halogen metal exchange, and quenching with tri-isopropyl borate. A Suzuki coupling between this pyridyl boronic acid and 2,4-dichloropyrimidine forms a 2-chloro-4-(4-fluoropyridin-3-yl)pyrimidine. This compound can be reacted with the C2 amine, which may contain a suitably protected primary or secondary amine substituent. Under basic conditions, the phenoxide derived from a suitably substituted 4-aminophenol can displace the 4-fluoro from the biaryl, to form a 4-aminophenyl ether. The aniline amino group can be sulfonated with a suitable sulfonyl halide, such as the chloride, to give a 4-sulfonamidophenoxy ether product, which may require a final deprotection of a C2-side chain amine to give the final product. Timing of individual steps may also vary depending on the chemistry needed to incorporate various substituents. For example, a sulfonamide bond may be formed to link the A³ and A⁴ rings before the S_{N}Ar reaction to link the A² and A³ rings, or the S_{N}Ar reaction may be carried out to link the A² and A³ rings before the Suzuki-Miyauri biaryl coupling to link the A¹ and A² rings.

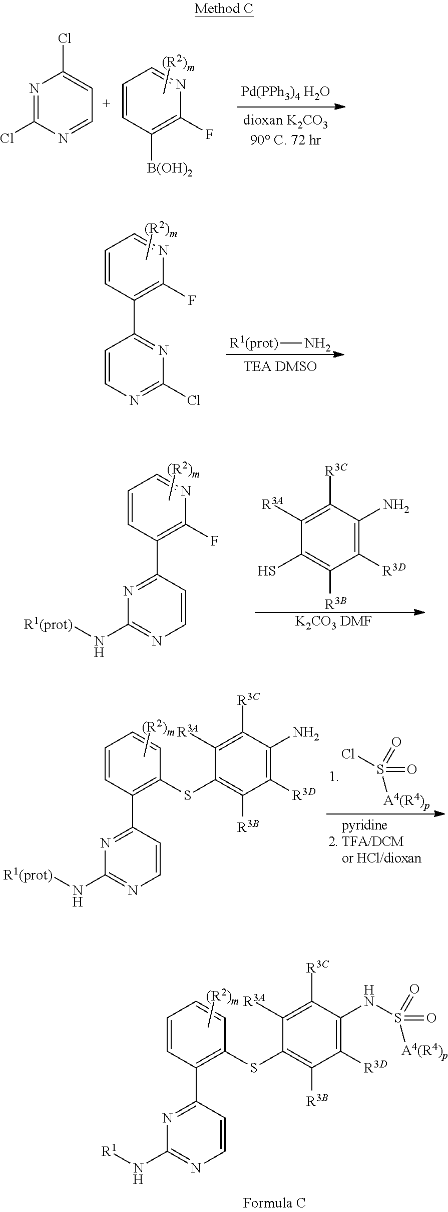

Compounds of general Formula C can be synthesized by routes familiar to one of skill in the art, such as that illustrated above. A Suzuki coupling between 2,4-dichloropyrimidine and a 2-fluoropyrid-3-yl boronic acid forms a 2-chloro-4-(2-fluoropyrid-3-yl)pyrimidine. This compound can be selectively reacted with the C2 amine, which may contain a suitably protected primary or secondary amine substituent, with displacement of the pyrimidyl chlorine. Under basic conditions, the thiolate anion, derived from a suitable 4-thioaniline this can then displace the 2-fluoro on the pyridyl ring to form a 4-aminothiophenyl ether. The aniline amino can be sulfonated with a suitable sulfonyl halide, such as the chloride, to give a 4-sulfonamidophenoxy ether product, which may require a final deprotection of a C2-side chain amine to give the final product. Timing of individual steps may also vary depending on the chemistry needed to incorporate various substituents. For example, a sulfonamide bond may be formed to link the A³ and A⁴ rings before the S_{N}Ar reaction to link the A² and A³ rings, or the S_{N}Ar reaction may be carried out to link the A² and A³ rings before the Suzuki-Miyauri biaryl coupling to link the A¹ and A² rings.

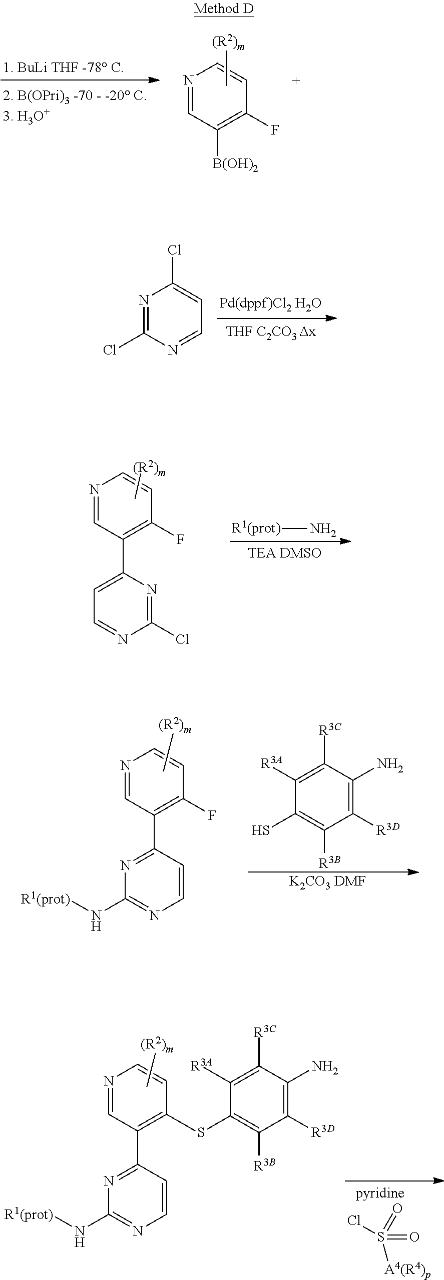

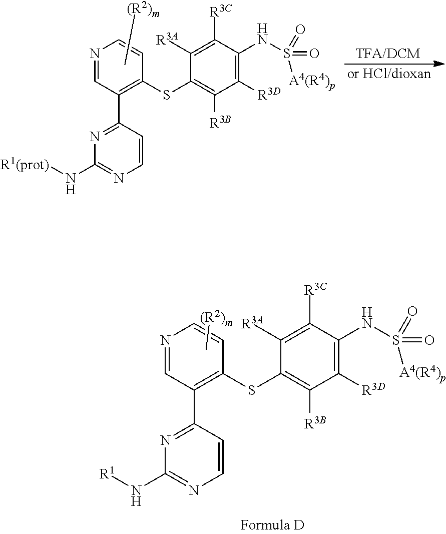

Compounds of general Formula D can be synthesized by routes familiar to one of skill in the art, such as that illustrated above. An optionally substituted 2-chloro-4-(4-fluoropyridin-3-yl)pyrimidine is made as described in General Method B. Under mildly basic conditions, the thiolate derived from a suitably substituted 4-aminothiophenol can displace the 4-fluoro from the biaryl, to form a 4-aminophenyl thioether. This compound can be reacted with the C2 amine, which may contain a suitably protected primary or secondary amine substituent. The aniline amino group can be sulfonated with a suitable sulfonyl halide, such as the chloride, to give a 4-sulfonamidothiophenoxy thioether product, which may require a final deprotection of a C2-side chain amine to give the final product. Timing of individual steps may also vary depending on the chemistry needed to incorporate various substituents. For example, a sulfonamide bond may be formed to link the A³ and A⁴ rings before the S_{N}Ar reaction to link the A² and A³ rings, or the S_{N}Ar reaction may be carried out to link the A² and A³ rings before the Suzuki-Miyauri biaryl coupling to link the A¹ and A² rings.

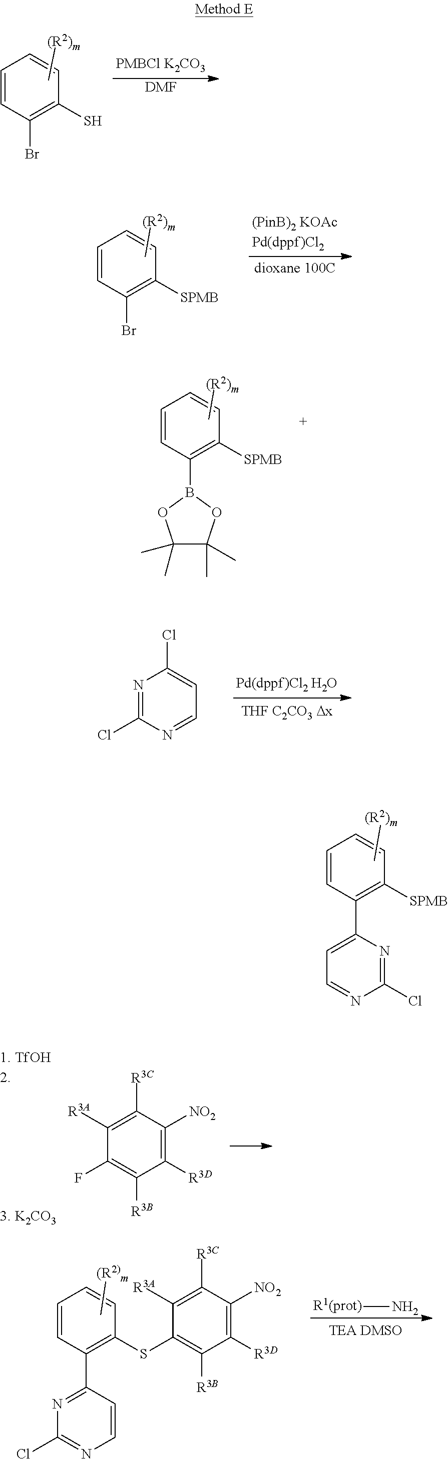

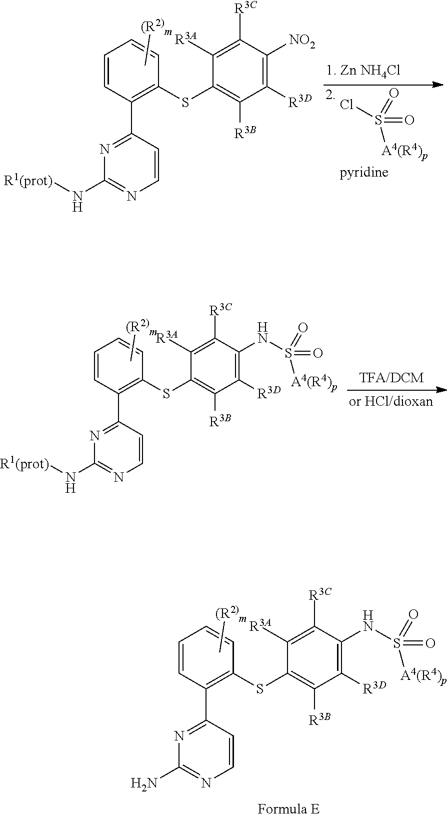

Compounds of general Formula E can be synthesized by routes familiar to one of skill in the art, such as that illustrated above. An optionally substituted 2-bromothiophenol is S-protected using p-methoxybenzyl chloride. This bromide is then borylated by known means to form, for example a pinacolborane species. This in turn is Suzuki coupled with 2,4-dichloropyrimidine to form the desired biaryl. The PMB is removed under strongly acidic conditions, and then an appropriately substituted 4-fluoronitrobenzene is added, and then the reaction is basified to induce the desired fluoride displacement S_{N}Ar reaction. This compound can be reacted with the C2 amine, which may contain a suitably protected primary or secondary amine substituent. The aniline nitro group can then be reduced, and the resultant amino group can be sulfonated with a suitable sulfonyl halide, such as the chloride, to give a 4-sulfonamidothiophenoxy thioether product, which may require a final deprotection of a C2-side chain amine to give the final product. Timing of individual steps may also vary depending on the chemistry needed to incorporate various substituents. For example, a sulfonamide bond may be formed to link the A³ and A⁴ rings before the S_{N}Ar reaction to link the A² and A³ rings, or the S_{N}Ar reaction may be carried out to link the A² and A³ rings before the Suzuki-Miyauri biaryl coupling to link the A¹ and A² rings.

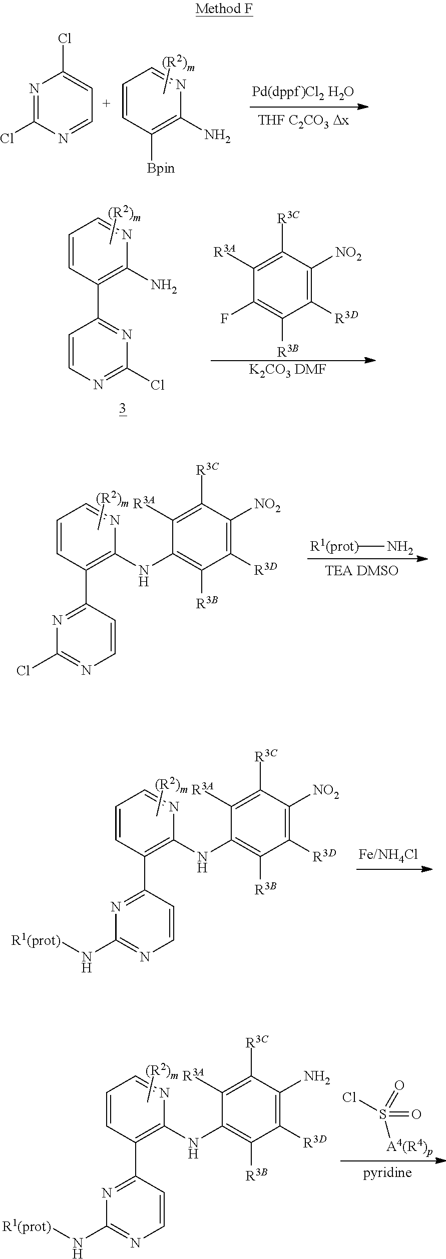

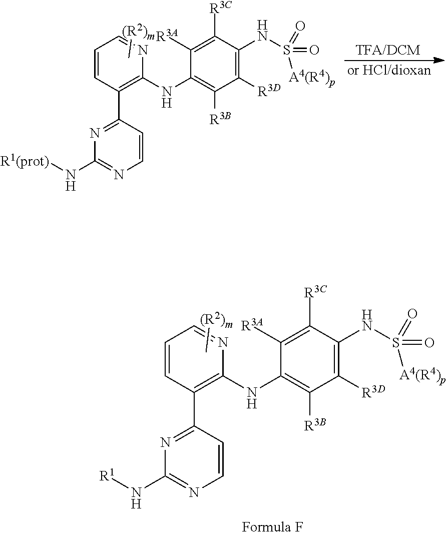

Compounds of general Formula F can be synthesized by routes familiar to one of skill in the art, such as that illustrated above. A Suzuki coupling between 2,4-dichloropyrimidine and a 2-aminopyrid-3-yl boronic acid forms a 2-chloro-4-(2-aminopyrid-3-yl)pyrimidine. Under basic conditions, the phenoxide derived from this can displace the 4-fluoro in a suitably substituted 4-fluoronitrobenzene, to form a 4-nitrophenyl diarylamine. This compound can be reacted with the C2 amine, which may contain a suitably protected primary or secondary amine substituent. Reduction of the nitro group can be carried out in many ways, including dissolving metal reductions, as illustrated herein, and the aniline amino can be sulfonated with a suitable sulfonyl halide, such as the chloride, to give a 4-sulfonamidophenoxy ether product, which may require a final deprotection of a C2-side chain amine to give the final product. Timing of individual steps may also vary depending on the chemistry needed to incorporate various substituents. For example, a sulfonamide bond may be formed to link the A³ and A⁴ rings before the S_{N}Ar reaction to link the A² and A³ rings, or the S_{N}Ar reaction may be carried out to link the A² and A³ rings before the Suzuki-Miyauri biaryl coupling to link the A¹ and A² rings.

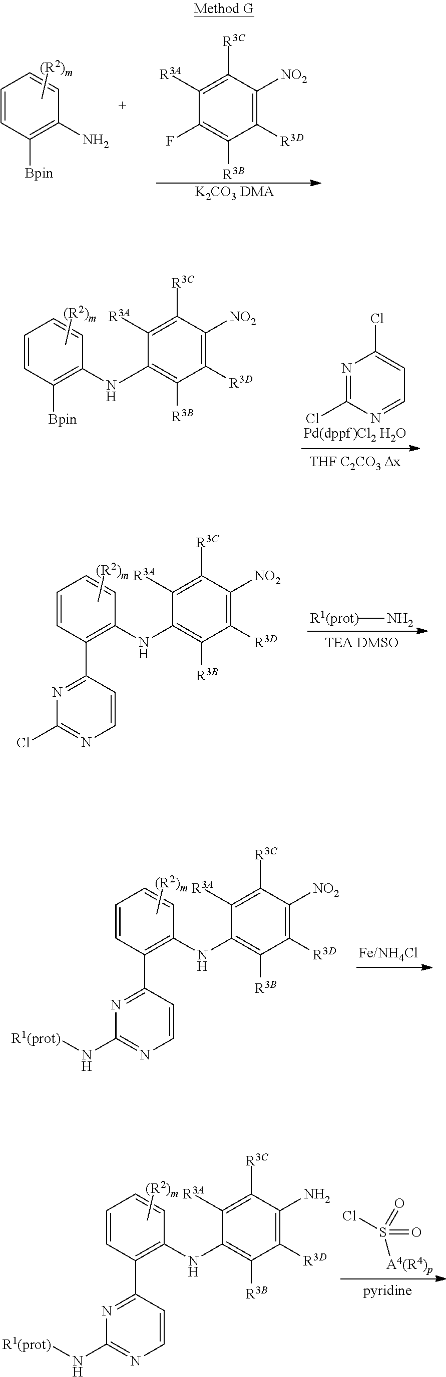

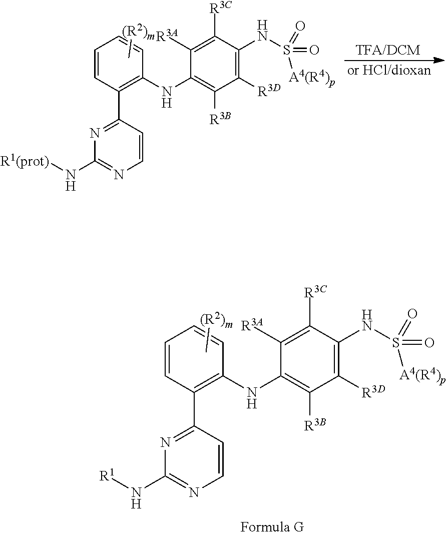

Compounds of general Formula G can be synthesized by routes familiar to one of skill in the art, such as that illustrated above. Under basic conditions, the amino group of a 2-aminophenyl boronic acid/ester can displace the 4-fluoro in a suitably substituted 4-fluoronitrobenzene, to form a 4-nitrophenyl diarylamine. A Suzuki coupling between this boronate and 2,4-dichloropyrimidine forms a 2-chloro-4-(2-anilinophenyl)pyrimidine. This compound can be reacted with the C2 amine, which may contain a suitably protected primary or secondary amine substituent. Reduction of the nitro group can be carried out in many ways, including dissolving metal reductions, as illustrated herein, and the aniline amino can be sulfonated with a suitable sulfonyl halide, such as the chloride, to give a 4-sulfonamidophenoxy ether product, which may require a final deprotection of a C2-side chain amine to give the final product. Timing of individual steps may also vary depending on the chemistry needed to incorporate various substituents. For example, a sulfonamide bond may be formed to link the A³ and A⁴ rings before the S_{N}Ar reaction to link the A² and A³ rings, or the S_{N}Ar reaction may be carried out to link the A² and A³ rings before the Suzuki-Miyauri biaryl coupling to link the A¹ and A² rings.

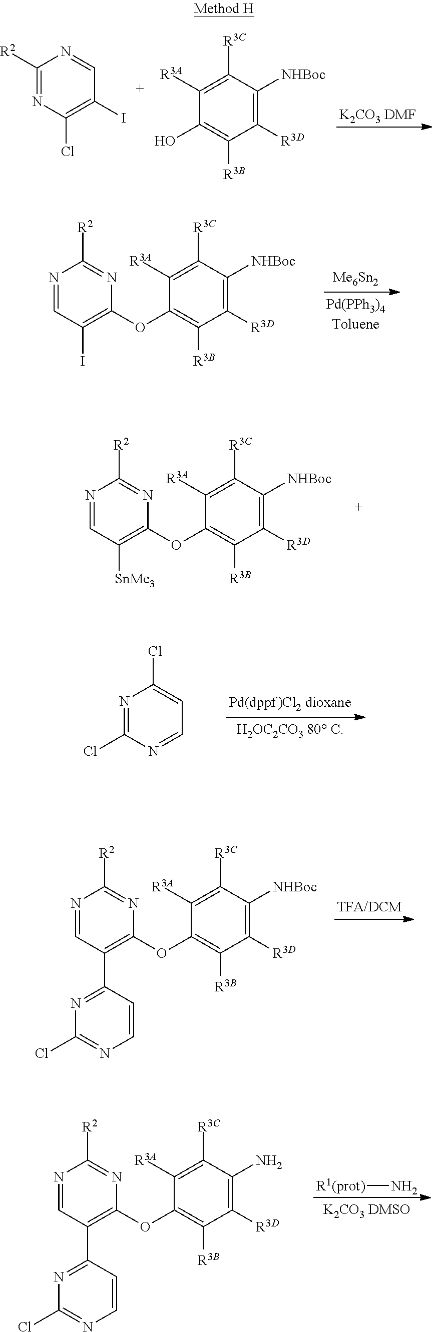

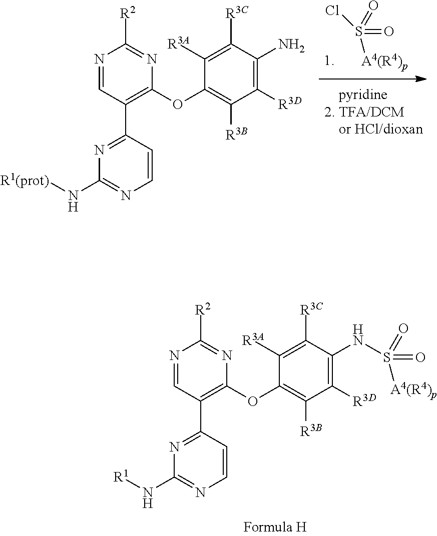

Compounds of general Formula H can be synthesized by routes familiar to one of skill in the art, such as that illustrated above. Displacement of the chloride of a 4-chloro-5-iodopyrimidine with a 4-Bocaminophenol under basic conditions give a pyrimidyl phenyl ether, which can be converted into the corresponding stannane with hexamethyl ditin. A Stille coupling of this with 2,4-dichloropyrimidine forms a 2-chloro-4-(4-phenoxypyrimid-5-yl)pyrimidine. This compound can be deBocced under standard conditions, and reacted with the C2 amine, which may contain a suitably protected primary or secondary amine substituent, with displacement of the pyrimidyl chlorine. The aniline amino can be sulfonated with a suitable sulfonyl halide, such as the chloride, to give a 4-sulfonamidophenoxy ether product, which may require a final deprotection of a C2-side chain amine to give the final product. Timing of individual steps may also vary depending on the chemistry needed to incorporate various substituents. For example, a sulfonamide bond may be formed to link the A³ and A⁴ rings before the S_{N}Ar reaction to link the A² and A³ rings, or the S_{N}Ar reaction may be carried out to link the A² and A³ rings before the Suzuki-Miyauri biaryl coupling to link the A¹ and A² rings.

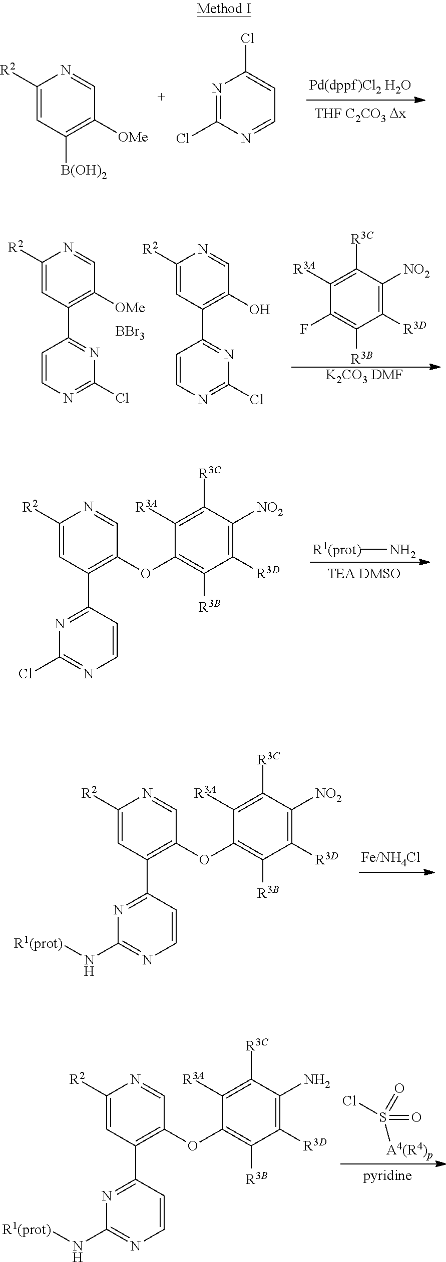

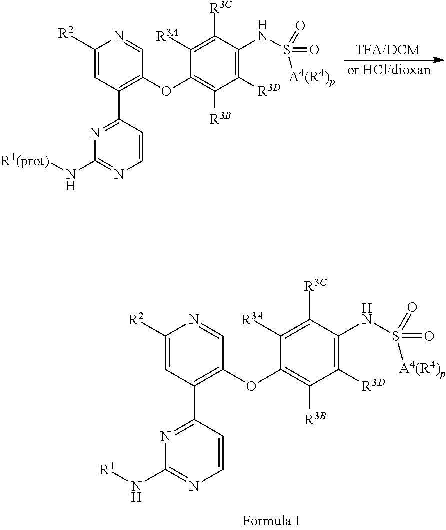

Compounds of general Formula I can be synthesized by routes familiar to one of skill in the art, such as that illustrated above. A Suzuki coupling between 2,4-dichloropyrimidine and a 3-methoxypyridine-4-boronic acid forms a 2-chloro-4-(3-methoxypyridin-4-yl)pyrimidine. Cleavage of the methyl ether under acidic conditions such as boron tribromide or trimethylsilyl iodide will give the corresponding phenol. Under basic conditions, the phenoxide derived from this can displace the 4-fluoro in a suitably substituted 4-fluoronitrobenzene, to form a 4-nitrophenyl ether. This compound can be reacted with the C2 amine, which may contain a suitably protected primary or secondary amine substituent. Reduction of the nitro group can be carried out in many ways, including dissolving metal reductions, as illustrated herein, and the aniline amino can be sulfonated with a suitable sulfonyl halide, such as the chloride, to give a 4-sulfonamidophenoxy ether product, which may require a final deprotection of a C2-side chain amine to give the final product. Timing of individual steps may also vary depending on the chemistry needed to incorporate various substituents. For example, a sulfonamide bond may be formed to link the A³ and A⁴ rings before the S_{N}Ar reaction to link the A² and A³ rings, or the S_{N}Ar reaction may be carried out to link the A² and A³ rings before the Suzuki-Miyauri biaryl coupling to link the A¹ and A² rings.

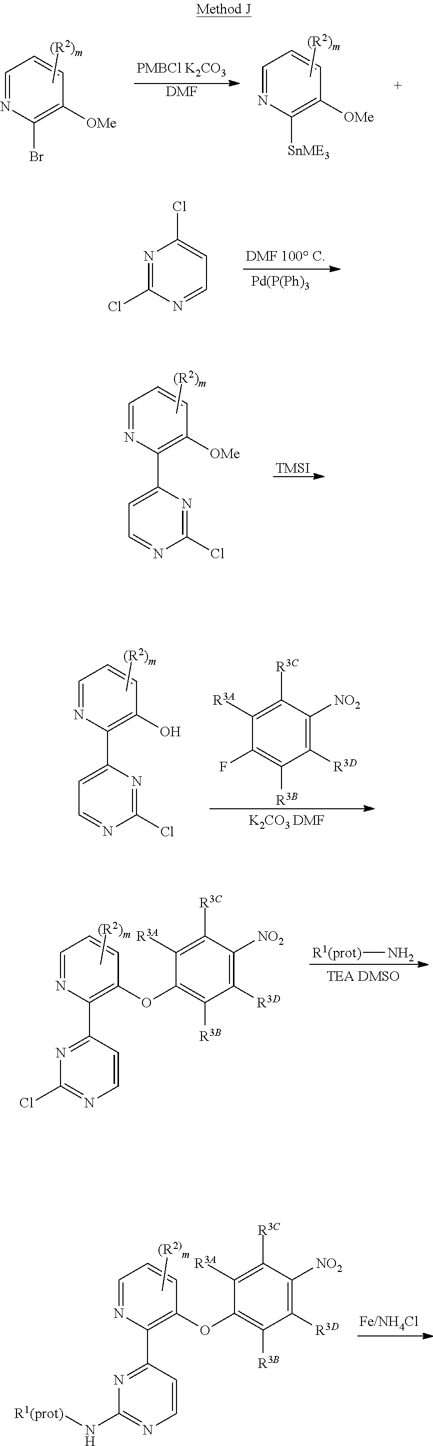

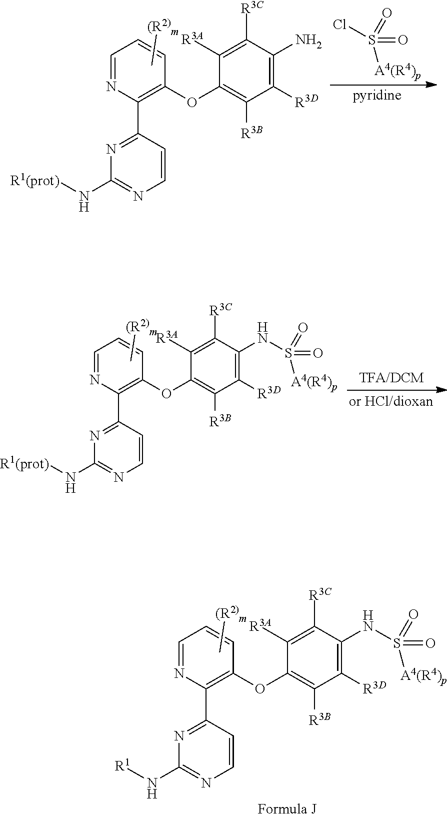

Compounds of general Formula J can be synthesized by routes familiar to one of skill in the art, such as that illustrated above. Conversion of a 3-methoxy-2-bromopyridine to the corresponding trimethyl stannane via a distannane stannylation, followed by a Stille coupling between 2,4-dichloropyrimidine and a 3-methoxy-2-stannylpyridine forms a 2-chloro-4-(3-methoxypyridin-2-yl)pyrimidine. Cleavage of the methyl ether under acidic conditions such as boron tribromide or trimethylsilyl iodide will give the corresponding phenol. Under basic conditions, the phenoxide derived from this can displace the 4-fluoro in a suitably substituted 4-fluoronitrobenzene, to form a 4-nitrophenyl ether. This compound can be reacted with the C2 amine, which may contain a suitably protected primary or secondary amine substituent. Reduction of the nitro group can be carried out in many ways, including dissolving metal reductions, as illustrated herein, and the aniline amino can be sulfonated with a suitable sulfonyl halide, such as the chloride, to give a 4-sulfonamidophenoxy ether product, which may require a final deprotection of a C2-side chain amine to give the final product. Timing of individual steps may also vary depending on the chemistry needed to incorporate various substituents. For example, a sulfonamide bond may be formed to link the A³ and A⁴ rings before the S_{N}Ar reaction to link the A² and A³ rings, or the S_{N}Ar reaction may be carried out to link the A² and A³ rings before the Suzuki-Miyauri biaryl coupling to link the A¹ and A² rings.

### Example 2. Syntheses of Compounds 1-23

The following compounds were made using Method A.
1. N-[4-[2-[2-[(1r,4r)(4-Aminocyclohexyl)amino]pyrimidin-4-yl]phenoxy]-3-fluoro-phenyl] benzenesulfonamide.

### Step 1: 2-(2-Chloropyrimidin-4-yl)phenol

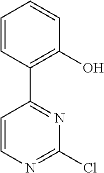 [SMILES: Oc1ccccc1-c1ccnc(Cl)n1]

A mixture of (2-hydroxyphenyl)boronic acid (10 g, 72.50 mmol, 1 eq), 2,4-dichloropyrimidine (10.80 g, 72.50 mmol, 1 eq), Cs₂CO₃ (47.24 g, 145.00 mmol, 2 eq), Pd(dppf)Cl₂ (5.30 g, 7.25 mmol, 0.1 eq) in THF (250 mL) and H₂O (50 mL) was degassed and purged with N₂ for 3 times, and then the mixture was stirred at 60° C. for 12 h under N₂ atmosphere. The mixture was concentrated and then water (300 mL) was added. The mixture was extracted with EtOAc (300 mL×3). The combined organic layers were washed with brine (500 mL), dried over Na₂SO₄, filtered and concentrated. The crude material was purified on silica gel column chromatography (PE/EtOAc=1/0 to 5/1, TLC: PE/EtOAc=5/1, R_{f}=0.3) to yield 2-(2-chloropyrimidin-4-yl)phenol (3 g, 7.84 mmol, 10.8% yield, 54.0% purity) as a white solid. ¹H NMR (500 MHz, CD₃OD-d₄) δ ppm 8.69 (d, J=5.5 Hz, 1H), 8.13 (d, J=5.5 Hz, 1H), 8.02 (dd, J=1.5, 8.2 Hz, 1H), 7.46-7.40 (m, 1H), 7.03-6.96 (m, 2H); ES-LCMS m/z 207.1 [M+H]⁺.

### 2-Chloro-4-[2-(2-fluoro-4-nitrophenoxy)phenyl]pyrimidine

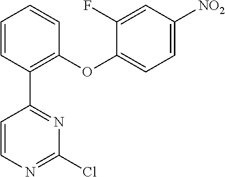 [SMILES: O=[N+]([O-])c1ccc(Oc2ccccc2-c2ccnc(Cl)n2)c(F)c1]

To a solution of 2-(2-chloropyrimidin-4-yl)phenol (700.00 mg, 3.02 mmol, 1 eq) and 1,2-difluoro-4-nitro-benzene (527.64 mg, 3.32 mmol, 366.41 μL, 1.1 eq) in DMF (5 mL) was added K₂CO₃ (625.07 mg, 4.52 mmol, 1.5 eq). The mixture was stirred at 60° C. for 1 h. The mixture was concentrated and then water (200 mL) was added. The mixture was extracted with EtOAc (200 mL×3). The combined organic layers were washed with brine (600 mL), dried over Na₂SO₄, filtered and concentrated. The crude material was purified on silica gel column chromatography (PE/EtOAc=1/0 to 10/1, TLC: PE/EtOAc=10/1, R_{f}=0.25) to yield 2-chloro-4-[2-(2-fluoro-4-nitrophenoxy)phenyl]pyrimidine (430 mg, 1.22 mmol, 40.4% yield, 98.0% purity) as a white solid. ¹H NMR (500 MHz, MeOD-d₄) δ ppm 8.69 (d, J=5.3 Hz, 1H), 8.21 (dd, J=2.7, 10.6 Hz, 1H), 8.10-8.05 (m, 2H), 7.93 (d, J=5.2 Hz, 1H), 7.68-7.63 (m, 1H), 7.50 (dt, J=0.9, 7.6 Hz, 1H), 7.24 (d, J=8.1 Hz, 1H), 7.12 (dd, J=8.2, 8.9 Hz, 1H); ES-LCMS m/z 346.1 [M+H]⁺.

### O-tert-Butyl N-[4-[[4-(1r,4r)[2-(2-fluoro-4-nitro-phenoxy)phenyl]pyrimidin-2-yl]amino]cyclohexyl]carbamate

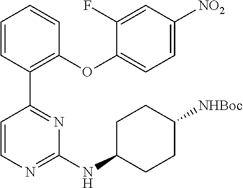 [SMILES: CC(C)(C)OC(=O)N[C@H]1CC[C@H](Nc2nccc(-c3ccccc3Oc3ccc([N+](=O)[O-])cc3F)n2)CC1]

To a solution of 2-chloro-4-[2-(2-fluoro-4-nitrophenoxy)phenyl]pyrimidine (150 mg, 425.21 μmol, 1 eq) and O-tert-butyl N-(1r,4r)(4-aminocyclohexyl) carbamate (118.46 mg, 552.77 μmol, 1.3 eq) in DMSO (5 mL) was added K₂CO₃ (88.15 mg, 637.81 μmol, 1.5 eq). The mixture was stirred at 120° C. for 12 h. The mixture was concentrated and then water (150 mL) was added. The mixture was extracted with EtOAc (150 mL×3). The combined organic layers were washed with brine (450 mL), dried over Na₂SO₄, filtered and concentrated. The crude material was purified on silica gel column chromatography (PE/EtOAc=1/0 to 1/1, TLC: PE/EtOAc=1/1, R_{f}=0.45) to yield O-tert-butyl N-[4-[[4-(1r,4r)[2-(2-fluoro-4-nitro-phenoxy)phenyl]pyrimidin-2-yl]amino]cyclohexyl]carbamate (120 mg, 206.28 μmol, 48.5% yield, 90.0% purity) as a brown solid. ¹H NMR (400 MHz, MeOD-d₄) δ ppm 8.21-8.09 (m, 2H), 8.01-7.94 (m, 1H), 7.89 (dd, J=1.7, 7.7 Hz, 1H), 7.57 (dt, J=1.7, 7.8 Hz, 1H), 7.47-7.39 (m, 1H), 7.20 (d, J=8.0 Hz, 1H), 6.97-6.88 (m, 2H), 3.76-3.61 (m, 2H), 1.98-1.85 (m, 4H), 1.44 (s, 9H), 1.40-1.27 (m, 4H); ES-LCMS m/z 524.3 [M+H]⁺.

### Step 4: O-tert-Butyl N-[4-[[4-(1r,4r)[2-(4-amino-2-fluoro-phenoxy)phenyl]pyrimidin-2-yl]amino]cyclohexyl] carbamate

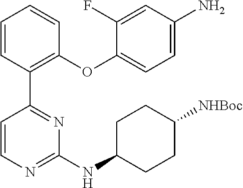 [SMILES: CC(C)(C)OC(=O)N[C@H]1CC[C@H](Nc2nccc(-c3ccccc3Oc3ccc(N)cc3F)n2)CC1]

A mixture of O-tert-butyl N-[4-[[4-(1r,4r)[2-(2-fluoro-4-nitro-phenoxy)phenyl]pyrimidin-2-yl]amino]cyclohexyl] carbamate (120 mg, 206.28 μmol, 1 eq), Fe (57.60 mg, 1.03 mmol, 5 eq), NH₄Cl (110.34 mg, 2.06 mmol, 10 eq) in THF (1.5 mL), H₂O (1.5 mL) and EtOH (1.5 mL) was degassed and purged with N₂ for 3 times, and then the mixture was stirred at 70° C. for 2 h. The mixture was concentrated and then water (150 mL) was added. The mixture was extracted with EtOAc (150 mL×3). The combined organic layers were washed with brine (450 mL), dried over Na₂SO₄, filtered and concentrated. The crude material was purified on silica gel column chromatography (PE/EtOAc=1/0 to 1/1, TLC: PE/EtOAc=1/1, R_{f}=0.60) to yield O-tert-butyl-N-[4-[[4-(1r,4r)[2-(4-amino-2-fluoro-phenoxy)phenyl┐pyrimidin-2-yl┐amino┐cyclohexyl┐ carbamate (70 mg, 141.82 μmol, 68.7% yield, 100.0% purity) as a yellow solid. ¹H NMR (500 MHz, MeOD-d₄) δ ppm 8.22 (d, J=5.3 Hz, 1H), 7.86 (d, J=7.3 Hz, 1H), 7.36-7.29 (m, 1H), 7.19 (d, J=5.3 Hz, 1H), 7.13 (t, J=7.2 Hz, 1H), 6.86 (t, J=8.9 Hz, 1H), 6.76 (d, J=8.4 Hz, 1H), 6.58 (d, J=2.6 Hz, 1H), 6.51-6.46 (m, 1H), 3.81 (s, 1H), 3.35 (s, 1H), 2.15-2.08 (m, 2H), 1.99-1.92 (m, 2H), 1.44 (s, 9H), 1.37 (d, J=11.1 Hz, 4H); ES-LCMS m/z 494.3 [M+H]⁺.

### Step 5: O-tert-Butyl N-[4-[[4-(1r,4r)[2-[4-(benzenesulfonamido)-2-fluorophenoxy]phenyl]pyrimidin-2-yl]amino]cyclohexyl] carbamate

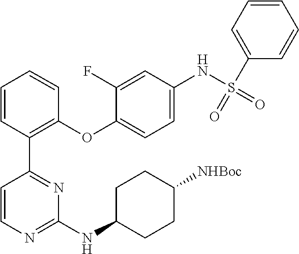 [SMILES: CC(C)(C)OC(=O)N[C@H]1CC[C@H](Nc2nccc(-c3ccccc3Oc3ccc(NS(=O)(=O)c4ccccc4)cc3F)n2)CC1]

A mixture of O-tert-butyl N-[4-[[4-(1r,4r)[2-(4-amino-2-fluorophenoxy)phenyl]pyrimidin-2-yl]amino]cyclohexyl]carbamate (65 mg, 131.69 μmol, 1 eq), benzenesulfonyl chloride (25.59 mg, 144.86 μmol, 18.54 μL, 1.1 eq), pyridine (83.34 mg, 1.05 mmol, 85.04 μL, 8 eq) in THF (3 mL) was degassed and purged with N₂ for 3 times, and then the mixture was stirred at 25° C. for 1 h. The mixture was concentrated and then water (150 mL) was added. The mixture was extracted with EtOAc (150 mL×3). The combined organic layers were washed with brine (500 mL), dried over Na₂SO₄, filtered and concentrated. The crude material was purified on silica gel column chromatography (PE/EtOAc=1/0 to 1/1, TLC: PE/EtOAc=1/1, R_{f}=0.3) to yield O-tert-butyl N-[4-[[4-(1r,4r)[2-[4-(benzenesulfonamido)-2-fluorophenoxy]phenyl]pyrimidin-2-yl]amino]cyclohexyl] carbamate (60 mg, 92.78 μmol, 70.4% yield, 98.0% purity) as a yellow solid. ¹H NMR (500 MHz, MeOD-d₄) δ ppm 8.18 (d, J=5.3 Hz, 1H), 7.87-7.83 (m, 1H), 7.77-7.73 (m, 2H), 7.63-7.58 (m, 1H), 7.54-7.50 (m, 2H), 7.42-7.38 (m, 1H), 7.26-7.22 (m, 1H), 7.04-6.99 (m, 2H), 6.87-6.83 (m, 1H), 6.82-6.79 (m, 2H), 3.79-3.73 (m, 1H), 3.35 (s, 1H), 2.09-2.05 (m, 2H), 1.93 (d, J=10.2 Hz, 2H), 1.44 (s, 9H), 1.38-1.32 (m, 4H); ES-LCMS m/z 634.3 [M+H]⁺.

### N-[4-[2-[2-[(1r,4r)(4-Aminocyclohexyl)amino]pyrimidin-4-yl]phenoxy]-3-fluorophenyl] benzenesulfonamide

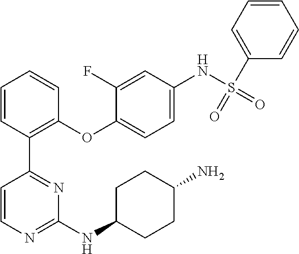 [SMILES: N[C@H]1CC[C@H](Nc2nccc(-c3ccccc3Oc3ccc(NS(=O)(=O)c4ccccc4)cc3F)n2)CC1]

To a solution of O-tert-butyl N-[4-[[4-(1r,4r)[2-[4-(benzenesulfonamido)-2-fluorophenoxy]phenyl]pyrimidin-2-yl]amino]cyclohexyl] carbamate (60 mg, 92.78 μmol, 1 eq) in DCM (3 mL) was added TFA (1.54 g, 13.51 mmol, 1 mL). The mixture was stirred at 25° C. for 1 h. The reaction mixture was filtered and concentrated under reduced pressure to give a residue. The residue was purified by preparative HPLC (column: Boston Prime C18 150*30 mm*5 μm; mobile phase: [water (NH₃H₂O+NH₄HCO₃)-ACN]; B %: 36%-66%, 10 min), followed by lyophilization to yield N-[4-[2-[2-[(1r,4r) (4-aminocyclohexyl)amino]pyrimidin-4-yl]phenoxy]-3-fluorophenyl] benzenesulfonamide (27.4 mg, 51.35 μmol, 55.3% yield, 100.0% purity) as a white solid. ¹H NMR (500 MHz, DMSO-d₆) δ ppm 8.26 (d, J=5.2 Hz, 1H), 7.80 (d, J=7.3 Hz, 1H), 7.74-7.70 (m, 2H), 7.46-7.41 (m, 3H), 7.36 (t, J=7.7 Hz, 1H), 7.20-7.05 (m, 2H), 7.00 (d, J=5.0 Hz, 1H), 6.83 (dd, J=2.4, 13.8 Hz, 2H), 6.73 (d, J=1.4 Hz, 1H), 6.66 (d, J=7.9 Hz, 1H), 3.62-3.61 (m, 1H), 2.91 (brs, 1H), 1.98-1.88 (m, 4H), 1.32 (q, J=9.8 Hz, 4H); ES-LCMS m/z 534.1 [M+H]⁺.
2. N-[3-Fluoro-4-[2-[2-[[(3S)-3-piperidyl]amino]pyrimidin-4-yl]phenoxy]phenyl] benzenesulfonamide

### Step 1: O-tert-Butyl N-((3S)-3-[[4-[2-(2-fluoro-4-nitrophenoxy)phenyl]pyrimidin-2-yl]amino]piperidin-1-yl) carbamate

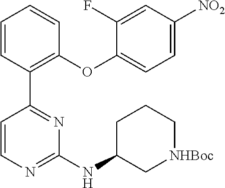

To a solution of 2-chloro-4-[2-(2-fluoro-4-nitrophenoxy)phenyl]pyrimidine (1.2 g, 2.53 mmol, 73.0% purity, 1 eq) and O-tert-butyl N-((3S)-3-aminopiperidin-1-yl) carbamate (761.22 mg, 3.80 mmol, 1.5 eq) in DMSO (5 mL) was added K₂CO₃ (525.30 mg, 3.80 mmol, 1.5 eq) which was stirred at 80° C. for 12 h. The mixture was concentrated and then water (150 mL) was added. The mixture was extracted with EtOAc (150 mL×3). The combined organic layers were washed with brine (500 mL), dried over Na₂SO₄, filtered and concentrated. The crude material was purified on silica gel column chromatography (PE/EtOAc=1/0 to 1/1, TLC: PE/EtOAc=1/1, R_{f}=0.65) to yield O-tert-butyl N-((3S)-3-[[4-[2-(2-fluoro-4-nitrophenoxy)phenyl]pyrimidin-2-yl]amino]piperidin-1-yl) carbamate (700 mg, 1.36 mmol, 53.6% yield, 99.0% purity) as a brown solid. ¹H NMR (500 MHz, MeOD-d₄) δ ppm 8.23 (d, J=5.0 Hz, 1H), 8.15 (dd, J=2.6, 10.7 Hz, 1H), 8.03-7.92 (m, 2H), 7.61-7.54 (m, 1H), 7.46-7.42 (m, 1H), 7.21 (d, J=8.1 Hz, 1H), 7.04-6.92 (m, 2H), 3.71 (brs, 2H), 3.60-3.33 (m, 1H), 3.27-3.09 (m, 1H), 3.08-2.61 (m, 1H), 1.96-1.86 (m, 1H), 1.68-1.55 (m, 1H), 1.49-1.44 (m, 2H), 1.43-1.18 (m, 9H); ES-LCMS m/z 510.3 [M+H]⁺.

### Step 2: O-tert-Butyl N-((3S)-3-[[4-[2-(4-amino-2-fluorophenoxy)phenyl]pyrimidin-2-yl]amino]piperidin-1-yl) carbamate

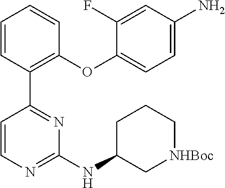

A mixture of O-tert-butyl N-((3S)-3-[[4-[2-(2-fluoro-4-nitrophenoxy)phenyl]pyrimidin-2-yl]amino]piperidin-1-yl) carbamate (700 mg, 1.36 mmol, 99.0% purity, 1 eq), Fe (379.77 mg, 6.80 mmol, 5 eq), NH₄Cl (727.53 mg, 13.60 mmol, 10 eq) in THF (3 mL), H₂O (3 mL) and EtOH (3 mL) was degassed and purged with N₂ for 3 times, and then the mixture was stirred at 70° C. for 4 h under N₂ atmosphere. The mixture was concentrated and then water (100 mL) was added. The mixture was extracted with EtOAc (100 mL×3). The combined organic layers were washed with brine (300 mL), dried over Na₂SO₄, filtered and concentrated to yield O-tert-butyl N-((3S)-3-[[4-[2-(4-amino-2-fluorophenoxy)phenyl]pyrimidin-2-yl]amino]piperidin-1-yl) carbamate (650 mg, 1.22 mmol, 89.6% yield, 90.0% purity) as a yellow oil. ¹H NMR (400 MHz, MeOD-d₄) δ ppm 8.26 (d, J=5.2 Hz, 1H), 8.06-7.88 (m, 1H), 7.37-7.26 (m, 2H), 7.13 (t, J=7.5 Hz, 1H), 6.86 (t, J=8.9 Hz, 1H), 6.76 (d, J=8.2 Hz, 1H), 6.57 (dd, J=2.6, 12.8 Hz, 1H), 6.52-6.47 (m, 1H), 3.97 (s, 1H), 3.88-3.69 (m, 1H), 3.66-3.33 (m, 2H), 3.20-2.83 (m, 1H), 2.03 (s, 1H), 1.83 (d, J=19.3 Hz, 1H), 1.63-1.50 (m, 2H), 1.33-1.23 (m, 9H); ES-LCMS m/z 480.2 [M+H]⁺.

### Step 3: O-tert-Butyl N-((3S)-3-[[4-[2-[4-(benzenesulfonamido)-2-fluorophenoxy]phenyl]pyrimidin-2-yl]amino]piperidin-1-yl) carbamate

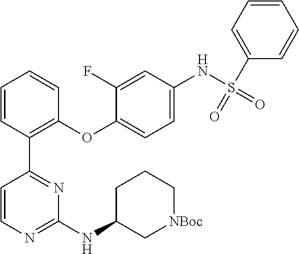 [SMILES: CC(C)(C)OC(=O)N1CCC[C@H](Nc2nccc(-c3ccccc3Oc3ccc(NS(=O)(=O)c4ccccc4)cc3F)n2)C1]

A mixture of O-tert-butyl N-((3S)-3-[[4-[2-(4-amino-2-fluorophenoxy)phenyl]pyrimidin-2-yl]amino]piperidin-1-yl) carbamate (140 mg, 262.75 μmol, 1 eq), benzenesulfonyl chloride (55.69 mg, 315.30 μmol, 40.35 μL, 1.2 eq), pyridine (166.27 mg, 2.10 mmol, 169.66 μL, 8 eq) in THF (3 mL) was degassed and purged with N₂ for 3 times, and then the mixture was stirred at 25° C. for 3 h under N₂ atmosphere. The mixture was concentrated and then water (75 mL) was added. The mixture was extracted with EtOAc (75 mL×3). The combined organic layers were washed with brine (250 mL), dried over Na₂SO₄, filtered and concentrated. The crude material was purified on silica gel column chromatography (PE/EtOAc=1/0 to 1/1, TLC: PE/EtOAc=1/1, R_{f}=0.25) to yield O-tert-butyl N-((3S)-3-[[4-[2-[4-(benzenesulfonamido)-2-fluorophenoxy]phenyl]pyrimidin-2-yl]amino]piperidin-1-yl) carbamate

(120 mg, 185.89 μmol, 70.7% yield, 96.0% purity) as a yellow solid. ¹H NMR (400 MHz, MeOD-d₄) δ ppm 8.23 (d, J=5.1 Hz, 1H), 8.00-7.88 (m, 1H), 7.78-7.74 (m, 2H), 7.63-7.58 (m, 1H), 7.55-7.49 (m, 2H), 7.47-7.35 (m, 2H), 7.27-7.21 (m, 1H), 7.12 (d, J=5.1 Hz, 1H), 7.02 (dd, J=2.4, 12.1 Hz, 1H), 6.80 (d, J=8.5 Hz, 2H), 3.93-3.87 (m, 1H), 3.84-3.68 (m, 1H), 3.61-3.46 (m, 1H), 3.35 (s, 2H), 1.99 (td, J=2.4, 5.0 Hz, 1H), 1.83-1.75 (m, 1H), 1.59-1.50 (m, 2H), 1.35-1.19 (m, 9H); ES-LCMS m/z 620.3 [M+H]⁺.

### Step 4: N-[3-Fluoro-4-[2-[2-[[(3S)-3-piperidyl]amino]pyrimidin-4-yl]phenoxy]phenyl] benzenesulfonamide

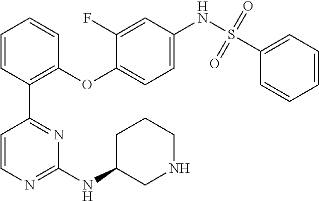 [SMILES: O=S(=O)(Nc1ccc(Oc2ccccc2-c2ccnc(N[C@H]3CCCNC3)n2)c(F)c1)c1ccccc1]

To a solution of O-tert-butyl N-((3S)-3-[[4-[2-[4-(benzenesulfonamido)-2-fluorophenoxy]phenyl]pyrimidin-2-yl]amino]piperidin-1-yl) carbamate (100 mg, 154.91 μmol, 1 eq) in DCM (1 mL) was added TFA (1.54 g, 13.51 mmol, 1 mL, 83.70 eq). The mixture was stirred at 25° C. for 1 h. The mixture was neutralized to pH=7 with NH₃·H₂O and concentrated. Then water (100 mL) was added. The mixture was extracted with EtOAc (100 mL×3). The combined organic layers were washed with brine (100 mL), dried over Na₂SO₄, filtered and concentrated. The residue was purified by preparative HPLC (column: Boston Prime C18 150*30 mm*5 μm; mobile phase: [water (NH₃H₂O+NH₄HCO₃)-ACN]; B %: 37%-67%, 10 min), followed by lyophilization to yield N-[3-fluoro-4-[2-[2-[[(3S)-3-piperidyl]amino]pyrimidin-4-yl]phenoxy]phenyl]benzenesulfonamide (60.9 mg, 117.21 μmol, 72.6% yield, 100.0% purity) as a white solid. ¹H NMR (500 MHz, DMSO-d₆) δ ppm 8.27 (d, J=5.0 Hz, 1H), 7.85 (slbrd, J=4.8 Hz, 1H), 7.76-7.72 (m, 2H), 7.57-7.48 (m, 3H), 7.39 (ddd, J=9.1, 7.6, 1.4 Hz, 1H), 7.20 (t, J=7.5 Hz, 1H), 7.09 (slbrd, J=7.6 Hz, 1H), 7.04 (d, J=5.2 Hz, 1H), 6.97-6.91 (m, 2H), 6.78-6.71 (m, 2H), 3.94-3.87 (m, 1H), 3.15 (dd, J=3.1, 11.8 Hz, 1H), 2.90 (d, J=12.2 Hz, 1H), 2.62-2.52 (m, 2H), 1.94-1.87 (m, 1H), 1.74-1/67 (m, 1H), 1.53-1.44 (m, 2H); ES-LCMS m/z 520.2 [M+H]⁺.

The following compounds have been made by processes similar to those described immediately above using Method A.
3. N-[4-[2-[2-[(1r,4r)(4-Aminocyclohexyl)amino]pyrimidin-4-yl]phenoxy]-3-fluorophenyl] 2-chlorobenzenesulfonamide. ES-LCMS m/z 568.2 [M+H]⁺
4. N-[4-[2-[2-[(1r,4r)(4-Aminocyclohexyl)amino]pyrimidin-4-yl]phenoxy]-3-fluorophenyl] naphthalene-1-sulfonamide. ES-LCMS m/z 584.2 [M+H]⁺
5. N-[3-Fluoro-4-[2-[2-[[(3S)-3-piperidyl]amino]pyrimidin-4-yl]phenoxy]phenyl] 2-chlorobenzenesulfonamide. ES-LCMS m/z 554.1 [M+H]⁺
6. N-[3-Fluoro-4-[2-[2-[[(3S)-3-piperidyl]amino]pyrimidin-4-yl]phenoxy]phenyl] naphthalene-1-sulfonamide. ES-LCMS m/z 570.2 [M+H]⁺
7. N-[4-[2-[2-[(1r,4r)(4-Aminocyclohexyl)amino]pyrimidin-4-yl]-4-methylphenoxy]-3-fluorophenyl] 2-chlorobenzenesulfonamide

### Step 1: 2-Chloro-4-(2-methoxy-5-methylphenyl)pyrimidine

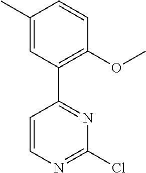

To a solution of 2,4-dichloropyrimidine (3 g, 20.14 mmol, 1 eq) in DME (50 mL) and H₂O (10 mL) was added Na₂CO₃ (3.20 g, 30.21 mmol, 1.5 eq), (2-methoxy-5-methylphenyl) boronic acid (3.68 g, 22.15 mmol, 1.1 eq), PPh₃ (264.09 mg, 1.01 mmol, 0.05 eq) and Pd(OAc)₂ (113.02 mg, 503.43 μmol, 0.025 eq). The mixture was stirred at 90° C. for 12 h under N₂. The reaction mixture was diluted with H₂O (50 mL) and extracted with EtOAc (50 mL×3). The combined organic layers were washed with brine (50 mL), dried over Na₂SO₄, filtered and concentrated under reduced pressure to yield a residue which was purified by flash silica gel chromatography (PE/EtOAc=1/0 to 3/1, TLC: PE/EtOAc=3/1, R_{f}=0.49) to yield 2-chloro-4-(2-methoxy-5-methylphenyl)pyrimidine (3.6 g, 15.34 mmol, 76.2% yield, 100.0% purity) as a yellow solid. ¹H NMR (400 MHz, DMSO-d₆) δ ppm 8.74 (d, J=5.3 Hz, 1H), 8.06 (d, J=5.3 Hz, 1H), 7.74 (d, J=2.1 Hz, 1H), 7.36 (dd, J=2.3, 8.5 Hz, 1H), 7.13 (d, J=8.5 Hz, 1H), 3.87 (s, 3H), 2.32 (s, 3H); ES-LCMS m/z 235.2, 237.2 [M+H]⁺.

### Step 2: 2-(2-Chloropyrimidin-4-yl)-4-methylphenol

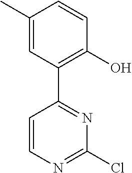

To a solution of 2-chloro-4-(2-methoxy-5-methylphenyl)pyrimidine (0.5 g, 2.13 mmol, 100.0% purity, 1 eq) in DCM (10 mL) was added BBr₃ (1 M, 21.3 mL, 10 eq). The mixture was stirred at 25° C. for 2 h. The reaction mixture was neutralized to pH=7 with saturated NaHCO₃ solution and extracted with DCM (20 mL×3). The combined organic layers were washed with brine (20 mL), dried over Na₂SO₄, filtered and concentrated under reduced pressure to yield 2-(2-chloropyrimidin-4-yl)-4-methylphenol (200 mg, 897.33 μmol, 42.1% yield, 99.0% purity) as a yellow solid. ¹H NMR (400 MHz, DMSO-d₆) δ ppm 8.76-8.61 (m, 1H), 8.42-8.29 (m, 1H), 7.81 (dd, J=1.8, 8.4 Hz, 1H), 7.18 (d, J=7.9 Hz, 1H), 6.89 (dd, J=2.9, 8.1 Hz, 1H), 2.26 (s, 3H); ES-LCMS m/z 221.1, 223.1 [M+H]⁺.

### Step 3: 2-Chloro-4-[2-(2-fluoro-4-nitrophenoxy)-5-methylphenyl]pyrimidine

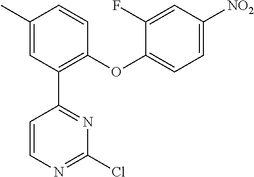 [SMILES: Cc1ccc(Oc2ccc([N+](=O)[O-])cc2F)c(-c2ccnc(Cl)n2)c1]

To a solution of 2-(2-chloropyrimidin-4-yl)-4-methylphenol (200 mg, 897.33 μmol, 99.0% purity, 1 eq) in DMSO (1 mL) was added K₂CO₃ (248.03 mg, 1.79 mmol, 2 eq) and 1,2-difluoro-4-nitro-benzene (142.76 mg, 897.33 μmol, 99.14 μL, 1 eq). The mixture was stirred at 25° C. for 12 h. The reaction mixture was diluted with H₂O (20 mL) and extracted with EtOAc (20 mL×3). The combined organic layers were washed with brine (20 mL), dried over Na₂SO₄, filtered and concentrated under reduced pressure to yield a residue which was purified by flash silica gel chromatography (PE/EtOAc=1/0 to 3/1, TLC: PE/EtOAc=3/1, R_{f}=0.51) to yield 2-chloro-4-[2-(2-fluoro-4-nitrophenoxy)-5-methylphenyl]pyrimidine (120 mg, 316.90 μmol, 35.3% yield, 95.0% purity) as a yellow solid. ¹H NMR (400 MHz, CDCl₃) δ ppm 8.82-8.70 (m, 1H), 8.32 (dd, J=2.6, 10.8 Hz, 1H), 8.07-8.01 (m, 1H), 7.90 (dd, J=5.2, 10.3 Hz, 1H), 7.82 (dd, J=1.9, 7.4 Hz, 1H), 7.50 (dd, J=1.9, 8.5 Hz, 1H), 7.23 (d, J=8.3 Hz, 1H), 7.08 (dt, J=4.7, 8.7 Hz, 1H), 2.43 (s, 3H); ES-LCMS m/z 360.2, 362.2 └M+H┘⁺.

### Step 4: O-tert-Butyl N-[4-(1r,4r)[[4-[2-(2-fluoro-4-nitrophenoxy)-5-methylphenyl]pyrimidin-2-yl]amino]cyclohexyl] carbamate

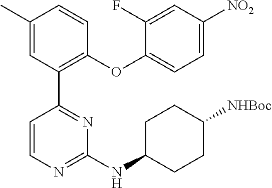 [SMILES: Cc1ccc(Oc2ccc([N+](=O)[O-])cc2F)c(-c2ccnc(N[C@H]3CC[C@H](NC(=O)OC(C)(C)C)CC3)n2)c1]

To a solution of 2-chloro-4-[2-(2-fluoro-4-nitrophenoxy)-5-methylphenyl]pyrimidine (120 mg, 316.90 μmol, 95.0% purity, 1 eq) in DMSO (1 mL) was added TEA (96.20 mg, 950.69 μmol, 132.32 μL, 3 eq) and O-tert-butyl N-((1r,4r)4-aminocyclohexyl) carbamate (122.24 mg, 570.41 μmol, 1.8 eq). The mixture was stirred at 110° C. for 12 h. The reaction mixture was diluted with H₂O (20 mL) and extracted with EtOAc (20 mL×3). The combined organic layers were washed with brine (20 mL), dried over Na₂SO₄, filtered and concentrated under reduced pressure to yield a residue which was purified by flash silica gel chromatography (from PE/EtOAc=1/0 to 1/1, TLC: PE/EtOAc=1/1, R_{f}=0.16) to yield O-tert-butyl N-[4-(1r,4r)[[4-[2-(2-fluoro-4-nitrophenoxy)-5-methylphenyl]pyrimidin-2-yl]amino]cyclohexyl] carbamate (140 mg, 236.99 μmol, 74.8% yield, 91.0% purity) as a yellow solid. ¹H NMR (400 MHz, CDCl₃) δ ppm 8.22 (d, J=5.0 Hz, 1H), 8.04 (dd, J=2.6, 10.4 Hz, 1H), 7.88 (d, J=9.5 Hz, 1H), 7.73 (d, J=1.8 Hz, 1H), 7.31 (dd, J=2.0, 8.3 Hz, 1H), 7.02 (d, J=8.3 Hz, 1H), 6.95 (d, J=5.3 Hz, 1H), 6.83-6.75 (m, 1H), 5.21-5.01 (m, 1H), 4.52-4.34 (m, 1H), 3.79-3.67 (m, 1H), 3.45 (d, J=3.4 Hz, 1H), 2.45 (s, 3H), 2.11 (d, J=10.4 Hz, 2H), 2.07-2.02 (m, 2H), 1.45 (s, 9H), 1.31-1.23 (m, 4H); ES-LCMS m/z 538.2 [M+H]⁺.

### Step 5: O-tert-Butyl N-[4-(1r,4r)[[4-[2-(4-amino-2-fluorophenoxy)-5-methylphenyl]pyrimidin-2-yl]amino]cyclohexyl] carbamate

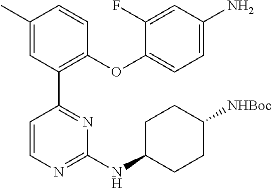 [SMILES: Cc1ccc(Oc2ccc(N)cc2F)c(-c2ccnc(N[C@H]3CC[C@H](NC(=O)OC(C)(C)C)CC3)n2)c1]

To a solution of O-tert-butyl N-[4-(1r,4r)[[4-[2-(2-fluoro-4-nitrophenoxy)-5-methylphenyl]pyrimidin-2-yl]amino]cyclohexyl] carbamate (140 mg, 236.99 μmol, 91% purity, 1 eq) and NH₄Cl (126.77 mg, 2.37 mmol, 10 eq) in EtOH (2.5 mL) and H₂O (0.5 mL) was added Fe (66.17 mg, 1.18 mmol, 5 eq). The mixture was stirred at 80° C. for 3 h. After filtration, the organic layer was concentrated under reduced pressure to yield a yellow oil which was purified by flash silica gel chromatography (PE/EtOAc=1/0 to 1/1, TLC: PE/EtOAc=1/1, R_{f}=0.59) to yield O-tert-butyl N-[4-(1r,4r)[[4-[2-(4-amino-2-fluorophenoxy)-5-methylphenyl]pyrimidin-2-yl]amino]cyclohexyl] carbamate (100 mg, 189.13 μmol, 79.8% yield, 96.0% purity) as a yellow solid. ¹H NMR (400 MHz, CDCl₃) δ ppm 8.27-8.19 (m, 1H), 7.75-7.69 (m, 1H), 7.30 (s, 1H), 7.14 (d, J=7.9 Hz, 1H), 6.86 (t, J=8.8 Hz, 1H), 6.70 (d, J=8.6 Hz, 1H), 6.52 (dd, J=2.7, 12.1 Hz, 1H), 6.41 (dd, J=1.5, 8.5 Hz, 1H), 4.49-4.39 (m, 1H), 3.92-3.84 (m, 1H), 3.75-3.67 (m, 1H), 3.56-3.45 (m, 1H), 2.37 (s, 3H), 2.21-2.16 (m, 2H), 2.11-2.06 (m, 2H), 1.46 (s, 9H), 1.42-1.31 (m, 4H); ES-LCMS m/z 508.2 [M+H]⁺.

### Step 6: O-tert-Butyl N-[4-(1r,4r)[[4-[2-(4-(2-chlorobenzenesulfonamido)-2-fluorophenoxy)-5-methylphenyl]pyrimidin-2-yl]amino]cyclohexyl] carbamate

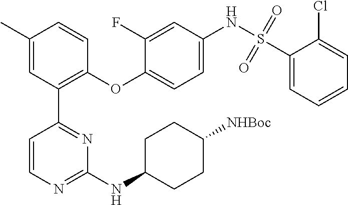 [SMILES: Cc1ccc(Oc2ccc(NS(=O)(=O)c3ccccc3Cl)cc2F)c(-c2ccnc(N[C@H]3CC[C@H](NC(=O)OC(C)(C)C)CC3)n2)c1]

To a solution of O-tert-butyl N-[4-(1r,4r)[[4-[2-(4-amino-2-fluorophenoxy)-5-methylphenyl]pyrimidin-2-yl]amino]cyclohexyl] carbamate (100 mg, 189.13 μmol, 96.0% purity, 1 eq) in THF (2 mL) was added pyridine (74.80 mg, 945.63 μmol, 76.33 μL, 5 eq) and 2-chlorobenzenesulfonyl chloride (119.75 mg, 567.38 μmol, 77.26 μL, 3 eq). The mixture was stirred at 25° C. for 1 h. The reaction mixture was diluted with H₂O (20 mL) and extracted with EtOAc (20 mL×3). The combined organic layers were washed with brine (10 mL), dried over Na₂SO₄, filtered and concentrated under reduced pressure to yield a residue which was purified by flash silica gel chromatography (DCM/MeOH=1/0 to 10/1, TLC: DCM/MeOH=10/1, R_{f}=0.58) to yield O-tert-butyl N-[4-(1r,4r)[[4-[2-(4-(2-chlorobenzenesulfonamido)-2-fluorophenoxy)-5-methylphenyl]pyrimidin-2-yl]amino]cyclohexyl] carbamate (100 mg, 142.19 μmol, 75.2% yield, 97.0% purity) as a yellow solid. ¹H NMR (400 MHz, DMSO-d₆) δ ppm 10.86-10.75 (m, 1H), 8.20 (d, J=5.0 Hz, 1H), 8.04 (d, J=7.8 Hz, 1H), 7.70-7.63 (m, 3H), 7.56-7.50 (m, 1H), 7.22-7.17 (m, 1H), 7.02 (dd, J=2.1, 11.9 Hz, 2H), 6.96 (d, J=8.9 Hz, 1H), 6.88 (d, J=5.1 Hz, 2H), 6.74-6.64 (m, 2H), 4.14-4.10 (m, 1H), 3.29-3.28 (m, 1H), 2.33-2.28 (m, 3H), 1.92-1.87 (m, 2H), 1.80-1.74 (m, 2H), 1.38 (s, 9H), 1.23 (brs, 4H); ES-LCMS m/z 682.3, 684.0 [M+H]⁺.

### Step 7: N-[4-[2-[2-[(1r,4r)(4-Aminocyclohexyl)amino]pyrimidin-4-yl]-4-methylphenoxy]-3-fluorophenyl] 2-chlorobenzenesulfonamide

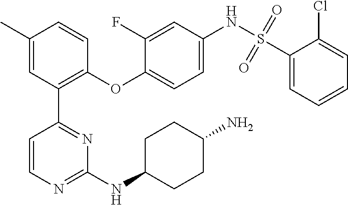 [SMILES: Cc1ccc(Oc2ccc(NS(=O)(=O)c3ccccc3Cl)cc2F)c(-c2ccnc(N[C@H]3CC[C@H](N)CC3)n2)c1]

To a solution of O-tert-butyl N-[4-(1r,4r)[[4-[2-(4-(2-chlorobenzenesulfonamido)-2-fluorophenoxy)-5-methylphenyl]pyrimidin-2-yl]amino]cyclohexyl] carbamate (100 mg, 142.19 μmol, 97.0% purity, 1 eq) in DCM (2 mL) was added HCl/dioxane (4 M, 1 mL, 28.13 eq). The mixture was stirred at 25° C. for 1 h. The reaction mixture was neutralized to pH=7 with NH₃·H₂O and the reaction mixture was concentrated to yield a residue which was purified by preparative HPLC (column: Waters Xbridge BEH C18 150*25 mm*5 μm; mobile phase: [water (NH₃H₂O)-ACN]; B %: 27%-57%, 10 min), followed by lyophilization to yield N-[4-[2-[2-[(1r,4r)(4-aminocyclohexyl)amino]pyrimidin-4-yl]-4-methylphenoxy]-3-fluorophenyl] 2-chlorobenzenesulfonamide (12.3 mg, 21.13 μmol, 14.9% yield, 100.0% purity) as a white solid. ¹H NMR (400 MHz, DMSO-d₆) δ ppm 8.23 (d, J=5.1 Hz, 1H), 7.91 (dd, J=1.7, 7.4 Hz, 1H), 7.59 (brs, 1H), 7.46-7.42 (m, 1H), 7.41-7.32 (m, 2H), 7.14 (d, J=7.3 Hz, 1H), 7.09-6.87 (m, 2H), 6.76-6.61 (m, 3H), 6.56 (d, J=7.8 Hz, 1H), 3.65-3.55 (m, 1H), 2.94-2.86 (m, 1H), 2.29 (s, 3H), 1.97-1.86 (m, 4H), 1.38-1.25 (m, 4H); ES-LCMS m/z 582.3, 584.3 [M+H]⁺.

The following compounds have been made by processes similar to those described immediately above using Method A.
8. N-[4-[2-[2-[(1r,4r)(4-Aminocyclohexyl)amino]pyrimidin-4-yl]-4-(trifluoromethyl)phenoxy]-3-fluorophenyl] 2-chlorobenzenesulfonamide. ES-LCMS m/z 636.3 [M+H]⁺
9. N-[4-[2-[2-[(1r,4r)(4-Aminocyclohexyl)amino]pyrimidin-4-yl]-5-methylphenoxy]-3-fluorophenyl] 2-chlorobenzenesulfonamide. ES-LCMS m/z 582.3, 584.3 [M+H]⁺.
10. N-[4-[2-[2-[(1r,4r)(4-Aminocyclohexyl)amino]pyrimidin-4-yl]-4-cyanophenoxy]-3-fluorophenyl] 2-chlorobenzenesulfonamide. ES-LCMS m/z 593.2, 595.1 [M+H]⁺.
11. N-[4-[2-[2-[(1r,4r)(4-Aminocyclohexyl)amino]pyrimidin-4-yl]-5-(1,1-dimethylethyl)phenoxy]-3-fluorophenyl] 2-chlorobenzenesulfonamide.

### Step 1: 2-Bromo-5-tert-butylphenol

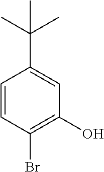

To a solution of 3-tert-butylphenol (5 g, 33.29 mmol, 1 eq) in DCM (50 mL) was added NBS (5.92 g, 33.29 mmol, 1 eq). The mixture was stirred at 25° C. for 2 h. The reaction mixture was diluted with H₂O (60 mL) and extracted with DCM (100 mL×3). The combined organic layers were washed with brine (40 mL), dried over Na₂SO₄, filtered and concentrated under reduced pressure to yield a residue which was purified by flash silica gel chromatography (PE/EtOAc=1/0 to 20/1, TLC: PE/EtOAc=20/1, R_{f}=0.54) to yield 2-bromo-5-tert-butylphenol (7.5 g, 31.10 mmol, 93.4% yield, 95.0% purity) as a white liquid. ¹H NMR (400 MHz, DMSO-d₆) δ ppm 10.00 (s, 1H), 7.35 (d, J=8.4 Hz, 1H), 6.96 (d, J=2.3 Hz, 1H), 6.76 (dd, J=2.3, 8.4 Hz, 1H), 1.22 (s, 9H).

### Step 2: 1-Bromo-4-tert-butyl-2-(2-fluoro-4-nitrophenoxy)benzene

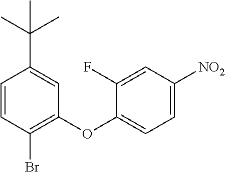 [SMILES: CC(C)(C)c1ccc(Br)c(Oc2ccc([N+](=O)[O-])cc2F)c1]

To a solution of 2-bromo-5-tert-butylphenol (4 g, 16.59 mmol, 95.0% purity, 1 eq) in DMSO (30 mL) was added K₂CO₃ (4.58 g, 33.17 mmol, 2 eq) and 1,2-difluoro-4-nitrobenzene (2.64 g, 16.59 mmol, 1.83 mL, 1 eq). The mixture was stirred at 80° C. for 12 h. The reaction mixture was diluted with H₂O (60 mL) and extracted with EtOAc (60 mL×3). The combined organic layers were washed with brine (60 mL), dried over Na₂SO₄, filtered and concentrated under reduced pressure to yield a residue which was purified by flash silica gel chromatography (PE/EtOAc=1/0 to 10/1, TLC: PE/EtOAc=10/1, R_{f}=0.51) to yield 1-bromo-4-tert-butyl-2-(2-fluoro-4-nitrophenoxy)benzene (4.5 g, 11.00 mmol, 66.3% yield, 90.0% purity) as a yellow solid. ¹H NMR (400 MHz, DMSO-d₆) δ ppm 8.35 (dd, J=2.7, 10.8 Hz, 1H), 8.10-8.05 (m, 1H), 7.73 (d, J=8.5 Hz, 1H), 7.41 (d, J=2.3 Hz, 1H), 7.34 (dd, J=2.3, 8.4 Hz, 1H), 6.87 (t, J=8.8 Hz, 1H), 1.27 (s, 9H).

### Step 3: 2-[4-tert-Butyl-2-(2-fluoro-4-nitrophenoxy)phenyl]-4,4,5,5-tetramethyl-1,3,2-dioxaborolane

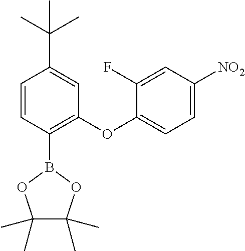 [SMILES: CC(C)(C)c1ccc(B2OC(C)(C)C(C)(C)O2)c(Oc2ccc([N+](=O)[O-])cc2F)c1]

To a solution of 1-bromo-4-tert-butyl-2-(2-fluoro-4-nitrophenoxy)benzene (1 g, 2.44 mmol, 1 eq) in 1,4-dioxane (20 mL) was added KOAc (719.68 mg, 7.33 mmol, 3 eq), 4,4,5,5-tetramethyl-2-(4,4,5,5-tetramethyl-1,3,2-dioxaborolan-2-yl)-1,3,2-dioxaborolane (1.24 g, 4.89 mmol, 2 eq) and Pd(dppf)Cl₂ (178.85 mg, 244.43 μmol, 0.1 eq). The mixture was stirred at 110° C. for 3 h under N₂. The reaction mixture was diluted with H₂O (30 mL) and extracted with EtOAc (30 mL×3). The combined organic layers were washed with brine (30 mL), dried over Na₂SO₄, filtered and concentrated under reduced pressure to yield a residue which was purified by flash silica gel chromatography (from PE/EtOAc=1/0 to 5/1, TLC: PE/EtOAc=5/1, R_{f}=0.48) to yield 2-[4-tert-butyl-2-(2-fluoro-4-nitrophenoxy)phenyl]-4,4,5,5-tetramethyl-1,3,2-dioxaborolane (450 mg, 866.92 μmol, 35.5% yield, 80.0% purity) as a yellow solid. ¹H NMR (400 MHz, DMSO-d₆) δ ppm 8.29 (dd, J=2.7, 10.8 Hz, 1H), 8.00 (dd, J=1.5, 9.1 Hz, 1H), 7.72 (d, J=7.9 Hz, 1H), 7.42 (dd, J=1.5, 7.9 Hz, 1H), 7.28 (d, J=1.4 Hz, 1H), 6.63 (t, J=8.8 Hz, 1H), 1.30 (s, 9H), 1.03 (s, 12H).

### Step 4: 4-[4-tert-Butyl-2-(2-fluoro-4-nitrophenoxy)phenyl]-2-chloropyrimidine

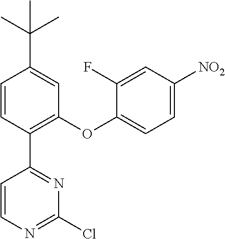 [SMILES: CC(C)(C)c1ccc(-c2ccnc(Cl)n2)c(Oc2ccc([N+](=O)[O-])cc2F)c1]

To a solution of 2-[4-tert-butyl-2-(2-fluoro-4-nitrophenoxy)phenyl]-4,4,5,5-tetramethyl-1,3,2-dioxaborolane (450 mg, 866.92 μmol, 1 eq) in dioxane (10 mL) and H₂O (3 mL) was added Cs₂CO₃ (564.92 mg, 1.73 mmol, 2 eq) 2,4-dichloropyrimidine (142.07 mg, 953.62 μmol, 1.1 eq) and Pd(dppf)Cl₂ (63.43 mg, 86.69 μmol, 0.1 eq). The mixture was stirred at 90° C. for 3 h under N₂. The reaction mixture was diluted with H₂O (50 mL) and extracted with EtOAc (50 mL×3). The combined organic layers were washed with brine (50 mL), dried over Na₂SO₄, filtered and concentrated under reduced pressure to yield a residue which was purified by flash silica gel chromatography (PE/EtOAc=1/0 to 3/1, TLC: PE/EtOAc=3/1, R_{f}=0.50) to yield 4-[4-tert-butyl-2-(2-fluoro-4-nitrophenoxy)phenyl]-2-chloropyrimidine (200 mg, 472.85 μmol, 54.5% yield, 95.0% purity) as a yellow solid. ¹H NMR (400 MHz, DMSO-d₆) δ ppm 8.78 (d, J=5.1 Hz, 1H), 8.33 (dd, J=2.6, 10.8 Hz, 1H), 8.06-8.02 (m, 1H), 7.99 (d, J=8.3 Hz, 1H), 7.88 (d, J=5.3 Hz, 1H), 7.57 (dd, J=1.8, 8.3 Hz, 1H), 7.35 (d, J=1.8 Hz, 1H), 7.02 (t, J=8.7 Hz, 1H), 1.30 (s, 9H); ES-LCMS m/z 402.2 [M+H]⁺.

### Step 5: O-t-Butyl N-[4-[(1r,4r)[4-[4-tert-butyl-2-[2-fluoro-4-nitrophenoxy]phenyl]pyrimidin-2-yl]amino]cyclohexyl] carbamate

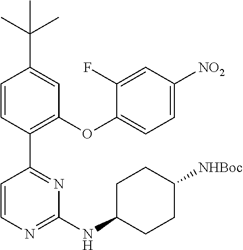 [SMILES: CC(C)(C)OC(=O)N[C@H]1CC[C@H](Nc2nccc(-c3ccc(C(C)(C)C)cc3Oc3ccc([N+](=O)[O-])cc3F)n2)CC1]

To a solution of 4-[4-tert-butyl-2-(2-fluoro-4-nitrophenoxy)phenyl]-2-chloropyrimidine (200 mg, 472.85 μmol, 1 eq) in DMSO (4 mL) was added TEA (143.54 mg, 1.42 mmol, 197.44 μL, 3 eq) and O-t-butyl N-((1r,4r)-4-aminocyclohexyl) carbamate (202.67 mg, 945.70 μmol, 2 eq). The mixture was stirred at 110° C. for 12 h. The reaction mixture was diluted with H₂O (20 mL) and extracted with EtOAc (20 mL×3). The combined organic layers were washed with brine (20 mL), dried over Na₂SO₄, filtered and concentrated under reduced pressure to yield a residue which was purified by flash silica gel chromatography (PE/EtOAc=1/0 to 1/1, TLC: PE/EtOAc=1/1, R_{f}=0.50) to yield O-t-butyl N-[4-[(1r,4r)[4-[4-tert-butyl-2-[2-fluoro-4-nitrophenoxy]phenyl]pyrimidin-2-yl]amino]cyclohexyl] carbamate (160 mg, 262.22 μmol, 55.5% yield, 95.0% purity) as a yellow solid. ¹H NMR (400 MHz, DMSO-d₆) δ ppm 8.29 (dd, J=2.7, 10.8 Hz, 1H), 8.21 (d, J=5.1 Hz, 1H), 8.05-7.98 (m, 1H), 7.85 (d, J=8.6 Hz, 1H), 7.56-7.46 (m, 1H), 7.29 (d, J=1.8 Hz, 1H), 7.01-6.94 (m, 1H), 6.93-6.87 (m, 1H), 6.79 (d, J=5.1 Hz, 1H), 6.71-6.62 (m, 1H), 3.57-3.48 (m, 1H), 3.19-3.11 (m, 1H), 1.85-1.79 (m, 2H), 1.79-1.71 (m, 2H), 1.38 (s, 9H), 1.30 (s, 9H), 1.27-1.12 (m, 4H); ES-LCMS m/z 580.5 [M+H]⁺.

### Step 6: O-t-Butyl N-[4-[(1r,4r)[4-[4-tert-butyl-2-[4-amino-2-fluorophenoxy]phenyl]pyrimidin-2-yl]amino]cyclohexyl] carbamate

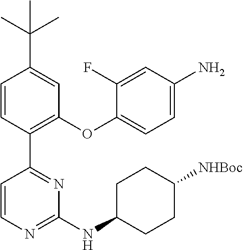 [SMILES: CC(C)(C)OC(=O)N[C@H]1CC[C@H](Nc2nccc(-c3ccc(C(C)(C)C)cc3Oc3ccc(N)cc3F)n2)CC1]

To a solution of O-t-butyl N-[4-[(1r,4r)[4-[4-tert-butyl-2-[2-fluoro-4-nitrophenoxy]phenyl]pyrimidin-2-yl]amino]cyclohexyl] carbamate (160 mg, 262.22 μmol, 1 eq) in EtOH (5 mL) and H₂O (1 mL) was added Fe (73.22 mg, 1.31 mmol, 5 eq) and NH₄Cl (140.27 mg, 2.62 mmol, 10 eq). The mixture was stirred at 80° C. for 2 h. After filtration, the organic layer was concentrated under reduced pressure to yield a yellow oil which was purified by flash silica gel chromatography (PE/EtOAc=1/0 to 1/1, TLC: PE/EtOAc=1/1, R_{f}=0.30) to yield O-t-butyl N-[4-[(1r,4r)[4-[4-tert-butyl-2-[4-amino-2-fluorophenoxy]phenyl]pyrimidin-2-yl]amino]cyclohexyl] carbamate (145 mg, 258.52 μmol, 98.6% yield, 98.0% purity) as a yellow solid. ¹H NMR (400 MHz, DMSO-d₆) δ ppm 8.25 (d, J=5.3 Hz, 1H), 7.81 (d, J=8.3 Hz, 1H), 7.20 (d, J=7.5 Hz, 1H), 7.08 (d, J=5.1 Hz, 1H), 6.95 (d, J=7.6 Hz, 1H), 6.89 (t, J=9.0 Hz, 1H), 6.70 (s, 2H), 6.50 (dd, J=2.6, 13.2 Hz, 1H), 6.39 (dd, J=1.9, 8.8 Hz, 1H), 5.33 (s, 2H), 3.72-3.61 (m, 1H), 3.19 (s, 1H), 1.97-1.89 (m, 2H), 1.82-1.76 (m, 2H), 1.38 (s, 9H), 1.32-1.23 (m, 4H), 1.18 (s, 9H); ES-LCMS m/z 550.4 [M+H]⁺.

### Step 7: O-t-Butyl N-[4-[(1r,4r)[4-[4-tert-butyl-2-[4-[2-chlorophenylsulfonamido]-2-fluorophenoxy]phenyl]pyrimidin-2-yl]amino]cyclohexyl] carbamate

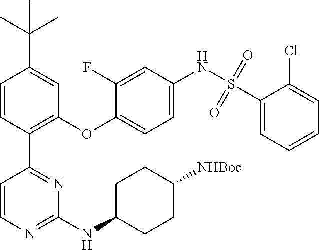 [SMILES: CC(C)(C)OC(=O)N[C@H]1CC[C@H](Nc2nccc(-c3ccc(C(C)(C)C)cc3Oc3ccc(NS(=O)(=O)c4ccccc4Cl)cc3F)n2)CC1]

To a solution of O-t-butyl N-[4-[(1r,4r)[4-[4-tert-butyl-2-[4-amino-2-fluorophenoxy]phenyl]pyrimidin-2-yl]amino]cyclohexyl] carbamate (145 mg, 258.52 μmol, 1 eq) in THF (2 mL) was added pyridine (163.59 mg, 2.07 mmol, 166.93 μL, 8 eq) and 2-chlorobenzenesulfonyl chloride (163.69 mg, 775.55 μmol, 105.74 μL, 3 eq). The mixture was stirred at 25° C. for 2 h. The reaction mixture was diluted with H₂O (30 mL) and extracted with EtOAc (20 mL×3). The combined organic layers were washed with brine (30 mL), dried over Na₂SO₄, filtered and concentrated under reduced pressure to yield a residue which was purified by flash silica gel chromatography (PE/EtOAc=1/0 to 1/1, TLC: PE/EtOAc=1/1, R_{f}=0.19) to yield O-t-butyl N-[4-[(1r,4r)[4-[4-tert-butyl-2-[4-[2-chlorophenylsulfonamido]-2-fluorophenoxy]phenyl]pyrimidin-2-yl]amino]cyclohexyl] carbamate (150 mg, 205.03 μmol, 79.3% yield, 99.0% purity) as a yellow solid. ¹H NMR (400 MHz, DMSO-d₆) δ ppm 10.91-10.64 (m, 1H), 8.19 (d, J=5.1 Hz, 1H), 8.03 (d, J=7.6 Hz, 1H), 7.80 (d, J=8.1 Hz, 1H), 7.63 (d, J=3.9 Hz, 2H), 7.55-7.46 (m, 1H), 7.27 (d, J=7.3 Hz, 1H), 7.05 (dd, J=2.4, 12.3 Hz, 1H), 7.00-6.93 (m, 2H), 6.93-6.86 (m, 2H), 6.70 (d, J=7.1 Hz, 1H), 6.64 (d, J=1.0 Hz, 1H), 3.65-3.59 (m, 1H), 3.18-3.17 (m, 1H), 1.94-1.88 (m, 2H), 1.78 (d, J=10.8 Hz, 2H), 1.38 (s, 9H), 1.30-1.21 (m, 4H), 1.15 (s, 9H); ES-LCMS m/z 724.2 [M+H]⁺.

### Step 8: N-[4-[2-[2-[(1r,4r)(4-Aminocyclohexyl)amino]pyrimidin-4-yl]-5-(1,1-dimethylethyl)phenoxy]-3-fluorophenyl] 2-chlorobenzenesulfonamide

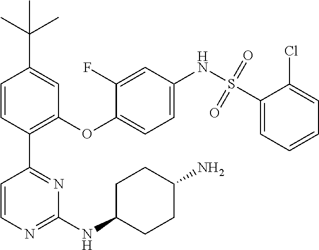 [SMILES: CC(C)(C)c1ccc(-c2ccnc(N[C@H]3CC[C@H](N)CC3)n2)c(Oc2ccc(NS(=O)(=O)c3ccccc3Cl)cc2F)c1]

To a solution of O-t-butyl N-[4-[(1r,4r)[4-[4-tert-butyl-2-[4-[(2-chlorophenyl)sulfonamido]-2-fluorophenoxy]phenyl]pyrimidin-2-yl]amino]cyclohexyl] carbamate (150 mg, 205.03 μmol, 1 eq) in DCM (2 mL) was added HCl/dioxane (4 M, 768.86 μL, 15 eq). The mixture was stirred at 25° C. for 2 h. The reaction mixture was neutralized to pH=7 with NH₃·H₂O, the reaction mixture was concentrated to yield a residue which was purified by preparative HPLC (column: Welch Ultimate C18 150*25 mm*5 μm; mobile phase: [water (FA)-ACN]; B %: 23%-53%, 10 min), followed by lyophilization to yield N-[4-[2-[2-[(1r,4r)(4-aminocyclohexyl)amino]pyrimidin-4-yl]-4-(1,1-dimethylethyl)phenoxy]-3-fluorophenyl] 2-chlorobenzenesulfonamide (28.8 mg, 42.93 μmol, 20.9% yield, 99.9% purity, FA) as a white solid. ¹H NMR (400 MHz, DMSO-d₆ 75° C.) δ ppm 8.25 (brs, 1H), 8.23 (d, J=5.6 Hz, 1H), 7.98 (d, J=7.8 Hz, 1H), 7.80 (d, J=8.0 Hz, 1H), 7.52-7.42 (m, 2H), 7.37 (t, J=7.2 Hz, 1H), 7.22 (d, J=8.1 Hz, 1H), 6.99 (d, J=5.0 Hz, 1H), 6.89 (slbrd, J=13.75 Hz, 1H), 6.80 (t, J=9.0 Hz, 1H), 6.77-6.71 (m, 2H), 6.64 (brs, 1H), 3.69-3.62 (m, 1H), 2.90-2.85 (m, 1H), 2.05-1.87 (m, 4H), 1.43-1.24 (m, 4H), 1.20 (s, 9H); ES-LCMS m/z 624.3 [M+H]⁺.
12. N-[4-[2-[2-[Oxetan-3-yl)amino]pyrimidin-4-yl]-5-(1-methylethyl)phenoxy]-3-fluorophenyl] 2-chlorobenzenesulfonamide

### Step 1: 2-Bromo-5-isopropylphenol

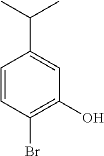

To a solution of 3-isopropylphenol (2.0 g, 14.68 mmol) in CS₂ (40 mL) was added NBS (2.61 g, 14.68 mmol, 1 eq). The reaction mixture was stirred at 0° C. for 1 h and then for an additional hour at room temperature. The resulting precipitate was filtered off and the filtrate was concentrated under reduced pressure to give a residue which was purified by silica gel flash column chromatography (0% to 10% EtOAc in Hex) to afford an isomeric mixture of 2-bromo-5-isopropylphenol and 2-bromo-3-isopropylphenol (3:1, 2.59 g, 82% yield) as a colorless oil. ¹H NMR (500 MHz, Chloroform-d) δ 7.35 (d, J=8.3 Hz, 1H), 6.91 (d, J=2.0 Hz, 1H), 6.69 (dd, J=8.3, 2.1 Hz, 1H), 5.43 (s, 1H), 2.84 (m, 1H), 1.22 (d, J=6.9 Hz, 6H).

### Step 2: 2-(2-(2-Fluoro-4-nitrophenoxy)-4-isopropylphenyl)-4,4,5,5-tetramethyl-1,3,2-dioxaborolane

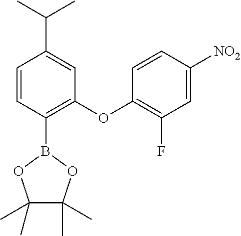 [SMILES: CC(C)c1ccc(B2OC(C)(C)C(C)(C)O2)c(Oc2ccc([N+](=O)[O-])cc2F)c1]

To a solution of an isomeric mixture of 2-bromo-5-isopropylphenol and 2-bromo-3-isopropylphenol (3:1, 2.59 g, 12.10 mmol) in DMF (30 mL) were added K₂CO₃ (3.34 g, 24.20 mmol, 2 eq) and 1,2-difluoro-4-nitrobenzene (1.34 mL, 12.10 mmol, 1 eq). The reaction mixture was stirred at 80° C. for 3 h. The resulting mixture was diluted with H₂O (60 mL) and extracted with EtOAc (80 mL×2). The combined organic layers were washed with water (80 mL×2) and brine (60 mL), dried over Na₂SO₄, filtered and concentrated under reduced pressure to give a crude material (3.82 g, 10.82 mmol), which was then dissolved in dioxane (80 mL). To this solution were added KOAc (3.19 g, 32.46 mmol, 3 eq), bis(pinacolato)diboron (5.49 g, 21.64 mmol, 2 eq) and Pd(dppf)Cl₂ (1.33 g, 1.82 mmol, 0.1 eq). The reaction mixture was stirred at 100° C. for 3 h under N₂. The resulting mixture was filtered through a pad of celite and the pad was washed with EtOAc (100 mL). The filtrate was concentrated under reduced pressure to give an oily residue which was purified by silica gel flash column chromatography (3% to 5% EtOAc in Hex) to afford 2-(2-(2-fluoro-4-nitrophenoxy)-4-isopropylphenyl)-4,4,5,5-tetramethyl-1,3,2-dioxaborolane (2.89 g, 7.21 mmol, 60% yield) as a yellow solid. ¹H NMR (500 MHz, Chloroform-d) δ 8.06 (dd, J=10.4, 2.6 Hz, 1H), 7.90-7.84 (m, 1H), 7.79 (d, J=7.7 Hz, 1H), 7.22-7.16 (m, 1H), 7.01 (s, tH), 6.61-6.54 (m, 1H), 2.95 (m, tH), 1.26 (d, J=6.9 Hz, 6H), 1.10 (s, 9H); ESI-MS rm/z 402 [M+H]⁺.

### Step 3: 2-Chloro-4-(2-(2-fluoro-4-nitrophenoxy)-4-isopropylphenyl)pyrimidine

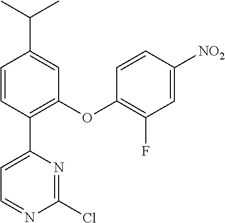 [SMILES: CC(C)c1ccc(-c2ccnc(Cl)n2)c(Oc2ccc([N+](=O)[O-])cc2F)c1]

To a solution of 2-(2-(2-fluoro-4-nitrophenoxy)-4-isopropylphenyl)-4,4,5,5-tetramethyl-1,3,2-dioxaborolane (1.0 g, 2.49 mmol) in dioxane (25 mL) and H₂O (8 mL) were added Cs₂CO₃ (1.62 mg, 4.98 mmol, 2 eq), 2,4-dichloropyrimidine (423 mg, 2.84 mmol, 1.1 eq) and Pd(dppf)Cl₂ (182 mg, 0.25 mmol, 0.1 eq). The reaction mixture was stirred at 90° C. for 3 h under N₂. The resulting mixture was diluted with H₂O (50 mL) and extracted with EtOAc (80 mL×2). The combined organic layer was washed with brine (50 mL), dried over Na₂SO₄, filtered and concentrated under reduced pressure to give a crude oil, which was purified by silica gel flash column chromatography (3% to 7% EtOAc in Hex) to afford 2-chloro-4-(2-(2-fluoro-4-nitrophenoxy)-4-isopropylphenyl)pyrimidine (380 mg, 0.982 mmol, 39% yield) as a yellow solid. ¹H NMR (500 MHz, Chloroform-d) δ 8.58 (d, J=5.2 Hz, 1H), 8.08 (d, J=8.0 Hz, 2H), 7.98-7.92 (m, 1H), 7.79 (d, J=5.2 Hz, 1H), 7.32 (d, J=7.5 Hz, 1H), 6.97 (s, 1H), 6.86 (t, J=8.5 Hz, 1H), 2.98 (m, 1H), 1.28 (d, J=6.9 Hz, 6H); ESI-MS m/z 388 [M+H]⁺.

### Step 4: 4-(2-(2-Chloropyrimidin-4-yl)-5-isopropylphenoxy)-3-fluoroaniline

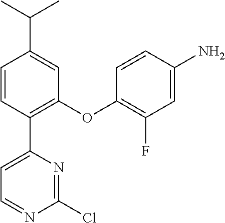 [SMILES: CC(C)c1ccc(-c2ccnc(Cl)n2)c(Oc2ccc(N)cc2F)c1]

To a solution of 2-chloro-4-(2-(2-fluoro-4-nitrophenoxy)-4-isopropylphenyl)pyrimidine (185 mg, 0.478 mmol) in THF was added water (7 mL), and then zinc (188 mg, 2.87 mmol, 6 eq) and ammonium chloride (153 mg, 2.87 mmol, 6 eq) were added. The reaction mixture was stirred at room temperature for 1 h. The same amounts of zinc and ammonium chloride were added and stirred for an additional hour. The resulting mixture was diluted with H₂O (30 mL) and extracted with EtOAc (40 mL×2). The combined organic layers were washed with brine (40 mL), dried over Na₂SO₄, filtered and concentrated under reduced pressure to yield the title compound which was used for the next reaction without purification; ESI-MS m/z 358 [M+H]⁺.

### Step 5: 2-Chloro-N-(4-(2-(2-chloropyrimidin-4-yl)-5-isopropylphenoxy)-3-fluorophenyl)benzenesulfonamide

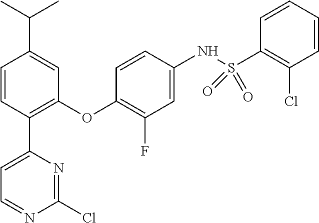 [SMILES: CC(C)c1ccc(-c2ccnc(Cl)n2)c(Oc2ccc(NS(=O)(=O)c3ccccc3Cl)cc2F)c1]

To a stirred solution of 4-(2-(2-chloropyrimidin-4-yl)-5-isopropylphenoxy)-3-fluoroaniline (65 mg, 0.182 mmol) and 2-chlorobenzenesulfonyl chloride (58 mg, 0.273 mmol, 1.5 eq) in DCM (2 mL) was added pyridine (43 mg, 0.546 mmol, 44 μL, 3 eq). The reaction mixture was stirred at room temperature overnight. The resulting mixture was diluted with H₂O (10 mL) and extracted with DCM (20 mL×2). The combined organic layers were washed with brine (20 mL), dried over Na₂SO₄, filtered and concentrated under reduced pressure to give a residue which was purified by silica gel flash column chromatography (10% to 30% EtOAc in Hex) to yield 2-chloro-N-(4-(2-(2-chloropyrimidin-4-yl)-5-isopropylphenoxy)-3-fluorophenyl)benzenesulfonamide (66 mg, 0.124 mmol, 68% yield) as a yellow solid; ESI-MS m/z 532 [M+H]⁺.

### Step 6: 2-Chloro-N-(3-fluoro-4-(5-isopropyl-2-(2-(oxetan-3-ylamino)pyrimidin-4-yl)phenoxy)phenyl)benzenesulfonamide

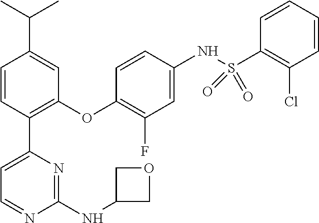 [SMILES: CC(C)c1ccc(-c2ccnc(NC3COC3)n2)c(Oc2ccc(NS(=O)(=O)c3ccccc3Cl)cc2F)c1]

To a solution of 2-chloro-N-(4-(2-(2-chloropyrimidin-4-yl)-5-isopropylphenoxy)-3-fluorophenyl)benzenesulfonamide (53 mg, 0.100 mmol) in DMSO (1 mL) was added 3-aminooxetane (15 mg, 0.200 mmol, 2 eq) and TEA (30 mg, 0.300 mmol, 3 eq). The reaction mixture was heated at 110° C. for 3 h. The resulting mixture was diluted with H₂O (10 mL) and extracted with EtOAc (20 mL×2). The combined organic layers were washed with water (40 mL) and brine (20 mL), dried over Na₂SO₄, filtered and concentrated under reduced pressure to give a residue which was purified by silica gel flash column chromatography (30% to 70% EtOAc in Hex) to yield 2-chloro-N-(3-fluoro-4-(5-isopropyl-2-(2-(oxetan-3-ylamino)pyrimidin-4-yl)phenoxy)phenyl)benzenesulfonamide (48 mg, 0.0845 mmol, 85% yield) as a light yellow sticky oil; ¹H NMR (500 MHz, Chloroform-d) δ 8.20 (d, J=5.3 Hz, 1H), 8.02 (dd, J=7.9, 1.2 Hz, 1H), 7.87 (d, J=8.0 Hz, 1H), 7.55-7.48 (m, 2H), 7.45 (bs, 1H), 7.37 (ddd, J=7.9, 6.9, 1.8 Hz, 1H), 7.18 (d, J=5.3 Hz, 1H), 7.11 (dd, J=8.1, 1.3 Hz, 1H), 7.05 (dd, J=11.3, 2.2 Hz, 1H), 6.83-6.75 (m, 2H), 6.60 (s, 1H), 6.00 (bs, 1H), 5.16 (m, J=6.9 Hz, 1H), 4.98 (t, J=7.0 Hz, 2H), 4.62 (t, J=6.5 Hz, 2H), 2.84 (m, 1H), 1.18 (d, J=6.9 Hz, 6H); ESI-MS m/z 569 [M+H]⁺.

The following compounds have been made by processes similar to those described immediately above using Method A.
13. N-[4-[2-[2-[Oxetan-3-yl)amino]pyrimidin-4-yl]-5-(1-methylethyl)phenoxy]-3-fluorophenyl] 2-methylbenzenesulfonamide. ES-LCMS m/z 549.1 [M+H]⁺.
14. N-[4-[2-[2-[Oxetan-3-yl)amino]pyrimidin-4-yl]-5-(1-methylethyl)phenoxy]-3-fluorophenyl] 2-cyanobenzenesulfonamide. ES-LCMS m/z 560.1 [M+H]⁺.
15. N-[4-[2-[2-[Oxetan-3-yl)amino]pyrimidin-4-yl]-5-(1-methylethyl)phenoxy]-3-fluorophenyl] 2-ethynylbenzenesulfonamide. ES-LCMS m/z 559.1 [M+H]⁺.
16. N-[4-[2-[2-[(1r,4r)(4-Aminocyclohexyl)amino]pyrimidin-4-yl]-5-iodophenoxy]-3-fluorophenyl] 2-chlorobenzenesulfonamide.

### Step 1: 3-Methoxy-4-(4,4,5,5-tetramethyl-1,3,2-dioxaborolan-2-yl)aniline

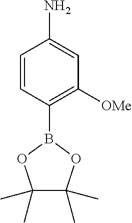 [SMILES: COc1cc(N)ccc1B1OC(C)(C)C(C)(C)O1]

To a stirred solution of 4-bromo-3-methoxyaniline (5 g, 24.75 mmol, 1 eq) and 4,4,5,5-tetramethyl-2-(4,4,5,5-tetramethyl-1,3,2-dioxaborolan-2-yl)-1,3,2-dioxaborolane (9.43 g, 37.12 mmol, 1.5 eq) in dioxane (100 mL) was added Pd(PPh₃)₄ (1.43 g, 1.24 mmol, 0.05 eq) and Cs₂CO₃ (24.19 g, 74.24 mmol, 3 eq). The reaction mixture was stirred at 110° C. for 12 h under N₂. The reaction mixture was diluted with H₂O (400 mL) and extracted with EtOAc (150 mL×3). The combined organic layers were washed with brine (150 mL), dried over Na₂SO₄, filtered and concentrated under reduced pressure to give a residue. The residue was purified by flash silica gel chromatography (PE/EtOAc=100/1 to 4/1, TLC: PE/EtOAc=1/1, R_{f}=0.45) to yield 3-methoxy-4-(4,4,5,5-tetramethyl-1,3,2-dioxaborolan-2-yl)aniline (5.7 g, 22.42 mmol, 30.2% yield, 98.0% purity) as a brown solid. ¹H NMR (400 MHz, DMSO-d₆) δ ppm 7.24 (d, J=7.8 Hz, 1H), 6.13-6.05 (m, 2H), 5.48 (s, 2H), 3.62 (s, 3H), 1.21 (s, 12H); ES-LCMS m/z 250.0 [M+H]⁺.

### Step 2: 4-(2-Chloropyrimidin-4-yl)-3-methoxyaniline

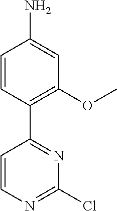 [SMILES: COc1cc(N)ccc1-c1ccnc(Cl)n1]

To a stirred solution of 3-methoxy-4-(4,4,5,5-tetramethyl-1,3,2-dioxaborolan-2-yl)aniline (6.7 g, 26.90 mmol, 1 eq) and 2,4-dichloropyrimidine (4.01 g, 26.90 mmol, 1 eq) in 1,4-dioxane (30 mL) and H₂O (9 mL) was added Pd(dppf)Cl₂ (1.97 g, 2.69 mmol, 0.1 eq) and Cs₂CO₃ (17.53 g, 53.79 mmol, 2 eq). The reaction mixture was stirred at 60° C. for 2 h under N₂. The reaction mixture was diluted with H₂O (200 mL) and extracted with EtOAc (100 mL×3). The combined organic layers were washed with brine (100 mL), dried over Na₂SO₄, filtered and concentrated under reduced pressure to give a residue. The residue was purified by flash silica gel chromatography (DCM/MeOH=100/1 to 10/1, TLC: PE/EtOAc=1/1, R_{f}=0.55) to yield 4-(2-chloropyrimidin-4-yl)-3-methoxyaniline (3.5 g, 14.70 mmol, 54.7% yield, 99.0% purity) as a yellow solid. ¹H NMR (400 MHz, DMSO-d₆) δ ppm 8.49 (d, J=5.5 Hz, 1H), 7.97 (d, J=5.6 Hz, 1H), 7.90 (d, J=9.1 Hz, 1H), 6.32-6.27 (m, 2H), 6.05 (s, 2H), 3.84 (s, 3H); ES-LCMS m/z 236.0 [M+H]⁺.

### Step 3: 2-Chloro-4-[4-iodo-25-methoxyphenyl]pyrimidine

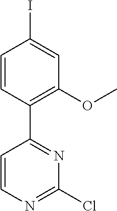 [SMILES: COc1cc(I)ccc1-c1ccnc(Cl)n1]

To a stirred solution of 4-(2-chloropyrimidin-4-yl)-3-methoxyaniline (3 g, 12.60 mmol, 1 eq) in MeCN (40 mL) and H₂O (12 mL) was added NaNO₂ (1.74 g, 25.20 mmol, 2 eq) and H₂SO₄ (4 mL). The reaction mixture was stirred at 0° C. for 0.5 h. KI (6.28 g, 37.81 mmol, 3 eq) was added. The reaction mixture was stirred at 0° C. for another 1.5 h. The residue was diluted with saturated Na₂SO₃ (100 mL) solution and extracted with EtOAc (100 mL×3). The combined organic layers were washed with brine (100 mL), dried over Na₂SO₄, filtered and concentrated under reduced pressure to yield 2-chloro-4-(4-iodo-2-methoxyphenyl)pyrimidine (4 g, 11.43 mmol, 90.7% yield, 99.0% purity) as a yellow solid. ¹H NMR (400 MHz, DMSO-d₆) δ ppm 8.76 (d, J=5.3 Hz, 1H), 8.04 (d, J=5.3 Hz, 1H), 7.69 (d, J=8.3 Hz, 1H), 7.58-7.49 (m, 2H), 3.92 (s, 3H); ES-LCMS m/z 346.8 [M+H]⁺.

### Step 4: 2-(2-Chloropyrimidin-4-yl)-5-iodophenol

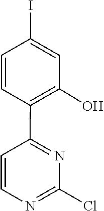 [SMILES: Oc1cc(I)ccc1-c1ccnc(Cl)n1]

To a stirred solution of 2-chloro-4-(4-iodo-2-methoxyphenyl)pyrimidine (3 g, 8.57 mmol, 1 eq) in DCM (150 mL) was added BBr₃ (10.74 g, 42.85 mmol, 4.13 mL, 5 eq) at 0° C. The reaction mixture was stirred at 40° C. for 4 h. The reaction mixture was diluted with H₂O (150 mL) and extracted with EtOAc (100 mL×3). The combined organic layers were washed with brine (100 mL), dried over Na₂SO₄, filtered and concentrated under reduced pressure to yield 2-(2-chloropyrimidin-4-yl)-5-iodophenol (2.7 g, 8.04 mmol, 93.8% yield, 99.0% purity) as a yellow solid. ¹H NMR (400 MHz, DMSO-d₆) δ ppm 11.26 (brs, 1H), 8.77 (d, J=5.3 Hz, 1H), 8.22 (d, J=5.4 Hz, 1H), 7.77 (d, J=8.3 Hz, 1H), 7.42-7.32 (m, 2H); ES-LCMS m/z 332.8 [M+H]⁺.

### Step 5: 2-Chloro-4-[2-(2-fluoro-4-nitrophenoxy)-4-iodophenyl]pyrimidine

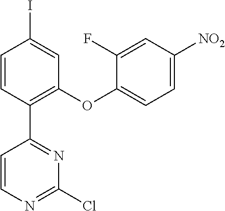 [SMILES: O=[N+]([O-])c1ccc(Oc2cc(I)ccc2-c2ccnc(Cl)n2)c(F)c1]

To a stirred solution of 2-(2-chloropyrimidin-4-yl)-5-iodophenol (3.6 g, 10.83 mmol, 1 eq) and 1,2-difluoro-4-nitrobenzene (1.72 g, 10.83 mmol, 1.20 mL, 1 eq) in DMSO (80 mL) was added K₂CO₃ (2.99 g, 21.65 mmol, 2 eq). The reaction mixture was stirred at 40° C. for 2 h. The reaction mixture was diluted with H₂O (100 mL) and extracted with EtOAc (100 mL×3). The combined organic layers were washed with brine (100 mL), dried over Na₂SO₄, filtered and concentrated under reduced pressure to give a residue which was purified by flash silica gel chromatography (PE/EtOAc=100/1 to 4/1, TLC: PE/EtOAc=3/1, R_{f}=0.45) to yield 2-chloro-4-[2-(2-fluoro-4-nitrophenoxy)-4-iodophenyl]pyrimidine (4.5 g, 9.45 mmol, 87.3% yield, 99.0% purity) as a yellow solid. ¹H NMR (400 MHz, DMSO-d₆) δ ppm 8.80 (d, J=5.3 Hz, 1H), 8.34 (dd, J=2.8, 10.6 Hz, 1H), 8.08 (td, J=1.3, 9.1 Hz, 1H), 7.93 (d, J=5.3 Hz, 1H), 7.88 (dd, J=1.5, 8.3 Hz, 1H), 7.81-7.76 (m, 1H), 7.68 (d, J=1.5 Hz, 1H), 7.28 (t, J=8.7 Hz, 1H); ES-LCMS m/z 471.8 [M+H]⁺.

### Step 6: O-t-Butyl N-[4-[(1r,4r)[4-[2-(2-fluoro-4-nitrophenoxy)-4-iodophenyl]pyrimidin-2-yl]amino]cyclohexyl] carbamate

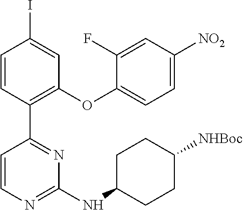 [SMILES: CC(C)(C)OC(=O)N[C@H]1CC[C@H](Nc2nccc(-c3ccc(I)cc3Oc3ccc([N+](=O)[O-])cc3F)n2)CC1]

To a stirred solution of 2-chloro-4-[2-(2-fluoro-4-nitrophenoxy)-4-iodophenyl]pyrimidine (4.5 g, 9.45 mmol, 1 eq) and O-t-butyl N-[(1r,4r)-4-aminocyclohexyl] carbamate (2.02 g, 9.45 mmol, 1 eq) in DMSO (80 mL) was added TEA (2.87 g, 28.34 mmol, 3.94 mL, 3 eq). The reaction mixture was stirred at 110° C. for 3 h. The reaction mixture was diluted with H₂O (100 mL) and extracted with EtOAc (100 mL×3). The combined organic layers were washed with brine (300 mL), dried over Na₂SO₄, filtered and concentrated under reduced pressure to give a residue which was purified by flash silica gel chromatography (PE/EtOAc=100/1 to 4/1, TLC: PE/EtOAc=1/1, R_{f}=0.50) to yield O-t-butyl N-[4-└(1r,4r)└4-└2-(2-fluoro-4-nitrophenoxy)-4-iodophenyl┘pyrimidin-2-yl┘amino┘cyclohexyl┘ carbamate (3.1 g, 4.73 mmol, 50.0% yield, 99.0% purity) as a yellow solid. ¹H NMR (400 MHz, DMSO-d₆) δ ppm 8.31 (dd, J=2.8, 10.6 Hz, 1H), 8.25 (d, J=5.1 Hz, 1H), 8.05 (td, J=1.3, 9.1 Hz, 1H), 7.83 (d, J=8.1 Hz, 1H), 7.71-7.59 (m, 2H), 7.16-7.02 (m, 2H), 6.81 (d, J=5.0 Hz, 1H), 6.74-6.60 (m, 1H), 3.66-3.42 (m, 1H), 3.24-3.07 (m, 1H), 1.87-1.70 (m, 4H), 1.38 (s, 9H), 1.31-1.13 (m, 4H); ES-LCMS m/z 650.1 [M+H]⁺.

### Step 7: O-t-Butyl N-[4-[(1r,4r)[4-[2-(4-amino-2-fluorophenoxy)-4-iodophenyl]pyrimidin-2-yl]amino]cyclohexyl] carbamate

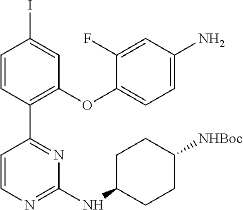 [SMILES: CC(C)(C)OC(=O)N[C@H]1CC[C@H](Nc2nccc(-c3ccc(I)cc3Oc3ccc(N)cc3F)n2)CC1]

To a stirred solution of O-t-butyl N-[4-[(1r,4r)[4-[2-(2-fluoro-4-nitrophenoxy)-4-iodophenyl]pyrimidin-2-yl]amino]cyclohexyl] carbamate (3.1 g, 4.73 mmol, 1 eq) in EtOH (50 mL) and H₂O (10 mL) was added Fe (1.32 g, 23.63 mmol, 5 eq) and NH₄Cl (2.53 g, 47.26 mmol, 10 eq). The reaction mixture was stirred at 80° C. for 3 h. The reaction mixture was filtered and concentrated under reduced pressure to yield O-t-butyl N-[4-[(1r,4r)[4-[2-(4-amino-2-fluorophenoxy)-4-iodophenyl]pyrimidin-2-yl]amino]cyclohexyl]carbamate (3 g, 4.70 mmol, 99.4% yield, 97.0% purity) as a yellow solid. ¹H NMR (400 MHz, DMSO-d₆) δ ppm 8.29 (d, J=5.1 Hz, 1H), 7.68-7.59 (m, 1H), 7.58-7.48 (m, 1H), 7.06 (d, J=5.1 Hz, 2H), 7.01-6.92 (m, 2H), 6.72 (d, J=7.9 Hz, 1H), 6.52 (dd, J=2.5, 13.3 Hz, 1H), 6.42 (dd, J=2.0, 8.6 Hz, 1H), 5.44 (s, 2H), 3.74-3.61 (m, 1H), 3.24-3.15 (m, 1H), 1.94 (d, J=9.8 Hz, 2H), 1.79 (d, J=10.0 Hz, 2H), 1.38 (s, 9H), 1.33-1.22 (m, 4H); ES-LCMS m/z 620.0 [M+H]⁺.

### Step 8: O-t-Butyl N-[4-[(1r,4r)[4-[2-[4-[2-chlorophenylsulfonylamido]-2-fluorophenoxy]-4-iodophenyl]pyrimidin-2-yl]amino]cyclohexyl] carbamate

To a stirred solution of O-t-butyl N-[4-[(1r,4r)[4-[2-(4-amino-2-fluorophenoxy)-4-iodophenyl]pyrimidin-2-yl]amino]cyclohexyl]carbamate (100 mg, 156.59 μmol, 1 eq) in THF (2 mL) was added 2-chlorobenzenesulfonyl chloride (99.15 mg, 469.76 μmol, 64.05 μL, 3 eq) and pyridine (123.86 mg, 1.57 mmol, 126.39 μL, 10 eq) at 0° C. The reaction mixture was stirred at 25° C. for 12 h. The reaction mixture was diluted with H₂O (50 mL) and extracted with EtOAc (20 mL×3). The combined organic layers were washed with brine (10 mL), dried over Na₂SO₄, filtered and concentrated under reduced pressure to give a residue. The residue was purified by preparative TLC (PE/EtOAc=1/1, TLC: PE/EtOAc=1/1, R_{f}=0.40) to yield O-t-butyl N-[4-[(1r,4r)[4-[2-[4-[2-chlorophenylsulfonylamido]-2-fluorophenoxy]-4-iodophenyl]pyrimidin-2-yl]amino]cyclohexyl] carbamate (60 mg, 71.78 μmol, 45.8% yield, 95.0% purity) as a yellow solid. ¹H NMR (400 MHz, DMSO-d₆) δ ppm 10.96-10.78 (m, 1H), 8.23 (d, J=5.0 Hz, 1H), 8.06 (d, J=7.6 Hz, 1H), 7.68-7.57 (m, 4H), 7.57-7.49 (m, 1H), 7.13-7.00 (m, 3H), 6.98-6.87 (m, 3H), 6.74-6.63 (m, 1H), 3.70-3.56 (m, 1H), 3.23-3.13 (m, 1H), 1.90 (d, J=8.8 Hz, 2H), 1.83-1.71 (m, 2H), 1.38 (s, 9H), 1.32-1.21 (m, 4H); ES-LCMS m/z 794.1, [M+H]⁺.

### Step 9: N-[4-[2-[2-[(1r,4r)(4-Aminocyclohexyl)amino]pyrimidin-4-yl]-5-iodophenoxy]-3-fluorophenyl] 2-chlorobenzenesulfonamide

To a stirred solution of O-t-butyl N-[4-[(1r,4r)[4-[2-[4-[2-chlorophenylsulfonylamido]-2-fluorophenoxy]-4-iodophenyl]pyrimidin-2-yl]amino]cyclohexyl] carbamate (60 mg, 71.78 μmol, 1 eq) in MeOH (1 mL) was added HCl/dioxane (4 M, 179.5 μL, 10 eq). The reaction mixture was stirred at 25° C. for 0.5 h. The reaction mixture was neutralized to pH=7 with NH₃·H₂O and extracted with EtOAc (30 mL×3). The combined organic layers were washed with brine (30 mL), dried over Na₂SO₄, filtered and concentrated under reduced pressure to give a residue. The residue was purified by preparative HPLC (column: Welch Ultimate C18 150*25 mm*5 μm; mobile phase: [water (FA)-ACN]; gradient: 22%-52% B over 10 min) followed by lyophilization to yield N-[4-[2-[2-[(1r,4r)(4-aminocyclohexyl)amino]pyrimidin-4-yl]-5-iodophenoxy]-3-fluorophenyl] 2-chlorobenzenesulfonamide (16.8 mg, 22.70 μmol, 31.6% yield, 100.0% purity, 1.0 FA) as a white solid. ¹H NMR (400 MHz, DMSO-d₆) δ ppm 8.29-8.23 (m, 2H), 7.97 (dd, J=1.6, 7.8 Hz, 1H), 7.61-7.53 (m, 2H), 7.49-7.36 (m, 3H), 7.18-7.08 (m, 1H), 7.01-6.81 (m, 4H), 6.71-6.63 (m, 1H), 3.66-3.63 (m, 1H), 2.99-2.95 (m, 1H), 1.99-1.90 (m, 4H), 1.41-1.27 (m, 4H); ES-LCMS m/z 694.1 [M+H]⁺.
17. N-[4-[2-[2-[(1r,4r)(4-Aminocyclohexyl)amino]pyrimidin-4-yl]-5-ethylphenoxy]-3-fluorophenyl] 2-chlorobenzenesulfonamide. ES-LCMS m/z 694.1, 695.8 └M+H┘⁺.

### Step 1: O-t-Butyl N-[4-[(1r,4r)[4-[2-(4-amino-2-fluorophenoxy)-4-ethenylphenyl]pyrimidin-2-yl]amino]cyclohexyl] carbamate

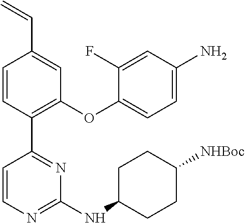 [SMILES: C=Cc1ccc(-c2ccnc(N[C@H]3CC[C@H](NC(=O)OC(C)(C)C)CC3)n2)c(Oc2ccc(N)cc2F)c1]

To a stirred solution of O-t-butyl N-[4-[(1r,4r)[4-[2-(4-amino-2-fluorophenoxy)-4-iodophenyl]pyrimidin-2-yl]amino]cyclohexyl]carbamate (100 mg, 156.59 μmol, 1 eq) and 4,4,5,5-tetramethyl-2-vinyl-1,3,2-dioxaborolane (24.12 mg, 156.59 μmol, 26.56 μL, 1 eq) in dioxane (2 mL) and H₂O (0.6 mL) was added Pd(dppf)Cl₂ (11.46 mg, 15.66 μmol, 0.1 eq) and Cs₂CO₃ (102.04 mg, 313.17 μmol, 2 eq). The reaction mixture was stirred at 90° C. for 12 h under N₂. The reaction mixture was diluted with H₂O (15 mL) and extracted with EtOAc (20 mL×3). The combined organic layers were washed with brine (15 mL), dried over Na₂SO₄, filtered and concentrated under reduced pressure to give a residue. The residue was purified by preparative TLC (PE/EtOAc=1/1, TLC: PE/EtOAc=1/1, R_{f}=0.45) to yield O-t-butyl N-[4-[(1r,4r)[4-[2-(4-amino-2-fluorophenoxy)-4-ethenylphenyl]pyrimidin-2-yl]amino]cyclohexyl] carbamate (80 mg, 147.80 μmol, 94.4% yield, 96.0% purity) as a yellow solid. ¹H NMR (400 MHz, DMSO-d₆) δ ppm 8.27 (d, J=5.0 Hz, 1H), 7.87 (d, J=8.0 Hz, 1H), 7.32 (d, J=7.6 Hz, 1H), 7.10 (d, J=5.0 Hz, 1H), 7.01 (d, J=6.4 Hz, 1H), 6.92 (t, J=9.1 Hz, 1H), 6.78-6.62 (m, 3H), 6.50 (dd, J=2.0, 13.1 Hz, 1H), 6.40 (d, J=8.6 Hz, 1H), 5.74 (d, J=17.5 Hz, 1H), 5.36 (s, 2H), 5.28 (d, J=10.9 Hz, 1H), 3.69 (d, J=2.3 Hz, 1H), 3.25-3.17 (m, 1H), 1.95 (d, J=8.3 Hz, 2H), 1.80 (d, J=9.3 Hz, 2H), 1.38 (s, 9H), 1.34-1.23 (m, 4H); ES-LCMS m/z 520.2 [M+H]⁺.

### Step 2: O-t-Butyl N-[4-[(1r,4r)[4-[2-(4-amino-2-fluorophenoxy)-4-ethylphenyl]pyrimidin-2-yl]amino]cyclohexyl] carbamate

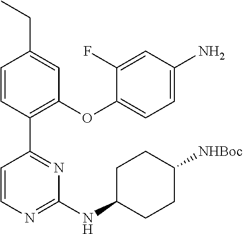 [SMILES: CCc1ccc(-c2ccnc(N[C@H]3CC[C@H](NC(=O)OC(C)(C)C)CC3)n2)c(Oc2ccc(N)cc2F)c1]

To a stirred solution of O-t-butyl N-[4-[(1r,4r)[4-[2-(4-amino-2-fluorophenoxy)-4-ethenylphenyl]pyrimidin-2-yl]amino]cyclohexyl] carbamate (80 mg, 147.80 μmol, 1 eq) in MeOH (5 mL) was added 10% Pd/C (100 mg). The reaction mixture was stirred at 25° C. for 3 h under H₂ (15 PSI). The reaction mixture was filtered and concentrated under reduced pressure to yield O-t-butyl N-[4-[(1r,4r)[4-[2-(4-amino-2-fluorophenoxy)-4-ethylphenyl]pyrimidin-2-yl]amino]cyclohexyl] carbamate (40 mg, 61.35 μmol, 41.5% yield, 80.0% purity) as a yellow solid. ¹H NMR (400 MHz, DMSO-d₆) δ ppm 8.25 (d, J=5.1 Hz, 1H), 7.79 (dd, J=2.0, 7.6 Hz, 1H), 7.09-6.87 (m, 4H), 6.72-6.62 (m, 1H), 6.62-6.28 (m, 3H), 3.77-3.59 (m, 1H), 3.24-3.20 (m, 1H), 2.54 (m, 2H), 1.99-1.90 (m, 2H), 1.84-1.75 (m, 2H), 1.38 (s, 9H), 1.34-1.24 (m, 4H), 1.13-1.07 (m, 3H); ES-LCMS m/z 522.2 ┌M+H┐⁺.

### Step 3: O-t-Butyl N-[4-[(1r,4r)[4-[2-[4-[2-chlorophenylsulfonylamido]-2-fluorophenoxy]-4-ethylphenyl]pyrimidin-2-yl]amino]cyclohexyl] carbamate

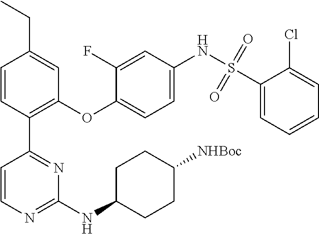 [SMILES: CCc1ccc(-c2ccnc(N[C@H]3CC[C@H](NC(=O)OC(C)(C)C)CC3)n2)c(Oc2ccc(NS(=O)(=O)c3ccccc3Cl)cc2F)c1]

To a stirred solution of O-t-butyl N-[4-[(1r,4r)[4-[2-(4-amino-2-fluorophenoxy)-4-ethylphenyl]pyrimidin-2-yl]amino]cyclohexyl] carbamate (40 mg, 61.35 μmol, 1 eq) and 2-chlorobenzenesulfonyl chloride (90.64 mg, 429.43 μmol, 58.55 μL, 7 eq) in THF (2 mL) was added pyridine (97.05 mg, 1.23 mmol, 99.03 μL, 20 eq). The reaction mixture was stirred at 25° C. for 12 h. The reaction mixture was diluted with H₂O (10 mL) and extracted with EtOAc (10 mL×3). The combined organic layers were washed with brine (10 mL), dried over Na₂SO₄, filtered and concentrated under reduced pressure to give a residue which was purified by preparative TLC (PE/EtOAc=1/1, TLC: PE/EtOAc=1/1, R_{f}=0.35) to yield O-t-butyl N-[4-[(1r,4r)[4-[2-[4-[2-chlorophenylsulfonylamido]-2-fluorophenoxy]-4-ethylphenyl]pyrimidin-2-yl]amino]cyclohexyl] carbamate (30 mg, 38.78 μmol, 63.2% yield, 90.0% purity) as a yellow solid. ¹H NMR (400 MHz, DMSO-d₆) δ ppm 8.19 (d, J=5.1 Hz, 1H), 8.02 (d, J=7.8 Hz, 1H), 7.78 (d, J=8.1 Hz, 1H), 7.64-7.53 (m, 2H), 7.52-7.40 (m, 1H), 7.07 (d, J=7.5 Hz, 1H), 7.02-6.88 (m, 4H), 6.87-6.78 (m, 1H), 6.70 (d, J=7.4 Hz, 1H), 6.53 (s, 1H), 3.67-3.60 (m, 1H), 3.23-3.18 (m, 1H), 2.57-2.52 (m, 2H), 1.91 (d, J=10.5 Hz, 2H), 1.78 (d, J=8.8 Hz, 2H), 1.38 (s, 9H), 1.31-1.20 (m, 4H), 1.08 (t, J=7.6 Hz, 3H); ES-LCMS m/z 696.3 [M+H]⁺.

### Step 4: N-[4-[2-[2-[(1r,4r)(4-Aminocyclohexyl)amino]pyrimidin-4-yl]-5-ethylphenoxy]-3-fluorophenyl] 2-chlorobenzenesulfonamide

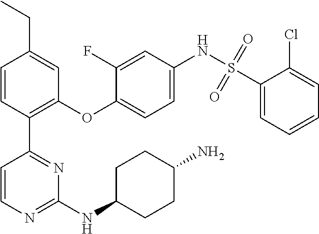 [SMILES: CCc1ccc(-c2ccnc(N[C@H]3CC[C@H](N)CC3)n2)c(Oc2ccc(NS(=O)(=O)c3ccccc3Cl)cc2F)c1]

To a solution of O-t-butyl N-[4-[(1r,4r)[4-[2-[4-[2-chlorophenylsulfonylamido]-2-fluorophenoxy]-4-ethylphenyl]pyrimidin-2-yl]amino]cyclohexyl] carbamate (30 mg, 38.78 μmol, 1 eq) in MeOH (1.0 mL) was added HCl/dioxane (4 M, 2.0 mL, 185.66 eq). The reaction mixture was stirred at 25° C. for 0.5 h. The reaction mixture was neutralized to pH=7 with NH₃·H₂O and extracted with EtOAc (10 mL×3). The combined organic layers were washed with brine (10 mL), dried over Na₂SO₄, filtered and concentrated under reduced pressure to give a residue which was purified by preparative HPLC (column: Welch Ultimate C18 150*25 mm*5 μm; mobile phase: [water (FA)-ACN]; gradient: 20%-50% B over 10 min) followed by lyophilization to yield N-[4-[2-[2-[(1r,4r)(4-aminocyclohexyl)amino]pyrimidin-4-yl]-4-ethylphenoxy]-3-fluorophenyl] 2-chlorobenzenesulfonamide (9.2 mg, 13.97 μmol, 36.0% yield, 97.5% purity, 1FA) as a white solid. ¹H NMR (400 MHz, DMSO-d₆, 75° C.) δ ppm 8.34 (s, 1H HCO₂H), 8.17 (d, J=5.1 Hz, 1H), 7.97 (d, J=7.7 Hz, 1H), 7.72 (d, J=7.7 Hz, 1H), 7.55-7.53 (m, 2H), 7.45 dq, J_{d}=8.0, Hz, J_{q}=4.6 Hz, 1H), 7.07 (dd, J=8.0, 1.6 Hz, 1H), 6.96 (dd, J_{d}=12.9, 2.5 Hz, 1H), 6.93 (d, J=4.8 Hz, 2H), 6.86 (t, J=8.8 Hz, 1H), 6.82 (dd, J=9.0, 2.5 Hz, 1H), 6.54 (s, 1H), 3.72-3.64 (m, 1H), 3.03-2.94 (m, 1H), 2.52 (q, J=7.6 Hz, 2H), 2.04-1.93 (m, 4H), 1.47-1.27 (m, 4H), 1.07 (t, J=7.6 Hz, 3H); ES-LCMS m/z 596.3 [M+H]⁺.
18. N-[4-[2-[2-[(1r,4r)(4-Aminocyclohexyl)amino]pyrimidin-4-yl]-5-phenylphenoxy]-3-fluorophenyl] 2-chlorobenzenesulfonamide. ES-LCMS m/z 644.2, 645.9 [M+H]⁺.

### Step 1: O-t-Butyl N-[4-[(1r,4r)[4-[2-(4-amino-2-fluorophenoxy)-4-phenylphenyl]pyrimidin-2-yl]amino]cyclohexyl] carbamate

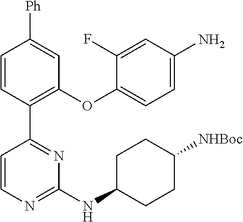 [SMILES: CC(C)(C)OC(=O)N[C@H]1CC[C@H](Nc2nccc(-c3ccc(-c4ccccc4)cc3Oc3ccc(N)cc3F)n2)CC1]

A mixture of O-t-butyl N-[4-[(1r,4r)[4-[2-(4-amino-2-fluorophenoxy)-4-iodophenyl]pyrimidin-2-yl]amino]cyclohexyl] carbamate (100 mg, 156.59 μmol, 1 eq), phenylboronic acid (19.09 mg, 156.59 μmol, 1 eq) and Cs₂CO₃ (153.06 mg, 469.76 μmol, 3 eq) in dioxane (2 mL) and H₂O (0.6 mL) was degassed and purged with N₂ for 3 times, and then Pd(dppf)Cl₂ (11.46 mg, 15.66 μmol, 0.1 eq) was added, the mixture was stirred at 90° C. for 12 h under N₂ atmosphere. The mixture was filtered, and then the filtrate was diluted with H₂O (20 mL) and extracted with EtOAc (20 mL×3). The combined organic layers were washed with brine (20 mL), dried over Na₂SO₄, filtered and concentrated under reduced pressure to yield a residue which was purified by prep-TLC (SiO₂, PE/EtOAc=1/1, TLC: PE/EtOAc=1/1, R_{f}=0.48) to yield O-t-butyl N-[4-[(1r,4r)[4-[2-(4-amino-2-fluorophenoxy)-4-phenylphenyl]pyrimidin-2-yl]amino]cyclohexyl] carbamate (60 mg, 101.11 μmol, 64.5% yield, 96.0% purity) as a yellow solid. ¹H NMR (400 MHz, DMSO-d₆) δ ppm 8.30 (d, J=5.1 Hz, 1H), 7.98 (d, J=8.1 Hz, 1H), 7.54-7.51 (m, 2H), 7.47-7.43 (m, 2H), 7.39-7.35 (m, 1H), 7.15 (d, J=5.1 Hz, 1H), 7.04-6.96 (m, 2H), 6.90 (s, 1H), 6.75-6.68 (m, 1H), 6.52 (dd, J=2.4, 13.3 Hz, 1H), 6.42 (dd, J=2.4, 8.8 Hz, 1H), 5.37 (s, 2H), 3.72 (brs, 1H), 3.17 (brd, J=5.3 Hz, 1H), 2.00-1.80 (m, 4H), 1.38 (s, 9H), 1.32-1.23 (m, 4H); ES-LCMS m/z 570.4 [M+H]⁺.

### Step 2: O-t-Butyl N-[4-[(1r,4r)[4-[2-[4-[2-chlorophenylsulfonylamido]-2-fluoro-phenoxy]-4-phenyl-phenyl]pyrimidin-2-yl]amino]cyclohexyl] carbamate

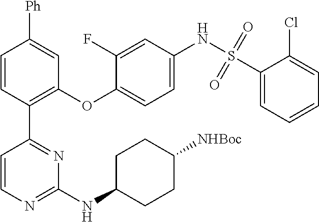 [SMILES: CC(C)(C)OC(=O)N[C@H]1CC[C@H](Nc2nccc(-c3ccc(-c4ccccc4)cc3Oc3ccc(NS(=O)(=O)c4ccccc4Cl)cc3F)n2)CC1]

To a solution of O-t-butyl N-[4-[(1r,4r)[4-[2-(4-amino-2-fluorophenoxy)-4-phenylphenyl]pyrimidin-2-yl]amino]cyclohexyl] carbamate (60 mg, 101.11 μmol, 1 eq) in THF (2 mL) was added pyridine (63.98 mg, 808.89 μmol, 65.29 μL, 8 eq) at 0° C., and then 2-chlorobenzenesulfonyl chloride (64.02 mg, 303.33 μmol, 41.36 μL, 3 eq) was added. The mixture was stirred at 25° C. for 12 h. The reaction mixture was diluted with H₂O (20 mL) and extracted with EtOAc (20 mL×3). The combined organic layers were washed with brine (20 mL), dried over Na₂SO₄, filtered and concentrated under reduced pressure to yield a residue which was purified by prep-TLC (SiO₂, PE/EtOAc=1/1, TLC: PE/EtOAc=1/1, R_{f}=0.47) to yield O-t-butyl N-[4-[(1r,4r)[4-[2-[4-[2-chlorophenylsulfonylamido]-2-fluoro-phenoxy]-4-phenyl-phenyl]pyrimidin-2-yl]amino]cyclohexyl] carbamate (60 mg, 74.17 μmol, 73.3% yield, 92.0% purity) as a yellow solid. ¹H NMR (400 MHz, DMSO-d₆) δ ppm 10.83 (s, 1H), 8.24 (d, J=5.0 Hz, 1H), 8.06-8.01 (m, 1H), 7.97 (d, J=8.0 Hz, 1H), 7.65-7.59 (m, 2H), 7.58-7.50 (m, 4H), 7.45 (d, J=7.8 Hz, 2H), 7.40 (d, J=7.3 Hz, 1H), 7.12-7.02 (m, 3H), 6.99-6.95 (m, 2H), 6.92-6.88 (m, 1H), 6.72 (d, J=8.0 Hz, 1H), 3.69-3.61 (m, 1H), 3.24 (s, 1H), 1.95-1.89 (m, 2H), 1.78 (d, J=9.3 Hz, 2H), 1.38 (s, 9H), 1.30-1.21 (m, 4H); ES-LCMS m/z 744.2 [M+H]⁺.

### Step 3: N-[4-[2-[2-[(1r,4r)(4-Aminocyclohexyl)amino]pyrimidin-4-yl]-5-phenylphenoxy]-3-fluorophenyl] 2-chlorobenzenesulfonamide

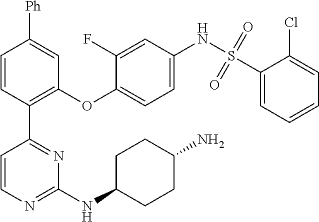 [SMILES: N[C@H]1CC[C@H](Nc2nccc(-c3ccc(-c4ccccc4)cc3Oc3ccc(NS(=O)(=O)c4ccccc4Cl)cc3F)n2)CC1]

To a solution of O-t-butyl N-[4-[(1r,4r)[4-[2-[4-[2-chlorophenylsulfonylamido]-2-fluoro-phenoxy]-4-phenyl-phenyl]pyrimidin-2-yl]amino]cyclohexyl] carbamate (60 mg, 74.17 μmol, 1 eq) in DCM (1 mL) was added HCl/dioxane (4 M, 2 mL, 107.87 eq). The mixture was stirred at 25° C. for 1 h. The reaction mixture was neutralized to pH=7 with NH₃·H₂O, and diluted with H₂O (20 mL) and extracted with EtOAc (20 mL×3). The combined organic layers were washed with brine (20 mL), dried over Na₂SO₄, filtered and concentrated under reduced pressure to yield a residue which was purified by prep-HPLC purification (column: Phenomenex luna C18 150*25 mm*10 μm; mobile phase: [water (FA)-ACN]; gradient: 25%-55% B over 10 min), followed by lyophilization to yield N-[4-[2-[2-[(1r,4r)(4-aminocyclohexyl)amino]pyrimidin-4-yl]-4-phenylphenoxy]-3-fluorophenyl] 2-chlorobenzenesulfonamide (18.1 mg, 27.56 μmol, 37.1% yield, 98.0% purity) as a yellow solid. ¹H NMR (400 MHz, DMSO-d₆) δ ppm 8.28-8.27 (m, 1H), 8.27 (s, 1H), 7.96-7.96 (m, 1H), 7.99-7.93 (m, 1H), 7.54-7.48 (m, 4H), 7.47-7.43 (m, 3H), 7.41-7.36 (m, 2H), 7.10 (s, 1H), 7.06 (d, J=5.0 Hz, 1H), 6.95 (s, 2H), 6.90-6.86 (m, 1H), 6.74-6.69 (m, 1H), 3.72-3.71 (m, 1H), 2.99-2.94 (m, 1H), 2.01-1.92 (m, 4H), 1.44-1.30 (m, 4H); ES-LCMS m/z 644.2 [M+H]⁺.

The following compounds have been made by processes similar to those described immediately above using Method A.
19. N-[4-[2-[2-[(1r,4r)(4-Aminocyclohexyl)amino]pyrimidin-4-yl]-5-cyclopropylphenoxy]-3-fluorophenyl] 2-chlorobenzenesulfonamide. ES-LCMS m/z 608.3, 610.3 [M+H]⁺.
20. N-[4-[2-[2-[(1r,4r)(4-Aminocyclohexyl)amino]pyrimidin-4-yl]-5-(1-methylethyl)phenoxy]-3-fluorophenyl] 2-chlorobenzenesulfonamide. ES-LCMS m/z 610.2, 612.1 [M+H]⁺.
21. N-[4-[2-[2-[(1r,4r)(4-Aminocyclohexyl)amino]pyrimidin-4-yl]-5-ethenylphenoxy]-3-fluorophenyl] 2-chlorobenzenesulfonamide. ES-LCMS m/z 594.3, 596.3 [M+H]⁺.
22. N-[4-[2-[2-[(1r,4r)(4-Aminocyclohexyl)amino]pyrimidin-4-yl]-5-cyclohexylphenoxy]-3-fluorophenyl] 2-chlorobenzenesulfonamide. ES-LCMS m/z 650.2, 652.0 [M+H]⁺.
23. N-[4-[2-[2-[(1r,4r)(4-Aminocyclohexyl)amino]pyrimidin-4-yl]naphthyl-3-oxy]-3-fluorophenyl] 2-chlorobenzenesulfonamide. ES-LCMS m/z 618.3, 620.2 [M+H]⁺.

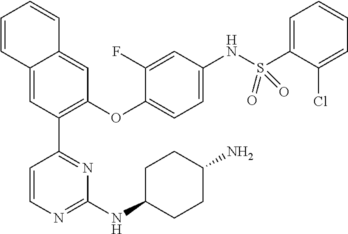 [SMILES: N[C@H]1CC[C@H](Nc2nccc(-c3cc4ccccc4cc3Oc3ccc(NS(=O)(=O)c4ccccc4Cl)cc3F)n2)CC1]

### Step 1: 2-Chloro-4-(3-methoxy-2-naphthyl)pyrimidine

To a solution of (3-methoxy-2-naphthyl)boronic acid (325.44 mg, 1.61 mmol, 1.2 eq) in H₂O (2 mL) and 1,4-dioxane (6 mL) was added Cs₂CO₃ (874.81 mg, 2.68 mmol, 2 eq), 2,4-dichloropyrimidine (200 mg, 1.34 mmol, 1 eq) and Pd(dppf)Cl₂ (49.11 mg, 67.12 μmol, 0.05 eq). The mixture was stirred at 80° C. for 12 h under N₂. The reaction mixture was diluted with H₂O (20 mL) and extracted with EtOAc (20 mL×3). The combined organic layers were washed with brine (10 mL), dried over Na₂SO₄, filtered and concentrated under reduced pressure to yield a residue which was purified by flash silica gel chromatography (PE/EtOAc=1/0 to 3/1, TLC: PE/EtOAc=3/1, R_{f}=0.50) to yield 2-chloro-4-(3-methoxy-2-naphthyl)pyrimidine (260 mg, 950.82 μmol, 70.8% yield, 99.0% purity) as a yellow solid. ¹H NMR (400 MHz, CDCl₃) δ ppm 8.66-8.56 (m, 2H), 8.02 (d, J=5.3 Hz, 1H), 7.92 (d, J=8.1 Hz, 1H), 7.77 (d, J=8.1 Hz, 1H), 7.57-7.50 (m, 1H), 7.44-7.38 (m, 1H), 7.27-7.26 (m, 1H), 4.03 (s, 3H); ES-LCMS m/z 271.2, 273.2 [M+H]⁺.

### Step 2: 3-(2-Chloropyrimidin-4-yl)naphthalen-2-ol

To a solution of 2-chloro-4-(3-methoxy-2-naphthyl)pyrimidine (260 mg, 950.82 μmol, 99.0% purity, 1 eq) in DCM (5 mL) was added BBr₃ (476.41 mg, 1.90 mmol, 183.23 μL, 2 eq). The mixture was stirred at 25° C. for 3 h. The reaction mixture was diluted with H₂O (20 mL) and extracted with DCM (20 mL×3). The combined organic layers were washed with brine (10 mL), dried over Na₂SO₄, filtered and concentrated under reduced pressure to yield 3-(2-chloropyrimidin-4-yl)naphthalen-2-ol (220 mg, 822.79 μmol, 86.5% yield, 96.0% purity) as a yellow solid. ¹H NMR (400 MHz, CDCl₃) δ ppm 11.98 (brs, 1H), 8.75 (d, J=5.5 Hz, 1H), 8.37 (s, 1H), 7.96 (d, J=5.6 Hz, 1H), 7.82 (d, J=8.3 Hz, 1H), 7.71 (d, J=8.4 Hz, 1H), 7.51 (dt, J=1.1, 7.6 Hz, 1H), 7.41 (s, 1H), 7.38-7.31 (m, 1H); ES-LCMS m/z 257.0 [M+H]⁺.

### Step 3: 2-Chloro-4-[3-(2-fluoro-4-nitrophenoxy)-2-naphthyl]pyrimidine

To a solution of 3-(2-chloropyrimidin-4-yl)naphthalen-2-ol (220 mg, 822.79 μmol, 96.0% purity, 1 eq) in DMSO (3 mL) was added K₂CO₃ (284.29 mg, 2.06 mmol, 2.5 eq) and 1,2-difluoro-4-nitrobenzene (130.90 mg, 822.79 μmol, 90.90 μL, 1 eq). The mixture was stirred at 25° C. for 12 h. The reaction mixture was diluted with H₂O (30 mL) and extracted with EtOAc (20 mL×3). The combined organic layers were washed with brine (30 mL), dried over Na₂SO₄, filtered and concentrated under reduced pressure to yield a residue which was purified by flash silica gel chromatography (PE/EtOAc=1/0 to 3/1, TLC: PE/EtOAc=3/1, R_{f}=0.40) to yield 2-chloro-4-[3-(2-fluoro-4-nitrophenoxy)-2-naphthyl]pyrimidine (300 mg, 682.21 μmol, 82.9% yield, 90.0% purity) as a yellow solid. ¹H NMR (400 MHz, DMSO-d₆) δ ppm 8.85 (d, J=5.3 Hz, 1H), 8.66 (s, 1H), 8.38 (dd, J=2.6, 10.6 Hz, 1H), 8.19 (d, J=7.9 Hz, 1H), 8.09-8.03 (m, 2H), 7.96 (d, J=8.1 Hz, 1H), 7.85-7.81 (m, 1H), 7.69-7.60 (m, 2H), 7.33-7.26 (m, 1H); ES-LCMS m/z 396.0 [M+H]⁺.

### Step 4: O-t-Butyl N-[4-[(1r,4r)[4-[3-(2-fluoro-4-nitrophenoxy)-2-naphthyl]pyrimidin-2-yl]amino]cyclohexyl] carbamate

To a solution of 2-chloro-4-[3-(2-fluoro-4-nitrophenoxy)-2-naphthyl]pyrimidine (300 mg, 682.21 μmol, 90.0% purity, 1 eq) in DMSO (3 mL) was added TEA (207.10 mg, 2.05 mmol, 284.87 μL, 3 eq) and tert-butyl N-(4-aminocyclohexyl)carbamate (263.16 mg, 1.23 mmol, 1.8 eq). The mixture was stirred at 110° C. for 12 h. The reaction mixture was diluted with H₂O (30 mL) and extracted with EtOAc (20 mL×3). The combined organic layers were washed with brine (30 mL×3), dried over Na₂SO₄, filtered and concentrated under reduced pressure to yield a residue which was purified by flash silica gel chromatography (PE/EtOAc=1/0 to 1/1, TLC: PE/EtOAc=1/1, R_{f}=0.20) to yield O-t-butyl N-[4-[(1r,4r)[4-[3-(2-fluoro-4-nitrophenoxy)-2-naphthyl]pyrimidin-2-yl]amino]cyclohexyl] carbamate (300 mg, 517.77 μmol, 75.9% yield, 99.0% purity) as a yellow solid. ¹H NMR (400 MHz, DMSO-d₆) δ ppm 8.49-8.39 (m, 1H), 8.37-8.25 (m, 2H), 8.13-8.01 (m, 2H), 7.94 (d, J=7.8 Hz, 1H), 7.79 (s, 1H), 7.64-7.55 (m, 2H), 7.16 (t, J=8.1 Hz, 1H), 7.10-7.01 (m, 1H), 6.91 (d, J=5.0 Hz, 1H), 6.79-6.58 (m, 1H), 3.65-3.49 (m, 1H), 3.21-3.13 (m, 1H), 1.89-1.80 (m, 2H), 1.80-1.71 (m, 2H), 1.37 (s, 9H), 1.29-1.18 (m, 4H); ES-LCMS m/z 574.2 [M+H]⁺.

### Step 5: O-t-Butyl N-[4-[(1r,4r)[4-[3-(4-amino-2-fluorophenoxy)-2-naphthyl]pyrimidin-2-yl]amino]cyclohexyl] carbamate

To a solution of O-t-butyl N-[4-[(1r,4r)[4-[3-(2-fluoro-4-nitrophenoxy)-2-naphthyl]pyrimidin-2-yl]amino]cyclohexyl] carbamate (150 mg, 258.89 μmol, 99% purity, 1 eq) in EtOH (5 mL) and H₂O (1 mL) was added Fe (72.29 mg, 1.29 mmol, 5 eq) and NH₄Cl (138.48 mg, 2.59 mmol, 10 eq). The mixture was stirred at 80° C. for 3 h. After filtration, the organic layer was concentrated under reduced pressure to yield a yellow oil which was purified by flash silica gel chromatography (from PE/EtOAc=1/0 to 0/1, TLC: PE/EtOAc=0/1, R_{f}=0.3) to yield O-t-butyl N-[4-[(1r,4r)[4-[3-(4-amino-2-fluorophenoxy)-2-naphthyl]pyrimidin-2-yl]amino]cyclohexyl] carbamate (110 mg, 192.23 μmol, 74.3% yield, 95.0% purity) as a yellow solid. ¹H NMR (400 MHz, DMSO-d₆) δ ppm 8.34 (s, 1H), 8.33 (d, J=5.1 Hz, 1H), 7.95 (d, J=8.1 Hz, 1H), 7.77 (d, J=8.1 Hz, 1H), 7.50-7.39 (m, 2H), 7.12 (d, J=5.1 Hz, 1H), 7.05 (s, 1H), 7.01 (t, J=9.1 Hz, 1H), 6.74 (d, J=7.8 Hz, 1H), 6.54 (dd, J=2.4, 13.2 Hz, 1H), 6.45 (dd, J=2.1, 8.6 Hz, 1H), 5.41 (s, 2H), 3.73 (s, 1H), 3.27-3.19 (m, 1H), 1.98 (s, 2H), 1.81 (d, J=10.3 Hz, 2H), 1.38 (s, 9H), 1.34-1.22 (m, 4H); ES-LCMS m/z 544.3 [M+H]⁺.

### Step 6: O-t-Butyl N-[4-[(1r,4r)[4-[3-(4-(2-chlorophenylsulfonamido)-2-fluorophenoxy)-2-naphthyl]pyrimidin-2-yl]amino]cyclohexyl] carbamate

To a stirred solution of O-t-butyl N-[4-[(1r,4r)[4-[3-(4-amino-2-fluorophenoxy)-2-naphthyl]pyrimidin-2-yl]amino]cyclohexyl] carbamate (110 mg, 192.23 μmol, 95.0% purity, 1 eq) in THF (2 mL) was added pyridine (121.64 mg, 1.54 mmol, 124.12 μL, 8 eq) and 2-chlorobenzenesulfonyl chloride (121.72 mg, 576.68 μmol, 78.53 μL, 3 eq). The reaction mixture was stirred at 20° C. for 1.5 h. The reaction mixture was diluted with H₂O (5 mL) and extracted with EtOAc (20 mL×3). The combined organic layers were washed with brine (10 mL), dried over Na₂SO₄, filtered and concentrated under reduced pressure to yield a residue which was purified by flash silica gel chromatography (from PE/EtOAc=1/0 to 1/2, TLC: PE/EtOAc=1/2, R_{f}=0.63) to O-t-butyl N-[4-[(1r,4r)[4-[3-(4-(2-chlorophenylsulfonamido)-2-fluorophenoxy)-2-naphthyl]pyrimidin-2-yl]amino]cyclohexyl] carbamate (100 mg, 137.84 μmol, 71.7% yield, 99.0% purity) as a light yellow solid. ¹H NMR (400 MHz, DMSO-d₆) δ ppm 10.89 (s, 1H), 8.35 (s, 1H), 8.27 (d, J=5.0 Hz, 1H), 8.08 (d, J=7.9 Hz, 1H), 7.98 (d, J=7.9 Hz, 1H), 7.78 (d, J=8.0 Hz, 1H), 7.68 (d, J=3.9 Hz, 2H), 7.58-7.43 (m, 3H), 7.18-7.12 (m, 2H), 7.09 (dd, J=2.4, 12.2 Hz, 2H), 6.98 (d, J=5.1 Hz, 1H), 6.95 (dd, J=1.6, 8.8 Hz, 1H), 6.71 (d, J=6.3 Hz, 1H), 3.67 (dd, J=3.6, 7.5 Hz, 1H), 3.21 (d, J=7.1 Hz, 1H), 1.93 (d, J=11.4 Hz, 2H), 1.79 (s, 2H), 1.38 (s, 9H), 1.33-1.23 (m, 4H); ES-LCMS m/z 718.2, 720.2 [M+H]⁺.

### Step 7: N-[4-[(1r,4r)[3-[2-[(4-Aminocyclohexyl)amino]pyrimidin-4-yl]-2-naphthyl]oxy]-3-fluorophenyl] 2-chlorobenzenesulfonamide

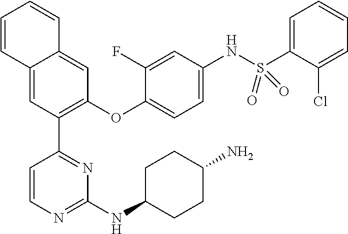 [SMILES: N[C@H]1CC[C@H](Nc2nccc(-c3cc4ccccc4cc3Oc3ccc(NS(=O)(=O)c4ccccc4Cl)cc3F)n2)CC1]

To a stirred solution of O-t-butyl N-[4-[(1r,4r)[4-[3-(4-(2-chlorophenylsulfonamido)-2-fluorophenoxy)-2-naphthyl]pyrimidin-2-yl]amino]cyclohexyl] carbamate (100 mg, 137.84 μmol, 99.0% purity, 1 eq) in DCM (1 mL) was added HCl/dioxane (4 M, 344.6 μL, 10 eq). The reaction mixture was stirred at 25° C. for 2 h. The reaction mixture was diluted with H₂O (50 mL) and extracted with EtOAc (50 mL×3). The combined organic layers were washed with brine (10 mL), dried over Na₂SO₄, filtered and concentrated under reduced pressure to yield a residue. The residue was purified by preparative HPLC (HCl column: Welch Xtimate C18 150*25 mm*5 μm; mobile phase: [water (NH₃H₂O)-ACN]; B %: 20%-50%, 10 min.) and followed by lyophilization to yield N-[4-[(1r,4r) [3-[2-[(4-aminocyclohexyl)amino]pyrimidin-4-yl]-2-naphthyl]oxy]-3-fluorophenyl] 2-chlorobenzenesulfonamide (14.9 mg, 23.86 μmol, 17.3% yield, 99.0% purity) as a white solid. ¹H NMR (400 MHz, DMSO-d₆, 75° C.) δ ppm 8.35 (s, 1H), 8.31 (d, J=4.8 Hz, 1H), 8.01 (d, J=7.5 Hz, 1H), 7.96 (d, J=8.2 Hz, 1H), 7.76 (d, J=8.1 Hz, 1H), 7.53-7.34 (m, 5H), 7.15 (s, 1H), 7.08 (d, J=4.8 Hz, 1H), 6.94-6.85 (m, 2H), 6.80 (brs, 1H), 6.73 (d, J=9.0 Hz, 1H), 3.76-3.67 (m, 1H), 2.93-2.83 (m, 1H), 2.08-1.88 (m, 4H), 1.44-1.24 (m, 4H); ES-LCMS m/z 618.3, 620.2 [M+H]⁺.

### Example 3. Syntheses of Compounds 24-107

The following compounds were made using Method B.
24. N-[4-[[3-[2-(1r,4r)[(4-aminocyclohexyl)amino]pyrimidin-4-yl]-4-pyridyl]oxy]-3-fluoro-phenyl]-2-chloro-benzenesulfonamide

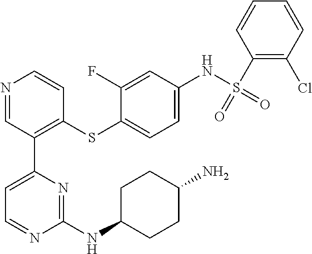 [SMILES: N[C@H]1CC[C@H](Nc2nccc(-c3cnccc3Sc3ccc(NS(=O)(=O)c4ccccc4Cl)cc3F)n2)CC1]

### Step 1: 4-Fluoropyridine-3-boronic Acid

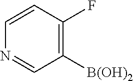

To a solution of 3-bromo-4-fluoropyridine (5.00 g, 28.41 mmol, 1 eq) in THF (75 mL) was added n-BuLi (2.5 M, 22.73 mL, 2 eq) at −78° C. under N₂ dropwise. After stirring at −78° C. for 30 min, triisopropyl borate (6.95 g, 36.93 mmol, 8.49 mL, 1.3 eq) was added dropwise below −65° C. After addition, the mixture was warmed to 20° C., and then was stirred for another 30 min. The reaction mixture was cooled to 0° C., quenched by addition of aq. HCl (1M, 50 mL), and rinsed with EtOAc (30 mL). The aqueous phase was used into the next step without further purification. ES-LCMS Rₜ=0.25, m/z 142.1 [M+H]⁺.

### Step 2: 2-Chloro-4-(4-fluoro-3-pyridyl)pyrimidine

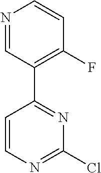 [SMILES: Fc1ccncc1-c1ccnc(Cl)n1]

To a solution of 2,4-dichloropyrimidine (4.23 g, 28.39 mmol, 1 eq) and 4-fluoropyridine-3-boronic acid (4 g, 28.39 mmol, 1 eq) and Cs₂CO₃ (27.75 g, 85.16 mmol, 3 eq) in 1,4-dioxane (400 mL) and H₂O (100 mL) was added Pd(dppf)Cl₂ (1.04 g, 1.42 mmol, 0.05 eq) under N₂. The mixture was stirred at 90° C. for 12 h under N₂. The reaction mixture was quenched by addition of water (100 mL), and extracted with EtOAc (80 mL×3). The combined organic layers were washed with brine (80 mL), dried over Na₂SO₄, filtered and concentrated under reduced pressure to yield a residue which was purified by flash silica gel chromatography (from PE/EtOAc=1/0 to 3/1, TLC: PE/EtOAc=3/1, R_{f}=0.20) to yield 2-chloro-4-(4-fluoropyrid-3-yl)pyrimidine (1.0 g, 4.53 mmol, 16.0% yield, 95.0% purity) as a yellow solid. ¹H NMR (500 MHz, DMSO-d₆) δ ppm 9.14 (d, J=10.5 Hz, 1H), 8.93 (d, J=5.0 Hz, 1H), 8.80 (dd, J=5.5, 7.7 Hz, 1H), 8.01 (dd, J=1.5, 5.2 Hz, 1H), 7.59 (dd, J=5.5, 11.5 Hz, 1H); ES-LCMS m/z 210.1, 212.1 [M+H]⁺.

### Step 3: 4-[3-(2-Chloropyrimidin-4-yl)pyrid-4-yloxy]-3-fluoro-aniline

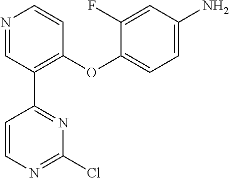 [SMILES: Nc1ccc(Oc2ccncc2-c2ccnc(Cl)n2)c(F)c1]

To a solution of 4-amino-2-fluoro-phenol (495.5 mg, 3.90 mmol, 1 eq) in THF (42 mL) was added t-BuOK (481.1 mg, 4.29 mmol, 1.1 eq) at 0° C. under N₂. After stirred at 0° C. for 30 min, 2-chloro-4-(4-fluoropyrid-3-yl)pyrimidine (860 mg, 3.90 mmol, 95.0%, 1 eq) was added and the mixture was stirred at 70° C. for 12 h. The reaction mixture was quenched by addition of water (50 mL), and extracted with EtOAc (30 mL×3). The combined organic layers were washed with brine (10 mL), dried over Na₂SO₄, filtered and concentrated under reduced pressure to yield a residue, which was purified by flash silica gel chromatography (from PE/EtOAc=1/0 to 3/1, TLC: PE/EtOAc=3/1, R_{f}=0.35) to yield 4-[3-(2-chloropyrimidin-4-yl)pyrid-4-yloxy]-3-fluoro-aniline (650 mg, 2.03 mmol, 52.1% yield, 98.7% purity) as a brown oil. ¹H NMR (400 MHz, CDCl₃) δ ppm 9.24 (s, 1H), 8.67 (d, J=5.2 Hz, 1H), 8.51 (d, J=5.8 Hz, 1H), 8.06 (d, J=5.2 Hz, 1H), 6.96 (t, J=8.8 Hz, 1H), 6.69 (dd, J=1.0, 5.8 Hz, 1H), 6.56 (dd, J=2.8, 11.8 Hz, 1H), 6.50 (ddd, J=1.2, 2.6, 8.6 Hz, 1H), 3.8-3.9 (brs, 2H); ES-LCMS m/z 317.1, 319.2 [M+H]⁺.

### Step 4: O-tert-Butyl N-[4-(1r,4r)-[[4-[4-(4-amino-2-fluoro-phenoxy)-3-pyridyl]pyrimidin-2-yl]amino]cyclohexyl] carbamate

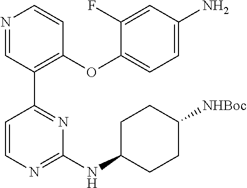 [SMILES: CC(C)(C)OC(=O)N[C@H]1CC[C@H](Nc2nccc(-c3cnccc3Oc3ccc(N)cc3F)n2)CC1]

To a solution of 4-[3-(2-chloropyrimidin-4-yl)pyrid-4-yloxy]-3-fluoro-aniline (300 mg, 0.95 mmol, 1 eq) in DMSO (5 mL) was added TEA (287.5 mg, 2.84 mmol, 395.5 μL, 3 eq) and tert-butyl N-(4-((1r,4r)aminocyclohexyl)carbamate (203 mg, 0.95 mmol, 1 eq). The mixture was stirred at 80° C. for 12 h. The mixture was cooled to room temperature, diluted with water (50 mL) and extracted with EtOAc (30 mL×3). The combined organic layers were washed with brine (20 mL), dried over Na₂SO₄, filtered and concentrated under reduced pressure to yield a residue which was purified by flash silica gel chromatography (from PE/EtOAc=1/0 to 4/1, TLC: PE/EtOAc=0/1, R_{f}=0.38) to yield O-tert-butyl N-[4-(1r,4r)-[[4-[4-(4-amino-2-fluoro-phenoxy)-3-pyridyl]pyrimidin-2-yl]amino]cyclohexyl] carbamate (280 mg, 566 μmol, 59.8% yield, 100.0% purity) as brown oil. ¹H NMR (400 MHz, CDCl₃) δ ppm 9.11 (slbrs, 1H), 8.43 (d, J=5.8 Hz, 1H), 8.32 (d, J=5.2 Hz, 1H), 7.27-7.25 (m, 1H), 6.95 (t, J=8.6 Hz, 1H), 6.65 (d, J=5.8 Hz, 1H), 6.55 (dd, J=2.6, 11.8 Hz, 1H), 6.48 (dd, J=1.8, 8.6 Hz, 1H), 5.10 (d, J=7.0 Hz, 1H), 4.42 (s, 1H), 3.93-3.76 (m, 3H), 3.50 (s, 1H), 2.22 (d, J=10.8 Hz, 2H), 2.05 (s, 4H), 1.46 (s, 9H), 1.33 (d, J=11.4 Hz, 2H); ES-LCMS m/z 495.3 [M+H]⁺.

### Step 5: O-tert-Butyl N-[4-(1r,4r)[[4-[4-[4-[(2-chlorophenyl)sulfonylamino]-2-fluorophenoxy]-3-pyridyl]pyrimidin-2-yl]amino]cyclohexyl] carbamate

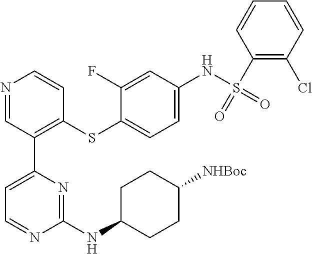 [SMILES: CC(C)(C)OC(=O)N[C@H]1CC[C@H](Nc2nccc(-c3cnccc3Sc3ccc(NS(=O)(=O)c4ccccc4Cl)cc3F)n2)CC1]

To a solution of O-tert-butyl N-[4,E-[[4-[4-(4-amino-2-fluoro-phenoxy)-3-pyridyl]pyrimidin-2-yl]amino]cyclohexyl] carbamate (90 mg, 182 μmol, 100.0%, 1 eq) in THF (2 mL) was added pyridine (115.16 mg, 1.46 mmol, 117.5 μL, 8 eq) and 2-chlorobenzenesulfonyl chloride (38.41 mg, 182 μmol, 24.78 μL, 1 eq). The mixture was stirred at 60° C. for 4 h. The mixture was diluted with water (30 mL) and extracted with EtOAc (20 mL×3). The combined organic layers were washed with brine (10 mL), dried over Na₂SO₄, filtered and concentrated under reduced pressure to yield a residue which was purified by preparative TLC (PE/EtOAc=1/1, TLC: PE/EtOAc=1/1, R_{f}=0.24) to yield O-tert-butyl N-[4,E-[[4-[4-[4-[(2-chlorophenyl)sulfonylamino]-2-fluorophenoxy]-3-pyridyl]pyrimidin-2-yl]amino]cyclohexyl]carbamate (90 mg, 130 μmol, 71.7% yield, 97.0% purity) as a yellow solid. ¹H NMR (400 MHz, CD₃OD) δ ppm 8.97 (s, 1H), 8.55-8.52 (m, 1H), 8.38 (d, J=5.8 Hz, 1H), 8.26 (d, J=5.2 Hz, 1H), 8.13 (d, J=7.8 Hz, 1H), 7.89-7.83 (m, 1H), 7.62-7.57 (m, 2H), 7.52-7.47 (m, 1H), 7.46-7.42 (m, 1H), 7.21-7.13 (m, 3H), 7.05 (d, J=8.8 Hz, 1H), 6.65 (slbrd, J=6.0 Hz, 1H), 3.80 (brs, 1H), 3.35 (brs, 1H), 2.15-2.05 (brd, J=10.8 Hz, 2H), 1.98-1.90 (brd, J=10.4 Hz, 2H), 1.44 (s, 9H), 1.38-1.29 (m, 4H); ES-LCMS m/z 669.1, 671.1 [M+H]⁺.

### Step 6: N-[4-[3-[2-(1r,4r)[(4-aminocyclohexyl)amino]pyrimidin-4-yl]-4-pyrid-4-yloxy]-3-fluoro-phenyl] 2-chlorobenzenesulfonamide

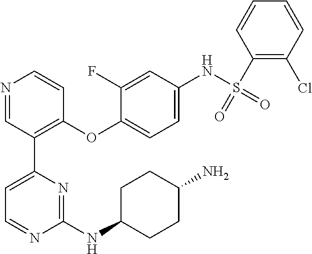 [SMILES: N[C@H]1CC[C@H](Nc2nccc(-c3cnccc3Oc3ccc(NS(=O)(=O)c4ccccc4Cl)cc3F)n2)CC1]

To a solution of tert-butyl N-[4,E-[[4-[4-[4-[(2-chlorophenyl)sulfonylamino]-2-fluoro-phenoxy]-3-pyridyl]pyrimidin-2-yl]amino]cyclohexyl]carbamate (90 mg, 130.5 μmol, 97.0%, 1 eq) in DCM (1 mL) was added HCl/1,4-dioxane (4 M, 0.5 mL, 15.33 eq). The mixture was stirred at 25° C. for 1 h. The mixture was concentrated under reduce pressure, and the residue was dissolved in DMF, adjusted pH to 7 with NH₃·H₂O slowly, then was purified by preparative HPLC (column: C18-1 150*30 mm*5 μm; mobile phase: [water (NH₃·H₂O+NH₄HCO₃)-ACN]; B %: 18%-48%, 15 min), followed by lyophilization to yield N-[4-[3-[2-(1r,4r)[(4-aminocyclohexyl)amino]pyrimidin-4-yl]-pyrid-4-yloxy]-3-fluoro-phenyl] 2-chlorobenzenesulfonamide (43.28 mg, 76 μmol, 58.3% yield, 100.0% purity) as a white solid. ¹H NMR (400 MHz, DMSO-d₆) δ ppm 8.89 (brs, 1H), 8.41 (d, J=5.8 Hz, 1H), 8.32 (d, J=5.2 Hz, 1H), 7.95 (dd, J=2.2, 7.2 Hz, 1H), 7.46-7.41 (m, 1H), 7.40-7.32 (m, 2H), 7.24 (brs, 1H), 7.09 (d, J=5.0 Hz, 1H), 6.94 (t, J=9.4 Hz, 1H), 6.76 (dd, J=2.4, 14.3 Hz, 1H), 6.68-6.58 (m, 2H), 3.71 (m, 1H), 3.05-2.85 (m, 1H), 2.05-1.91 (m, 4H), 1.44-1.28 (m, 4H); ES-LCMS m/z 569.2, 571.1 [M+H]⁺.
25. N-[2-Fluoro-4-[[3-[2-[[(3S)-3-piperidyl]amino]pyrimidin-4-yl]-4-pyridyl]oxy]phenyl] 2-chlorobenzenesulfonamide

### 4-[[3-(2-Chloropyrimidin-4-yl)-4-pyridyl]oxy]-2-fluoro-aniline

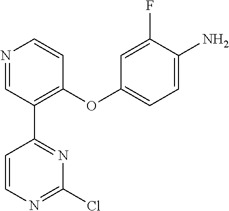 [SMILES: Nc1ccc(Oc2ccncc2-c2ccnc(Cl)n2)cc1F]

To a stirred solution of 2-chloro-4-(4-fluoro-3-pyridyl)pyrimidine (500 mg, 2.36 mmol, 99.0% purity, 1 eq) and 4-amino-3-fluoro-phenol (300.19 mg, 2.36 mmol, 1 eq) in DMSO (8 mL) was added K₂CO₃ (979.14 mg, 7.08 mmol, 3 eq). The reaction mixture was stirred at 25° C. for 1 h. To the mixture was added water (80 mL) and extracted with EtOAc (50 mL×3). The combined organic layers were washed with brine (50 mL), dried over Na₂SO₄, filtered and concentrated under reduced pressure to give a residue which was purified by preparative TLC (PE/EtOAc=0/1, TLC: PE/EtOAc=0/1, R_{f}=0.68) to yield 4-[[3-(2-chloropyrimidin-4-yl)-4-pyridyl]oxy]-2-fluoro-aniline (480 mg, 1.44 mmol, 60.9% yield, 95.0% purity) as a colorless oil. ¹H NMR (500 MHz, DMSO-d₆) δ ppm 8.96 (s, 1H), 8.86 (d, J=5.34 Hz, 1H), 8.51 (d, J=5.80 Hz, 1H), 8.17 (d, J=5.19 Hz, 1H), 7.14-7.10 (m, 1H), 6.86-6.84 (m, 1H), 6.77 (d, J=5.95 Hz, 2H), 5.27-5.21 (m, 2H); ES-LCMS m/z 316.8 [M+H]⁺.

### O-tert-Butyl (3S)-3-[[4-[4-(4-amino-3-fluoro-phenoxy)-3-pyridyl]pyrimidin-2-yl]amino]piperidine-1-carboxylate

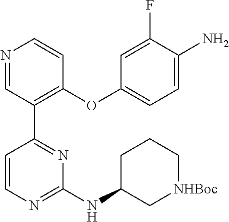

To a stirred solution of 4-[[3-(2-chloropyrimidin-4-yl)-4-pyridyl]oxy]-2-fluoro-aniline (120 mg, 359.9 μmol, 95.0% purity, 1 eq) and tert-butyl (3S)-3-aminopiperidine-1-carboxylate (72.1 mg, 359.9 μmol, 1 eq) in DMSO (6 mL) was added K₂CO₃ (149.2 mg, 1.08 mmol, 3 eq). The reaction mixture was stirred at 120° C. for 2 h. To the mixture was added water (80 mL) and extracted with EtOAc (50 mL×3). The combined organic layers were washed with brine (50 mL), dried over Na₂SO₄, filtered and concentrated under reduced pressure to give a residue which was purified by preparative TLC (PE/EtOAc=0/1, TLC: PE/EtOAc=0/1, R_{f}=0.60) to yield O-tert-butyl (3S)-3-[[4-[4-(4-amino-3-fluoro-phenoxy)-3-pyridyl]pyrimidin-2-yl]amino]piperidine-1-carboxylate (40 mg, 78.25 μmol, 21.7% yield, 94.0% purity) as a colorless oil. ¹H NMR (400 MHz, DMSO-d₆) (ppm 8.98 (brs, 1H), 8.43 (d, J=5.7 Hz, 1H), 8.37 (d, J=5.0 Hz, 1H), 7.27-7.19 (m, 2H), 7.03 (d, J=10.9 Hz, 1H), 6.82 (td, J=8.8, 17.5 Hz, 2H), 6.70 (d, J=5.8 Hz, 1H), 5.23-5.18 (m, 2H), 3.80 (s, 1H), 3.62 (s, 1H), 3.31 (s, 1H), 3.12-2.76 (m, 2H), 1.91 (s, 1H), 1.75 (s, 1H), 1.57 (brs, 2H), 1.38-1.23 (m, 9H); ES-LCMS m/z 481.3 [M+H]⁺.

### O-tert-Butyl N-(3S)-3-[[4-[4-[4-[(2-chlorophenyl)sulfonylamino]-3-fluoro-phenoxy]-3-pyridyl]pyrimidin-2-yl]amino]piperidine-1-carboxylate

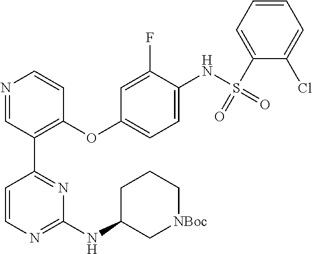 [SMILES: CC(C)(C)OC(=O)N1CCC[C@H](Nc2nccc(-c3cnccc3Oc3ccc(NS(=O)(=O)c4ccccc4Cl)c(F)c3)n2)C1]

To a stirred solution of tert-butyl (3S)-3-[[4-[4-(4-amino-3-fluoro-phenoxy)-3-pyridyl]pyrimidin-2-yl]amino]piperidine-1-carboxylate (40 mg, 74.92 μmol, 90.0% purity, 1 eq) in THF (3 mL) were added pyridine (490.00 mg, 6.19 mmol, 0.5 mL, 82.69 eq), DMAP (4.58 mg, 37.46 μmol, 0.5 eq) and 2-chlorobenzenesulfonyl chloride (63.25 mg, 299.67 μmol, 40.81 μL, 4 eq). The reaction mixture was stirred at 25° C. for 3 h under N₂ atmosphere. To the mixture was added water (80 mL) and extracted with EtOAc (50 mL×3). The combined organic layers were washed with brine (50 mL), dried over Na₂SO₄, filtered and concentrated under reduced pressure to give a residue which was purified by preparative HPLC (column: Boston Green ODS 150*30 mm*5 μm; mobile phase: [water (FA)-ACN]; B %: 40%-70%, 14 min), followed by lyophilization to yield O-tert-butyl N-(3S)-3-[[4-[4-[4-[(2-chlorophenyl)sulfonylamino]-3-fluoro-phenoxy]-3-pyridyl]pyrimidin-2-yl]amino]piperidine-1-carboxylate (20 mg, 29.92 μmol, 39.9% yield, 98.0% purity) as a white solid. ¹H NMR (500 MHz, MeOD-d₄) δ ppm 9.02 (s, 1H), 8.45 (d, J=5.95 Hz, 1H), 8.30 (d, J=5.19 Hz, 1H), 7.99-7.95 (m, 1H), 7.62-7.55 (m, 2H), 7.48-7.41 (m, 2H), 7.16 (d, J=5.19 Hz, 1H), 6.96 (d, J=9.00 Hz, 1H), 6.91-6.83 (m, 2H), 3.92 (s, 1H), 3.81-3.52 (m, 1H), 3.29-3.16 (m, 2H), 3.11-2.87 (m, 1H), 2.03 (s, 1H), 1.79 (s, 1H), 1.71-1.51 (m, 2H), 1.46-1.26 (m, 9H); ES-LCMS m/z 655.3 [M+H]⁺.

### N-[2-Fluoro-4-[[3-[2-[[(3S)-3-piperidyl]amino]pyrimidin-4-yl]-4-pyridyl]oxy]phenyl] 2-chlorobenzenesulfonamide

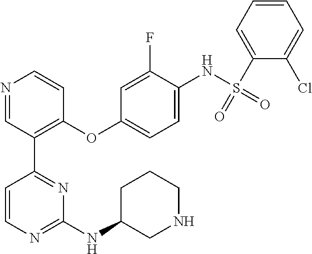 [SMILES: O=S(=O)(Nc1ccc(Oc2ccncc2-c2ccnc(N[C@H]3CCCNC3)n2)cc1F)c1ccccc1Cl]

To a stirred solution of O-tert-butyl N-(3S)-3-[[4-[4-[4-[(2-chlorophenyl)sulfonylamino]-3-fluoro-phenoxy]-3-pyridyl]pyrimidin-2-yl]amino]piperidine-1-carboxylate (40 mg, 50.1 μmol, 82.0% purity, 1 eq) in DCM (1 mL) was added TFA (462.00 mg, 4.05 mmol, 0.3 mL, 81 eq). The reaction mixture was stirred at 25° C. for 0.5 h. The reaction mixture was neutralized to pH=7 with NH₃·H₂O at 0° C. and concentrated to yield a residue which was purified by preparative HPLC (column: Boston Prime C18 150*30 mm*5 μm; mobile phase: [water (NH₃H₂O+NH₄HCO₃)-ACN]; B %: 20%-50%, 10 min), followed by lyophilization to yield N-[2-fluoro-4-[[3-[2-[[(3S)-3-piperidyl]amino]pyrimidin-4-yl]-4-pyridyl]oxy]phenyl] 2-chlorobenzenesulfonamide (11 mg, 19.82 μmol, 39.6% yield, 100.0% purity) as a white solid.

¹H NMR (400 MHz, MeOD-d₄) δ ppm 8.98 (s, 1H), 8.41 (d, J=6.0 Hz, 1H), 8.30 (d, J=5.2 Hz, 1H), 8.00 (dd, J=1.4, 7.9 Hz, 1H), 7.56-7.47 (m, 2H), 7.40-7.32 (m, 2H), 7.19 (d, J=5.3 Hz, 1H), 6.88 (dd, J=2.7, 10.9 Hz, 1H), 6.82 (d, J=5.8 Hz, 1H), 6.78 (td, J=1.3, 8.8 Hz, 1H), 4.12-4.05 (m, 1H), 3.37 (dd, J=4.1, 12.8 Hz, 1H), 3.12-3.06 (m, 1H), 2.81-2.68 (m, 2H), 2.09 (d, J=12.2 Hz, 1H), 1.93-1.87 (m, 1H), 1.77-1.57 (m, 2H); ES-LCMS m/z 555.2 [M+H]⁺.
26. N-[4-[[3-[2-[(1r,4r)(4-Aminocyclohexyl)amino]pyrimidin-4-yl]-4-pyridyl]oxy]-3-fluoro-phenyl] 2,6-difluoro-benzenesulfonamide

### Step 1. O-tert-Butyl N-[4-(1r,4r)[[4-[4-[4-[(2,6-difluorophenyl)sulfonylamino]-2-fluoro-phenoxy]-3-pyridyl]pyrimidin-2-yl]amino]cyclohexyl] carbamate

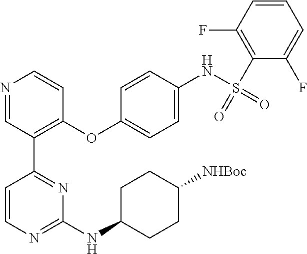 [SMILES: CC(C)(C)OC(=O)N[C@H]1CC[C@H](Nc2nccc(-c3cnccc3Oc3ccc(NS(=O)(=O)c4c(F)cccc4F)cc3)n2)CC1]

To a solution of O-tert-butyl N-[4-(1r,4r)-[[4-[4-(4-amino-2-fluoro-phenoxy)-3-pyridyl]pyrimidin-2-yl]amino]cyclohexyl] carbamate (40 mg, 80.07 μmol, 99.0%, 1 eq) in THF (1 mL) was added pyridine (31.67 mg, 400.4 μmol, 32.3 μL, 5 eq) and 2,6-difluorobenzenesulfonyl chloride (34.05 mg, 160.1 μmol, 21.7 μL, 2 eq). The mixture was stirred at 60° C. for 12 h. The mixture was diluted with water (20 mL) and extracted with EtOAc (15 mL×3). The combined organic layers were washed with brine (20 mL), dried over Na₂SO₄, filtered and concentrated under reduced pressure to yield O-tert-butyl N-[4-(1r,4r)[[4-[4-[4-[(2,6-difluorophenyl)sulfonylamino]-2-fluoro-phenoxy]-3-pyridyl]pyrimidin-2-yl]amino]cyclohexyl] carbamate (30 mg, 44.7 μmol, 55.9% yield, N/A purity) as a yellow oil which was used in the next step without further purification; ES-LCMS m/z 671.2 [M+H]⁺.

### Step 2. N-[4-[3-[2-(1r,4r)[(4-Aminocyclohexyl)amino]pyrimidin-4-yl]pyrid-4-yloxy]-3-fluoro-phenyl] 2,6-difluorobenzenesulfonamide

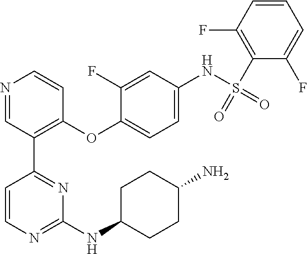 [SMILES: N[C@H]1CC[C@H](Nc2nccc(-c3cnccc3Oc3ccc(NS(=O)(=O)c4c(F)cccc4F)cc3F)n2)CC1]

To a solution of O-tert-butyl N-[4-(1r,4r)[[4-[4-[4-[(2,6-difluorophenyl)sulfonylamino]-2-fluoro-phenoxy]-3-pyridyl]pyrimidin-2-yl]amino]cyclohexyl] carbamate (30 mg, 44.73 μmol, N/A purity, 1 eq) in DCM (1 mL) was added HCl/dioxane (4 M, 0.5 mL, 44.71 eq). The mixture was stirred at 25° C. for 1 h. The reaction mixture was neutralized to pH=7, and concentrated to yield a residue which was purified by preparative HPLC (column: Boston Prime C18 150*30 mm*5 μm; mobile phase: [water (NH₃H₂O+NH₄HCO₃)-ACN]; B %: 15%-45%, 10 min) to yield N-[4[[3-[2-(1r,4r)[(4-aminocyclohexyl)amino]pyrimidin-4-yl]pyrid-4-yloxy]-3-fluoro-phenyl] 2,6-difluorobenzenesulfonamide (18.40 mg, 31.4 μmol, 70.2% yield, 97.3% purity) as a white solid. ¹H NMR (500 MHz, DMSO-d₆) δ ppm 8.91 (brs, 1H), 8.41 (d, J=6.0 Hz, 1H), 8.33 (d, J=5.0 Hz, 1H), 7.39 (tt, J=6.0, 8.5 Hz, 1H), 7.31-7.18 (m, 1H), 7.11 (d, J=5.0 Hz, 1H), 7.06-6.94 (m, 3H), 6.85 (dd, J=2.5, 14.5 Hz, 1H), 6.68-6.61 (m, 2H), 3.83-3.65 (brs, 1H), 3.06-2.96 (m, 1H), 2.10-1.92 (m, 4H), 1.47-1.29 (m, 4H); ES-LCMS m/z 571.1 [M+H]⁺.
27. N-(4-((3-(2-((1r,4r)-4-aminocyclohexyl)amino)pyrimidin-4-yl)pyridin-4-yloxy)-3-fluorophenyl) 2-methoxybenzenesulfonamide

### Step 1. tert-Butyl ((1r,4r)-4-((4-(2-fluoro-4-((2-methoxyphenyl)sulfonamido)phenoxy)-[3,4′-bipyridin]-2′-yl)amino)cyclohexyl)carbamate

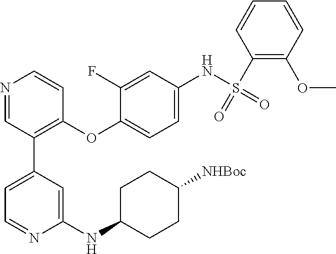 [SMILES: COc1ccccc1S(=O)(=O)Nc1ccc(Oc2ccncc2-c2ccnc(N[C@H]3CC[C@H](NC(=O)OC(C)(C)C)CC3)c2)c(F)c1]

A mixture of O-tert-butyl N-[4-(1r,4r)-[[4-[4-(4-amino-2-fluoro-phenoxy)-3-pyridyl]pyrimidin-2-yl]amino]cyclohexyl] carbamate (40 mg, 76.84 μmol, 95.0% purity, 1 eq), pyridine (48.62 mg, 615 μmol, 49.6 μL, 8 eq), DMAP (4.7 mg, 38.4 μmol, 0.5 eq) in THF (4 mL) was degassed and purged with N₂ for 3 times, then to the mixture was added 2-methoxybenzenesulfonyl chloride (31.76 mg, 153.7 μmol, 2 eq) and stirred at 25° C. for 1 h under N₂ atmosphere. The reaction mixture was quenched by the addition of saturated NaHCO₃ solution (30 mL), and extracted with EtOAc (30 mL×3). The combined organic layers were washed with brine (30 mL), dried over Na₂SO₄, filtered and concentrated to yield tert-butyl ((1r,4r)-4-((4-(2-fluoro-4-((2-methoxyphenyl)sulfonamido)phenoxy)-[3,4′-bipyridin]-2′-yl)amino)cyclohexyl)carbamate (40 mg, crude) as a yellow oil. ES-LCMS m/z 665.4 [M+H]⁺.

### Step 2. N-(4-((3-(2-(((1r,4r)-4-aminocyclohexyl)amino)pyrimidin-4-yl)pyridin-4-yl)oxy)-3-fluorophenyl)-2-methoxybenzenesulfonamide

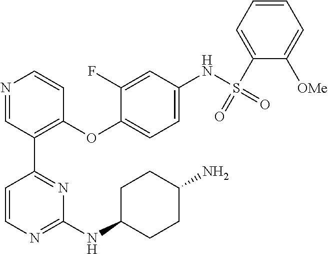 [SMILES: COc1ccccc1S(=O)(=O)Nc1ccc(Oc2ccncc2-c2ccnc(N[C@H]3CC[C@H](N)CC3)n2)c(F)c1]

To a solution of tert-butyl ((1r,4r)-4-((4-(2-fluoro-4-((2-methoxyphenyl)sulfonamido)phenoxy)-[3,4′-bipyridin]-2′-yl)amino)cyclohexyl)carbamate (40 mg, 60.17 μmol, N/A purity, 1 eq) in DCM (3 mL) was added TFA (686 mg, 6.02 mmol, 446 μL, 100 eq). The mixture was stirred at 25° C. for 30 min. The residue was purified by prep-HPLC (column: Boston Prime C18 150*30 mm*5 μm; mobile phase: [water (NH₃H₂O+NH₄HCO₃)-ACN]; B %: 35%-65%, 10 min) to yield N-(4-((3-(2-(((1r,4r)-4-aminocyclohexyl)amino)pyrimidin-4-yl)pyridin-4-yl)oxy)-3-fluorophenyl) 2-methoxybenzenesulfonamide (18.2 mg, 32.2 μmol, 53.5% yield, 100.0% purity) as a white solid. ¹H NMR (500 MHz, DMSO-d6) δ ppm 9.01-8.80 (m, 1H), 8.44 (d, J=5.8 Hz, 1H), 8.31 (d, J=4.9 Hz, 1H), 7.81 (d, J=6.6 Hz, 1H), 7.60 (t, J=7.4 Hz, 1H), 7.31-7.17 (m, 2H), 7.20 (d, J=13.4 Hz, 1H), 7.13-6.91 (m, 2H), 6.97 (dd, J=7.7, 2.3 Hz, 1H), 6.59 (d, J=5.8 Hz, 1H), 3.88 (s, 3H), 3.71 (brs, 1H), 2.96 (s, 1H), 2.07-1.90 (m, 4H), 1.46-1.27 (m, 4H); ES-LCMS m/z 565.2 [M+H]⁺.
28. N-(4-((3-(2-((1r,4r)-4-aminocyclohexyl)amino)pyrimidin-4-yl)pyridin-4-yloxy)-3-fluorophenyl) 5-bromo-2-methoxybenzenesulfonamide

### Step 1. O-tert-butyl ((1r,4r)-4-((4-(4-(4-(5-bromo-2-methoxyphenylsulfonamido)-2-fluorophenoxy)pyridin-3-yl)pyrimidin-2-yl)amino)cyclohexyl Carbamate

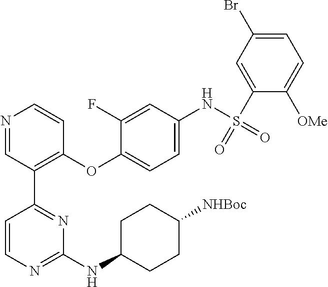 [SMILES: COc1ccc(Br)cc1S(=O)(=O)Nc1ccc(Oc2ccncc2-c2ccnc(N[C@H]3CC[C@H](NC(=O)OC(C)(C)C)CC3)n2)c(F)c1]

A mixture of O-tert-butyl N-[4-(1r,4r)-[[4-[4-(4-amino-2-fluoro-phenoxy)-3-pyridyl]pyrimidin-2-yl]amino]cyclohexyl] carbamate (100 mg, 200.2 μmol, 99.0% purity, 1 eq), 5-bromo-2-methoxy-benzenesulfonyl chloride (114.32 mg, 400.4 μmol, 2 eq), pyridine (126.7 mg, 1.60 mmol, 129.3 μL, 8 eq), DMAP (12.23 mg, 100.1 μmol, 0.5 eq) in THF (3 mL) was degassed and purged with N₂ for 3 times, and then to the mixture was added 5-bromo-2-methoxy-benzenesulfonyl chloride (114.3 mg, 400.4 μmol, 2 eq). The reaction mixture was stirred at 25° C. for 30 min under N₂ atmosphere. The reaction mixture was quenched by the addition of saturated NaHCO₃ solution (30 mL), and extracted with EtOAc (30 mL×3). The combined organic layers were washed with brine (30 mL), dried over Na₂SO₄, filtered and concentrated under reduced pressure to yield O-tert-butyl ((1r,4r)-4-((4-(4-(4-(5-bromo-2-methoxyphenylsulfonamido)-2-fluorophenoxy)pyridin-3-yl)pyrimidin-2-yl)amino)cyclohexyl) carbamate (80 mg, 107.58 μmol, 53.7% yield, N/A purity) as a yellow oil. ES-LCMS m/z 743.3 [M+H]⁺.

### Step 2. N-(4-((3-(2-((1r,4r)-4-aminocyclohexyl)amino)pyrimidin-4-yl)pyridin-4-yloxy)-3-fluorophenyl) 5-bromo-2-methoxybenzenesulfonamide

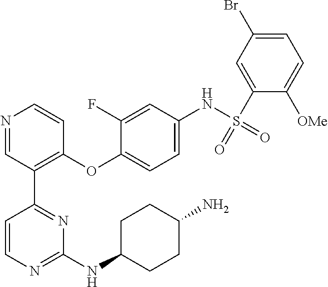 [SMILES: COc1ccc(Br)cc1S(=O)(=O)Nc1ccc(Oc2ccncc2-c2ccnc(N[C@H]3CC[C@H](N)CC3)n2)c(F)c1]

To a solution of O-tert-butyl ((1r,4r)-4-((4-(4-(4-(5-bromo-2-methoxyphenylsulfonamido)-2-fluorophenoxy)pyridin-3-yl)pyrimidin-2-yl)amino)cyclohexyl) carbamate (80 mg, 107.58 μmol, N/A purity, 1 eq) in DCM (3 mL) was added TFA (2.79 g, 24.5 mmol, 1.81 mL, 227.49 eq). The mixture was stirred at 25° C. for 30 min. The residue was purified by prep-HPLC (column: Welch Xtimate C18 150*25 mm*5 μm; mobile phase: [water (NH₄HCO₃)-ACN]; B %: 20%-50%, 11 min) to yield N-(4-((3-(2-((1r,4r)-4-aminocyclohexyl)amino)pyrimidin-4-yl)pyridin-4-yloxy)-3-fluorophenyl) 5-bromo-2-methoxybenzenesulfonamide (57.3 mg, 89 μmol, 82.7% yield, 100.0% purity) as a white solid. ¹H NMR (500 MHz, DMSO-d6) δ ppm 8.91 (brs, 1H), 8.43 (d, J=5.6 Hz, 1H), 8.33 (d, J=5.0 Hz, 1H), 7.80 (d, J=2.6 Hz, 1H), 7.55 (dd, J=2.5, 8.8 Hz, 1H), 7.23 (s, 1H), 7.10 (d, J=5.1 Hz, 1H), 7.06-6.94 (m, 2H), 6.87 (dd, J=2.2, 14.2 Hz, 1H), 6.70-6.62 (m, 2H), 3.75 (s, brs, 3H, 1H), 2.91 (brs, 1H), 2.04-1.89 (m, 4H), 1.40-1.28 (m, 4H); ES-LCMS m/z 645.2 [M+H]⁺.
29. N-[4-[[3-[2-(1r,4r)[(4-Aminocyclohexyl)amino]pyrimidin-4-yl]pyridyl-4-oxy]-3-fluoro-phenyl]thiophene-2-sulfonamide

### Step 1. O-tert-butyl N-[4-(1r,4r)[[4-[4-[2-fluoro-4-(2-thienylsulfonylamino)phenoxy]-3-pyridyl]pyrimidin-2-yl]amino]cyclohexyl] carbamate

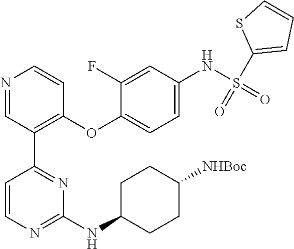 [SMILES: CC(C)(C)OC(=O)N[C@H]1CC[C@H](Nc2nccc(-c3cnccc3Oc3ccc(NS(=O)(=O)c4cccs4)cc3F)n2)CC1]

To a solution of O-tert-butyl N-[4-(1r,4r)-[[4-[4-(4-amino-2-fluoro-phenoxy)-3-pyridyl]pyrimidin-2-yl]amino]cyclohexyl]carbamate (40 mg, 80.1 μmol, 99.0% purity, 1 eq) in THF (2 mL) was added pyridine (31.7 mg, 400 μmol, 32.3 μL, 5 eq) and thiophene-2-sulfonyl chloride (29.25 mg, 160 μmol, 2 eq). The reaction mixture was stirred at 25° C. for 12 h. The mixture was diluted with water (20 mL) and extracted with EtOAc (15 mL×3). The combined organic layers were washed with brine (20 mL), dried over Na₂SO₄, filtered and concentrated under reduced pressure to yield O-tert-butyl N-[4-(1r,4r)[[4-[4-[2-fluoro-4-(2-thienylsulfonylamino)phenoxy]-3-pyridyl]pyrimidin-2-yl]amino]cyclohexyl]carbamate (30 mg, 46.82 μmol, 58.5% yield, N/A purity) as a white oil which was used in the next step without further purification. ES-LCMS m/z 641.3 [M+H]⁺.

### Step 2. N-[4-[[3-[2-(1r,4r)[(4-Aminocyclohexyl)amino]pyrimidin-4-yl]pyridyl-4-oxy]-3-fluoro-phenyl] thiophene-2-sulfonamide

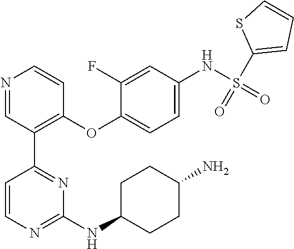 [SMILES: N[C@H]1CC[C@H](Nc2nccc(-c3cnccc3Oc3ccc(NS(=O)(=O)c4cccs4)cc3F)n2)CC1]

To a solution of tert-butyl N-[4-[[4-[4-[2-fluoro-4-(2-thienylsulfonylamino)phenoxy]-3-pyridyl]pyrimidin-2-yl]amino]cyclohexyl]carbamate (30 mg, 46.8 μmol, N/A, 1 eq) in DCM (1 mL) was added HCl/dioxane (4 M, 0.5 mL, 42.7 eq). The mixture was stirred at 25° C. for 1 h. Then the reaction mixture was neutralized to pH=7 and concentrated under vacuum to yield a residue which was purified by preparative HPLC (column: Boston Prime C18 150*30 mm*5 μm; mobile phase: └water (NH₃H₂O+NH₄HCO₃)-ACN┘; B %: 25%-55%, 10 min). N-└4-└└3-└2-(1r,4r)└(4-Aminocyclohexyl)amino]pyrimidin-4-yl]pyridyl-4-oxy]-3-fluoro-phenyl] thiophene-2-sulfonamide (13.50 mg, 24.6 μmol, 52.5% yield, 98.4% purity) was obtained as a white solid. ¹H NMR (500 MHz, DMSO-d₆) δ ppm 8.90 (brs, 1H), 8.42 (d, J=5.5 Hz, 1H), 8.34 (d, J=5.0 Hz, 1H), 7.52 (dd, J=1.0, 5.0 Hz, 1H), 7.25 (dd, brs, J=1.0, 3.5 Hz, 1H, 1H), 7.11 (d, J=5.0 Hz, 1H), 7.00-6.94 cc (m, 2H), 6.84 (dd, J=2.5, 14.5 Hz, 1H), 6.69-6.62 (m, 2H), 3.77-3.67 (m, 1H), 3.01-2.93 (m, 1H), 2.08-1.90 (m, 4H), 1.44-1.29 (m, 4H); ES-LCMS m/z 541.0 [M+H]⁺.
30. N-(4-((3-(2-((1r,4r)-4-aminocyclohexyl)amino)pyrimidin-4-yl)pyridin-4-yloxy)-3-fluorophenyl) 4-cyanobenzenesulfonamide

### Step 1. O-tert-butyl N-((1r,4r)-4-((4-(4-(4-(4-cyanophenylsulfonamido)-2-fluorophenoxy)pyridin-3-yl)pyrimidin-2-yl)amino)cyclohexyl) carbamate

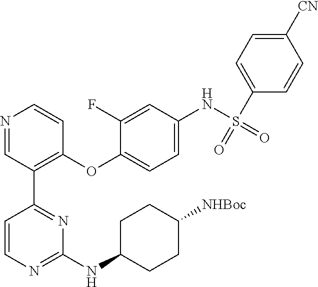 [SMILES: CC(C)(C)OC(=O)N[C@H]1CC[C@H](Nc2nccc(-c3cnccc3Oc3ccc(NS(=O)(=O)c4ccc(C#N)cc4)cc3F)n2)CC1]

A mixture of O-tert-butyl N-[4-(1r,4r)-[[4-[4-(4-amino-2-fluoro-phenoxy)-3-pyridyl]pyrimidin-2-yl]amino]cyclohexyl] carbamate (100 mg, 200 μmol, 99.0% purity, 1 eq), pyridine (126.7 mg, 1.60 mmol, 129.3 μL, 8 eq), DMAP (12.23 mg, 100 μmol, 0.5 eq) in THF (4 mL) was degassed and purged with N₂ for 3 times, then to the mixture was added 4-cyanobenzenesulfonyl chloride (80.7 mg, 400.4 μmol, 2 eq) and stirred at 25° C. for 1 h under N₂ atmosphere. The reaction mixture was quenched by the addition of saturated NaHCO₃ solution (30 mL), and extracted with EtOAc (30 mL×3). The combined organic layers were washed with brine (30 mL), dried over Na₂SO₄, filtered and concentrated to yield O-tert-butyl N-((1r,4r)-4-((4-(4-(4-(4-cyanophenylsulfonamido)-2-fluorophenoxy)pyridin-3-yl)pyrimidin-2-yl)amino)cyclohexyl) carbamate (50 mg, 75.8 μmol, 37.8% yield, N/A purity) as a yellow oil. ES-LCMS m/z 660.3 [M+H]⁺.

### Step 2. N-(4-((3-(2-((1r,4r)-4-aminocyclohexyl)amino)pyrimidin-4-yl)pyridin-4-yloxy)-3-fluorophenyl) 4-cyanobenzenesulfonamide

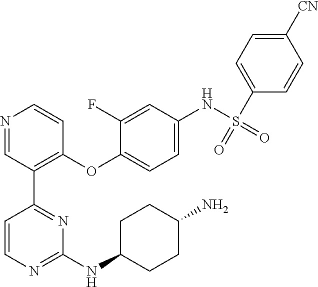 [SMILES: N#Cc1ccc(S(=O)(=O)Nc2ccc(Oc3ccncc3-c3ccnc(N[C@H]4CC[C@H](N)CC4)n3)c(F)c2)cc1]

To a solution of O-tert-butyl N-((1r,4r)-4-((4-(4-(4-(4-cyanophenylsulfonamido)-2-fluorophenoxy)pyridin-3-yl)pyrimidin-2-yl)amino)cyclohexyl) carbamate (50 mg, 75.8 μmol, N/A purity, 1 eq) in DCM (3 mL) was added TFA (864 mg, 7.58 mmol, 561 μL, 100 eq). The mixture was stirred at 25° C. for 30 min. The residue was purified by prep-HPLC (column: Boston Prime C18 150*30 mm*5 μm; mobile phase: [water (NH₃H₂O+NH₄HCO₃)-ACN]; B %: 21%-51%, 10 min) to yield N-(4-((3-(2-((1r,4r)-4-aminocyclohexyl)amino)pyrimidin-4-yl)pyridin-4-yloxy)-3-fluorophenyl) 4-cyanobenzenesulfonamide (38.9 mg, 69.2 μmol, 91.3% yield, 99.5% purity) as a white solid. ¹H NMR (400 MHz, 75° C., DMSO-d6) δ ppm 8.93 (s, 1H), 8.43 (d, J=5.8 Hz, 1H), 8.33 (d, J=5.2 Hz, 1H), 7.88, 7.82 (AA′BB′, J=8 Hz, 2H, 2H), 7.66 (brs, 1H), 7.26 (s, 1H), 711 (d, J=4.7 Hz, 1H), 6.94 (t, J=9.4 Hz, 1H), 6.92-6.84 (m, 2H), 6.74-6.66 (m, 2H), 3.77 (brs, 1H), 3.05-2.93 (m, 1H), 2.05-1.92 (m, 4H), 1.42-1.31 (m, 4H); ES-LCMS m/z 560.1 [M+H]⁺.
31. N-(4-((3-(2-((1r,4r)-4-aminocyclohexyl)amino)pyrimidin-4-yl)pyridin-4-yloxy)-3-fluorophenyl) benzo[b]thiophene-3-sulfonamide

### Step 1. O-tert-butyl N-((1r,4r)-4-((4-(4-(4-(benzo[b]thiophene-3-sulfonamido)-2-fluorophenoxy)pyridin-3-yl)pyrimidin-2-yl)amino)cyclohexyl) carbamate

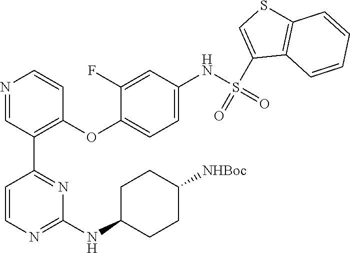 [SMILES: CC(C)(C)OC(=O)N[C@H]1CC[C@H](Nc2nccc(-c3cnccc3Oc3ccc(NS(=O)(=O)c4csc5ccccc45)cc3F)n2)CC1]

A mixture of O-tert-butyl N-[4-(1r,4r)-[[4-[4-(4-amino-2-fluoro-phenoxy)-3-pyridyl]pyrimidin-2-yl]amino]cyclohexyl]carbamate (40.0 mg, 80 μmol, 99.0% purity, 1 eq), pyridine (50.7 mg, 640.6 μmol, 51.70 μL, 8 eq), DMAP (4.89 mg, 40 μmol, 0.5 eq) in THF (3 mL) was degassed and purged with N₂ for 3 times, and then to the mixture was added benzothiophene-3-sulfonyl chloride (37.27 mg, 160.14 μmol, 2 eq). The reaction mixture was stirred at 25° C. for 1 h under N₂ atmosphere. The reaction mixture was quenched by the addition of saturated NaHCO₃ solution (30 mL), extracted with EtOAc (30 mL×3). The combined organic layers were washed with brine (30 mL), dried over Na₂SO₄, filtered and concentrated to yield O-tert-butyl N-((1r,4r)-4-((4-(4-(4-(benzo[b]thiophene-3-sulfonamido)-2-fluorophenoxy)pyridin-3-yl)pyrimidin-2-yl)amino)cyclohexyl) carbamate (30 mg, 37.70 μmol, 47.0% yield, 86.8% purity) as a yellow oil. ES-LCMS m/z 691.1 [M+H]⁺.

### Step 2. N-(4-((3-(2-((1r,4r)-4-aminocyclohexyl)amino)pyrimidin-4-yl)pyridin-4-yloxy)-3-fluorophenyl) benzo[b]thiophene-3-sulfonamide

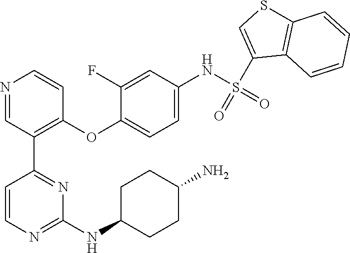 [SMILES: N[C@H]1CC[C@H](Nc2nccc(-c3cnccc3Oc3ccc(NS(=O)(=O)c4csc5ccccc45)cc3F)n2)CC1]

To a solution of O-tert-butyl N-((1r,4r)-4-((4-(4-(4-(benzo[b]thiophene-3-sulfonamido)-2-fluorophenoxy)pyridin-3-yl)pyrimidin-2-yl)amino)cyclohexyl) carbamate (30 mg, 37.70 μmol, 86.8% purity, 1 eq) in DCM (3 mL) was added TFA (430 mg, 0.38 mmol, 279 μL, 100 eq). The mixture was stirred at 25° C. for 30 min. The residue was purified by prep-HPLC (column: Boston Prime C18 150*30 mm*5 μm; mobile phase: [water (NH₃H₂O+NH₄HCO₃)-ACN]; B %: 26%-56%, 10 min) to yield N-(4-((3-(2-((1r,4r)-4-aminocyclohexyl)amino)pyrimidin-4-yl)pyridin-4-yloxy)-3-fluorophenyl) benzo[b]thiophene-3-sulfonamide (20.30 mg, 34.4 μmol, 91.1% yield, 100.0% purity) as a white solid. ¹H NMR (500 MHz, DMSO-d6) δ ppm 8.89 (brs, 1H), 8.39 (d, J=5.8 Hz, 1H), 8.31 d, J=5.2 Hz, H), −8.25 (m, 2H), 8.09 (s, 1H), 7.95 (d, J=7.9 Hz, 1H), 7.53 (brs, 1H), 7.44-7.33 (m, 2H), 7.23 (s, 1H), 7.07 (d, J=5.0 Hz, 1H), 6.95-6.80 (m, 2H), 6.74-6.53 (m, 2H), 3.70 (brd, J=7.5 Hz, 1H), 3.03-2.88 (m, 1H), 2.06-1.88 (m, 4H), 1.44-1.26 (m, 4H); ES-LCMS m/z 591.1 [M+H]⁺.
32. N-(4-((3-(2-(((1r,4r)-4-Aminocyclohexyl)amino)pyrimidin-4-yl)pyridin-4-yl)oxy)-2-(trifluoromethyl)phenyl) benzenesulfonamide

### Step 1: N-(4-Hydroxy-2-(trifluoromethyl)phenyl)benzenesulfonamide

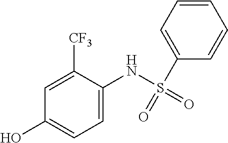 [SMILES: O=S(=O)(Nc1ccc(O)cc1C(F)(F)F)c1ccccc1]

A mixture of 4-amino-3-(trifluoromethyl)phenol (1 g, 5.65 mmol, 1 eq), NaHCO₃ (474.28 mg, 5.65 mmol, 219.58 μL, 1 eq) in THF (3 mL) was degassed and purged with N₂ for 3 times, and then the mixture was added benzenesulfonyl chloride (1.99 g, 11.29 mmol, 1.45 mL, 2 eq). The mixture was stirred at 25° C. for 12 h under N₂ atmosphere. The reaction mixture was quenched by the addition of water (50 ml), extracted with EtOAc (50 mL×3). The combined organic layers were washed with brine (50 mL), dried over Na₂SO_{4,} filtered and concentrated under reduced pressure to yield a residue which was purified by prep-HPLC column: (Boston Green ODS 150*30 mm*5 μm; mobile phase: [water (FA)-ACN]; B %: 42%-72%, 12 min) to yield N-(4-hydroxy-2-(trifluoromethyl)phenyl) benzenesulfonamide (1.2 g, 3.71 mmol, 65.6% yield, 98.0% purity) as a white solid. ¹H NMR (500 MHz, DMSO-d₆) δ ppm 10.31 (s, 1H), 9.62 (s, 1H), 7.78-7.71 (m, 2H), 7.69-7.64 (m, 1H), 7.62-7.56 (m, 2H), 7.02 (d, J=2.7 Hz, 1H), 6.88-6.86 (m, 1H), 6.71 (d, J=8.7 Hz, 1H).

### Step 2: N-(4-((3-(2-Chloropyrimidin-4-yl)pyridin-4-yl)oxy)-2-(trifluoromethyl)phenyl) benzenesulfonamide

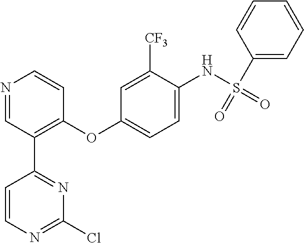 [SMILES: O=S(=O)(Nc1ccc(Oc2ccncc2-c2ccnc(Cl)n2)cc1C(F)(F)F)c1ccccc1]

To a solution of 2-chloro-4-(4-fluoro-3-pyridyl)pyrimidine (250 mg, 1.19 mmol, 1 eq) and N-(4-hydroxy-2-(trifluoromethyl)phenyl)benzenesulfonamide (424.76 mg, 1.31 mmol, 98.0% purity, 1.1 eq) in DMA (0.5 mL) was added K₂CO₃ (247.26 mg, 1.79 mmol, 1.5 eq). The mixture was stirred at 50° C. for 4 h. The reaction mixture was quenched by the addition H₂O (20 mL) and extracted with DCM (30 mL×3). The combined organic layers were washed with brine (30 mL), dried over Na₂SO₄, filtered and concentrated under reduced pressure to yield a residue which was purified by flash silica gel chromatography (PE/EtOAc=1/1, TLC: PE/EtOAc=1/1, R_{f}=0.3) to yield N-(4-((3-(2-chloropyrimidin-4-yl)pyridin-4-yl)oxy)-2-(trifluoromethyl)phenyl) benzenesulfonamide (180 mg, 323.15 μmol, 27.1% yield, 91.0% purity) as a yellow solid. ¹H NMR (400 MHz, DMSO-d₆) δ ppm 10.10 (s, 1H), 9.00 (s, 1H), 8.84 (d, J=5.2 Hz, 1H), 8.59 (d, J=5.8 Hz, 1H), 8.17 (d, J=5.1 Hz, 1H), 7.79 (d, J=7.3 Hz, 2H), 7.74-7.57 (m, 4H), 7.49 (d, J=7.9 Hz, 1H), 7.12 (d, J=8.7 Hz, 1H), 6.88 (d, J=5.7 Hz, 1H); ES-LCMS m/z 506.9 ┌M+H┐⁺.

### Step 3: O-tert-Butyl N-((1r,4r)-4-((4-(4-(4-(phenylsulfonamido)-3-(trifluoromethyl)phenoxy)pyridin-3-yl)pyrimidin-2-yl)amino)cyclohexyl) carbamate

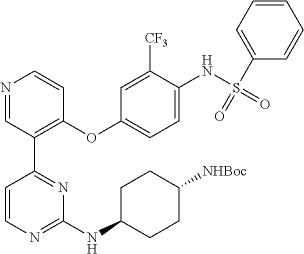 [SMILES: CC(C)(C)OC(=O)N[C@H]1CC[C@H](Nc2nccc(-c3cnccc3Oc3ccc(NS(=O)(=O)c4ccccc4)c(C(F)(F)F)c3)n2)CC1]

To a solution of tert-butyl N-(4-aminocyclohexyl)carbamate (138.50 mg, 646.30 μmol, 2 eq) and N-(4-((3-(2-chloropyrimidin-4-yl)pyridin-4-yl)oxy)-2-(trifluoromethyl)phenyl) benzenesulfonamide (180 mg, 323.15 μmol, 91.0% purity, 1 eq) in DMSO (2 mL) was added K₂CO₃ (133.98 mg, 969.45 μmol, 3 eq). The mixture was stirred at 120° C. for 2 h. The reaction mixture was quenched by the addition H₂O (20 mL) and extracted with EtOAc (30 mL×3). The combined organic layers were washed with brine (30 mL), dried over Na₂SO₄, filtered and concentrated under reduced pressure to yield a residue which was purified by flash silica gel chromatography (PE/EtOAc=1/1, TLC: PE/EtOAc=1/1, R_{f}=0.3) to yield O-tert-butyl N-((1r,4r)-4-((4-(4-(4-(phenylsulfonamido)-3-(trifluoromethyl)phenoxy)pyridin-3-yl)pyrimidin-2-yl)amino)cyclohexyl)carbamate (100 mg, 144.58 μmol, 44.7% yield, 99.0% purity) as a yellow solid. ¹H NMR (400 MHz, CD₃OD-d₄) δ ppm 8.99 (s, 1H), 8.49 (d, J=5.8 Hz, 1H), 8.27 (d, J=5.2 Hz, 1H), 7.83-7.77 (m, 2H), 7.67-7.62 (m, 1H), 7.58-7.49 (m, 3H), 7.44 (d, J=2.9 Hz, 1H), 7.34 (dd, J=2.9, 8.9 Hz, 1H), 7.10 (d, J=5.2 Hz, 1H), 6.91 (d, J=5.8 Hz, 1H), 4.58 (s, 1H), 3.77 (s, 1H), 2.08 (d, J=11.4 Hz, 2H), 1.94 (br d, J=11.8 Hz, 2H), 1.44 (s, 9H), 1.40-1.28 (m, 4H); ES-LCMS m/z 685.2 [M+H]⁺.

### Step 4. N-(4-((3-(2-(((1r,4r)-4-Aminocyclohexyl)amino)pyrimidin-4-yl)pyridin-4-yl)oxy)-2-(trifluoromethyl)phenyl) benzenesulfonamide

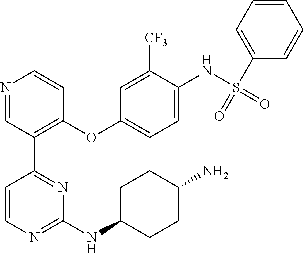 [SMILES: N[C@H]1CC[C@H](Nc2nccc(-c3cnccc3Oc3ccc(NS(=O)(=O)c4ccccc4)c(C(F)(F)F)c3)n2)CC1]

To a solution of O-tert-butyl N-((1r,4r)-4-((4-(4-(4-amino-3-(trifluoromethyl)phenoxy)pyridin-3-yl)pyrimidin-2-yl)amino)cyclohexyl) carbamate (50.51 mg, 73.02 μmol, 99.0% purity, 1 eq) in DCM (3 mL) was added TFA (1.54 g, 13.51 mmol, 1 mL, 184.96 eq). The mixture was stirred at 25° C. for 30 min. The residue was purified by prep-HPLC (column: Boston Prime C18 150*30 mm*5 μm; mobile phase: [water (NH₃H₂O+NH₄HCO₃)-ACN]; B %: 20%-50%, 10 min) to yield N-(4-((3-(2-(((1r,4r)-4-aminocyclohexyl)amino)pyrimidin-4-yl)pyridin-4-yl)oxy)-2-(trifluoromethyl)phenyl) benzenesulfonamide (7.4 mg, 12.53 μmol, 17.16% yield, 99.0% purity) as a white solid. ¹H NMR (500 MHz, DMSO-d₆) δ ppm 8.88 (brs, 1H), 8.40 (d, J=5.5 Hz, 1H), 8.31 (d, J=5.2 Hz, 1H), 7.71 (dd, J=1.9, 7.2 Hz, 4H), 7.39-7.32 (m, 4H), 7.22 (s, 1H), 7.15-7.08 (m, 2H), 6.96 (dd, J=2.7, 9.0 Hz, 1H), 6.66 (s, 1H), 3.68 (brs, 1H), 3.00 (t, J=10.9 Hz, 1H), 2.00 (d, J=11.4 Hz, 2H), 1.95 (d, J=11.3 Hz, 2H), 1.42-1.29 (m, 4H); ES-LCMS m/z 585.2 [M+H]⁺.
33. N-[4-[[3-[2-(1r,4r)[(4-Aminocyclohexyl)amino]pyrimidin-4-yl]-4-pyridyl]oxy]-3-fluoro-phenyl]-2-ethenylbenzenesulfonamide.

### Step 1. O-t-Butyl N-[(1r,4r)-4-[[4-[4-[2-fluoro-4-[(2-iodophenyl)sulfonylamino]phenoxy]-3-pyridyl]pyrimidin-2-yl]amino]cyclohexyl] carbamate

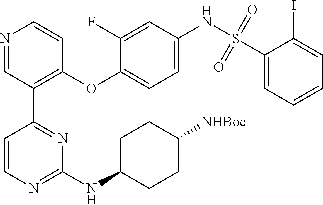 [SMILES: CC(C)(C)OC(=O)N[C@H]1CC[C@H](Nc2nccc(-c3cnccc3Oc3ccc(NS(=O)(=O)c4ccccc4I)cc3F)n2)CC1]

To a stirred solution of O-t-butyl N-[4-[[14-[(1r,4r)-4-(4-amino-2-fluoro-phenoxy)-3-pyridyl]pyrimidin-2-yl]amino]cyclohexyl] carbamate (130 mg, 233.95 μmol, 1 eq) in THF (6 mL) was added pyridine (3.02 g, 38.23 mmol, 3.09 mL, 163.39 eq) and 2-iodobenzenesulfonyl chloride (353.86 mg, 1.17 mmol, 5 eq). The reaction mixture was stirred at 25° C. for 5 h. To the mixture was added water (80 mL) and extracted with EtOAc (50 mL×3). The combined organic layers were washed with brine (50 mL), dried over Na₂SO₄, filtered and concentrated under reduced pressure to give a residue which was purified by flash silica gel chromatography (PE/EtOAc=1/0 to 2/1, TLC: PE/EtOAc=1/1, R_{f}=0.50) to yield O-t-butyl N-[(1r,4r)-4-[[4-[4-[(2-fluoro-4-[(2-iodophenyl)sulfonylamino]phenoxy]-3-pyridyl]pyrimidin-2-yl]amino]cyclohexyl] carbamate (80 mg, 99.92 μmol, 42.7% yield, 95.0% purity) as a white solid. ¹H NMR (400 MHz, DMSO-d₆) δ ppm 11.01 (brs, 1H), 8.92 (brs, 1H), 8.45 (d, J=5.7 Hz, 1H), 8.31 (d, J=4.9 Hz, 1H), 8.16-8.08 (m, 2H), 7.60 (t, J=7.6 Hz, 1H), 7.36-7.26 (m, 2H), 7.16 (s, 1H), 7.10-7.02 (m, 2H), 6.94 (d, J=8.9 Hz, 1H), 6.73 (d, J=7.3 Hz, 1H), 6.66 (d, J=5.6 Hz, 1H), 3.69 (brs, 1H), 3.26-3.18 (m, 1H), 1.93 (s, 2H), 1.79 (d, J=11.8 Hz, 2H), 1.39 (s, 9H), 1.32-1.23 (m, 4H); ES-LCMS m/z 761.3 [M+H]⁺.

### Step 2. O-t-Butyl N-[(1r,4r)-4-[[4-[4-[2-fluoro-4-[(2-ethenylphenyl)sulfonylamino]phenoxy]-3-pyridyl]pyrimidin-2-yl]amino]cyclohexyl] carbamate

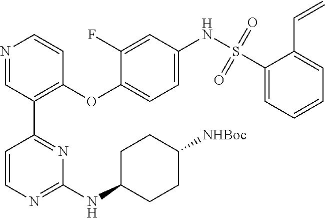 [SMILES: C=Cc1ccccc1S(=O)(=O)Nc1ccc(Oc2ccncc2-c2ccnc(N[C@H]3CC[C@H](NC(=O)OC(C)(C)C)CC3)n2)c(F)c1]

To a stirred solution of O-t-butyl N-[(1r,4r)-4-[[4-[4-[2-fluoro-4-[(2-iodophenyl)sulfonylamino]phenoxy]-3-pyridyl]pyrimidin-2-yl]amino]cyclohexyl] carbamate (40 mg, 49.96 μmol, 1 eq) and 4,4,5,5-tetramethyl-2-vinyl-1,3,2-dioxaborolane (38.47 mg, 249.80 μmol, 42.37 μL, 5 eq), Cs₂CO₃ (32.56 mg, 99.92 μmol, 2 eq) in THF (3 mL) and H₂O (0.6 mL) was added Pd(PPh₃)₄ (11.55 mg, 9.99 μmol, 0.2 eq). The reaction mixture was stirred at 60° C. for 16 h under N₂ atmosphere. To the mixture was added water (80 mL) and extracted with EtOAc (50 mL×3). The combined organic layers were washed with brine (50 mL), dried over Na₂SO₄, filtered and concentrated under reduced pressure to give a residue which was purified by preparative TLC (PE/EtOAc=3/1, TLC: PE/EtOAc=0/1, R_{f}=0.65) to yield O-t-butyl N-[(1r,4r)-4-[[4-[4-[2-fluoro-4-[(2-ethenylphenyl)sulfonylamino]phenoxy]-3-pyridyl]pyrimidin-2-yl]amino]cyclohexyl]carbamate (30 mg, 31.78 μmol, 63.6% yield, 70.0% purity) as a yellow solid. ¹H NMR (400 MHz, CD₃OD-d₄) δ ppm 8.97 (s, 1H), 8.38 (d, J=5.8 Hz, 1H), 8.26 (d, J=5.3 Hz, 1H), 8.00 (dd, J=1.0, 7.9 Hz, 1H), 7.70 (d, J=7.8 Hz, 1H), 7.62-7.60 (m, 1H), 7.54 (d, J=3.0 Hz, 1H), 7.43 (t, J=7.2 Hz, 1H), 7.17-7.09 (m, 2H), 7.03 (dd, J=2.4, 12.1 Hz, 1H), 6.92 (d, J=8.7 Hz, 1H), 6.64 (d, J=5.8 Hz, 1H), 5.76 (dd, J=1.0, 17.3 Hz, 1H), 5.47 (d, J=0.8 Hz, 1H), 3.80 (brs, 1H), 3.39-3.35 (m, 1H), 2.10 (d, J=10.3 Hz, 2H), 1.94 (d, J=10.0 Hz, 2H), 1.44 (s, 9H), 1.39-1.32 (m, 4H); ES-LCMS m/z 661.4 [M+H]⁺.

### Step 3. N-[4-[[3-[2-(1r,4r)[(4-Aminocyclohexyl)amino]pyrimidin-4-yl]-4-pyridyl]oxy]-3-fluoro-phenyl]-2-ethenylbenzenesulfonamide

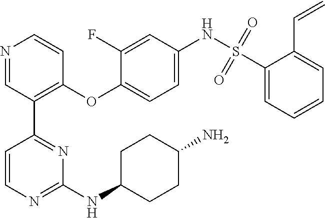 [SMILES: C=Cc1ccccc1S(=O)(=O)Nc1ccc(Oc2ccncc2-c2ccnc(N[C@H]3CC[C@H](N)CC3)n2)c(F)c1]

To a stirred solution of O-t-butyl N-|(1r,4r)-4-∥4-|4-|2-fluoro-4-|(2-ethenylphenyl)sulfonylamino]phenoxy]-3-pyridyl]pyrimidin-2-yl]amino]cyclohexyl] carbamate (30 mg, 31.78 μmol, 1 eq) in DCM (3 mL) was added TFA (767.50 mg, 6.73 mmol, 0.5 mL, 211.79 eq). The reaction mixture was stirred at 25° C. for 0.5 h. The reaction mixture was neutralized to pH=7 with NH₃·H₂O and concentrated to yield a residue which was purified by preparative HPLC (column: Boston Green ODS 150*30 mm*5 μm; mobile phase: [water (FA)-ACN]; gradient: 12%-42%, 12 min), followed by lyophilization to yield N-[4-[[3-[2-(1r,4r)[(4-aminocyclohexyl)amino]pyrimidin-4-yl]-4-pyridyl]oxy]-3-fluoro-phenyl]-2-ethenylbenzenesulfonamide (15.8 mg, 26.04 μmol, 82.0% yield, 100.0% purity, 0.71FA) as a white solid. ¹H NMR (400 MHz, DMSO-d₆, 75° C.) δ ppm 8.93 (s, 1H), 8.43 (d, J=5.7 Hz, 1H), 8.33 (d, J=5.0 Hz, 1H), 8.22 (slbrs, 0.71H, Formate), 7.93 (dd, J=8.1, 1.5 Hz, 1H), 7.77 (dd, J=17.4, 10.8 Hz, 1H), 7.68 (dd, J=8.1, 1.5 Hz, 1H), 7.47 (t, J=8.0 Hz, 1H), 7.38-7.32 (slbrt, J=7.4 Hz, 1H), 7.09 (d, J=5.3 Hz, 1H), 7.01 (t, J=9.0 Hz, 1H), 6.91-6.85 (m, 2H), 6.76 (slbrd, J=8.2 Hz, 1H), 6.66 (d, J=5.9 Hz, 1H), 5.74 (dd, J=17.4, 1.9 Hz, 1H), 5.32 (dd, J=10.8, 1.9 Hz, 1H), 3.80-3.69 (brs, 1H), 2.98-2.88 (brs, 1H), 2.10-1.92 (m, 4H), 1.46-1.31 (m, 4H); ES-LCMS 561.4 m/z [M+H]⁺.

The following compounds have been made by processes similar to those described immediately above using Method B.
34. N-[3-Fluoro-4-[[3-[2-[[(3S)-3-piperidyl]amino]pyrimidin-4-yl]-4-pyridyl]oxy]phenyl] 2-chlorobenzenesulfonamide. ES-LCMS m/z 555.2 [M+H]⁺.
35. N-(4-((3-(2-(((1r,4r)-4-aminocyclohexyl)amino)pyrimidin-4-yl)pyridin-4-yl)oxy)-3-fluorophenyl) 5-cyano-2-methylbenzenesulfonamide. ES-LCMS m/z 574.3 [M+H]⁺.
36. N-[4-[[3-[2-[(1r,4r)(4-Aminocyclohexyl)amino]pyrimidin-4-yl]-4-pyridyl]oxy]-3-fluorophenyl] cyclopropanesulfonamide. ES-LCMS m/z 499.2 [M+H]⁺.
37. N-[4-[[3-[2-[(1r,4r)(4-Aminocyclohexyl)amino]pyrimidin-4-yl]-4-pyridyl]oxy]-3-fluorophenyl] benzenesulfonamide. ES-LCMS m/z 535.2 [M+H]⁺.
38. 4-(N-(4-((3-(2-(((1r,4r)-4-aminocyclohexyl)amino)pyrimidin-4-yl)pyridin-4-yl)oxy)-3-fluorophenyl)sulfamoyl) benzamide. ES-LCMS m/z 578.2 [M+H]⁺.
39. N-[4-[[3-[2-[(1r,4r)(4-Aminocyclohexyl)amino]pyrimidin-4-yl]-4-pyridyl]oxy]-3-fluorophenyl]cyclobutanesulfonamide. ES-LCMS m/z 513.1 [M+H]⁺.
40. N-(4-((3-(2-(((1r,4r)-4-aminocyclohexyl)amino)pyrimidin-4-yl)pyridin-4-yl)oxy)-3-fluorophenyl) methanesulfonamide. ES-LCMS m/z 473.2 [M+H]⁺.
41. N-[4-[[3-[2-[(1r,4r)(4-aminocyclohexyl)amino]pyrimidin-4-yl]-4-pyridyl]oxy]-3-fluorophenyl]1,3-benzodioxol-5-ylsulfonamide. ES-LCMS m/z 579.2 [M+H]⁺.
42. N-[4-[[3-[2-[(1r,4r)(4-aminocyclohexyl)amino]pyrimidin-4-yl]-4-pyridyl]oxy]-3-fluorophenyl]quinoline-8-sulfonamide. ES-LCMS m/z 586.2 [M+H]+
43. N-[4-[[3-[2-[(1r,4r)(4-aminocyclohexyl)amino]pyrimidin-4-yl]-4-pyridyl]oxy]-3-fluorophenyl] 4-methoxy-2,3-dimethyl-benzenesulfonamide. ES-LCMS m/z 593.2 [M+H]⁺
44. N-[4-[[3-[2-[(1r,4r)(4-Aminocyclohexyl)amino]pyrimidin-4-yl]-4-pyridyl]oxy]-3-fluorophenyl] phenylmethanesulfonamide. ES-LCMS m/z 549.2 [M+H]⁺.
45. N-[4-[[3-[2-[(1r,4r)(4-aminocyclohexyl)amino]pyrimidin-4-yl]-4-pyridyl]oxy]-3-fluorophenyl] 2-methyl-1H-indole-5-sulfonamide. ES-LCMS m/z 588.2 [M+H]⁺
46. N-[4-[[3-[2-[(1r,4r)(4-Aminocyclohexyl)amino]pyrimidin-4-yl]-4-pyridyl]oxy]-2-fluorophenyl] benzenesulfonamide. ES-LCMS m/z 535.2 [M+H]⁺.
47. N-[4-[[3-[2-[(1r,4r)(4-Aminocyclohexyl)amino]pyrimidin-4-yl]-4-pyridyl]oxy]-2-chloro-5-fluorophenyl] 2-chlorobenzenesulfonamide. ES-LCMS m/z 603.1 [M+H]⁺.
48. N-[4-[[3-[2-[(1r,4r)(4-Aminocyclohexyl)amino]pyrimidin-4-yl]-4-pyridyl]oxy]-2-chloro-5-fluorophenyl] benzenesulfonamide. ES-LCMS m/z 569.2 [M+H]⁺.
49. N-[4-[[3-[2-[(1r,4r)(4-Aminocyclohexyl)amino]pyrimidin-4-yl]-4-pyridyl]oxy]-2-chloro-5-fluorophenyl] phenylmethanesulfonamide. ES-LCMS m/z 583.2 [M+H]⁺.
50. N-[4-[[3-[2-[(1r,4r)(4-Aminocyclohexyl)amino]pyrimidin-4-yl]-4-pyridyl]oxy]-2-chlorophenyl] cyclopropanesulfonamide. ES-LCMS m/z 515.2 [M+H]⁺.
51. N-[4-[[3-[2-[(1r,4r)(4-Aminocyclohexyl)amino]pyrimidin-4-yl]-4-pyridyl]oxy]-2-chlorophenyl] phenylmethanesulfonamide. ES-LCMS m/z 565.2 [M+H]⁺.
52. N-[4-[[3-[2-[(1r,4r)(4-Aminocyclohexyl)amino]pyrimidin-4-yl]-4-pyridyl]oxy]-2-chloro-3-fluorophenyl] benzenesulfonamide. ES-LCMS m/z 569.2 [M+H]⁺.
53. N-[4-[[3-[2-[(1r,4r)(4-Aminocyclohexyl)amino]pyrimidin-4-yl]-4-pyridyl]oxy]-3-fluorophenyl] 2-bromobenzenesulfonamide. ES-LCMS m/z 615.0 [M+H]⁺.
54. N-[4-[[3-[2-[(1r,4r)(4-Aminocyclohexyl)amino]pyrimidin-4-yl]-4-pyridyl]oxy]-3-fluorophenyl] 2-methylbenzenesulfonamide. ES-LCMS m/z 549.3 [M+H]⁺.
55. N-[4-[[3-[2-[(1r,4r)(4-Aminocyclohexyl)amino]pyrimidin-4-yl]-4-pyridyl]oxy]-3-fluorophenyl] 2,4,6-trimethylbenzenesulfonamide. ES-LCMS m/z 577.2 [M+H]⁺.
56. N-[4-[[3-[2-[(1r,4r)(4-Aminocyclohexyl)amino]pyrimidin-4-yl]-4-pyridyl]oxy]-3-fluorophenyl] quinoxalin-5-ylsulfonamide ES-LCMS m/z 587.2 [M+H]⁺.
57. N-[4-[[3-[2-[(1r,4r)(4-Aminocyclohexyl)amino]pyrimidin-4-yl]-4-pyridyl]oxy]-3-fluorophenyl] tetralin-2-ylsulfonamide. ES-LCMS m/z 589.4 [M+H]⁺.
58. N-[4-[[3-[2-[(1r,4r)(4-Aminocyclohexyl)amino]pyrimidin-4-yl]-4-pyridyl]oxy]-3-fluorophenyl] 4-bromo-5-chloro-2-fluorobenzenesulfonamide. ES-LCMS m/z 667.0 [M+H]⁺.
59. N-[3-Fluoro-4-[[3-[2-[[(3S)-3-piperidyl]amino]pyrimidin-4-yl]-4-pyridyl]oxy]phenyl] benzenesulfonamide. ES-LCMS m/z 521.2 [M+H]⁺
60. N-[4-[[3-[2-[(1r,4r)(4-Aminocyclohexyl)amino]pyrimidin-4-yl]-4-pyridyl]oxy]-2-chloro-5-fluorophenyl] cyclopropylsulfonamide. ES-LCMS m/z 533.1 [M+H]⁺.
61. N-[4-[[3-[2-[(1r,4r)(4-Aminocyclohexyl)amino]pyrimidin-4-yl]-4-pyridyl]oxy]-2-chiorophenyl] benzenesulfonamide. ES-LCMS m/z 551.1 [M+H]⁺.
62. N-[4-[[3-[2-[(1r,4r)(4-Aminocyclohexyl)amino]pyrimidin-4-yl]-4-pyridyl]oxy]-2-bromophenyl] benzenesulfonamide. ES-LCMS m/z 595.1, 597.0 [M+H]⁺.
63. N-[4-[[3-[2-[(1r,4r)(4-Aminocyclohexyl)amino]pyrimidin-4-yl]-4-pyridyl]oxy]-2,6-dichlorophenyl] benzenesulfonamide. ES-LCMS m/z 585.3 [M+H]⁺.
64. N-[4-[[3-[2-[(1r,4r)(4-Aminocyclohexyl)amino]pyrimidin-4-yl]-4-pyridyl]oxy]-3-fluorophenyl] 2-fluorobenzenesulfonamide. ES-LCMS m/z 553.2 [M+H]⁺.
65. N-[4-[[3-[2-[(1r,4r)(4-Aminocyclohexyl)amino]pyrimidin-4-yl]-4-pyridyl]oxy]-3-fluorophenyl] 2-iodobenzenesulfonamide. ES-LCMS m/z 661.1 [M+H]⁺.
66. N-[4-[[3-[2-[(1r,4r)(4-Aminocyclohexyl)amino]pyrimidin-4-yl]-4-pyridyl]oxy]-3-fluorophenyl] 2-tert-butylbenzenesulfonamide. ES-LCMS m/z 591.4 [M+H]⁺.
67. N-[4-[[3-[2-[(1r,4r)(4-Aminocyclohexyl)amino]pyrimidin-4-yl]-4-pyridyl]oxy]-3-fluorophenyl] 2-cyanobenzenesulfonamide. ES-LCMS m/z 560.2 [M+H]⁺.
68. N-[4-[[3-[2-[(1r,4r)(4-Aminocyclohexyl)amino]pyrimidin-4-yl]-4-pyridyl]oxy]-3-fluorophenyl] cyclohexylsulfonamide. ES-LCMS m/z 541.3 [M+H]⁺.
69. N-[4-[[3-[2-[(1r,4r)(4-Aminocyclohexyl)amino]pyrimidin-4-yl]-4-pyridyl]oxy]-3-fluorophenyl] naphthalene-1-sulfonamide. ES-LCMS m/z 585.2 [M+H]⁺.
70. N-[3-Fluoro-4-[[3-[2-[[(3S)-3-piperidyl]amino]pyrimidin-4-yl]-4-pyridyl]oxy]phenyl] naphthalene-1-sulfonamide. ES-LCMS m/z 571.2 [M+H]⁺.
71. N-[4-[[3-[2-(1r,4r)[(4-Aminocyclohexyl)amino]pyrimidin-4-yl]-4-pyridyl]oxy]-3-fluorophenyl] 2-ethylbenzenesulfonamide. ES-LCMS 563.4 m/z [M+H]⁺.
72. N-[4-[[3-[2-(1r,4r)[(4-Aminocyclohexyl)amino]pyrimidin-4-yl]-4-pyridyl]oxy]-3-fluorophenyl] 2-phenylbenzenesulfonamide. ES-LCMS 611.4 m/z [M+H]⁺.
73. N-[4-[[3-[2-(1r,4r)[(4-Aminocyclohexyl)amino]pyrimidin-4-yl]-4-pyridyl]oxy]-3-fluorophenyl] 2-ethynylbenzenesulfonamide. ES-LCMS 559.3 m/z [M+H]⁺.
74. N-[4-[[3-[2-(1r,4r)[(4-Aminocyclohexyl)amino]pyrimidin-4-yl]-4-pyridyl]oxy]-3-fluorophenyl] adamant-1-ylsulfonamide. ES-LCMS 593.3 m/z [M+H]⁺.
75. N-[4-[[3-[2-(1r,4r)[(4-Aminocyclohexyl)amino]pyrimidin-4-yl]-4-pyridyl]oxy]-3-fluorophenyl] [R,S]-exo-norborn-2-ylsulfonamide. ES-LCMS 553.2 m/z [M+H]⁺.
76. N-[4-[[3-[2-[(1r,4r)(4-(N,N-Dimethylamino)cyclohexyl)amino]pyrimidin-4-yl]-4-pyridyl]oxy]-3-fluorophenyl] 2-chlorobenzamide. ES-LCMS m/z 561.2, 563.2 [M+H]⁺.
77. N-[3-Fluoro-4-[[3-[2-[[N-methylpiperid-4-yl]amino]pyrimidin-4-yl]-4-pyridyl]oxy]phenyl] 2-chlorobenzenesulfonamide. ES-LCMS m/z 569.2, 571.0 [M+H]⁺
78. N-[4-[[3-[2-[(1r,4r)(4-(N,N-Dimethylamino)cyclohexyl)amino]pyrimidin-4-yl]-4-pyridyl]oxy]-3-fluorophenyl] 2-chlorobenzenesulfonamide. ES-LCMS m/z 597.2, 599.2 [M+H]⁺.
79. N-[4-[[3-[2-[(1r,4r)(4-Aminocyclohexyl)amino]pyrimidin-4-yl]-4-pyridyl]oxy]-2-fluorophenyl] 2-chlorobenzenesulfonamide. ES-LCMS m/z 569.1, 571.1 [M+H]⁺.
80. N-[4-[[3-[2-[(1r,4r)(4-Aminocyclohexyl)amino]pyrimidin-4-yl]-4-pyridyl]oxy]-3-fluorophenyl] 3-fluoro-4-nitrobenzenesulfonamide. ES-LCMS m/z 598.1.
81. N-[4-[[3-[2-(1r,4r)[(4-Aminocyclohexyl)amino]pyrimidin-4-yl]-4-pyridyl]oxy]-2-fluorophenyl] 2-fluoro-4-(hydroxymethyl)benzenesulfonamide. ES-LCMS m/z 82583.2.
82. N-82[4-[[3-[2-(1r,4r)[(4-Aminocyclohexyl)amino]pyrimidin-4-yl]-4-pyridyl]oxy]-2-fluorophenyl] 2-fluoro-4-(hydroxymethyl)benzenesulfonamide. ES-LCMS m/z 583.1.
83. N-[4-[[3-[2-(1r,4r)[(4-Aminocyclohexyl)amino]pyrimidin-4-yl]-4-pyridyl]oxy]-3-fluorophenyl] 2-fluoro-4-(2-hydroxyethyl)benzenesulfonamide. ES-LCMS m/z 597.2.
84. N-[4-[[3-[2-(1r,4r)[(4-Aminocyclohexyl)amino]pyrimidin-4-yl]-4-pyridyl]oxy]-2-fluorophenyl] 2-fluoro-4-(2-hydroxyethyl)benzenesulfonamide. ES-LCMS m/z 597.1.
85. N-[4-[[3-[2-(2-(N,N-Dimethylamino)ethylamino)]pyrimidin-4-yl]-4-pyridyl]oxy]-3-fluorophenyl] 2-chlorobenzenesulfonamide. ES-LCMS m/z 566.0.
86. N-[4-[[3-[2-(2-(2-Morpholin-1-yl)ethylamino)]pyrimidin-4-yl]-4-pyridyl]oxy]-3-fluorophenyl] 2-chlorobenzenesulfonamide. ES-LCMS m/z 583.3.
87. N-[4-[[3-[2-(2-(2-Hydroxyethoxy)ethylamino)]pyrimidin-4-yl]-4-pyridyl]oxy]-3-fluorophenyl] 2-chlorobenzenesulfonamide. ES-LCMS m/z 560.1.
88. N-[4-[[3-[2-(Oxetan-3-ylamino)]pyrimidin-4-yl]-4-pyridyl]oxy]-3-fluorophenyl] 2-chlorobenzenesulfonamide. ES-LCMS m/z 528.0,
89. N-[4-[[3-[2-([RS]-Pyrrolidin-3-ylamino)]pyrimidin-4-yl]-4-pyridyl]oxy]-3-fluorophenyl] 2-chlorobenzenesulfonamide. ES-LCMS m/z 541.0.
90. N-[4-[[3-[2-(2-(Pyrrolidin-1-yl)ethylamino)]pyrimidin-4-yl]-4-pyridyl]oxy]-3-fluorophenyl] 2-chlorobenzenesulfonamide. ES-LCMS m/z 569.3.
91. N-[4-[[3-[2-(2-(2-Methoxyethoxy)ethylamino)]pyrimidin-4-yl]-4-pyridyl]oxy]-3-fluorophenyl] 2-chlorobenzenesulfonamide. ES-LCMS m/z 574.1.
92. N-[4-[[3-[2-(2-Azaspiro[3.3]hept-6-ylamino)]pyrimidin-4-yl]-4-pyridyl]oxy]-3-fluorophenyl] 2-chlorobenzenesulfonamide. ES-LCMS m/z 567.1.
93. N-[4-[[3-[2-(2-(1,N-Methylpyrrolidin-3-yl)ethylamino)]pyrimidin-4-yl]-4-pyridyl]oxy]-3-fluorophenyl] 2-chlorobenzenesulfonamide. ES-LCMS m/z 555.0.
94. N-[4-[[3-[2-(2-(Azetidin-1-yl)ethylamino)]pyrimidin-4-yl]-4-pyridyl]oxy]-3-fluorophenyl] 2-chlorobenzenesulfonamide. ES-LCMS m/z 555.0
95. N-[4-[[3-[2-(2-(Imidazol-1-yl)ethylamino)]pyrimidin-4-yl]-4-pyridyl]oxy]-3-fluorophenyl] 2-chlorobenzenesulfonamide ES-LCMS m/z 566.1.
96. N-[4-[[3-[2-(2-(Imidazol-2-yl)ethylamino)]pyrimidin-4-yl]-4-pyridyl]oxy]-3-fluorophenyl] 2-chlorobenzenesulfonamide ES-LCMS m/z 566.1.
97. N-[4-[[3-[2-(2-(2-Hydroxyethylamino)ethylamino)]pyrimidin-4-yl]-4-pyridyl]oxy]-3-fluorophenyl] 2-chlorobenzenesulfonamide. ES-LCMS m/z 559.0.
98. N-[4-[[3-[2-(Azetidin-3-ylamino)]pyrimidin-4-yl]-4-pyridyl]oxy]-3-fluorophenyl] 2-chlorobenzenesulfonamide. ES-LCMS m/z 527.0
99. N-[4-[[3-[2-(2,N-Methylazaspiro[3.3]hept-6-ylamino)]pyrimidin-4-yl]-4-pyridyl]oxy]-3-fluorophenyl] 2-chlorobenzenesulfonamide. ES-LCMS m/z 581.1.
100. N-[4-[[3-[2-(2-(N-Methyl-N-methanesulfonylamino)ethylamino)]pyrimidin-4-yl]-4-pyridyl]oxy]-3-fluorophenyl] 2-chlorobenzenesulfonamide. ES-LCMS m/z 607.0.
101. N-[4-[[3-[2-(1,N-(Oxetan-3-yl)azetidin-3-ylamino)]pyrimidin-4-yl]-4-pyridyl]oxy]-3-fluorophenyl] 2-chlorobenzenesulfonamide. ES-LCMS m/z 582.9.
102. N-[4-[[3-[2-(1,N-(Methyl)azetidin-3-ylamino)]pyrimidin-4-yl]-4-pyridyl]oxy]-3-fluorophenyl] 2-chlorobenzenesulfonamide. ES-LCMS m/z 541.1.
103. N-[4-[[3-[2-([S]-1,N-Methylsulfonylpiperidin-3-ylamino)]pyrimidin-4-yl]-4-pyridyl]oxy]-3-fluorophenyl] 2-chlorobenzenesulfonamide. ES-LCMS m/z 633.1.
104. N-[4-[[3-[2-(2-(2-Methoxyethylamino)ethylamino)]pyrimidin-4-yl]-4-pyridyl]oxy]-3-fluorophenyl] 2-chlorobenzenesulfonamide. ES-LCMS m/z 573.1.
105. N-[4-[[3-[2-(1,N-(2-Methoxyethyl)azetidin-3-ylamino)]pyrimidin-4-yl]-4-pyridyl]oxy]-3-fluorophenyl] 2-chlorobenzenesulfonamide. ES-LCMS m/z 585.1.
106. N-[4-[[3-[2-(1,N-(2-Hydroxyethyl)azetidin-3-ylamino)]pyrimidin-4-yl]-4-pyridyl]oxy]-3-fluorophenyl] 2-chlorobenzenesulfonamide. ES-LCMS m/z 571.0.
107. N-[4-[[3-[2-(1,N-(2-(2-Methoxyethoxy)ethyl)azetidin-3-ylamino)]pyrimidin-4-yl]-4-pyridyl]oxy]-3-fluorophenyl] 2-chlorobenzenesulfonamide. ES-LCMS m/z 629.3.

### Example 4. Syntheses of Compounds 108-115

The following compounds were made using Method C.
108. N-(4-((3-(2-(((1r,4r)-4-Aminocyclohexyl)amino)pyrimidin-4-yl)pyridin-2-yl)thio)-3-fluorophenyl) 2-chlorobenzenesulfonamide

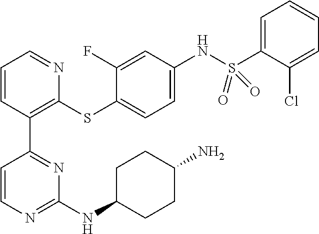 [SMILES: N[C@H]1CC[C@H](Nc2nccc(-c3cccnc3Sc3ccc(NS(=O)(=O)c4ccccc4Cl)cc3F)n2)CC1]

### Step 1: 2-Chloro-4-(2-fluoro-3-pyridyl)pyrimidine

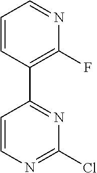 [SMILES: Fc1ncccc1-c1ccnc(Cl)n1]

To a solution of 2,4-dichloropyrimidine (10 g, 67.12 mmol, 1 eq) and 2-fluoropyridin-3-yl) boronic acid (9.46 g, 67.12 mmol, 1 eq) in THF (300 mL) and H₂O (100 mL) was added Pd(dppf)Cl₂ (2.46 g, 3.36 mmol, 0.05 eq) and Cs₂CO₃ (43.74 g, 134.25 mmol, 2 eq) under N₂. The mixture was stirred at 60° C. for 12 h. The reaction mixture was quenched by the addition of water (250 mL), and extracted with EtOAc (200 mL×3). The combined organic layers were washed with brine (50 mL), dried over Na₂SO₄, filtered and concentrated under reduced pressure to yield a residue which was purified by flash silica gel chromatography (PE/EtOAc=1/0 to 5/1, TLC: PE/EtOAc=5/1, R_{f}=0.38) to yield 2-chloro-4-(2-fluoro-3-pyridyl)pyrimidine (13.5 g, 63.05 mmol, 93.9% yield, 97.9% purity) as a white solid. ¹H NMR (400 MHz, CDCl₃) δ ppm 8.83-8.70 (m, 2H), 8.43-8.33 (m, 1H), 7.90 (dd, J=1.2, 5.2 Hz, 1H), 7.42 (ddd, J=2.0, 5.2, 7.6 Hz, 1H); ES-LCMS m/z 210.2 └M+H┘⁺.

### Step 2: O-t-Butyl N-((1r,4r)-4-((4-(2-fluoropyridin-3-yl)pyrimidin-2-yl)amino)cyclohexyl) carbamate

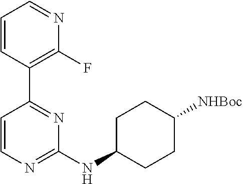 [SMILES: CC(C)(C)OC(=O)N[C@H]1CC[C@H](Nc2nccc(-c3cccnc3F)n2)CC1]

To a solution of 2-chloro-4-(2-fluoro-3-pyridyl)pyrimidine (2 g, 8.34 mmol, 87.4%, 1 eq) in DMSO (20 mL) was added TEA (1.27 g, 12.51 mmol, 1.74 mL, 1.5 eq) and tert-butyl N-(4-aminocyclohexyl)carbamate (1.79 g, 8.34 mmol, 1 eq). The mixture was stirred at 80° C. for 4 h. The reaction mixture was quenched by the addition of water (100 mL), extracted with EtOAc (50 mL×3). The combined organic layers were washed with brine (20 mL), dried over Na₂SO₄, filtered and concentrated under reduced pressure to give a residue which was purified by preparative HPLC (column: Phenomenex C18 150*40 mm*5 μm; mobile phase: [water (NH₄HCO₃)-ACN]; B %: 32%-62%, 10 min), followed by lyophilization to yield O-t-butyl N-((1r,4r)-4-((4-(2-fluoropyridin-3-yl)pyrimidin-2-yl)amino)cyclohexyl) carbamate (1.2 g, 3.10 mmol, 37.1% yield, 100% purity) as a white solid. ¹H NMR (400 MHz, CDCl₃) δ ppm 8.57 (t, J=7.6 Hz, 1H), 8.38 (d, J=5.2 Hz, 1H), 8.32-8.28 (m, 1H), 7.36-7.32 (m, 1H), 7.14 (dd, J=2.4, 5.2 Hz, 1H), 5.08 (d, J=7.6 Hz, 1H), 4.43 (s, 1H), 3.92-3.79 (m, 1H), 3.49 (s, 1H), 2.23-2.06 (m, 4H), 1.46 (s, 9H), 1.39-1.29 (m, 4H).

### Step 3: Bis-(2-fluoro-4-nitrophenyl)disulfane

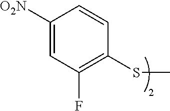

To a solution of 1,2-difluoro-4-nitro-benzene (5 g, 31.43 mmol, 3.47 mL, 1 eq) in DMF (100 mL) was added Na₂S (3.51 g, 44.94 mmol, 1.43 eq). The mixture was stirred at 20° C. for 4 h. The reaction mixture was quenched by the addition of aqueous HCl (100 mL, pH=3) solution. The crude product was precipitated from the solution. Then the filter cake was dried under reduced pressure to yield bis-(2-fluoro-4-nitrophenyl)disulfane (6.6 g, 19.17 mmol, 60.9% yield, 100% purity) as a yellow solid. ¹H NMR (400 MHz, DMSO-d₆) δ ppm 8.08 (dd, J=2.4, 9.6 Hz, 2H), 7.99 (dd, J=2.0, 8.8 Hz, 2H), 7.73 (approx t, J=8.4 Hz, 2H).

### Step 4: 4-Amino-2-fluoro-benzenethiol

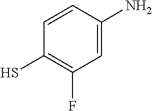

To a solution of 1 bis-(2-fluoro-4-nitrophenyl)disulfane (5 g, 13.07 mmol, 100% purity, 1 eq) in THF (40 mL) was added Zn (5.13 g, 78.42 mmol, 6 eq) and HCl (3 M, 13.07 mL, 3 eq) dropwise at 0° C. The mixture was stirred at 20° C. for 4 h. 37% HCl (20 mL) was added to the reaction. And the reaction mixture was filtrated, and concentrated under reduced pressure. The residue was purified by preparative HPLC (column: Agela DuraShell C18 150*25 mm*5 μm; mobile phase: [water (0.04% NH₃H₂O+10 mM NH₄HCO₃)-ACN]; B %: 60%-90%, 10 min), followed by lyophilization to yield 4-amino-2-fluoro-benzenethiol (1.5 g, 9.74 mmol, 74.6% yield, 93.0% purity) as a white solid. ¹H NMR (400 MHz, DMSO-d₆) δ ppm 7.29 (t, J=8.4 Hz, 1H), 6.75-6.56 (m, 2H); ES-LCMS m/z 144.3 [M+H]⁺.

### Step 5: O-t-Butyl N-((1r,4r)-4-((4-(2-((4-amino-2-fluorophenyl)thio)pyridin-3-yl)pyrimidin-2-yl)amino)cyclohexyl) carbamate

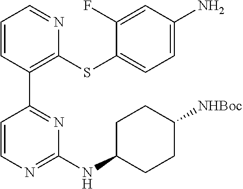 [SMILES: CC(C)(C)OC(=O)N[C@H]1CC[C@H](Nc2nccc(-c3cccnc3Sc3ccc(N)cc3F)n2)CC1]

To a solution of O-t-butyl N-((1r,4r)-4-((4-(2-fluoropyridin-3-yl)pyrimidin-2-yl)amino)cyclohexyl) carbamate (500 mg, 1.16 mmol, 90%, 1 eq) in NMP (5 mL) was added 4-amino-2-fluoro-benzenethiol (831.48 mg, 5.81 mmol, 5 eq) dropwise at 140° C. The reaction was stirred at 140° C. for 2 h. The reaction mixture was added Boc₂O (253.48 mg, 1.16 mmol, 266.82 μL, 1 eq), and then was stirred at 20° C. for 1 h. The mixture was quenched with water (10 mL). The aqueous phase was neutralized to pH=8 with saturated NaHCO₃ solution at 0° C. and extracted with ethyl acetate (20 mL×3). The combined organic phase was washed with brine (50 mL), dried over anhydrous Na₂SO₄, filtered and concentrated under vacuum to yield a residue which was purified by flash silica gel chromatography (PE/EtOAc=1/0 to 1/1, TLC: PE/EtOAc=1/1, R_{f}=0.45) to yield O-t-butyl N-((1r,4r)-4-((4-(2-((4-amino-2-fluorophenyl)thio)pyridin-3-yl)pyrimidin-2-yl)amino)cyclohexyl) carbamate (310 mg, 607.10 μmol, 52.2% yield, 100% purity) as a yellow solid. ¹H NMR (400 MHz, DMSO-d₆) δ ppm 8.50-8.27 (m, 2H), 8.08-7.74 (m, 1H), 7.21 (t, J=4.8, 7.6 Hz, 2H), 7.10 (t, J=8.0 Hz, 1H), 6.93 (br s, 1H), 6.76 (d, J=5.6 Hz, 1H), 6.46-6.31 (m, 2H), 3.98-3.65 (m, 1H), 3.25-3.17 (m, 1H), 2.05-1.91 (m, 2H), 1.86-1.72 (m, 2H), 1.38 (s, 9H), 1.35-1.15 (m, 4H); ES-LCMS m/z 511.3 [M+H]⁺.

### Step 6: O-t-Butyl N-((1r,4r)-4-((4-(2-((4-(2-chlorophenylsulfonamido)-2-fluorophenyl)thio)pyridin-3-yl)pyrimidin-2-yl)amino)cyclohexyl) carbamate

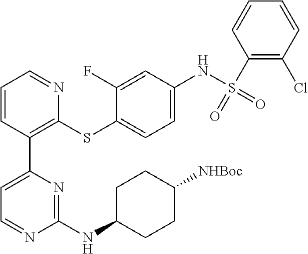 [SMILES: CC(C)(C)OC(=O)N[C@H]1CC[C@H](Nc2nccc(-c3cccnc3Sc3ccc(NS(=O)(=O)c4ccccc4Cl)cc3F)n2)CC1]

A mixture of O-t-butyl N-((1r,4r)-4-((4-(2-((4-amino-2-fluorophenyl)thio)pyridin-3-yl)pyrimidin-2-yl)amino)cyclohexyl) carbamate (55 mg, 107.71 μmol, 100%, 1 eq), benzenesulfonyl chloride (19.02 mg, 107.71 μmol, 13.79 μL, 1 eq), pyridine (68.16 mg, 861.69 μmol, 69.55 μL, 8 eq), DMAP (6.58 mg, 53.86 μmol, 0.5 eq) in THF (3 mL) was degassed and purged with N₂ for 3 times, and then the mixture was stirred at 20° C. for 2 h. The mixture was quenched with water (20 mL) and extracted with ethyl acetate (20 mL×3). The combined organic phase was washed with brine (50 mL), dried over anhydrous Na₂SO₄, filtered and concentrated under vacuum to yield a residue which was purified by preparative TLC (PE/EtOAc=1/1, TLC: PE/EtOAc=1/1, R_{f}=0.40) to yield O-t-butyl N-((1r,4r)-4-((4-(2-((4-(2-chlorophenylsulfonamido)-2-fluorophenyl)thio)pyridin-3-yl)pyrimidin-2-yl)amino)cyclohexyl) carbamate (60 mg, 77.44 μmol, 71.9% yield, 84.0% purity) as a yellow solid. ¹H NMR (400 MHz, DMSO-d₆) δ ppm 11.20 (s, 1H), 8.37 (brd, J=2.9 Hz, 1H), 8.32-8.23 (m, 1H), 8.14 (d, J=7.9 Hz, 1H), 8.10-7.98 (m, 1H), 7.72-7.62 (m, 2H), 7.61-7.51 (m, 1H), 7.39-7.34 (m, 1H), 7.29-7.20 (m, 2H), 7.03-6.82 (m, 3H), 6.74 (d, J=7.3 Hz, 1H), 4.04-3.82 (m, 1H), 3.22-3.12 (m, 1H), 1.99-1.90 (m, 2H), 1.82-1.70 (m, 2H), 1.38 (s, 9H), 1.32-1.21 (m, 4H); ES-LCMS m/z 651.3 [M+H]⁺.

### Step 7: N-(4-((3-(2-(((1r,4r)-4-Aminocyclohexyl)amino)pyrimidin-4-yl)pyridin-2-yl)thio)-3-fluorophenyl) 2-chlorobenzenesulfonamide

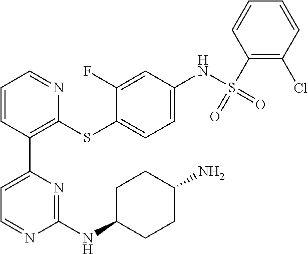 [SMILES: N[C@H]1CC[C@H](Nc2nccc(-c3cccnc3Sc3ccc(NS(=O)(=O)c4ccccc4Cl)cc3F)n2)CC1]

To a solution of O-t-butyl N-((1r,4r)-4-((4-(2-((4-(2-chlorophenylsulfonamido)-2-fluorophenyl)thio)pyridin-3-yl)pyrimidin-2-yl)amino)cyclohexyl) carbamate (55 mg, 71.65 μmol, 89.27%, 1 eq) in DCM (2 mL) was added HCl/1,4-dioxane (4 M, 35.83 μL, 2 eq). The mixture was stirred at 20° C. for 1 h. The residue was purified by preparative HPLC (column: C18-1 150*30 mm*5 μm; mobile phase: [water (NH₃H₂O+NH₄HCO₃)-ACN]; B %: 18%-48%, 15 min), followed by lyophilization to yield N-(4-((3-(2-(((1r,4r)-4-aminocyclohexyl)amino)pyrimidin-4-yl)pyridin-2-yl)thio)-3-fluorophenyl) 2-chlorobenzenesulfonamide (7.5 mg, 12.82 μmol, 17.9% yield, 100% purity) as a white solid. ¹H NMR (400 MHz, DMSO-d₆, 75° C.) δ ppm 8.38 (d, J=4.8 Hz, 1H), 8.33 (dd J=5.1, 1.7 Hz), 1H), 8.03 (dd, J=1.8, 7.4 Hz, 1H), 7.90 (dd, J=7.8, 1.7 Hz, 1H), 7.47-7.34 (m, 3H), 7.19 (dd, J=4.8, 7.6 Hz, 1H), 7.06 (t, J=8.5 Hz, 1H), 6.93 (J=5.1 Hz, 1H), 6.93-6.90 (brs, 1H), 6.72-6.63 (m, 2H), 3.97-3.85 (m, 1H), 2.99-2.90 (m, 1H), 2.16-1.88 (m, 4H), 1.47-1.31 (m, 4H); ES-LCMS m/z 551.3 [M+H]⁺.
109. N-[3-Fluoro-4-[[3-[2-[[(3S)-3-piperidyl]amino]pyrimidin-4-yl]-2-pyridyl]thio]phenyl] 2-chlorobenzenesulfonamide

### Step 1. O-t-Butyl N-((3S)-3-[[4-(2-fluoro-3-pyridyl)pyrimidin-2-yl]amino]piperidin-1-yl) carbamate

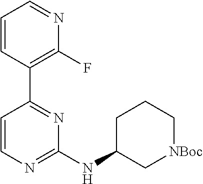 [SMILES: CC(C)(C)OC(=O)N1CCC[C@H](Nc2nccc(-c3cccnc3F)n2)C1]

To a solution of 2-chloro-4-(2-fluoro-3-pyridyl)pyrimidine (500 mg, 2.39 mmol, 100.0% purity, 1 eq) and O-t-butyl N-((3S)-3-aminopiperidin-1-yl) carbamate (573.29 mg, 2.86 mmol, 1.2 eq) in DMSO (6 mL) was added K₂CO₃ (494.52 mg, 3.58 mmol, 1.5 eq) which was stirred at 70° C. for 2 h. The mixture was concentrated and then water (150 mL) was added. The mixture was extracted with EtOAc (150 mL×3). The combined organic layers were washed with brine (450 mL), dried over Na₂SO₄, filtered and concentrated. The residue was purified by preparative HPLC (column: Boston Prime C18 150*30 mm*5 μm; mobile phase: [water (NH₃H₂O+NH₄HCO₃)-ACN]; B %: 49%-79%, 10 min) to yield O-t-butyl \hat{ }-((3S)-3-[[4-(2-fluoro-3-pyridyl)pyrimidin-2-yl]amino]piperidin-1-yl) carbamate, 411.33 μmol, 17.2% yield, 96.0% purity) as a white solid. ¹H NMR (500 MHz, CD₃OD-d₄) δ ppm 8.81-8.59 (m, 1H), 8.39 (d, J=5.0 Hz, 1H), 8.31 (d, J=4.6 Hz, 1H), 7.47 (s, 1H), 7.16 (d, J=2.6 Hz, 1H), 3.96 (d, J=6.6 Hz, 1H), 3.88-3.75 (m, 1H), 3.65-3.46 (m, 1H), 3.27-2.70 (m, 2H), 2.08-2.03 (m, 1H), 1.82 (d, J=0.9 Hz, 1H), 1.63-1.54 (m, 2H), 1.42-1.20 (m, 9H); ES-LCMS m/z 374.2 [M+H]⁺.

### Step 2. O-t-Butyl N-((3S)-3-[[4-[2-(4-amino-2-fluoro-phenyl)sulfanyl-3-pyridyl]pyrimidin-2-yl]amino]piperidin-1-yl) carbamate

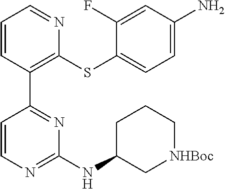

To a solution of O-t-butyl N-((3S)-3-[[4-(2-fluoro-3-pyridyl)pyrimidin-2-yl]amino]piperidin-1-yl) carbamate (500 mg, 1.21 mmol, 90.0% purity, 1 eq) in NMP (10 mL) was added 4-amino-2-fluorobenzenethiol (862.72 mg, 6.03 mmol, 5 eq) dropwise at 20° C. The reaction mixture was stirred at 140° C. for 2 h. Boc₂O (263.00 mg, 1.21 mmol, 276.84 μL, 1 eq) was added to the mixture and stirred at 20° C. for 1 h. The mixture was quenched with water (10 mL). The aqueous phase was neutralized to pH=8 with saturated NaHCO₃ solution and extracted with ethyl acetate (10 mL×3). The combined organic phase was washed with brine (50 mL), dried over anhydrous Na₂SO₄, filtered and concentrated under vacuum to yield a residue which was purified by flash silica gel chromatography (PE/EtOAc=1/0 to 1/1, TLC: PE/EtOAc=1/1, R_{f}=0.45) to yield O-t-butyl N-((3S)-3-[[4-[2-(4-amino-2-fluorophenyl)sulfanyl]-3-pyridyl]pyrimidin-2-yl]amino]piperidin-1-yl) carbamate (320 mg, 644.38 μmol, 53.5% yield, 100% purity) as a yellow solid. ¹H NMR (400 MHz, DMSO-d₆) δ ppm 8.42 (d, J=4.9 Hz, 1H), 8.33 (d, J=3.7 Hz, 1H), 8.01-7.78 (m, 1H), 7.32 (d, J=5.1 Hz, 1H), 7.20 (dd, J=4.8, 7.5 Hz, 1H), 7.07 (t, J=8.2 Hz, 1H), 6.99 (d, J=1.1 Hz, 1H), 6.37 (d, J=9.7 Hz, 2H), 5.72 (s, 2H), 3.89-3.76 (m, 1H), 3.70-3.55 (m, 1H), 3.30 (s, 1H), 3.14-2.75 (m, 2H), 1.92 (s, 1H), 1.72 (dt, J=1.8, 3.5 Hz, 1H), 1.64-1.42 (m, 2H), 1.42-1.20 (m, 9H); ES-LCMS m/z 497.2 [M+H]⁺.

### Step 3. O-t-Butyl N-((3S)-3-[[4-[2-[4-[(2-chlorophenyl)sulfonylamino]-2-fluoro-phenyl]sulfanyl]-3-pyridyl]pyrimidin-2-yl]amino]piperidin-1-yl) carbamate

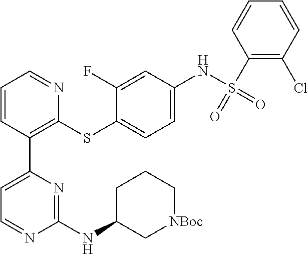 [SMILES: CC(C)(C)OC(=O)N1CCC[C@H](Nc2nccc(-c3cccnc3Sc3ccc(NS(=O)(=O)c4ccccc4Cl)cc3F)n2)C1]

A mixture of O-t-butyl N-((3S)-3-[[4-[2-(4-amino-2-fluorophenyl)sulfanyl]-3-pyridyl]pyrimidin-2-yl]amino]piperidin-1-yl) carbamate (50 mg, 100.68 μmol, 100%, 1 eq), 2-chlorobenzenesulfonyl chloride (31.88 mg, 151.03 μmol, 20.57 μL, 1.5 eq), pyridine (63.71 mg, 805.48 μmol, 65.01 μL, 8 eq), DMAP (6.15 mg, 50.34 μmol, 0.5 eq) in THF (3 mL) was degassed and purged with N₂ for 3 times, and then the mixture was stirred at 20° C. for 2 h under N₂ atmosphere. The mixture was quenched with water (20 mL) and extracted with ethyl acetate (50 mL×3). The combined organic phases were washed with brine (50 mL), dried over anhydrous Na₂SO₄, filtered and concentrated under vacuum to yield a residue which was purified by preparative TLC (PE/EtOAc=1/1, TLC: PE/EtOAc=1/1, R_{f}=0.40) to yield O-t-butyl N-((3S)-3-[[4-[2-[4-[(2-chlorophenyl)sulfonylamino]-2-fluoro-phenyl]sulfanyl]-3-pyridyl]pyrimidin-2-yl]amino]piperidin-1-yl) carbamate (60 mg, 78.66 μmol, 78.1% yield, 88.0% purity) as a yellow solid. ¹H NMR (400 MHz, DMSO-d₆) δ ppm 8.43-8.36 (m, 1H), 8.35-8.27 (m, 1H), 8.17-7.91 (m, 2H), 7.88-7.73 (m, 1H), 7.70-7.57 (m, 2H), 7.57-7.46 (m, 1H), 7.43-7.25 (m, 2H), 7.24-7.18 (m, 1H), 7.06-6.95 (m, 1H), 6.91-6.70 (m, 2H), 4.08-3.93 (m, 1H), 3.92-3.74 (m, 1H), 3.73-3.57 (m, 1H), 3.24-3.09 (m, 2H), 1.97-1.92 (m, 1H), 1.79-1.70 (m, 1H), 1.62-1.41 (m, 2H), 1.39-1.20 (m, 9H); ES-LCMS m/z 671.1 [M+H]⁺.

### Step 4. N-[3-fluoro-4-[[3-[2-[[(3S)-3-piperidyl]amino]pyrimidin-4-yl]-2-pyridyl]sulfanyl]phenyl] 2-chlorobenzenesulfonamide

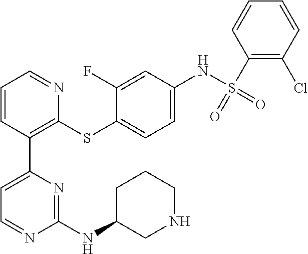 [SMILES: O=S(=O)(Nc1ccc(Sc2ncccc2-c2ccnc(N[C@H]3CCCNC3)n2)c(F)c1)c1ccccc1Cl]

To a solution of t O-t-butyl N-((3S)-3-[[4-[2-[4-[(2-chlorophenyl)sulfonylamino]-2-fluoro-phenyl]sulfanyl]-3-pyridyl]pyrimidin-2-yl]amino]piperidin-1-yl) carbamate (41.59 mg, 54.53 μmol, 88% purity, 1 eq) in DCM (2 mL) was added HCl/1,4-dioxane (4 M, 136.3 μL, 10 eq). The mixture was stirred at 20° C. for 1 h. The reaction mixture was concentrated to yield a residue which was purified by preparative HPLC (column: C18-1 150*30 mm*5 μm; mobile phase: [water (NH₃H₂O+NH₄HCO₃)-ACN]; B %: 18%-48%, 15 min), followed by lyophilization to yield N-[3-fluoro-4-[[3-[2-[[(3S)-3-piperidyl]amino]pyrimidin-4-yl]-2-pyridyl]sulfanyl]phenyl] 2-chlorobenzenesulfonamide (10.10 mg, 17.39 μmol, 31.9% yield, 98.3% purity) as a white solid. ¹H NMR (400 MHz, DMSO-d₆) δ ppm 8.44 (d, J=5.2 Hz, 1H), 8.36 (dd, J=1.6, 4.8 Hz, 1H), 8.01 (dd, J=1.6, 7.2 Hz, 1H), 7.97-7.86 (brs, 1H), 7.51-7.37 (m, 3H), 7.21 (dd, J=4.8, 7.6 Hz, 1H), 7.09 (brt, J=8.4 Hz, 1H), 7.01 (d, J=5.2 Hz, 1H), 6.68-6.60 (m, 2H), 4.23-4.07 (m, 1H), 3.26-3.18 (m, 1H), 3.10 (d, J=12.8 Hz, 1H), 2.89-2.71 (m, 2H), 2.08-1.98 (m, 1H), 1.89-1.80 (m, 1H), 1.69-1.52 (m, 2H); ES-LCMS m/z 571.2 [M+H]⁺.

The following compounds have been made by processes similar to those described immediately above using Method C.
110. N-(4-((3-(2-(((1r,4r)-4-Aminocyclohexyl)amino)pyrimidin-4-yl)pyridin-2-yl)thio)-3-fluorophenyl) benzenesulfonamide. ES-LCMS m/z 551.3 [M+H]⁺.
111. N-(4-((3-(2-(((1r,4r)-4-Aminocyclohexyl)amino)pyrimidin-4-yl)pyridin-2-yl)thio)-3-fluorophenyl) naphthalene-1-sulfonamide. ES-LCMS m/z 601.1 [M+H]⁺.
112. N-[3-fluoro-4-[[3-[2-[[(3S)-3-piperidyl]amino]pyrimidin-4-yl]-2-pyridyl]sulfanyl]phenyl] benzenesulfonamide. ES-LCMS m/z 537.2 [M+H]⁺.
113. N-[3-fluoro-4-[[3-[2-[[(3S)-3-piperidyl]amino]pyrimidin-4-yl]-2-pyridyl]sulfanyl]phenyl] naphthalene-1-sulfonamide. ES-LCMS m/z 587.1 [M+H]⁺.
114. N-[3-fluoro-4-[[3-[2-[oxetan-3-yl]amino]pyrimidin-4-yl]-2-pyridyl]sulfanyl]phenyl] 2-chlorobenzenesulfonamide.

### Step 1: (2-Fluoro-4-nitrophenyl)(4-methoxybenzyl)sulfane

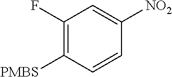

To a solution of 1,2-difluoro-4-nitrobenzene (2.06 g, 13.0 mmol) in DMF (40 mL) were added (4-methoxyphenyl)methanethiol (2.0 g, 13.0 mmol, 1.0 eq) and potassium carbonate (3.58 g, 26.0 mmol, 2 eq), and then the reaction mixture was heated at 80° C. for 3 h. The reaction mixture was diluted with water (150 mL) to give a white precipitate, which was filtered and dried to afford the title compound (3.60 g, 12.3 mmol, 95% yield); ¹H NMR (500 MHz, Chloroform-d) δ 7.95 (m, 1H), 7.89 (dd, J=9.5, 2.3 Hz, 1H), 7.36 (dd, J=8.5, 7.4 Hz, 1H), 7.29 (d, J=8.6 Hz, 2H), 6.89-6.83 (m, 2H), 4.21 (s, 2H), 3.80 (s, 3H).

### Step 2: 3-Fluoro-4-((4-methoxybenzyl)thio)aniline

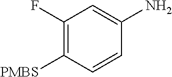

To a solution of (2-fluoro-4-nitrophenyl)(4-methoxybenzyl)sulfane (2.0 g, 6.83 mmol) in THF (70 mL) was added water (60 mL), and then zinc (2.68 g, 41.0 mmol, 6 eq) and ammonium chloride (2.20 g, 41.0 mmol, 6 eq) were added. The reaction mixture was stirred at room temperature for 1 h. The same amounts of zinc and ammonium chloride were added and stirred for an additional hour. The resulting mixture was diluted with water (100 mL) and extracted with EtOAc (120 mL×2). The combined organic layers were washed with brine (100 mL), dried over Na₂SO₄, filtered and concentrated under reduced pressure to afford the title compound (1.81 g, 92% purity) which was used for the next reaction without purification; ¹H NMR (500 MHz, Chloroform-d) δ 7.10 (d, J=8.4 Hz, 2H), 7.03 (t, J=8.2 Hz, 1H), 6.78 (d, J=8.4 Hz, 2H), 6.39 (dd, J=10.8, 2.0 Hz, 1H), 6.31 (dd, J=8.3, 2.2 Hz, 1H), 3.89 (s, 2H), 3.82 (s, 2H), 3.78 (s, 3H); ESI-MS m/z 264 [M+H]⁺.

### Step 3: 2-Chloro-N-(3-fluoro-4-((4-methoxybenzyl)thio)phenyl)benzenesulfonamide

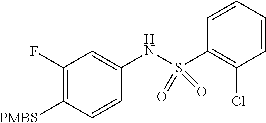

To a stirred solution of 3-fluoro-4-((4-methoxybenzyl)thio)aniline (1.81 g, 6.88 mmol) and 2-chlorobenzenesulfonyl chloride (1.89 g, 8.94 mmol, 1.3 eq) in DCM (70 mL) was added pyridine (1.67 mL, 20.6 mmol, 3 eq). The reaction mixture was stirred at room temperature overnight. The resulting mixture was diluted with water (120 mL) and extracted with DCM (100 mL×2). The combined organic layers were washed with brine (100 mL), dried over Na₂SO₄, filtered and concentrated under reduced pressure to afford the title compound (3.0 g, 89% purity) which was used for the next reaction without purification; ¹H NMR (500 MHz, Chloroform-d) δ 8.04 (d, J=8.0 Hz, 1H), 7.50 (d, J=3.9 Hz, 2H), 7.45 (s, 1H), 7.37 (m, 1H), 7.07 (d, J=8.4 Hz, 2H), 6.95 (dd, J=10.3, 2.2 Hz, 1H), 6.80-6.71 (m, 3H), 3.94 (s, 2H), 3.78 (s, 3H); ESI-MS m/z 455 |M+H₂O|⁺.

### Step 4: 2-Chloro-N-(3-fluoro-4-mercaptophenyl)benzenesulfonamide

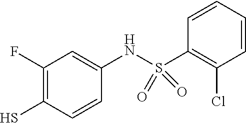 [SMILES: O=S(=O)(Nc1ccc(S)c(F)c1)c1ccccc1Cl]

To a solution of 2-chloro-N-(3-fluoro-4-((4-methoxybenzyl)thio)phenyl)benzene-sulfonamide (500 mg, 1.14 mmol) in TFA (6 mL) were added anisole (0.62 mL, 6.84 mmol, 5 eq) and TfOH (0.50 mL, 6.84 mmol, 5 eq) at 0° C. The reaction mixture was stirred at 0° C. for 10 min, poured into an icy solution of 30% NaOH (6 mL) and sat. NaHCO₃ (100 mL), and then extracted with EtOAc (100 mL). The organic layer was washed with brine, dried, and concentrated to afford the title compound (360 mg, 75% purity) which was used for the next reaction without purification; ESI-MS m/z 318 └M+H┘⁺, 335 [M+H₂O]⁺.

### Step 5: 4-(2-Fluoropyridin-3-yl)-N-(oxetan-3-yl)pyrimidin-2-amine

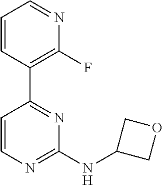 [SMILES: Fc1ncccc1-c1ccnc(NC2COC2)n1]

To a solution of 2-chloro-4-(2-fluoro-3-pyridyl)pyrimidine (800 mg, 3.83 mmol) in DMSO (15 mL) was added 3-aminooxetane (280 mg, 3.83 mmol, 1 eq) and TEA (0.80 mL, 5.75 mmol, 1.5 eq). The reaction mixture was heated at 80° C. for 6 h. The resulting mixture was diluted with water (100 mL) and extracted with EtOAc (100 mL×2). The combined organic layers were washed with water (100 mL×2) and brine (100 mL), dried over Na₂SO₄, filtered and concentrated under reduced pressure to give a residue which was triturated with 20% DCM in Hex, filtered, and dried to afford the title compound as an off white solid (410 mg, 1.67 mmol, 44% yield); ¹H NMR (500 MHz, DMSO-d₆) δ 8.54 (s, 1H), 8.44 (d, J=4.9 Hz, 1H), 8.36 (d, J=3.8 Hz, 1H), 8.12 (t, J=7.7 Hz, 1H), 7.54 (m, 1H), 7.10 (s, 1H), 4.98 (m, 1H), 4.79 (t, J=6.6 Hz, 2H), 4.56 (t, J=6.2 Hz, 2H); ESI-MS m/z 247 [M+H]⁺.

### Step 6: 2-Chloro-N-(3-fluoro-4-((3-(2-(oxetan-3-ylamino)pyrimidin-4-yl)pyridin-2-yl)thio)phenyl)benzenesulfonamide

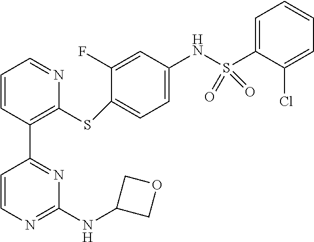 [SMILES: O=S(=O)(Nc1ccc(Sc2ncccc2-c2ccnc(NC3COC3)n2)c(F)c1)c1ccccc1Cl]

To a solution of 2-chloro-N-(3-fluoro-4-mercaptophenyl)benzenesulfonamide (342 mg, 1.08 mmol, 75% purity) in DMF (6 mL) were added 4-(2-fluoropyridin-3-yl)-N-(oxetan-3-yl)pyrimidin-2-amine (200 mg, 0.813 mmol, 1 eq), DMAP (20 mg, 0.163 mmol, 0.2 eq) and Cs₂CO₃ ((400 mg, 1.22 mmol, 1.5 eq). The reaction mixture was irradiated in a microwave reactor at 100° C. for 30 min. After transferred to a separatory funnel, the mixture was diluted with water (50 mL) and extracted with EtOAc (50 mL×2). The combined organic layers were washed with water (50 mL×2) and brine (50 mL), dried over Na₂SO₄, filtered and concentrated under reduced pressure to give a residue which was then purified by silica gel flash column chromatography (25% to 85% EtOAc in Hex) to afford the title compound (170 mg, 0.313 mmol, 38% yield) as an off white solid; ¹H NMR (500 MHz, DMSO-d₆) δ 11.17 (s, 1H), 8.43 (d, J=4.3 Hz, 1H), 8.29 (d, J=3.6 Hz, 1H), 8.14 (d, J=7.6 Hz, 2H), 7.99 (s, 1H), 7.67 (d, J=3.3 Hz, 2H), 7.63-7.51 (m, 1H), 7.41 (s, 1H), 7.29-7.19 (m, 1H), 7.04 (s, 1H), 6.93 (d, J=8.8 Hz, 2H), 5.07 (s, 1H), 4.78 (t, J=6.2 Hz, 2H), 4.55 (t, J=6.1 Hz, 2H); ESI-MS m/z 544 [M+H]⁺.
115. N-[3-fluoro-4-[[3-[2-[oxetan-3-yl]amino]pyrimidin-4-yl]-2-pyridyl]sulfanyl]phenyl]-N-methyl 2-chlorobenzenesulfonamide.

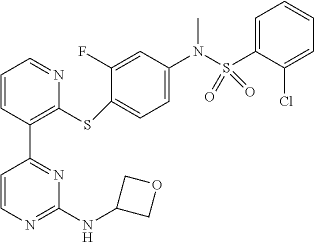 [SMILES: CN(c1ccc(Sc2ncccc2-c2ccnc(NC3COC3)n2)c(F)c1)S(=O)(=O)c1ccccc1Cl]

To a solution of 2-chloro-N-(3-fluoro-4-((3-(2-(oxetan-3-ylamino)pyrimidin-4-yl)pyridin-2-yl)thio)phenyl)benzenesulfonamide (70 mg, 0.129 mmol) in DMF (1 mL) was added 1.0 M LiHMDS in THF (0.142 mL, 0.142 mmol, 1.1 eq) dropwise at 0° C. After the solution was stirred for 5 min at 0° C., Mel (10 μL, 0.168 mmol, 1.3 eq) was added at 0° C. The reaction mixture was stirred at room temperature overnight, and then diluted with water (30 mL) and extracted with EtOAc (50 mL×2). The combined organic layers were washed with water (50 mL×2) and brine (50 mL), dried over Na₂SO₄, filtered and concentrated under reduced pressure to give a residue which was then purified by silica gel flash column chromatography (0% to 5% MeOH in DCM) to afford the title compound (40 mg, 0.0718 mmol, 56% yield) as an off white solid; ¹H NMR (500 MHz, DMSO-d₆) δ 8.45 (d, J=5.0 Hz, 1H), 8.32 (dd, J=4.7, 1.6 Hz, 1H), 8.16 (s, 1H), 8.04 (d, J=6.2 Hz, 1H), 7.97 (d, J=7.6 Hz, 1H), 7.70 (dd, J=3.9, 1.5 Hz, 2H), 7.60-7.47 (m, 2H), 7.26 (ddd, J=21.2, 9.1, 3.5 Hz, 2H), 7.13 (dd, J=8.4, 2.1 Hz, 1H), 7.08 (d, J=4.7 Hz, 1H), 5.10 (s, 1H), 4.80 (t, J=6.4 Hz, 2H), 4.57 (t, J=6.3 Hz, 2H), 3.37 (s, 3H); ESI-MS m/z 558 [M+H]⁺.

### Example 5. Syntheses of Compounds 116-118

The following compounds were made using Method D.
116. N-[4-[[3-[2-[((1r,4r)4 aminocyclohexyl)amino]pyrimidin-4-yl]-4-pyridyl]thio]-3-fluoro-phenyl] 2-chlorobenzenesulfonamide

### Step 1: 4-[[3-(2-Chloropyrimidin-4-yl)-4-pyridyl]sulfanyl]-3-fluoro-aniline

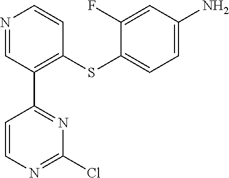 [SMILES: Nc1ccc(Sc2ccncc2-c2ccnc(Cl)n2)c(F)c1]

To a solution of 2-chloro-4-(4-fluoro-3-pyridyl)pyrimidine (350 mg, 1.50 mmol, 1 eq) in DMA (10 mL) was added 4-amino-2-fluoro-benzenethiol (286.90 mg, 1.80 mmol, 1.2 eq). The mixture was stirred at 20° C. for 1 h. TLC (PE/EtOAc=0/1, R_{f}=0.20) indicated the starting material was consumed completely. To the reaction mixture was added water (20 mL) and extracted with EtOAc (20 mL×3). The combined organic layers were washed with brine (20 mL×3), dried over Na₂SO₄, filtered and concentrated under reduced pressure to give a residue which was purified by flash silica gel chromatography (PE/EtOAc=0/1 to 0/1, TLC: PE/EtOAc=0/1, R_{f}=0.2) to yield 4-┌┌3-(2-chloropyrimidin-4-yl)-4-pyridyl]sulfanyl]-3-fluoroaniline (400 mg, 1.08 mmol, 82.0% yield, 90% purity) as a yellow oil. ¹H NMR (400 MHz, DMSO-d₆) δ ppm 8.94 (d, J=5.2 Hz, 1H), 8.41 (d, J=5.5 Hz, 1H), 8.11 (d, J=5.2 Hz, 1H), 7.29-7.19 (m, 1H), 6.76 (d, J=5.5 Hz, 1H), 6.53-6.47 (m, 2H), 6.06 (s, 2H); ES-LCMS m/z 333.1 [M+H]⁺.

### Step 2: O-tert-Butyl N-[4-(1r,4r)[[4-[4-(4-amino-2-fluorophenyl)sulfanyl-3-pyridyl]pyrimidin-2-yl]amino]cyclohexyl] carbamate

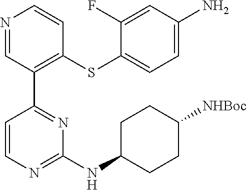 [SMILES: CC(C)(C)OC(=O)N[C@H]1CC[C@H](Nc2nccc(-c3cnccc3Sc3ccc(N)cc3F)n2)CC1]

To a solution of 4-[[3-(2-chloropyrimidin-4-yl)-4-pyridyl]sulfanyl]-3-fluoroaniline (400 mg, 1.08 mmol, 1 eq) in DMSO (10 mL) was added TEA (218.93 mg, 2.16 mmol, 301.14 μL, 2 eq) and tert-butyl N-((1r,4r)-4-aminocyclohexyl)carbamate (463.66 mg, 2.16 mmol, 2 eq). The mixture was stirred at 110° C. for 2 h. TLC (PE/EtOAc=0/1, R_{f}=0.15) indicated the starting material was consumed completely. The mixture was diluted with water (20 mL) and extracted with EtOAc (20 mL×3). The combined organic layers were washed with brine (20 mL×3), dried over Na₂SO₄, filtered and concentrated under reduced pressure to give a residue which was purified by flash silica gel chromatography (PE/EtOAc=10/0 to 0/1, TLC: PE/EtOAc=0/1, R_{f}=0.15) to yield O-tert-butyl N-[4-(1r,4r)[[4-[4-(4-amino-2-fluoro-phenyl)sulfanyl-3-pyridyl]pyrimidin-2-yl]amino]cyclohexyl]carbamate (500 mg, 881.27 μmol, 81.5% yield, 90.0% purity) as a white solid. ¹H NMR (400 MHz, CDCl₃) δ ppm 8.68 (s, 1H), 8.39 (d, J=4.9 Hz, 1H), 8.28 (d, J=5.6 Hz, 1H), 7.32-7.28 (m, 1H), 6.93 (d, J=5.1 Hz, 1H), 6.75 (d, J=5.4 Hz, 1H), 6.55-6.49 (m, 2H), 5.21 (d, J=7.7 Hz, 1H), 4.48-4.34 (m, 1H), 4.03-3.88 (m, 1H), 3.48 (brs, 1H), 2.24 (brd, J=11.2 Hz, 2H), 2.08 (brs, 2H), 1.46 (s, 9H), 1.40-1.30 (m, 4H); ES-LCMS m/z 511.3 [M+H]⁺.

### Step 3: O-tert-Butyl N-[4-(1r,4r)[[4-[4-[4-[(2-chlorophenyl)sulfonylamino]-2-fluorophenyl]sulfanyl-3-pyridyl]pyrimidin-2-yl]amino]cyclohexyl] carbamate

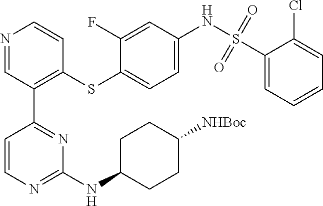 [SMILES: CC(C)(C)OC(=O)N[C@H]1CC[C@H](Nc2nccc(-c3cnccc3Sc3ccc(NS(=O)(=O)c4ccccc4Cl)cc3F)n2)CC1]

To a solution of O-tert-butyl N-|4-(1r,4r)∥4-|4-(4-amino-2-fluorophenyl)sulfanyl-3-pyridyl]pyrimidin-2-yl]amino]cyclohexyl] carbamate (50 mg, 97.92 μmol, 1 eq) in THF (2 mL) was added pyridine (980.00 mg, 12.39 mmol, 1 mL, 126.53 eq) and 2-chlorobenzenesulfonyl chloride (62.00 mg, 293.76 μmol, 40.05 μL, 3 eq). The mixture was stirred at 25° C. for 12 h. The reaction mixture was quenched by the addition of water (50 mL), extracted with DCM (30 mL×3). The combined organic layers were washed with brine (10 mL), dried over Na₂SO₄, filtered and concentrated under reduced pressure to give a residue which was purified by preparative TLC (PE/EtOAc=1/1, TLC: PE/EtOAc=1/1, R_{f}=0.05) to yield O-tert-butyl N-[4-(1r,4r)[[4-[4-[4-[(2-chlorophenyl)sulfonylamino]-2-fluorophenyl]sulfanyl-3-pyridyl]pyrimidin-2-yl]amino]cyclohexyl] carbamate (40 mg, 52.54 μmol, 53.7% yield, 90% purity) as a white solid. ¹H NMR (400 MHz, DMSO-d₆) δ ppm 11.37 (brs, 1H), 8.77 (s, 1H), 8.38 (d, J=4.2 Hz, 1H), 8.30 (d, J=5.5 Hz, 1H), 8.17 (d, J=7.7 Hz, 1H), 7.70 (d, J=4.1 Hz, 2H), 7.62-7.48 (m, 2H), 7.27 (s, 1H), 7.04 (d, J=9.8 Hz, 3H), 6.80-6.67 (m, 1H), 6.52 (s, 1H), 3.86 (brs, 1H), 3.17 (brd, J=5.1 Hz, 1H), 1.95-1.72 (m, 4H), 1.37 (s, 9H), 1.34-1.21 (m, 4H); ES-LCMS m/z 685.3 [M+H]⁺.

### Step 4: N-[4-(1r,4r)[[3-[2-[(4-Aminocyclohexyl)amino]pyrimidin-4-yl]-4-pyridyl]sulfanyl]-3-fluoro-phenyl] 2-chlorobenzenesulfonamide

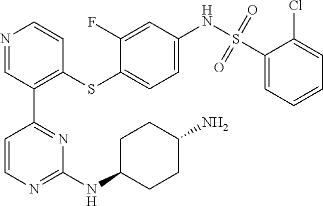 [SMILES: N[C@H]1CC[C@H](Nc2nccc(-c3cnccc3Sc3ccc(NS(=O)(=O)c4ccccc4Cl)cc3F)n2)CC1]

To a solution of O-tert-butyl N-[4-[[4-[4-[4-[(2-chlorophenyl)sulfonylamino]-2-fluorophenyl]sulfanyl-3-pyridyl]pyrimidin-2-yl]amino]cyclohexyl] carbamate (40 mg, 52.54 μmol, 1 eq) in DCM (1.5 mL) was added TFA (690.75 mg, 6.06 mmol, 450.00 μL, 115.31 eq). The mixture was stirred at 20° C. for 30 min. The reaction mixture was concentrated to yield a residue which was purified by preparative HPLC (column: Boston Green ODS 150*30 mm*5 μm; mobile phase: [water (FA)-ACN]; gradient: 11%-41%, 10 min), followed by lyophilization to yield N-[4-(1r,4r)[[3-[2-[(4-aminocyclohexyl)amino]pyrimidin-4-yl]-4-pyridyl]sulfanyl]-3-fluorophenyl] 2-chlorobenzenesulfonamide (22.2 mg, 37.75 μmol, 71.7% yield, 99.5%, 0.70FA) as a white solid. ¹H NMR (400 MHz, DMSO-d₆, 75° C.) δ ppm 8.766 (s, 1H), 8.39 (d, J=5.0 Hz, 1H), 8.30 (d, J=5.5 Hz, 1H), 8.19 (s, 0.70H, HCO₂H), 8.03 (dd, J=7.2, 2.2 Hz, 1H), 7.47-7.34 (m, 3H), 7.13 (t, J=8.6 Hz, 1H), 7.00 (d, J=5.1 Hz, 1H), 6.98-6.94 (m, 1H) 6.82-6.74 (m, 2H), 6.69 (d, J=5.4 Hz), 3.89 (brs, 1H), 2.99 (brs, 1H), 2.10-1.90 (m, 4H), 1.348-1.35 (m, 4H); ES-LCMS m/z 585.3 [M+H]⁺.
117. N-[4-(1r,4r)[[3-[2-[(4-aminocyclohexyl)amino]pyrimidin-4-yl]-4-pyridyl]sulfanyl]-3-fluorophenyl] benzenesulfonamide

### tert-Butyl N-[4-(1r,4r)[[4-[4-[4-(benzenesulfonamido)-2-fluoro-phenyl]sulfanyl-3-pyridyl]pyrimidin-2-yl]amino]cyclohexyl]carbamate

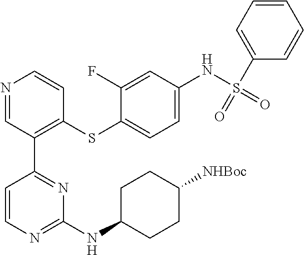 [SMILES: CC(C)(C)OC(=O)N[C@H]1CC[C@H](Nc2nccc(-c3cnccc3Sc3ccc(NS(=O)(=O)c4ccccc4)cc3F)n2)CC1]

To a solution of O-tert-butyl N-[4-(1r,4r)[[4-[4-(4-amino-2-fluorophenyl)sulfanyl-3-pyridyl]pyrimidin-2-yl]amino]cyclohexyl] carbamate (50 mg, 88.13 μmol, 1 eq) in THF (2 mL) was added pyridine (69.71 mg, 881.27 μmol, 71.13 μL, 10 eq) and benzenesulfonyl chloride (46.70 mg, 264.38 μmol, 33.74 μL, 3 eq). The mixture was stirred at 25° C. for 12 h. The reaction mixture was quenched by the addition of water (50 mL), extracted with DCM (30 mL×3). The combined organic layers were washed with brine (10 mL), dried over Na₂SO₄, filtered and concentrated under reduced pressure to give a residue which was purified by preparative TLC (PE/EtOAc=1/1, TLC: PE/EtOAc=1/1, R_{f}=0.05) to yield O-tert-butyl N-[4-(1r,4r)[[4-[4-[4-(benzenesulfonamido)-2-fluorophenyl]sulfanyl-3-pyridyl]pyrimidin-2-yl]amino]cyclohexyl] carbamate (40 mg, 61.10 μmol, 69.3% yield, 99.4%) as a white solid. ¹H NMR (400 MHz, DMSO-d₆) δ ppm 8.76 (s, 1H), 8.38 (d, J=4.3 Hz, 1H), 8.30 (d, J=5.5 Hz, 1H), 7.85 (d, J=7.4 Hz, 2H), 7.67-7.56 (m, 3H), 7.47 (s, 1H), 7.26 (s, 1H), 7.06-6.96 (m, 3H), 6.73 (d, J=8.2 Hz, 1H), 6.54 (s, 1H), 3.86 (brs, 1H), 3.17 (d, J=4.1 Hz, 1H), 1.97-1.77 (m, 4H), 1.37 (s, 9H), 1.34-1.21 (m, 4H); ES-LCMS m/z 651.3 [M+H]⁺.

### N-[4-(1r,4r)[[3-[2-[(4-Aminocyclohexyl)amino]pyrimidin-4-yl]-4-pyridyl]sulfanyl]-3-fluoro-phenyl]benzenesulfonamide

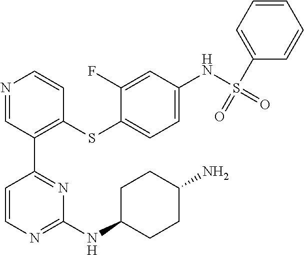 [SMILES: N[C@H]1CC[C@H](Nc2nccc(-c3cnccc3Sc3ccc(NS(=O)(=O)c4ccccc4)cc3F)n2)CC1]

To a solution of O-tert-butyl N-[4-(1r,4r)[[4-[4-[4-(benzenesulfonamido)-2-fluorophenyl]sulfanyl-3-pyridyl]pyrimidin-2-yl]amino]cyclohexyl] carbamate (40 mg, 61.10 μmol, 1 eq) in DCM (1.5 mL) was added TFA (877.14 mg, 7.69 mmol, 571.43 μL, 125.91 eq). The mixture was stirred at 20° C. for 30 min. The reaction mixture was concentrated to yield a residue which was purified by preparative HPLC (column: Boston Green ODS 150*30 mm*5 μm; mobile phase: [water (FA)-ACN]; gradient: 12%-42%, 10 min), followed by lyophilization to yield N-[4-(1r,4r)[[3-[2-[(4-aminocyclohexyl)amino]pyrimidin-4-yl]-4-pyridyl]sulfanyl]-3-fluoro-phenyl]benzenesulfonamide (29.7 mg, 53.93 μmol, 88.3% yield, 100% pure) as a white solid. ¹H NMR (400 MHz, DMSO-d₆, 75° C.) δ ppm 8.68 (s, 1H), 8.40 (d, J=4.9 Hz, 1H), 8.31 (d, J=5.6 Hz, 1H), 8.17 (s, 0.45H, HCO₂H), 7.83-7.79 (m, 2H), 7.55-7.47 (m, 3H), 7.26 (t, J=8.6 Hz, 1H), 7.00 (d, J=5 Hz, 1H), 6.99-6.86 (m, 3H), 6.65 (d, J=5.5 Hz, 1H), 3.93-3.81 (m, 1H), 3.04-2.96 (m, 1H), 2.12-1.92 (m, 4H), 1.48-1.36 (m, 4H); ES-LCMS m/z 551.3 [M+H]⁺.

The following compound has been made by processes similar to those described immediately above using Method D.
118. N-[4-(1r,4r)[[3-[2-[(4-Aminocyclohexyl)amino]pyrimidin-4-yl]-4-pyridyl]sulfanyl]-3-fluorophenyl]naphthalene-1-sulfonamide. ES-LCMS m/z 601.3 [M+H]⁺.

### Example 6. Syntheses of Compounds 119-122

The following compounds were made using Method E.
119. N-[Oxetan-3-ylamino]pyrimidin-4-yl]-1-phenyl]sulfanyl]-3-fluoro-phenyl] 2-chlorobenzenesulfonamide.

### Step 1: (2-Bromophenyl)(4-methoxybenzyl)sulfane

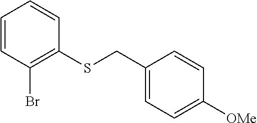 [SMILES: COc1ccc(CSc2ccccc2Br)cc1]

To a solution of 2-bromobenzenethiol (2.0 g, 10.6 mmol) in DMF (22 mL) were added 4-methoxybenzyl chloride (1.83 g, 11.7 mmol, 1.1 eq) and potassium carbonate (2.93 g, 21.2 mmol, 2 eq), and the reaction mixture was heated at 40° C. for 4 h. The reaction mixture was diluted with water to give a white precipitate, which was filtered and dried to afford the title compound (3.23 g, 10.5 mmol, 99% yield). ¹H NMR (500 MHz, Chloroform-d) δ 7.54 (d, J=8.4 Hz, 1H), 7.28 (d, J=8.5 Hz, 2H), 7.22 (dd, J=6.4, 1.3 Hz, 2H), 7.02 (ddd, J=8.5, 6.6, 2.4 Hz, 1H), 6.84 (d, J=8.6 Hz, 2H), 4.11 (s, 2H), 3.79 (s, 3H).

### Step 2: 2-(2-((4-Methoxybenzyl)thio)phenyl)-4,4,5,5-tetramethyl-1,3,2-dioxaborolane

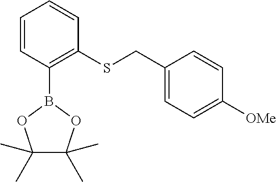 [SMILES: COc1ccc(CSc2ccccc2B2OC(C)(C)C(C)(C)O2)cc1]

To a solution of (2-bromophenyl)(4-methoxybenzyl)sulfane (2.46 g, 7.99 mmol) in dioxane (80 mL) were added KOAc (2.35 g, 24.0 mmol, 3 eq), bis(pinacolato)diboron (6.09 g, 24.0 mmol, 3 eq) and Pd(dppf)Cl₂ (584 mg, 0.799 mmol, 0.1 eq). The reaction mixture was stirred at 100° C. for 2 h under N₂. The resulting mixture was filtered through a pad of celite and the pad was washed with EtOAc (100 mL). The filtrate was concentrated under reduced pressure to give an oily residue which was dissolved in DCM (10 mL) and then diluted with Hexanes (100 mL) to give a precipitate. The precipitate was then filtered and dried to afford the title compound (2.0 g, 5.62 mmol, 70% yield); ¹H NMR (500 MHz, DMSO-d₆) δ 7.51 (d, J=6.4 Hz, 1H), 7.37-7.29 (m, 2H), 7.26 (d, J=8.6 Hz, 2H), 7.11 (t, J=7.2 Hz, 1H), 6.85 (d, J=8.6 Hz, 2H), 4.11 (s, 2H), 3.70 (s, 3H), 1.27 (s, 12H); ESI-MS m/z 379 [M+Na]⁺.

### Step 3: 2-Chloro-4-(2-((4-methoxybenzyl)thio)phenyl)pyrimidine

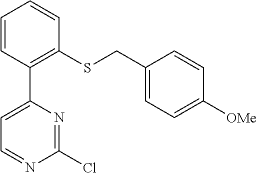 [SMILES: COc1ccc(CSc2ccccc2-c2ccnc(Cl)n2)cc1]

To a solution of 2-(2-((4-methoxybenzyl)thio)phenyl)-4,4,5,5-tetramethyl-1,3,2-dioxaborolane (2.53 g, 7.09 mmol) in dioxane (50 mL) and H₂O (10 mL) were added Cs₂CO₃ (4.62 g, 14.2 mmol, 2 eq), 2,4-dichloropyrimidine (1.16 g, 7.80 mmol, 1.1 eq) and Pd(dppf)Cl₂ (520 mg, 0.71 mmol, 0.1 eq). The reaction mixture was stirred at 100° C. for 2 h under N₂. The resulting mixture was diluted with H₂O (100 mL) and extracted with EtOAc (100 mL×2). The combined organic layer was washed with brine (100 mL), dried over Na₂SO₄, filtered and concentrated under reduced pressure to give a crude oil, which was purified by silica gel flash column chromatography (10% to 20% EtOAc in Hexanes) to afford the title compound (1.73 g, 5.06 mmol, 71% yield) as a yellow solid; ¹H NMR (500 MHz, Chloroform-d) δ 8.58 (d, J=4.2 Hz, 1H), 7.52 (dd, J=27.0, 7.1 Hz, 3H), 7.39 (d, J=7.1 Hz, 1H), 7.34 (d, J=7.1 Hz, 1H), 7.07 (d, J=7.4 Hz, 2H), 6.77 (d, J=7.5 Hz, 2H), 3.98 (s, 2H), 3.78 (s, 3H); ESI-MS m/z 343 [M+H]⁺.

### Step 4: 2-Chloro-4-(2-((2-fluoro-4-nitrophenyl)thio)phenyl)pyrimidine

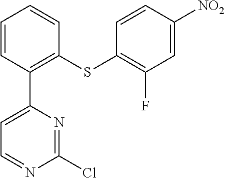 [SMILES: O=[N+]([O-])c1ccc(Sc2ccccc2-c2ccnc(Cl)n2)c(F)c1]

To a solution of 2-chloro-4-(2-((4-methoxybenzyl)thio)phenyl)pyrimidine (610 mg, 1.78 mmol) in TFA (7 mL) were added anisole (0.98 mL, 8.92 mmol, 5 eq) and TfOH (0.79 mL, 8.92 mmol, 5 eq) at 0° C. The reaction mixture was stirred at 0° C. for 10 min, poured into an icy solution of 30% NaOH (7 mL) and sat. NaHCO₃ (100 mL), and then extracted with EtOAc (100 mL). The organic layer was washed with brine, dried, and concentrated to give a yellow sticky oil, which was dissolved in DMF. 3,4-Difluoronitrobenzene (313 mg, 1.96 mmol, 1.1 eq) and K₂CO₃ (490 mg, 3.56 mmol, 2 eq) were added and the reaction mixture was heated at 60° C. for 10 min. The resulting mixture was cooled, diluted with water (100 mL), and extracted with EtOAc (100 mL×2). The combined organic layer was washed with water (100 mL), brine (100 mL), dried over Na₂SO₄, filtered and concentrated under reduced pressure to give a crude oil, which was purified by silica gel flash column chromatography (10% to 30% EtOAc in Hexanes) to afford the title compound (333 mg, 0.922 mmol, 52% yield) as a yellow solid; ¹H NMR (500 MHz, Chloroform-d) δ 8.64 (d, J=5.1 Hz, 1H), 7.88 (d, J=8.4 Hz, 2H), 7.76-7.69 (m, 1H), 7.63-7.55 (m, 2H), 7.57-7.50 (m, 2H), 7.07-7.00 (m, 1H); ESI-MS m/z 362 [M+H]⁺.

### Step 5: 4-((2-(2-Chloropyrimidin-4-yl)phenyl)thio)-3-fluoroaniline

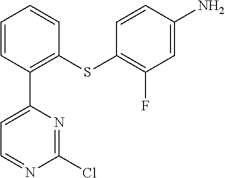 [SMILES: Nc1ccc(Sc2ccccc2-c2ccnc(Cl)n2)c(F)c1]

To a solution of 2-chloro-4-(2-((2-fluoro-4-nitrophenyl)thio)phenyl)pyrimidine (633 mg, 1.75 mmol) in THF (30 mL) was added water (30 mL), and then zinc (687 mg, 10.5 mmol, 6 eq) and ammonium chloride (562 mg, 10.5 mmol, 6 eq) were added. The reaction mixture was stirred at room temperature for 1 h. The same amounts of zinc and ammonium chloride were added and stirred for an additional hour. The resulting mixture was diluted with water (60 mL) and extracted with EtOAc (80 mL×2). The combined organic layers were washed with brine (80 mL), dried over Na₂SO₄, filtered and concentrated under reduced pressure to yield the title compound (630 mg, 90% purity) which was used for the next reaction without purification; ¹H NMR (500 MHz, DMSO-d₆) δ 8.87 (d, J=5.2 Hz, 1H), 7.93 (d, J=5.2 Hz, 1H), 7.64 (d, J=7.7 Hz, 1H), 7.42-7.34 (m, 1H), 7.28 (t, J=7.5 Hz, 1H), 7.16 (t, J=8.6 Hz, 1H), 6.90 (d, J=8.0 Hz, 1H), 6.49-6.35 (m, 2H), 5.90 (s, 2H); ESI-MS m/z 332 └M+H┘⁺.

### Step 6: 4-(2-((4-Amino-2-fluorophenyl)thio)phenyl)-N-(oxetan-3-yl)pyrimidin-2-amine

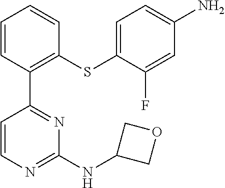 [SMILES: Nc1ccc(Sc2ccccc2-c2ccnc(NC3COC3)n2)c(F)c1]

To a solution of 4-((2-(2-chloropyrimidin-4-yl)phenyl)thio)-3-fluoroaniline (211 mg, 0.637 mmol) in DMSO (2 mL) was added 3-aminooxetane (93 mg, 1.27 mmol, 2 eq) and TEA (0.27 mL, 1.91 mmol, 3 eq). The reaction mixture was heated at 110° C. for 5 h. The resulting mixture was diluted with H₂O (20 mL) and extracted with EtOAc (40 mL×2). The combined organic layers were washed with water (80 mL) and brine (40 mL), dried over Na₂SO₄, filtered and concentrated under reduced pressure to give the title compound (229 mg, 80% purity) which was used for the next reaction without purification; ESI-MS m/z 369 [M+H]⁺.

### Step 7: N-(3-Fluoro-4-((2-(2-(oxetan-3-ylamino)pyrimidin-4-yl)phenyl)thio)phenyl) 2-chlorobenzenesulfonamide

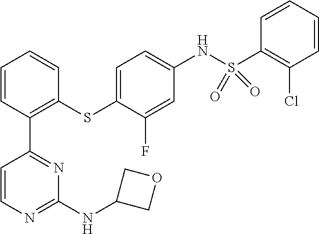 [SMILES: O=S(=O)(Nc1ccc(Sc2ccccc2-c2ccnc(NC3COC3)n2)c(F)c1)c1ccccc1Cl]

To a stirred solution of 4-(2-((4-amino-2-fluorophenyl)thio)phenyl)-N-(oxetan-3-yl)pyrimidin-2-amine (115 mg, 0.313 mmol) and 2-chlorobenzenesulfonyl chloride (86 mg, 0.406 mmol, 1.3 eq) in DCM (3 mL) was added pyridine (76 μL, 0.939 mmol, 3 eq). The reaction mixture was stirred at room temperature overnight. The resulting mixture was diluted with water (20 mL) and extracted with DCM (30 mL×2). The combined organic layers were washed with brine (30 mL), dried over Na₂SO₄, filtered and concentrated under reduced pressure to give a residue which was purified by silica gel flash column chromatography (20% to 70% EtOAc in Hex) to afford the title compound (70 mg, 0.129 mmol, 51% yield) as a yellow solid; ¹H NMR (500 MHz, Methanol-d₄) δ 8.28 (d, J=5.1 Hz, 1H), 8.18-8.09 (m, 1H), 7.64-7.55 (m, 2H), 7.53-7.45 (m, 2H), 7.30-7.18 (m, 3H), 6.93 (tdd, J=8.1, 6.7, 1.7 Hz, 3H), 6.86 (d, J=5.1 Hz, 1H), 5.17-5.10 (m, 1H), 4.94 (t, J=7.0 Hz, 2H), 4.66 (t, J=6.5 Hz, 2H); ESI-MS m/z 543.1 [M+H]⁺.

The following compounds have been made by processes similar to those described immediately above using Method E.
120. N-[4-(1r,4r)[[2-[2-[(4-Aminocyclohexyl)amino]pyrimidin-4-yl]-1-phenyl]sulfanyl]-3-fluoro-phenyl] 2-chlorobenzenesulfonamide. ES-LCMS m/z 584.1 [M+H]⁺
121. N—[(S)[[2-[2-[(Piperidin-3-yl)amino]pyrimidin-4-yl]-1-phenyl]sulfanyl]-3-fluoro-phenyl] 2-chlorobenzenesulfonamide. ES-LCMS m/z 570.1.1 [M+H]⁺
122. N-[Oxetan-3-ylamino]pyrimidin-4-yl]-1-phenyl]sulfanyl]-3-fluoro-phenyl] 2-ethynylbenzenesulfonamide. ES-LCMS m/z 533.1 [M+H]⁺

### Example 7. Syntheses of Compounds 123-125

The following compounds were made using Method F.
123. N-[4-[[3-[2-[(1r,4r)(4-aminocyclohexyl)amino]pyrimidin-4-yl]-2-pyridyl]amino]-3-fluorophenyl] 2-chlorobenzenesulfonamide

### Step 1: 3-(2-Chloropyrimidin-4-yl)pyridin-2-ylamine

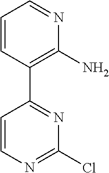 [SMILES: Nc1ncccc1-c1ccnc(Cl)n1]

To a solution of 2,4-dichloropyrimidine (100 mg, 671.24 μmol, 1 eq) in THF (2 mL) and H₂O (0.4 mL) was added Pd(dppf)Cl₂ (49.12 mg, 67.12 μmol, 0.1 eq) and Cs₂CO₃ (437.41 mg, 1.34 mmol, 2 eq) and 3-(4,4,5,5-tetramethyl-1,3,2-dioxaborolan-2-yl)pyridin-2-amine (147.72 mg, 671.24 μmol, 1 eq). The mixture was stirred at 60° C. for 12 h under N₂. The reaction mixture was quenched by addition of water (50 mL), extracted with EtOAc (30 mL×3). The combined organic layers were washed with brine (10 mL), dried over Na₂SO₄, filtered and concentrated under reduced pressure to yield a residue which was purified by flash silica gel chromatography (from PE/EtOAc=1/0 to 3/1, TLC: PE/EtOAc=3/1, R_{f}=0.29) to yield 3-(2-chloropyrimidin-4-yl)pyridin-2-ylamine (120 mg, 522.67 μmol, 77.9% yield, 90.0% purity) as a yellow solid. ES-LCMS m/z 207.1, 209.0 [M+H]⁺.

### Step 2: N-(2-Fluoro-4-nitrophenyl) 3-(2-chloropyrimidin-4-yl)pyridin-2-ylamine

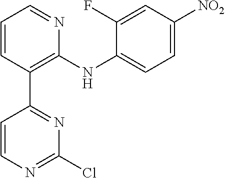 [SMILES: O=[N+]([O-])c1ccc(Nc2ncccc2-c2ccnc(Cl)n2)c(F)c1]

To a solution of 3-(2-chloropyrimidin-4-yl)pyridin-2-ylamine (400 mg, 1.94 mmol, 1 eq) in THF (10 mL) was added NaH (774.25 mg, 19.36 mmol, 60.0%, 10 eq) stirred at 0° C. for 0.5 h and then 1,2-difluoro-4-nitro-benzene (338.77 mg, 2.13 mmol, 235.25 μL, 1.1 eq) was added. The mixture was stirred at 20° C. for 1.5 h. To the mixture was added water (30 mL), filtered and the filter cake was collected to yield the compound and concentrated under reduced pressure to yield N-(2-fluoro-4-nitrophenyl) 3-(2-chloropyrimidin-4-yl)pyridin-2-ylamine (400 mg, 1.04 mmol, 53.8% yield, 90.0% purity) as a yellow solid which was used in the next step without further purification. ¹H NMR (400 MHz, DMSO-d₆) δ ppm 12.06 (s, 1H), 9.05-8.76 (m, 2H), 8.61 (d, J=7.6 Hz, 1H), 8.52 (s, 1H), 8.47-8.33 (m, 1H), 8.23-8.06 (m, 2H), 7.19 (s, 1H); ES-LCMS m/z 346.1, 348.1 [M+H]⁺.

### Step 3: O-tert-Butyl N-[4-[(1r,4r)[4-[2-(2-fluoro-4-nitroanilino)-3-pyridyl]pyrimidin-2-yl]amino]cyclohexyl] carbamate

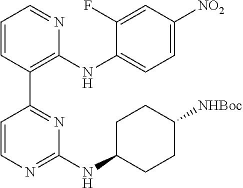 [SMILES: CC(C)(C)OC(=O)N[C@H]1CC[C@H](Nc2nccc(-c3cccnc3Nc3ccc([N+](=O)[O-])cc3F)n2)CC1]

To a solution of N-(2-fluoro-4-nitrophenyl) 3-(2-chloropyrimidin-4-yl)pyridin-2-ylamine (180 mg, 468.59 μmol, 90.0%, 1 eq) in DMSO (3 mL) was added TEA (142.25 mg, 1.41 mmol, 195.67 μL, 3 eq) and O-tert-butyl N-(4-aminocyclohexyl)carbamate (100.42 mg, 468.59 μmol, 1 eq). The mixture was stirred at 80° C. for 12 h. The reaction mixture was filtered and the filtrate was concentrated to yield a residue, and the filter cake was collected, washed by MeOH, and concentrated under reduced pressure to yield O-tert-butyl N-[4-[(1r,4r)[4-[2-(2-fluoro-4-nitroanilino)-3-pyridyl]pyrimidin-2-yl]amino]cyclohexyl] carbamate (200 mg, 382.00 μmol, 81.5% yield, 100.0% purity) as a yellow solid which was used in the next step without further purification. ¹H NMR (400 MHz, DMSO-d₆) δ ppm 12.52 (s, 1H), 11.34 (s, 1H), 8.89 (s, 1H), 8.48-8.40 (m, 2H), 8.35 (s, 1H), 8.24-8.14 (m, 2H), 7.16 (dd, J=4.8, 7.6 Hz, 1H), 6.74 (d, J=7.6 Hz, 1H), 3.77 (brs, 1H), 3.23 (brs, 1H), 1.99-1.77 (m, 4H), 1.38 (s, 9H), 1.36-1.20 (m, 4H); ES-LCMS m/z 524.3 [M+H]⁺.

### Step 4: O-tert-Butyl N-[4-[(1r,4r)[4-[2-(4-amino2-fluoroanilino)-3-pyridyl]pyrimidin-2-yl]amino]cyclohexyl] carbamate

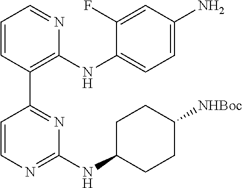 [SMILES: CC(C)(C)OC(=O)N[C@H]1CC[C@H](Nc2nccc(-c3cccnc3Nc3ccc(N)cc3F)n2)CC1]

To a solution of O-tert-butyl N-[4-[(1r,4r)[4-[2-(2-fluoro-4-nitroanilino)-3-pyridyl]pyrimidin-2-yl]amino]cyclohexyl] carbamate (100 mg, 171.90 μmol, 90.0%, 1 eq) in EtOH (2.5 mL) and H₂O (0.5 mL) was added Fe (76.80 mg, 1.38 mmol, 8 eq) and NH₄Cl (73.56 mg, 1.38 mmol, 8 eq). The mixture was stirred at 90° C. for 4 h. The reaction mixture was filtered and the filtrate was concentrated to yield O-tert-butyl N-[4-[(1r,4r)[4-[2-(4-amino-2-fluoroanilino)-3-pyridyl]pyrimidin-2-yl]amino]cyclohexyl] carbamate (80 mg, crude) as a yellow solid which was used in the next step without further purification. ¹H NMR (400 MHz, CD₃OD) δ ppm 8.33 (d, J=4.4 Hz, 2H), 8.05 (d, J=4.4 Hz, 1H), 7.40 (s, 4H), 7.16 (d, J=5.6 Hz, 1H), 6.87-6.79 (m, 1H), 6.64-6.53 (m, 2H), 3.75 (s, 1H), 2.10-1.86 (m, 5H), 1.44 (s, 9H), 1.38 (s, 4H); ES-LCMS m/z 494.3 [M+H]⁺.

### Step 5: O-tert-Butyl N-[4-[(1r,4r)[4-[2-(4-(2-chlorobenzenesulfonamido)-2-fluoroanilino)-3-pyridyl]pyrimidin-2-yl]amino]cyclohexyl] carbamate

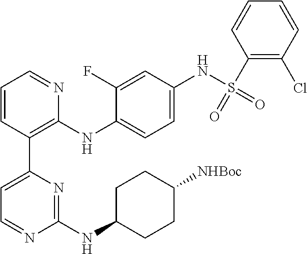 [SMILES: CC(C)(C)OC(=O)N[C@H]1CC[C@H](Nc2nccc(-c3cccnc3Nc3ccc(NS(=O)(=O)c4ccccc4Cl)cc3F)n2)CC1]

To a solution of O-tert-butyl N-[4-[(1r,4r)[4-[2-(4-amino-2-fluoroanilino)-3-pyridyl]pyrimidin-2-yl]amino]cyclohexyl] carbamate (80 mg, 145.87 μmol, 90.0%, 1 eq) in THF (2 mL) was added pyridine (92.31 mg, 1.17 mmol, 94.19 μL, 8 eq) and 2-chlorobenzenesulfonyl chloride (30.79 mg, 145.87 μmol, 19.86 μL, 1 eq). The mixture was stirred at 60° C. for 2 h. The mixture was diluted with water (15 mL) and extracted with EtOAc (8 mL×3). The combined organic layers were washed with brine (15 mL), dried over Na₂SO₄, filtered and concentrated under reduced pressure to yield a residue which was purified by preparative TLC (PE/EtOAc=1/1, TLC: PE/EtOAc=1/1, R_{f}=0.24) to yield O-tert-butyl N-[4-[(1r,4r)[4-[2-(4-(2-chlorobenzenesulfonamido)-2-fluoroanilino)-3-pyridyl]pyrimidin-2-yl]amino]cyclohexyl]carbamate (40 mg, 59.86 μmol, 41.0% yield, 100.0% purity) as yellow oil. ¹H NMR (400 MHz, CDCl₃) δ ppm 12.40 (s, 1H), 8.78-8.50 (m, 1H), 8.34 (d, J=5.4 Hz, 1H), 8.27 (dd, J=1.6, 4.8 Hz, 1H), 7.99 (dd, J=1.6, 7.8 Hz, 2H), 7.56-7.50 (m, 1H), 7.50-7.41 (m, 1H), 7.37-7.28 (m, 2H), 7.09 (dd, J=1.6, 12.6 Hz, 1H), 6.92 (d, J=4.8 Hz, 1H), 6.87-6.79 (m, 2H), 4.49 (d, J=7.5 Hz, 1H), 3.93-3.82 (m, 1H), 2.20-2.05 (m, 4H), 1.47 (s, 9H), 1.40-1.33 (m, 4H); ES-LCMS m/z 668.2 [M+H]⁺.

### Step 6: N-[4-[[3-[2-[(1r,4r)(4-aminocyclohexyl)amino]pyrimidin-4-yl]-2-pyridyl]amino]-3-fluorophenyl] 2-chlorobenzenesulfonamide

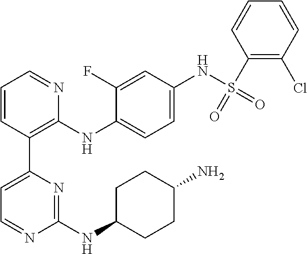 [SMILES: N[C@H]1CC[C@H](Nc2nccc(-c3cccnc3Nc3ccc(NS(=O)(=O)c4ccccc4Cl)cc3F)n2)CC1]

To a solution of O-tert-butyl N-[4-[(1r,4r)[4-[2-(4-(2-chlorobenzenesulfonamido)-2-fluoroanilino)-3-pyridyl]pyrimidin-2-yl]amino]cyclohexyl] carbamate (40 mg, 59.86 μmol, 100.0%, 1 eq) in DCM (1 mL) was added HCl/dioxane (4 M, 0.5 mL, 33.41 eq). The mixture was stirred at 20° C. for 0.5 h. Adjust the pH with NH₃·H₂O to 7, the reaction mixture was concentrated to yield a residue which was purified by preparative HPLC (column: C18-1 150*30 mm*5 μm; mobile phase: [water (NH₃H₂O+NH₄HCO₃)-ACN]; B %: 27%-57%, 15 min), followed by lyophilization to yield N-[4-[[3-[2-[(4-aminocyclohexyl)amino]pyrimidin-4-yl]-2-pyridyl]amino]-3-fluoro-phenyl]-2-chlorobenzenesulfonamide (7.19 mg, 12.66 μmol, 21.1% yield, 100.0% purity) as a yellow solid. ¹H NMR (400 MHz, DMSO-d₆, 75° C.) δ ppm 10.94 (brs, 1H), 8.37 (d, J=5.4 Hz, 1H), 8.19-8.13 (m, 2H), 8.03 (dd, J=1.7, 7.8 Hz, 1H), 7.73 (brt, J=9.5 Hz, 1H), 7.60-7.40 (m, 3H), 7.11 (d, J=5.4 Hz, 1H), 6.91-6.74 (m, 3H), 3.79-3.69 (m, 1H), 2.83-2.71 (m, 1H), 2.04-1.80 (m, 4H), 1.43-1.12 (m, 4H); ES-LCMS m/z 568.2, 570.1 [M+H]⁺.
124. N-[3-Fluoro-4-[[3-[2-[[(3S)-3-piperidyl]amino]pyrimidin-4-yl]-2-pyridyl]amino]phenyl] 2-chlorobenzenesulfonamide

### Step 1: O-tert-Butyl N-[(3S)-3-[[4-[2-(2-fluoro-4-nitroanilino)-3-pyridyl]pyrimidin-2-yl]amino]piperidin-1-yl] carbamate

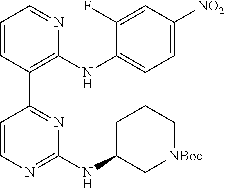 [SMILES: CC(C)(C)OC(=O)N1CCC[C@H](Nc2nccc(-c3cccnc3Nc3ccc([N+](=O)[O-])cc3F)n2)C1]

To a solution of 3-(2-chloropyrimidin-4-yl)-N-(2-fluoro-4-nitro-phenyl)pyridin-2-amine (200 mg, 520.66 μmol, 90.0%, 1 eq) in DMSO (2 mL) was added TEA (158.06 mg, 1.56 mmol, 217.41 μL, 3 eq) and O-tert-butyl N-[(3S)-3-aminopiperidin-1-yl] carbamate (125.13 mg, 624.79 μmol, 1.2 eq). The mixture was stirred at 80° C. for 2 h. The reaction mixture was filtered and the filtrate was concentrated to yield a residue which was concentrated to yield O-tert-butyl N-[(3S)-3-[[4-[2-(2-fluoro-4-nitroanilino)-3-pyridyl]pyrimidin-2-yl]amino]piperidin-1-yl] carbamate (200 mg, crude) as a yellow solid. ¹H NMR (400 MHz, CD₃OD) δ ppm 9.16-8.97 (m, 1H), 8.54-8.39 (m, 2H), 8.29 (s, 1H), 8.10 (d, J=10.6 Hz, 2H), 7.29-7.03 (m, 2H), 3.98 (d, J=10.4 Hz, 2H), 3.84 (d, J=13.2 Hz, 2H), 2.79 (s, 1H), 1.95 (d, J=9.2 Hz, 2H), 1.70 (d, J=13.6 Hz, 2H), 1.29 (s, 9H); ES-LCMS m/z 510.3 └M+H┘⁺.

### Step 2: O-tert-Butyl N-[(3S)-3-[[4-[2-(4-amino-2-fluoroanilino)-3-pyridyl]pyrimidin-2-yl]amino]piperidin-1-yl] carbamate

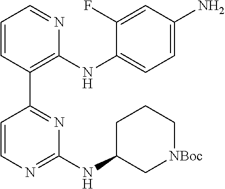 [SMILES: CC(C)(C)OC(=O)N1CCC[C@H](Nc2nccc(-c3cccnc3Nc3ccc(N)cc3F)n2)C1]

To a solution of O-tert-butyl N-[(3S)-3-[[4-[2-(2-fluoro-4-nitroanilino)-3-pyridyl]pyrimidin-2-yl]amino]piperidin-1-yl] carbamate (200 mg, 294.39 μmol, 75.0%, 1 eq) in EtOH (2.5 mL) and H₂O (0.5 mL) was added Fe (131.52 mg, 2.36 mmol, 8 eq) NH₄Cl (125.98 mg, 2.36 mmol, 8 eq). The mixture was stirred at 90° C. for 2 h. The reaction mixture was filtered and the filtrate was concentrated to yield a residue which was concentrated to yield O-tert-butyl N-[(3S)-3-[[4-[2-(4-amino-2-fluoroanilino)-3-pyridyl]pyrimidin-2-yl]amino]piperidin-1-yl] carbamate (150 mg, 250.24 μmol, 85.0% yield, 80.0% purity) as yellow oil. ES-LCMS m/z 480.2 └M+H┘⁺.

### Step 3: O-tert-Butyl N-[(3S)-3-[[4-[2-(4-(2-chlorobenzenesulfonamido)-2-fluoroanilino)-3-pyridyl]pyrimidin-2-yl]amino]piperidin-1-yl] carbamate

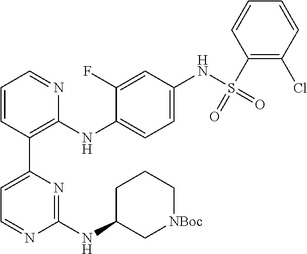 [SMILES: CC(C)(C)OC(=O)N1CCC[C@H](Nc2nccc(-c3cccnc3Nc3ccc(NS(=O)(=O)c4ccccc4Cl)cc3F)n2)C1]

To a solution of O-tert-butyl N-[(3S)-3-[[4-[2-(4-amino-2-fluoroanilino)-3-pyridyl]pyrimidin-2-yl]amino]piperidin-1-yl] carbamate (70 mg, 116.78 μmol, 80.0%, 1 eq) in THF (2 mL) was added pyridine (55.42 mg, 700.66 μmol, 56.55 μL, 6 eq) and 2-chlorobenzenesulfonyl chloride (49.29 mg, 233.55 μmol, 31.80 μL, 2 eq). The mixture was stirred at 60° C. for 2 h. The mixture was diluted with water (20 mL) and extracted with EtOAc (15 mL×3). The combined organic layers were washed with brine (15 mL), dried over Na₂SO₄, filtered and concentrated under reduced pressure to yield a residue which was purified by preparative TLC (PE/EtOAc=0/1, TLC: PE/EtOAc=0/1, R_{f}=0.40) to yield O-tert-butyl N-└(3S)-3-└└4-└2-(4-(2-chlorobenzenesulfonamido)-2-fluoroanilino)-3-pyridyl]pyrimidin-2-yl]amino]piperidinyl-1-yl]carbamate (70 mg, 102.73 μmol, 88.0% yield, 96.0% purity) as yellow oil. ¹H NMR (400 MHz, CDCl₃) δ ppm 8.84-8.50 (m, 1H), 8.38 (d, J=5.2 Hz, 1H), 8.30 (dd, J=2.0, 4.8 Hz, 1H), 8.07-7.94 (m, 2H), 7.55-7.51 (m, 1H), 7.50-7.45 (m, 1H), 7.37-7.31 (m, 1H), 7.11 (d, J=12.8 Hz, 1H), 7.02-6.96 (m, 2H), 6.88-6.79 (m, 2H), 5.31 (s, 1H), 4.19-3.99 (m, 1H), 3.81 (s, 1H), 3.37 (s, 2H), 2.04 (d, J=14.8 Hz, 1H), 1.79 (d, J=7.6 Hz, 3H), 1.52-1.29 (m, 8H); ES-LCMS m/z 654.2, 656.1 [M+H]⁺.

### Step 4: N-[3-Fluoro-4-[[3-[2-[[(3S)-3-piperidyl]amino]pyrimidin-4-yl]-2-pyridyl]amino]phenyl] 2-chlorobenzenesulfonamide

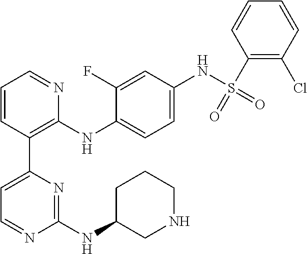 [SMILES: O=S(=O)(Nc1ccc(Nc2ncccc2-c2ccnc(N[C@H]3CCCNC3)n2)c(F)c1)c1ccccc1Cl]

To a solution of O-tert-butyl N-[(3S)-3-[[4-[2-(4-(2-chlorobenzenesulfonamido)-2-fluoroanilino)-3-pyridyl]pyrimidin-2-yl]amino]piperidin-1-yl] carbamate (70 mg, 102.73 μmol, 96.0%, 1 eq) in DCM (1 mL) was added HCl/dioxane (4 M, 0.5 mL, 19.47 eq). The mixture was stirred at 20° C. for 0.5 h. The mixture was adjusted to pH 7 with NH₃·H₂O, and the reaction mixture was concentrated to yield a residue which was purified by preparative HPLC (column: C18-1 150*30 mm*5 μm; mobile phase: [water (CH₃COOH+CH₃COONH₄)-ACN]; B %: 38%-68%, 15 min), followed by lyophilization to yield N-[3-fluoro-4-[[3-[2-[[(3S)-3-piperidyl]amino]pyrimidin-4-yl]-2-pyridyl]amino]phenyl] 2-chlorobenzenesulfonamide (37.82 mg, 68.26 μmol, 66.5% yield, 100.0% purity) as a yellow solid. ¹H NMR (400 MHz, DMSO-d₆, 75° C.) δ ppm 11.10 (brs, 1H), 8.39 (d, J=5.2 Hz, 1H), 8.18 (d, J=6.4 Hz, 2H), 8.05 (dd, J=8, 1.4 Hz, 1H), 7.95 (brt, J=7.5 Hz, 1H), 7.64-7.53 (m, 2H), 7.51-7.44 (m, 1H), 7.15 (d, J=5.6 Hz, 1H), 6.94 (d, J=12.8 Hz, 1H), 6.89-6.75 (m, 3H), 3.95-3.83 (m, 1H), 2.87-2.76 (m, 1H), 2.61-2.51 (m, 3H), 1.96-1.86 (m, 1H), 1.73-1.60 (m, 1H), 1.56-1.32 (m, 2H); ES-LCMS m/z 554.2, 556.1 [M+H]⁺.

The following compound was also made using Method F.
125. N-[4-[[3-[2-[(1r,4r)(4-aminocyclohexyl)amino]pyrimidin-4-yl]-2-pyridyl]amino]-3-fluorophenyl]benzenesulfonamide. ES-LCMS m/z 534.1 [M+H]⁺.

### Example 8. Syntheses of Compound 126

The following compound was made using Method G.
126. N-(4-((2-(2-(((1r,4r)-4-Aminocyclohexyl)amino)pyrimidin-4-yl)phenyl)amino)-3-fluorophenyl) 2-chlorobenzenesulfonamide

### Step 1: 2-Fluoro-4-nitro-N-(2-(4,4,5,5-tetramethyl-1,3,2-dioxaborolan-2-yl)phenyl)aniline

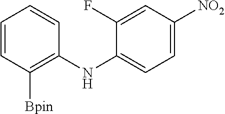

To a solution of 2-(4,4,5,5-tetramethyl-1,3,2-dioxaborolan-2-yl)aniline (1 g, 4.56 mmol, 1 eq) and 1,2-difluoro-4-nitro-benzene (1.45 g, 9.13 mmol, 1.01 mL, 2 eq) in DMA (5 mL) was added K₂CO₃ (1.26 g, 9.13 mmol, 2 eq). The mixture was stirred at 80° C. for 2 h. The reaction mixture was quenched by the addition of water (20 ml), and extracted with EtOAc (20 mL×3). The combined organic layers were washed with brine (20 mL), dried over Na₂SO₄, filtered and concentrated under reduced pressure to yield a residue which was purified by flash silica gel chromatography (from PE/EtOAc=100/1 to 1/3, TLC: PE/EtOAc=1/3, R_{f}=0.25) to yield 2-fluoro-4-nitro-N-(2-(4,4,5,5-tetramethyl-1,3,2-dioxaborolan-2-yl)phenyl)aniline (1 g, 2.74 mmol, 59.9% yield, 98.0% purity) as a yellow solid. ¹H NMR (400 MHz, DMSO-d₆) δ ppm 8.72 (d, J=2.5 Hz, 1H), 8.10 (dd, J=2.6, 11.8 Hz, 1H), 7.96 (dd, J=2.4, 9.2 Hz, 1H), 7.71 (dd, J=1.5, 7.4 Hz, 1H), 7.56 (dt, J=1.6, 7.7 Hz, 1H), 7.45-7.38 (m, 1H), 7.23-7.11 (m, 2H), 1.22-1.20 (m, 12H).

### Step 2: N-(2-(2-chloropyrimidin-4-yl)phenyl)-2-fluoro-4-nitroaniline

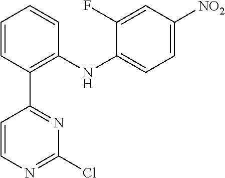 [SMILES: O=[N+]([O-])c1ccc(Nc2ccccc2-c2ccnc(Cl)n2)c(F)c1]

A mixture of 2-fluoro-4-nitro-N-(2-(4,4,5,5-tetramethyl-1,3,2-dioxaborolan-2-yl)phenyl)aniline (1 g, 2.74 mmol, 98.0% purity, 1 eq), 2,4-dichloropyrimidine (448.38 mg, 3.01 mmol, 1.1 eq), Pd(dppf)Cl₂ (200.20 mg, 273.61 μmol, 0.1 eq), Cs₂CO₃ (1.34 g, 4.10 mmol, 1.5 eq) in H₂O (1 mL) and THF (3 mL) was degassed and purged with N₂ for 3 times. The mixture was stirred at 60° C. for 1 h under N₂ atmosphere. The reaction mixture was quenched by the addition of H₂O (20 mL) and extracted with DCM (30 mL×3). The combined organic layers were washed with brine (30 mL), dried over Na₂SO₄, filtered and concentrated under reduced pressure to yield a residue which was purified by flash silica gel chromatography (DCM/EtOAc=5/1, TLC: DCM/EtOAc=5/1, R_{f}=0.3) to yield N-(2-(2-chloropyrimidin-4-yl)phenyl)-2-fluoro-4-nitroaniline (400 mg, 1.16 mmol, 42.4% yield, 100.0% purity) as a yellow solid. ¹H NMR (500 MHz, DMSO-d₆) δ ppm 9.76 (d, J=1.8 Hz, 1H), 8.78 (d, J=5.3 Hz, 1H), 8.11 (dd, J=2.5, 11.7 Hz, 1H), 7.96-7.86 (m, 3H), 7.65-7.55 (m, 2H), 7.43-7.37 (m, 1H), 7.10 (t, J=8.9 Hz, 1H); ES-LCMS m/z 345.2 [M+H]⁺.

### Step 3: O-tert-Butyl ((1r,4r)-4-((4-(2-((2-fluoro-4-nitrophenyl)amino)phenyl)pyrimidin-2-yl)amino)cyclohexyl)carbamate

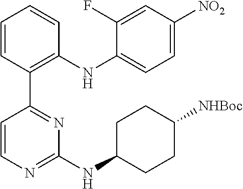 [SMILES: CC(C)(C)OC(=O)N[C@H]1CC[C@H](Nc2nccc(-c3ccccc3Nc3ccc([N+](=O)[O-])cc3F)n2)CC1]

To a solution of N-(2-(2-chloropyrimidin-4-yl)phenyl)-2-fluoro-4-nitroaniline (400 mg, 1.16 mmol, 100.0% purity, 1 eq) and O-tert-butyl N-(1r,4r)(4-aminocyclohexyl)carbamate (298.40 mg, 1.39 mmol, 1.2 eq) in DMSO (3 mL) was added TEA (352 mg, 3.48 mmol, 484.51 μL, 3 eq). The mixture was stirred at 80° C. for 12 h. The reaction mixture was quenched by the addition of H₂O (20 mL) and extracted with DCM (30 mL×3). The combined organic layers were washed with brine (30 mL), dried over Na₂SO₄, filtered and concentrated under reduced pressure to yield a residue which was purified by flash silica gel chromatography (PE/EtOAc=1/1, TLC: PE/EtOAc=1/1, R_{f}=0.3) to yield O-tert-butyl ((1r,4r)-4-((4-(2-((2-fluoro-4-nitrophenyl)amino)phenyl)pyrimidin-2-yl)amino)cyclohexyl)carbamate (300 mg, 574.09 μmol, 49.48% yield, 100.0% purity) as a yellow solid. ¹H NMR (500 MHz, DMSO-d₆) 3 ppm 10.53-9.86 (m, 1H), 8.33 (d, J=5.0 Hz, 1H), 8.11 (dd, J=2.4, 11.6 Hz, 1H), 7.96 (dd, J=2.4, 9.1 Hz, 1H), 7.76 (d, J=7.5 Hz, 1H), 7.51 (d, J=3.8 Hz, 2H), 7.33-6.95 (m, 3H), 6.88 (d, J=5.2 Hz, 1H), 6.70 (d, J=7.5 Hz, 1H), 3.71 (brs, 1H), 3.27-3.15 (m, 1H), 1.90-1.79 (m, 4H), 1.38 (s, 9H), 1.35-1.21 (m, 4H); ES-LCMS m/z 523.3 [M+H]⁺.

### Step 4: O-tert-Butyl ((1r,4r)-4-((4-(2-((4-amino-2-fluorophenyl)amino)phenyl)pyrimidin-2-yl)amino)cyclohexyl)carbamate

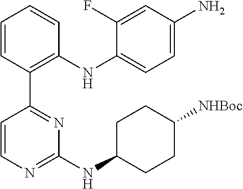 [SMILES: CC(C)(C)OC(=O)N[C@H]1CC[C@H](Nc2nccc(-c3ccccc3Nc3ccc(N)cc3F)n2)CC1]

To a solution of O-tert-butyl ((1r,4r)-4-((4-(2-((2-fluoro-4-nitrophenyl)amino)phenyl)pyrimidin-2-yl)amino)cyclohexyl)carbamate (300 mg, 574.09 μmol, 1 eq) in EtOH (0.5 mL), THF (0.5 mL) and H₂O (0.5 mL) was added NH₄Cl (307.09 mg, 5.74 mmol, 10 eq) and Fe (160.30 mg, 2.87 mmol, 5 eq). The mixture was stirred at 60° C. for 1 h. The reaction mixture was quenched by the addition H₂O (20 mL) and extracted with EtOAc (30 mL×3). The combined organic layers were washed with brine (30 mL), dried over Na₂SO₄, filtered and concentrated under reduced pressure to yield O-tert-butyl ((1 r, 4r)-4-((4-(2-((4-amino-2-fluorophenyl)amino)phenyl)pyrimidin-2-yl)amino)cyclohexyl)carbamate (100 mg, 160.38 μmol, 27.9% yield, 79.0% purity) as a brown oil. ¹H NMR (400 MHz, DMSO-d₆) δ ppm 10.06 (s, 1H), 8.30 (d, J=5.5 Hz, 1H), 7.73 (s, 1H), 7.36-7.08 (m, 2H), 7.09-6.91 (m, 2H), 6.80-6.62 (m, 2H), 6.49-6.40 (m, 2H), 5.33 (s, 2H), 3.63 (brs, 1H), 3.18 (s, 1H), 1.96-1.70 (m, 4H), 1.37 (s, 9H), 1.34-1.19 (m, 4H); ES-LCMS m/z 493.3 [M+H]⁺.

### Step 5: O-tert-Butyl ((1r,4r)-4-((4-(2-((4-((2-chlorophenyl)sulfonamido)-2-fluorophenyl)amino)phenyl)pyrimidin-2-yl)amino)cyclohexyl)carbamate

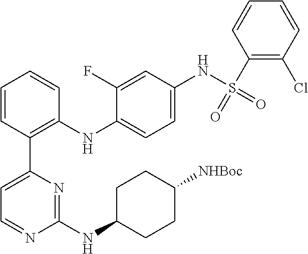 [SMILES: CC(C)(C)OC(=O)N[C@H]1CC[C@H](Nc2nccc(-c3ccccc3Nc3ccc(NS(=O)(=O)c4ccccc4Cl)cc3F)n2)CC1]

A mixture of O-tert-butyl ((1r,4r)-4-((4-(2-((4-amino-2-fluorophenyl)amino)phenyl)pyrimidin-2-yl)amino)cyclohexyl)carbamate (100 mg, 160.38 μmol, 79.0% purity, 1 eq), pyridine (101.49 mg, 1.28 mmol, 103.56 μL, 8 eq), 2-chlorobenzenesulfonyl chloride (67.70 mg, 320.76 μmol, 43.68 μL, 2 eq) in THF (1 mL) was degassed and purged with N₂ for 3 times. The reaction mixture was stirred at 25° C. for 30 min. The reaction mixture was quenched by the addition of H₂O (20 mL) and extracted with EtOAc (30 mL×3). The combined organic layers were washed with brine (30 mL), dried over Na₂SO₄, filtered and concentrated under reduced pressure to yield a residue which was purified by flash silica gel chromatography (PE/EtOAc=1/1, TLC: PE/EtOAc=1/1, R_{f}=0.3) to yield O-tert-butyl ((1r,4r)-4-((4-(2-((4-((2-chlorophenyl)sulfonamido)-2-fluorophenyl)amino)phenyl)pyrimidin-2-yl)amino)cyclohexyl)carbamate (70 mg, 95.47 μmol, 59.53% yield, 91.0% purity) as a yellow oil. ¹H NMR (500 MHz, DMSO-d₆) δ ppm 10.79 (brs, 1H), 8.30 (d, J=5.3 Hz, 1H), 8.07 (s, 1H), 7.85-7.61 (m, 3H), 7.54 (s, 1H), 7.32-7.12 (m, 3H), 7.05-6.72 (m, 5H), 3.70-3.61 (m, 1H), 3.33 (s, 1H), 1.94-1.68 (m, 4H), 1.38 (s, 9H), 1.31-1.21 (m, 4H); ES-LCMS m/z 667.3 [M+H]⁺.

### Step 6: N-(4-((2-(2-(((1r,4r)-4-Aminocyclohexyl)amino)pyrimidin-4-yl)phenyl)amino)-3-fluorophenyl) 2-chlorobenzenesulfonamide

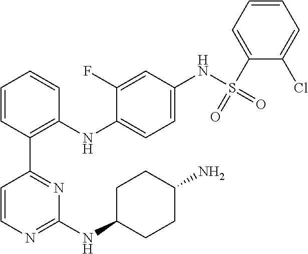 [SMILES: N[C@H]1CC[C@H](Nc2nccc(-c3ccccc3Nc3ccc(NS(=O)(=O)c4ccccc4Cl)cc3F)n2)CC1]

To a solution of O-tert-butyl ((1r,4r)-4-((4-(2-((4-((2-chlorophenyl)sulfonamido)-2-fluorophenyl)amino)phenyl)pyrimidin-2-yl)amino)cyclohexyl)carbamate (70 mg, 95.47 μmol, 91.0% purity, 1 eq) in DCM (3 mL) was added TFA (817.48 mg, 7.17 mmol, 530.83 μL, 75.09 eq). The mixture was stirred at 25° C. for 30 min. The residue was purified by prep-HPLC (column: Boston Prime C18 150*30 mm*5 μm; mobile phase: [water (NH₃H₂O+NH₄HCO₃)-ACN]; B %: 40%-70%, 10 min) to yield N-(4-((2-(2-(((1r,4r)-4-aminocyclohexyl)amino)pyrimidin-4-yl)phenyl)amino)-3-fluorophenyl) 2-chlorobenzenesulfonamide (7.7 mg, 13.31 μmol, 13.94% yield, 98.0% purity) as a yellow solid. ¹H NMR (400 MHz, DMSO-d₆) δ ppm 10.19-9.81 (m, 1H), 8.30 (d, J=5.4 Hz, 1H), 7.96 (dd, J=1.8, 7.4 Hz, 1H), 7.70 (s, 1H), 7.47-7.42 (m, 1H), 7.37 (m, J=5.7, 7.5 Hz, 2H), 7.25 (s, 1H), 7.16 (s, 1H), 7.07-6.88 (m, 2H), 6.73 (d, J=13.9 Hz, 2H), 6.64 (d, J=8.6 Hz, 2H), 3.66 (brs, 1H), 2.89 (s, 1H), 1.99-1.84 (m, 4H), 1.30 (s, 4H); ES-LCMS m/z 567.1 [M+H]⁺.

### Example 9. Syntheses of Compounds 127-130

The following compounds were made using Method H.
127. N-[3-fluoro-4-[5-[2-[[(3S)-3-piperidyl]amino]pyrimidin-4-yl]pyrimidin-4-yl]oxyphenyl] 2-chlorobenzenesulfonamide

### Step 1: O-tert-Butyl N-(3-fluoro-4-hydroxyphenyl) carbamate

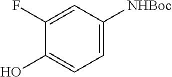 [SMILES: CC(C)(C)OC(=O)Nc1ccc(O)c(F)c1]

To a stirred solution of 4-amino-2-fluorophenol (17 g, 133.74 mmol, 1 eq) in THF (500 mL) were added TEA (40.60 g, 401.21 mmol, 55.84 mL, 3 eq) and Boc₂O (32.11 g, 147.11 mmol, 33.80 mL, 1.1 eq) at 0° C. The reaction mixture was stirred at 25° C. for 2 h under N₂ atmosphere. The reaction mixture was concentrated to yield a residue which was purified by flash silica gel chromatography (from PE/EtOAc=1/0 to 2/1, TLC: PE/EtOAc=2/1, R_{f}=0.60) to yield O-tert-butyl N-(3-fluoro-4-hydroxyphenyl) carbamate (18 g, 74.46 mmol, 55.7% yield, 94.0% purity) as a yellow solid. ¹H NMR (400 MHz, DMSO-d₆) δ 9.39 (brs, 1H), 9.20 (brs, 1H), 7.29 (d, J=13.4 Hz, 1H), 6.99 (d, J=8.3 Hz, 1H), 6.77-6.85 (m, 1H), 1.45 (s, 9H);

### Step 2: O-tert-Butyl N-[3-fluoro-4-(5-iodopyrimidin-4-yloxy)phenyl] carbamate

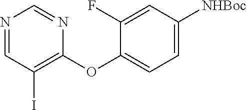 [SMILES: CC(C)(C)OC(=O)Nc1ccc(Oc2ncncc2I)c(F)c1]

To a stirred solution of 4-chloro-5-iodo-pyrimidine (12 g, 49.91 mmol, 1 eq) in DMSO (500 mL) were added O-tert-butyl N-(3-fluoro-4-hydroxyphenyl) carbamate (12.07 g, 49.91 mmol, 94.0% purity, 1 eq) and K₂CO₃ (20.69 g, 149.73 mmol, 3 eq). The reaction mixture was stirred at 120° C. for 1 h under N₂ atmosphere. To the mixture was added water (1000 mL) and extracted with EtOAc (900 mL×3). The combined organic layers were washed with brine (1000 mL), dried over Na₂SO₄, filtered and concentrated under reduced pressure to give a residue which was purified by flash silica gel chromatography (from PE/EtOAc=1/0 to 2/1, TLC: PE/EtOAc=3/1, R_{f}=0.70) to yield O-tert-butyl N-[3-fluoro-4-(5-iodopyrimidin-4-yloxy)phenyl] carbamate (16.6 g, 34.65 mmol, 69.4% yield, 90.0% purity) as a yellow oil. ¹H NMR (500 MHz, DMSO-d₆) δ 9.65 (s, 1H), 9.07-9.02 (m, 1H), 8.67-8.62 (m, 1H), 7.55 (d, J=13.4 Hz, 1H), 7.32-7.23 (m, 2H), 1.48 (s, 9H); ES-LCMS m/z 432.1 [M+H]⁺

### Step 3: O-tert-Butyl N-[3-fluoro-4-(5-(trimethylstannyl)pyrimidin-4-yloxy)phenyl] carbamate

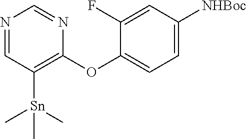 [SMILES: CC(C)(C)OC(=O)Nc1ccc(Oc2ncnc[c]2[Sn]([CH3])([CH3])[CH3])c(F)c1]

A mixture of O-tert-butyl N-[3-fluoro-4-(5-iodopyrimidin-4-yloxy)phenyl] carbamate (10 g, 20.87 mmol, 90.0% purity, 1 eq) and 1,1,1,2,2,2-hexamethyldistannane (16.270 g, 49.66 mmol, 10.30 mL, 2.38 eq) in toluene (100 mL) was added Pd(PPh₃)₄ (1.21 g, 1.04 mmol, 0.05 eq). The mixture was stirred at 110° C. for 16 h under N₂ atmosphere. The reaction mixture was quenched with aqueous KF (200 mL, 2M) at 0° C. and extracted with EtOAc (800 mL×3). The organic layer was washed with water (200 mL×2) and brine (800 mL), dried over Na₂SO₄, filtered and concentrated under reduced pressure to give a residue which was purified by flash silica gel chromatography (from PE/EtOAc=1/0 to 2/1, TLC: PE/EtOAc=2/1, R_{f}=0.45) to yield O-tert-butyl N-[3-fluoro-4-(5-(trimethylstannyl)pyrimidin-4-yloxy)phenyl| carbamate (5.8 g, 11.40 mmol, 54.6% yield, 92.0% purity) as a light yellow solid. ¹H NMR (400 MHz, DMSO-d₆) δ 9.62 (s, 1H), 8.65 (s, 1H), 8.60 (s, 1H), 7.50 (d, J=13.4 Hz, 1H), 7.27-7.21 (m, 2H), 1.48 (s, 9H), 0.39 (s, 9H); ES-LCMS m/z 470.1 [M+H]⁺

### Step 4: O-tert-Butyl-N-[4-[5-(2-chloropyrimidin-4-yl)pyrimidin-4-yloxy]-3-fluorophenyl] carbamate

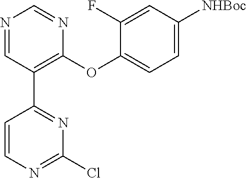 [SMILES: CC(C)(C)OC(=O)Nc1ccc(Oc2ncncc2-c2ccnc(Cl)n2)c(F)c1]

To a stirred solution of O-tert-butyl N-[3-fluoro-4-(5-(trimethylstannyl)pyrimidin-4-yloxy)phenyl] carbamate (2.4 g, 4.72 mmol, 92.0% purity, 1 eq) and 2,4-dichloropyrimidine (2.11 g, 14.15 mmol, 3 eq) in DMF (20 mL) was added Pd(PPh₃)₄ (545.06 mg, 471.69 μmol, 0.1 eq). The reaction mixture was stirred at 100° C. for 16 h under N₂ atmosphere. To the mixture was added water (100 mL) and extracted with EtOAc (80 mL×3). The combined organic layers were washed with brine (100 mL), dried over Na₂SO₄, filtered and concentrated under reduced pressure to give a residue which was purified by flash silica gel chromatography (from PE/EtOAc=1/0 to 1/1, TLC: PE/EtOAc=2/1, R_{f}=0.63) to yield O-tert-butyl N-[4-[5-(2-chloropyrimidin-4-yl)pyrimidin-4-yloxy]-3-fluorophenyl] carbamate (400 mg, 804.17 μmol, 17.1% yield, 84.0% purity) as a light yellow solid. ¹H NMR (500 MHz, DMSO-d₆) δ 9.67 (s, 1H), 9.27 (s, 1H), 8.94 (d, J=5.2 Hz, 1H), 8.89 (s, 1H), 8.23 (d, J=5.2 Hz, 1H), 7.59-7.54 (m, 1H), 7.41-7.36 (m, 1H), 7.29 (d, J=7.5 Hz, 1H), 1.49 (s, 9H); ES-LCMS m/z 417.9 [M+H]⁺.

### Step 5: 4-[5-(2-Chloropyrimidin-4-yl)pyrimidin-4-yloxy]-3-fluoroaniline

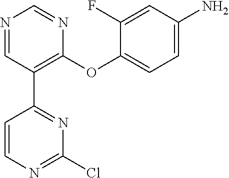 [SMILES: Nc1ccc(Oc2ncncc2-c2ccnc(Cl)n2)c(F)c1]

A mixture of O-tert-butyl N-[4-[5-(2-chloropyrimidin-4-yl)pyrimidin-4-yloxy]-3-fluorophenyl] carbamate (1.6 g, 1.91 mmol, 84.0% purity, 1 eq) in DCM (10 mL) was added TFA (7.70 g, 67.53 mmol, 5 mL, 35.27 eq) at 0° C. The mixture was stirred at 0° C. for 4 h. The reaction mixture was neutralized to pH=7 with sat NaHCO₃ at 0° C., then extracted with DCM (50 mL×3). The combined organic layers were washed with brine (80 mL), dried over Na₂SO₄, filtered and concentrated under reduced pressure to give a residue which was purified by preparative TLC (PE/EtOAc=3/1, TLC: PE/EtOAc=2/1, R_{f}=0.30) to yield 4-[5-(2-chloropyrimidin-4-yl)pyrimidin-4-yloxy]-3-fluoroaniline (600 mg, 1.85 mmol, 96.7% yield, 98.0% purity) as a yellow solid. ¹H NMR (400 MHz, DMSO-d₆) δ 9.24 (s, 1H), 8.93 (d, J=5.2 Hz, 1H), 8.89 (s, 1H), 8.20 (d, J=5.3 Hz, 1H), 7.07 (t, J=8.9 Hz, 1H), 6.49 (dd, J=2.5, 13.0 Hz, 1H), 6.41 (dd, J=2.0, 8.7 Hz, 1H), 5.47-5.41 (brs, 2H); ES-LCMS m/z 318.2 [M+H]⁺.

### Step 6: O-tert-Butyl N-(3S)-3-[[4-[4-(4-amino-2-fluoro-phenoxy)pyrimidin-5-yl]pyrimidin-2-yl]amino]piperidine-1-carboxylate

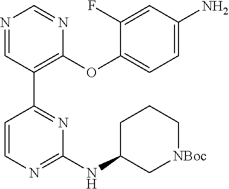 [SMILES: CC(C)(C)OC(=O)N1CCC[C@H](Nc2nccc(-c3cncnc3Oc3ccc(N)cc3F)n2)C1]

To a stirred solution of 4-[5-(2-chloropyrimidin-4-yl)pyrimidin-4-yloxy]-3-fluoroaniline (600 mg, 1.85 mmol, 98.0% purity, 1 eq) and O-tert-butyl N-((3S)-3-aminopiperidine)-1-carboxylate (370.67 mg, 1.85 mmol, 1 eq) in DMSO (6 mL) was added K₂CO₃ (767.36 mg, 5.55 mmol, 3 eq). The reaction mixture was stirred at 120° C. for 1 h. To the mixture was added water (80 mL) and extracted with EtOAc (50 mL×3). The combined organic layers were washed with brine (50 mL), dried over Na₂SO₄, filtered and concentrated under reduced pressure to give a residue which was purified by preparative TLC (PE/EtOAc=3/1, TLC: PE/EtOAc=1/1, R_{f}=0.60) to yield O-tert-butyl N-((3S)-3-[[4-[4-(4-amino-2-fluoro-phenoxy)pyrimidin-5-yl]pyrimidin-2-yl]amino]piperidine-)1-carboxylate (180 mg, 355.12 μmol, 19.2% yield, 95.0% purity) as a light yellow solid. ¹H NMR (400 MHz, DMSO-d₆) δ 9.37-9.03 (m, 1H), 8.80 (s, 1H), 8.44 (d, J=5.1 Hz, 1H), 7.37 (d, J=7.2 Hz, 1H), 7.27 (brs, 1H), 7.03 (t, J=8.8 Hz, 1H), 6.48 (dd, J=2.4, 12.9 Hz, 1H), 6.40 (dd, J=2.1, 8.6 Hz, 1H), 5.43-5.38 (m, 2H), 3.80 (brs, 2H), 3.48 (s, 1H), 3.08-2.71 (m, 2H), 1.93 (s, 1H), 1.76 (brs, 1H), 1.57 (s, 2H), 1.42-1.22 (m, 9H); ES-LCMS m/z 317.1, 482.3 [M+H]⁺.

### Step 7: O-tert-Butyl N-((3S)-3-[[4-[4-[4-[(2-chlorophenyl)sulfonylamino]-2-fluoro-phenoxy]pyrimidin-5-yl]pyrimidin-2-yl]amino]piperidine)-1-carboxylate

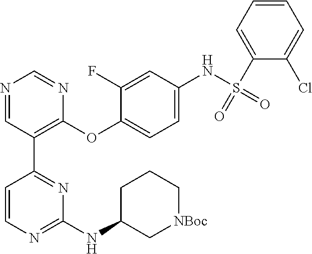 [SMILES: CC(C)(C)OC(=O)N1CCC[C@H](Nc2nccc(-c3cncnc3Oc3ccc(NS(=O)(=O)c4ccccc4Cl)cc3F)n2)C1]

To a stirred solution of O-tert-butyl N-((3S)-3-[[4-[4-(4-amino-2-fluoro-phenoxy)pyrimidin-5-yl]pyrimidin-2-yl]amino]piperidine)-1-carboxylate (100 mg, 197.29 μmol, 95.0% purity, 1 eq) in THF (3 mL) were added pyridine (2.94 g, 37.17 mmol, 3 mL, 188.39 eq) and 2-chlorobenzenesulfonyl chloride (166.57 mg, 789.17 μmol, 107.46 μL, 4 eq). The reaction mixture was stirred at 25° C. for 1 h. To the mixture was added water (80 mL) and extracted with EtOAc (50 mL×3). The combined organic layers were washed with brine (50 mL), dried over Na₂SO₄, filtered and concentrated under reduced pressure to give a residue which was purified by flash silica gel chromatography (from PE/EtOAc=1/0 to 2/1, TLC: PE/EtOAc=0/1, R_{f}=0.72) to yield O-tert-butyl N-((3S)-3-[[4-[4-[4-[(2-chlorophenyl)sulfonylamino]-2-fluoro-phenoxy]pyrimidin-5-yl]pyrimidin-2-yl]amino]piperidine)-1-carboxylate (100 mg, 144.79 μmol, 73.4% yield, 95.0% purity) as a light yellow solid. ¹H NMR (400 MHz, DMSO-d₆) δ 11.03 (s, 1H), 9.36-9.10 (m, 1H), 8.78 (s, 1H), 8.41 (d, J=5.0 Hz, 1H), 8.13 (d, J=7.6 Hz, 1H), 7.70-7.67 (m, 2H), 7.58 (dt, J=3.0, 5.3 Hz, 1H), 7.40-7.33 (m, 2H), 7.23 (s, 1H), 7.07 (dd, J=2.4, 11.8 Hz, 1H), 6.99 (d, J=8.8 Hz, 1H), 3.78 (s, 2H), 3.64 (s, 1H), 3.20-2.75 (m, 2H), 1.91 (s, 1H), 1.73 (s, 1H), 1.56 (s, 2H), 1.42-1.22 (m, 9H); ES-LCMS m/z 656.3 [M+H]⁺.

### Step 8: N-[3-Fluoro-4-[5-[2-[[(3S)-3-piperidyl]amino]pyrimidin-4-yl]pyrimidin-4-yloxy]phenyl] 2-chlorobenzenesulfonamide

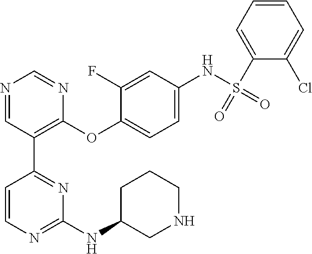 [SMILES: O=S(=O)(Nc1ccc(Oc2ncncc2-c2ccnc(N[C@H]3CCCNC3)n2)c(F)c1)c1ccccc1Cl]

To a stirred solution of O-tert-butyl N-((3S)-3-└└4-└4-└4-└(2-chlorophenyl)sulfonylamino]-2-fluoro-phenoxy]pyrimidin-5-yl]pyrimidin-2-yl]amino]piperidine)-1-carboxylate (50 mg, 72.39 μmol, 95.0% purity, 1 eq) in DCM (3 mL) was added TFA (1.54 g, 13.51 mmol, 1 mL, 186.56 eq). The reaction mixture was stirred at 25° C. for 0.5 h. The reaction mixture was neutralized to pH=7 with NH₃·H₂O, and concentrated to yield a residue which was purified by preparative HPLC (column: Boston Prime C18 150*30 mm*5 μm; mobile phase: [water (NH₃H₂O+NH₄HCO₃)-ACN]; B %: 20%-50%, 10 min), followed by lyophilization to yield N-[3-fluoro-4-[5-[2-[[(3S)-3-piperidyl]amino]pyrimidin-4-yl]pyrimidin-4-yloxy]phenyl] 2-chlorobenzenesulfonamide (21.5 mg, 38.67 μmol, 53.4% yield, 100.0% purity) as a white solid. ¹H NMR (400 MHz, CD₃OD) δ 9.33 (s, 1H), 8.69 (s, 1H), 8.40 (d, J=5.1 Hz, 1H), 8.13 (dd, J=7.6, 1.4 Hz, 1H), 7.61-7.56 (m, 2H), 7.51-7.45 (m, 1H), 7.17 (t, J=8.9 Hz, 1H), 7.08 (dd, J=12.0, 2.5 Hz, 1H), 6.86 (d, J=12.2 Hz, 1H), 7.01 (ddd, J=8.7, 2.6, 1.2 Hz, 1H), 4.68-4.53 (m, 1H), 4.21-4.11 (m, 1H), 3.49-3.42 (m, 1H), 3.18-3.12 (m, 1H), 2.87-2.74 (m, 2H), 2.17-2.10 (m, 1H), 1.2.01-1.92 (m, 1H), 1.84-1.61 (m, 2H); ES-LCMS m/z 556.1 [M+H]⁺.
128. N-[4-[5-[2-[(1r,4r)(4-aminocyclohexyl)amino]pyrimidin-4-yl]pyrimidin-4-yl]oxy-3-fluorophenyl] 2-chlorobenzenesulfonamide

### O-tert-Butyl N-[4-[[4-[(1r,4r)4-(4-amino-2-fluoro-phenoxy)pyrimidin-5-yl]pyrimidin-2-yl]amino]cyclohexyl] carbamate

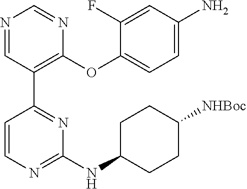 [SMILES: CC(C)(C)OC(=O)N[C@H]1CC[C@H](Nc2nccc(-c3cncnc3Oc3ccc(N)cc3F)n2)CC1]

To a stirred solution of 4-[5-(2-chloropyrimidin-4-yl)pyrimidin-4-yloxy]-3-fluoroaniline (600 mg, 1.85 mmol, 98.0% purity, 1 eq) and O-tert-butyl N-((1r,4r)4-aminocyclohexyl) carbamate (396.63 mg, 1.85 mmol, 1 eq) in DMSO (8 mL) was added K₂CO₃ (767.36 mg, 5.55 mmol, 3 eq). The reaction mixture was stirred at 100° C. for 2 h. To the mixture was added water (100 mL) and extracted with EtOAc (80 mL×3). The combined organic layers were washed with brine (100 mL), dried over Na₂SO₄, filtered and concentrated under reduced pressure to give a residue which was purified by preparative TLC (PE/EtOAc=3/1, TLC: PE/EtOAc=1/1, R_{f}=0.50) to yield O-tert-butyl N-[4-[[4-[(1r,4r)4-(4-amino-2-fluoro-phenoxy)pyrimidin-5-yl]pyrimidin-2-yl]amino]cyclohexyl]carbamate (240 mg, 474.63 μmol, 25.6% yield, 98.0% purity) as a light yellow solid. ¹H NMR (400 MHz, DMSO-d₆) δ ppm 9.18 (brs, 1H), 8.79 (s, 1H), 8.40 (d, J=4.9 Hz, 1H), 7.26 (d, J=7.8 Hz, 1H), 7.18 (d, J=4.1 Hz, 1H), 7.02 (t, J=8.9 Hz, 1H), 6.74 (d, J=8.0 Hz, 1H), 6.48 (dd, J=2.4, 13.0 Hz, 1H), 6.40 (dd, J=2.1, 8.6 Hz, 1H), 5.43-5.38 (m, 2H), 3.71 (brs, 1H), 3.20 (brs, 1H), 1.96 (s, 2H), 1.80 (d, J=10.3 Hz, 2H), 1.38 (s, 9H), 1.33-1.23 (m, 4H); ES-LCMS m/z 496.2 [M+H]⁺

### Step 2: O-tert-Butyl N-[4-(1r,4r)[[4-[4-[4-[(2-chlorophenyl)sulfonylamino]-2-fluoro-phenoxy]pyrimidin-5-yl]pyrimidin-2-yl]amino]cyclohexyl] carbamate

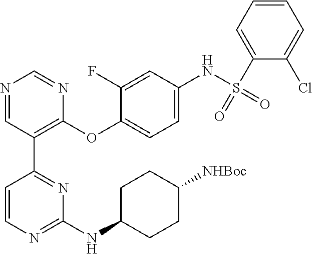 [SMILES: CC(C)(C)OC(=O)N[C@H]1CC[C@H](Nc2nccc(-c3cncnc3Oc3ccc(NS(=O)(=O)c4ccccc4Cl)cc3F)n2)CC1]

To a stirred solution of O-tert-butyl N-[4-[[4-[(1r,4r)4-(4-amino-2-fluoro-phenoxy)pyrimidin-5-yl]pyrimidin-2-yl]amino]cyclohexyl] carbamate (100 mg, 197.76 μmol, 98.0% purity, 1 eq) in THF (2 mL) were added pyridine (3.92 g, 49.56 mmol, 4 mL, 250.59 eq) and 2-chlorobenzenesulfonyl chloride (41.74 mg, 197.76 μmol, 26.93 μL, 1 eq). The reaction mixture was stirred at 25° C. for 0.5 h. To the mixture was added water (80 mL) and extracted with EtOAc (50 mL×3). The combined organic layers were washed with brine (50 mL), dried over Na₂SO₄, filtered and concentrated under reduced pressure to give a residue which was purified by flash silica gel chromatography (from PE/EtOAc=1/0 to 2/1, TLC: PE/EtOAc=1/1, R_{f}=0.42) to yield O-tert-butyl N-[4-(1r,4r)[[4-[4-[4-[(2-chlorophenyl)sulfonylamino]-2-fluorophenoxy]pyrimidin-5-yl]pyrimidin-2-yl]amino]cyclohexyl] carbamate (120 mg, 170.11 μmol, 86.0% yield, 95.0% purity) as a light yellow solid. ¹H NMR (400 MHz, DMSO-d₆) δ ppm 11.02 (brs, 1H), 9.18 (brs, 1H), 8.77 (s, 1H), 8.37 (d, J=4.8 Hz, 1H), 8.12 (d, J=7.5 Hz, 1H), 7.70-7.64 (m, 2H), 7.60-7.53 (m, 1H), 7.34 (t, J=8.8 Hz, 1H), 7.25 (d, J=7.8 Hz, 1H), 7.14 (d, J=4.9 Hz, 1H), 7.07 (dd, J=2.4, 11.9 Hz, 1H), 7.01-6.95 (m, 1H), 6.72 (d, J=8.0 Hz, 1H), 3.70 (brs, 1H), 3.19 (brs, 1H), 2.00-1.86 (m, 2H), 1.78 (d, J=9.1 Hz, 2H), 1.38 (s, 9H), 1.34-1.22 (m, 4H); ES-LCMS m/z 670.3 [M+H]⁺

### Step 3: N-[4-[5-[2-[(1r,4r)(4-Aminocyclohexyl)amino]pyrimidin-4-yl]pyrimidin-4-yloxy]-3-fluorophenyl] 2-chlorobenzenesulfonamide

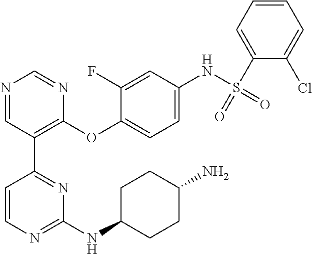 [SMILES: N[C@H]1CC[C@H](Nc2nccc(-c3cncnc3Oc3ccc(NS(=O)(=O)c4ccccc4Cl)cc3F)n2)CC1]

To a stirred solution of O-tert-butyl N-[4-(1r,4r)[[4-[4-[4-[(2-chlorophenyl)sulfonylamino]-2-fluorophenoxy]pyrimidin-5-yl]pyrimidin-2-yl]amino]cyclohexyl] carbamate (60 mg, 85.06 μmol, 95.0% purity, 1 eq) in DCM (3 mL) was added TFA (1.54 g, 13.51 mmol, 1 mL, 158.80 eq). The reaction mixture was stirred at 25° C. for 0.5 h. The reaction mixture was neutralized to pH=7 with NH₃·H₂O, and concentrated to yield a residue which was purified by preparative HPLC (column: Boston Green ODS 150*30 mm*5 μm; mobile phase: [water (FA)-ACN]; B %: 12%-42%, 12 min), followed by lyophilization to yield N-[4-[5-[2-[(1r,4r)(4-aminocyclohexyl)amino]pyrimidin-4-yl]pyrimidin-4-yloxy]-3-fluorophenyl] 2-chlorobenzenesulfonamide (25.1 mg, 44.03 μmol, 51.8% yield, 100.0% purity) as a white solid. ¹H NMR (400 MHz, DMSO-d₆) δ ppm 11.05 (s, 1H), 9.24 (m, 1H), 8.78 (s, 1H), 8.39 (d, J=5.0 Hz, 1H), 8.13 (dd, J=7.8, 1.4 Hz, 1H), 7.77 (brs, 2H), 7.71-7.67 (m, 2H), 7.58 (ddd, J=3.3, 5.3, 8.1 Hz, 1H), 7.40-7.31 (m, 2H), 7.18 (brs, 1H), 7.08 (dd, J=2.4, 11.9 Hz, 1H), 7.00 (brd, J=8.8 Hz, 1H), 3.79-3.63 (m, 1H), 3.07-2.96 (m, 1H), 2.06-1.92 (m, 4H), 1.47-1.30 (m, 4H); ES-LCMS m/z 570.2 [M+H]⁺.

The following compounds have been made by processes similar to those described immediately above using Method H.
129. N-[3-fluoro-4-[5-[2-[[(3S)-3-piperidyl]amino]pyrimidin-4-yl]pyrimidin-4-yl]oxy-phenyl] benzenesulfonamide. ES-LCMS m/z 522.3 [M+H]⁺.
130. N-[4-[5-[2-[(1r,4r)(4-aminocyclohexyl)amino]pyrimidin-4-yl]pyrimidin-4-yl]oxy-3-fluoro-phenyl] benzenesulfonamide. ES-LCMS m/z 536.3 [M+H]⁺.

### Example 10. Syntheses of Compound 131

The following compound was made using Method I.
131. N-[4-(1r,4r)[[4-[2-[(4-aminocyclohexyl)amino]pyrimidin-4-yl]-3-pyridyl]oxy]-3-fluorophenyl] 2-chlorobenzenesulfonamide

### Step 1: 2-Chloro-4-(3-methoxypyrid-4-yl)pyrimidine

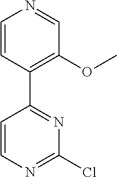

To a stirred solution of (3-methoxypyrid-4-yl) boronic acid (400 mg, 2.62 mmol, 1 eq), 2,4-dichloropyrimidine (584.44 mg, 3.92 mmol, 1.5 eq) and Cs₂CO₃ (1.28 g, 3.92 mmol, 1.5 eq) in THF (6 mL) and H₂O (3 mL) was added Pd(dppf)Cl₂ (191.37 mg, 261.53 μmol, 0.1 eq). The reaction mixture was stirred at 60° C. for 2 h under N₂ atmosphere. To the mixture was added water (80 mL) and extracted with EtOAc (50 mL×3). The combined organic layers were washed with brine (50 mL), dried over Na₂SO₄, filtered and concentrated under reduced pressure to give a residue which was purified by preparative TLC (PE/EtOAc=3/1, TLC: PE/EtOAc=1/1, R_{f}=0.20) to yield 2-chloro-4-(3-methoxy-pyrid-4-yl)pyrimidine (400 mg, 1.73 mmol, 66.2% yield, 96.0% purity) as a light yellow solid. ¹H NMR (400 MHz, DMSO-d₆) δ ppm 8.88 (d, J=5.1 Hz, 1H), 8.64 (s, 1H), 8.40 (d, J=4.9 Hz, 1H), 8.13 (d, J=5.2 Hz, 1H), 7.83 (d, J=4.9 Hz, 1H), 4.03 (s, 3H); ES-LCMS m/z 222.1 [M+H]⁺

### Step 2: 4-(2-Chloropyrimidin-4-yl)pyridin-3-ol

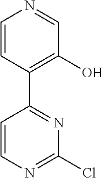 [SMILES: Oc1cnccc1-c1ccnc(Cl)n1]

To a stirred solution of 2-chloro-4-(3-methoxypyrid-4-yl)pyrimidine (2 g, 8.93 mmol, 99.0% purity, 1 eq) in DMF (20 mL) was added iodo(trimethyl)silane (14.70 g, 73.47 mmol, 10 mL, 8.22 eq) at 0° C. The reaction mixture was stirred at 80° C. for 3 h. To the mixture was added water (150 mL) and extracted with EtOAc (100 mL×3). The combined organic layers were washed with brine (150 mL), dried over Na₂SO₄, filtered and concentrated under reduced pressure to give a residue which was purified by flash silica gel chromatography (PE/EtOAc=1/0 to 2/1, TLC: PE/EtOAc=1/1, R_{f}=0.18) to yield 4-(2-chloropyrimidin-4-yl)pyridin-3-ol (150 mg, 433.49 μmol, 4.85% corrected yield, 60% purity) as a brown solid. ¹H NMR (400 MHz, DMSO-d₆) δ ppm 8.91-8.88 (m, 1H), 8.31 (d, J=5.3 Hz, 1H), 7.95 (d, 2H), 7.93-7.89 (m, 1H); ES-LCMS m/z 208.1 [M+H]⁺

### Step 3: 2-Chloro-4-[3-(2-fluoro-4-nitrophenoxy)pyrid-4-yl]pyrimidine

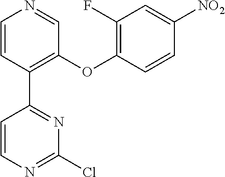 [SMILES: O=[N+]([O-])c1ccc(Oc2cnccc2-c2ccnc(Cl)n2)c(F)c1]

To a stirred solution of 4-(2-chloropyrimidin-4-yl)pyridin-3-ol (50 mg, 185.44 μmol, 60% purity, 1 eq) and 1,2-difluoro-4-nitro-benzene (29.50 mg, 185.44 μmol, 20.49 L, 1 eq) in DMSO (3 mL) was added K₂CO₃ (76.89 mg, 556.31 μmol, 3 eq). The reaction mixture was stirred at 25° C. for 1 h. TLC (PE/EtOAc=0/1) indicated the starting material was consumed completely. To the mixture was added water (80 mL) and extracted with EtOAc (50 mL×3). The combined organic layers were washed with brine (50 mL), dried over Na₂SO₄, filtered and concentrated under reduced pressure to give a residue which was purified by preparative TLC (PE/EtOAc=0/1, TLC: PE/EtOAc=0/1, R_{f}=0.30) to yield 2-chloro-4-[3-(2-fluoro-4-nitrophenoxy) pyrid-4-yl]pyrimidine (10 mg, 28.27 μmol, 15.2% yield, 98.0% purity) as a white solid. ¹H NMR (500 MHz, DMSO-d₆) δ ppm 8.92 (d, J=5.0 Hz, 1H), 8.74 (d, J=5.0 Hz, 1H), 8.68 (s, 1H), 8.37 (dd, J=2.6, 10.7 Hz, 1H), 8.08-8.04 (m, 1H), 8.03 (d, J=5.2 Hz, 1H), 8.01 (d, J=5.0 Hz, 1H), 7.27 (t, J=8.7 Hz, 1H); ES-LCMS 347.0 m/z [M+H]⁺.

### Step 4: O-tert-Butyl N-[4-(1r,4r)[[4-[3-(2-fluoro-4-nitrophenoxy) pyrid-4-yl]pyrimidin-2-yl]amino]cyclohexyl] carbamate

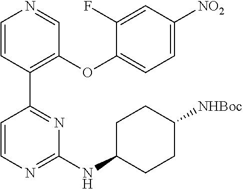 [SMILES: CC(C)(C)OC(=O)N[C@H]1CC[C@H](Nc2nccc(-c3ccncc3Oc3ccc([N+](=O)[O-])cc3F)n2)CC1]

To a stirred solution of 2-chloro-4-[3-(2-fluoro-4-nitro-phenoxy)-4-pyridyl]pyrimidine (50 mg, 137.01 μmol, 1 eq) and tert-butyl N-(4-aminocyclohexyl)carbamate (58.72 mg, 274.01 μmol, 2 eq) in DMSO (1 mL) was added TEA (41.59 mg, 411.02 μmol, 57.21 μL, 3 eq). The reaction mixture was stirred at 120° C. for 2 h. To the mixture was added water (50 mL) and extracted with EtOAc (30 mL×3). The combined organic layers were washed with brine (50 mL), dried over Na₂SO₄, filtered and concentrated under reduced pressure to give a residue which was purified by preparative TLC (PE/EtOAc=0/1, TLC: PE/EtOAc=0/1, R_{f}=0.45) to yield O-tert-Butyl N-[4-(1r,4r)[[4-[3-(2-fluoro-4-nitrophenoxy) pyrid-4-yl]pyrimidin-2-yl]amino]cyclohexyl] carbamate (30 mg, 57.19 μmol, 41.7% yield, 95.0% purity) as a white solid. ¹H NMR (400 MHz, CD₃OD-d₄) δ ppm 8.62 (d, J=5.1 Hz, 1H), 8.50 (s, 1H), 8.27 (d, J=5.0 Hz, 1H), 8.19 (dd, J=2.7, 10.6 Hz, 1H), 8.03 (dd, J=1.4, 9.1 Hz, 1H), 7.93 (d, J=5.0 Hz, 1H), 7.11-7.05 (m, 1H), 6.99 (d, J=5.1 Hz, 1H), 3.73-3.63 (m, 1H), 3.35 (brs, 1H), 2.04-1.98 (m, 2H), 1.95-1.90 (m, 2H), 1.44 (s, 9H), 1.38-1.28 (m, 4H).

### Step 5: O-tert-Butyl N-[4-(1r,4r)[[4-[3-(4-amino-2-fluorophenoxy) pyrid-4-yl]pyrimidin-2-yl]amino]cyclohexyl] carbamate

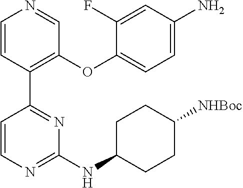 [SMILES: CC(C)(C)OC(=O)N[C@H]1CC[C@H](Nc2nccc(-c3ccncc3Oc3ccc(N)cc3F)n2)CC1]

To a stirred solution of tert-N-└4-└4-└3-(2-fluoro-4-nitro-phenoxy)-4-pyridyl]pyrimidin-2-yl]amino]cyclohexyl]carbamate (30 mg, 54.33 μmol, 1 eq) in THF (0.8 mL), EtOH (0.8 mL) and H₂O (0.8 mL) were added Fe (30.34 mg, 543.33 μmol, 10 eq) and NH₄Cl (29.06 mg, 543.33 μmol, 10 eq). The reaction mixture was stirred at 50° C. for 2 h. The reaction mixture was filtered. Water (100 mL) was added and extracted with EtOAc (80 mL×3). The combined organic layers were washed with brine (100 mL), dried over Na₂SO₄, filtered and concentrated under reduced pressure to give tert-butyl O-tert-Butyl N-[4-(1r,4r)[[4-[3-(4-amino-2-fluorophenoxy) pyrid-4-yl]pyrimidin-2-yl]amino]cyclohexyl] carbamate (28 mg, 50.95 μmol, 93.8% yield, 90.0% purity) as a yellow solid. ¹H NMR (500 MHz, CD₃OD) δ ppm 8.33 (d, J=6.56 Hz, 2H), 8.09-7.89 (m, 2H), 7.25 (s, 1H), 6.95 (t, J=7.86 Hz, 1H), 6.62-6.47 (m, 2H), 3.81 (brs, 1H), 3.35 (brs, 1H), 2.10 (s, 2H), 1.97 (s, 2H), 1.44 (s, 9H), 1.39 (s, 4H); ES-LCMS m/z 495.3 [M+H]⁺.

### Step 6: O-tert-Butyl N-[4-(1r,4r)[[4-[3-[4-[(2-chlorophenyl)sulfonylamino]-2-fluorophenoxy]pyrid-4-yl]pyrimidin-2-yl]amino]cyclohexyl] carbamate

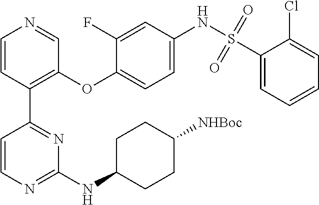 [SMILES: CC(C)(C)OC(=O)N[C@H]1CC[C@H](Nc2nccc(-c3ccncc3Oc3ccc(NS(=O)(=O)c4ccccc4Cl)cc3F)n2)CC1]

To a stirred solution of tert-butyl N-[4-[[4-[3-(4-amino-2-fluoro-phenoxy)-4-pyridyl]pyrimidin-2-yl]amino]cyclohexyl]carbamate (28 mg, 50.95 μmol, 1 eq) in THF (1 mL) was added pyridine (980.00 mg, 12.39 mmol, 1 mL, 243.15 eq) and 2-chlorobenzenesulfonyl chloride (21.51 mg, 101.91 μmol, 13.89 μL, 2 eq). The reaction mixture was stirred at 25° C. for 1 h. To the mixture was added water (80 mL) and extracted with EtOAc (50 mL×3). The combined organic layers were washed with brine (80 mL), dried over Na₂SO₄, filtered and concentrated under reduced pressure to give a residue which was purified by preparative TLC (PE/EtOAc=0/1, TLC: PE/EtOAc=0/1, R_{f}=0.76) to yield O-tert-Butyl N-[4-(1r,4r)[[4-[3-[4-[(2-chlorophenyl)sulfonylamino]-2-fluorophenoxy]pyrid-4-yl]pyrimidin-2-yl]amino]cyclohexyl]carbamate (35 mg, 48.64 μmol, 95.5% yield, 93.0% purity) as a yellow solid. ¹H NMR (400 MHz, CD₃OD-d₄) δ ppm 8.41 (d, J=5.0 Hz, 1H), 8.25 (d, J=5.1 Hz, 1H), 8.09-8.01 (m, 2H), 7.90 (d, J=5.0 Hz, 1H), 7.60-7.56 (m, 2H), 7.48-7.43 (m, 1H), 7.11-7.05 (m, 2H), 6.99-6.91 (m, 2H), 3.80-3.71 (m, 1H), 3.39-3.35 (m, 1H), 2.09-2.03 (m, 2H), 1.94 (d, J=10.0 Hz, 2H), 1.44 (s, 9H), 1.40-1.32 (m, 4H); ES-LCMS m/z 669.4 [M+H]⁺.

### Step 7: N-[4-(1r,4r)[[4-[2-[(4-aminocyclohexyl)amino]pyrimidin-4-yl]-3-pyridyl]oxy]-3-fluorophenyl] 2-chlorobenzenesulfonamide

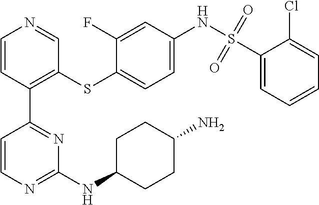 [SMILES: N[C@H]1CC[C@H](Nc2nccc(-c3ccncc3Sc3ccc(NS(=O)(=O)c4ccccc4Cl)cc3F)n2)CC1]

To a stirred solution of O-tert-Butyl N-[4-(1r,4r)[[4-[3-[4-[(2-chlorophenyl)sulfonylamino]-2-fluorophenoxy]pyrid-4-yl]pyrimidin-2-yl]amino]cyclohexyl]carbamate (30 mg, 41.69 μmol, 1 eq) in DCM (0.3 mL) was added TFA (153.50 mg, 1.35 mmol, 0.1 mL, 32.29 eq). The reaction mixture was stirred at 25° C. for 0.5 h. The reaction mixture was neutralized to pH=7 with NH₃·H₂O and concentrated to yield a residue which was purified by preparative HPLC (column: Boston Green ODS 150*30 mm*5 μm; mobile phase: [water (FA)-ACN]; B %: 10%-40%, 12 min), followed by lyophilization to yield N-[4-(1r,4r)[[4-[2-[(4-aminocyclohexyl)amino]pyrimidin-4-yl]-3-pyridyl]oxy]-3-fluorophenyl] 2-chlorobenzenesulfonamide (10.8 mg, 17.56 μmol, 42.1% yield, 100.0% purity, 0.20 FA) as a white solid. ¹H NMR (400 MHz, DMSO-d₆, 75° C.) δ ppm 8.46 (d, J=4.9 Hz, 1H), 8.33 (d, J=5.0 Hz, 1H), 8.22 (s, 0.20H, HCO₂H), 8.14 (s, 1H), 8.01 (dd, J=7.9, 1.6 Hz, 1H), 7.79 (d, J=4.9 Hz, 1H), 7.57-7.42 (m, 3H), 7.03 (d, J=5.0 Hz, 1H), 7.00-6.88 (m, 3H), 6.81 (brd, J=8.7 Hz, c1H), 3.76-3.64 (m, 1H), 3.02-2.92 (m, 1H), 2.05-1.92 (m 4H), 1.28-1.45 (m, 4H); ES-LCMS m/z 569.3 [M+H]⁺.

### Example 11. Synthesis of Compound 132

The following compound was made using Method J.
132. N-[4-[[2-[2-(1r,4r)[(4-Aminocyclohexyl)amino]pyrimidin-4-yl]-3-pyridyl]oxy]-3-fluoro-phenyl] 2-chlorobenzenesulfonamide

### Step 1: 2-Chloro-4-(3-methoxy-2-pyridyl)pyrimidine

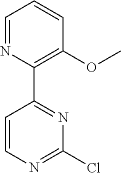

To a stirred solution of 2-bromo-3-methoxypyridine (6 g, 31.91 mmol, 1 eq) and 2,4-dichloropyrimidine (9.51 g, 63.82 mmol, 2 eq) in toluene (700 mL) were added 1,1,1,2,2,2-hexamethyldistannane (21.430 g, 65.41 mmol, 13.56 mL, 2.05 eq) and Pd(PPh₃)₄ (3.69 g, 3.19 mmol, 0.1 eq). The reaction mixture was stirred at 110° C. for 16 h under N₂ atmosphere. TLC (PE/EtOAc=2/1) indicated the starting material was consumed completely. The reaction mixture was quenched with aqueous KF (300 mL, 2M) at 0° C. and extracted with EtOAc (200 mL×3). The combined organic layers were washed with brine (300 mL), dried over Na₂SO₄, filtered and concentrated under reduced pressure to give a residue. The residue was purified by flash silica gel chromatography (PE/EtOAc=1/0 to 2/1, TLC: PE/EtOAc=2/1, R_{f}=0.35) to yield 2-chloro-4-(3-methoxy-2-pyridyl)pyrimidine (1.7 g, 7.44 mmol, 23.3% yield, 97.0% purity) as a brown solid. ¹H NMR (400 MHz, DMSO-d₆) δ ppm 8.87 (d, J=5.1 Hz, 1H), 8.33 (dd, J=1.0, 4.5 Hz, 1H), 7.93 (d, J=5.1 Hz, 1H), 7.74-7.68 (m, 1H), 7.58 (dd, J=4.5, 8.5 Hz, 1H), 3.88 (s, 3H); ES-LCMS m/z 222.1 [M+H]⁺.

### Step 2: 2-(2-Chloropyrimidin-4-yl)pyridin-3-ol

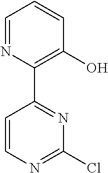 [SMILES: Oc1cccnc1-c1ccnc(Cl)n1]

To a stirred solution of 2-chloro-4-(3-methoxy-2-pyridyl)pyrimidine (1.7 g, 7.44 mmol, 97.0% purity, 1 eq) in DMF (15 mL) was added TMSI (29.40 g, 146.93 mmol, 20 mL, 19.75 eq) at 0° C. The reaction mixture was stirred at 80° C. for 8 h. To the mixture was added water (200 mL) and extracted with EtOAc (150 mL×3). The combined organic layers were washed with brine (200 mL), dried over Na₂SO₄, filtered and concentrated under reduced pressure to give a residue which was purified by flash silica gel chromatography (PE/EtOAc=1/0 to 2/1, TLC: PE/EtOAc=1/1, R_{f}=0.45) to yield 2-(2-chloropyrimidin-4-yl)pyridin-3-ol (580 mg, 2.26 mmol, 30.4% yield, 81.0% purity) as a yellow solid. ¹H NMR (500 MHz, DMSO-d₆) δ ppm 11.85 (s, 1H), 8.94 (d, J=5.3 Hz, 1H), 8.37 (d, J=5.3 Hz, 1H), 8.34-8.32 (m, 1H), 7.57-7.50 (m, 2H); ES-LCMS m/z 208.1 [M+H]⁺.

### Step 3: 2-Chloro-4-[3-(2-fluoro-4-nitrophenoxy) pyrid-2-yl]pyrimidine

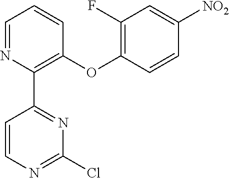 [SMILES: O=[N+]([O-])c1ccc(Oc2cccnc2-c2ccnc(Cl)n2)c(F)c1]

To a stirred solution of 2-(2-chloropyrimidin-4-yl)pyridin-3-ol (800 mg, 3.12 mmol, 1 eq) and 1,2-difluoro-4-nitrobenzene (496.54 mg, 3.12 mmol, 345.54 μL, 1 eq) in DMSO (10 mL) was added K₂CO₃ (1.29 g, 9.36 mmol, 3 eq). The reaction mixture was stirred at 25° C. for 3 h. To the mixture was added water (80 mL) and extracted with EtOAc (50 mL×3). The combined organic layers were washed with brine (80 mL), dried over Na₂SO₄, filtered and concentrated under reduced pressure to give a residue which was purified by flash silica gel chromatography (from PE/EtOAc=1/0 to 2/1, TLC: PE/EtOAc=3/1, R_{f}=0.30) to yield 2-chloro-4-[3-(2-fluoro-4-nitrophenoxy)pyrid-2-yl]pyrimidine (1 g, 2.60 mmol, 83.2% yield, 90.0% purity) as a white solid. ¹H NMR (400 MHz, DMSO-d₆) δ ppm 8.90 (d, J=5.1 Hz, 1H), 8.78 (dd, J=1.2, 4.5 Hz, 1H), 8.36 (dd, J=2.7, 10.8 Hz, 1H), 8.15 (d, J=5.1 Hz, 1H), 8.03-7.98 (m, 2H), 7.81 (dd, J=4.5, 8.3 Hz, 1H), 7.06 (t, J=8.7 Hz, 1H); ES-LCMS 347.1 m/z [M+H]⁺.

### Step 4: O-tert-Butyl N-[4-[(1r,4r)[4-[3-(2-fluoro-4-nitrophenoxy)-2-pyridyl]pyrimidin-2-yl]amino]cyclohexyl] carbamate

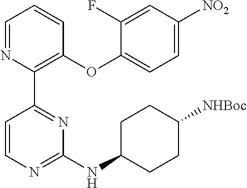 [SMILES: CC(C)(C)OC(=O)N[C@H]1CC[C@H](Nc2nccc(-c3ncccc3Oc3ccc([N+](=O)[O-])cc3F)n2)CC1]

To a stirred solution of 2-chloro-4-[3-(2-fluoro-4-nitro-phenoxy) pyrid-2-yl]pyrimidine (1 g, 2.60 mmol, 1 eq) and tert-butyl N-(1r,4r)(4-aminocyclohexyl) carbamate (834.47 mg, 3.89 mmol, 1.5 eq) in DMSO (10 mL) was added TEA (788.03 mg, 7.79 mmol, 1.08 mL, 3 eq). The reaction mixture was stirred at 120° C. for 5 h. To the mixture was added water (80 mL) and extracted with EtOAc (50 mL×3). The combined organic layers were washed with brine (80 mL), dried over Na₂SO₄, filtered and concentrated under reduced pressure to give a residue which was purified by preparative TLC (PE/EtOAc=0/1, TLC: PE/EtOAc=0/1, R_{f}=0.60) to yield tert-butyl N-(1r,4r)[4-[[4-[3-(2-fluoro-4-nitrophenoxy)-2-pyridyl]pyrimidin-2-yl]amino]cyclohexyl]carbamate (1.2 g, 2.29 mmol, 88.1% yield, 100.0% purity) as a yellow solid. ¹H NMR (400 MHz, DMSO-d₆) δ ppm 8.65 (d, J=3.93 Hz, 1H), 8.34 (d, J=5.0 Hz, 2H), 8.03 (d, J=8.8 Hz, 1H), 7.83 (dd, J=1.1, 8.3 Hz, 1H), 7.65 (dd, J=4.5, 8.3 Hz, 1H), 7.11 (t, J=8.7 Hz, 1H), 7.04-6.53 (m, 3H), 3.31 (brs, 1H), 3.07 (brs, 1H), 1.68-1.55 (m, 4H), 1.37 (s, 9H), 1.25-1.14 (m, 3H), 1.05-0.79 (m, 1H); ES-LCMS m/z 525.3 [M+H]⁺.

### Step 5: O-tert-Butyl N-(1r,4r)[4-[[4-[3-(4-amino-2-fluorophenoxy)-2-pyridyl]pyrimidin-2-yl]amino]cyclohexyl] carbamate

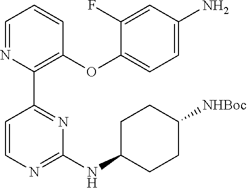 [SMILES: CC(C)(C)OC(=O)N[C@H]1CC[C@H](Nc2nccc(-c3ncccc3Oc3ccc(N)cc3F)n2)CC1]

To a stirred solution of O-tert-butyl N-(1r,4r)[4-[[4-[3-(2-fluoro-4-nitrophenoxy)-2-pyridyl]pyrimidin-2-yl]amino]cyclohexyl] carbamate (1.2 g, 2.24 mmol, 1 eq) in THF (5 mL), EtOH (5 mL) and H₂O (5 mL) were added Fe (1.25 g, 22.42 mmol, 10 eq) and NH₄Cl (1.20 g, 22.42 mmol, 10 eq). The reaction mixture was stirred at 50° C. for 3 h. The reaction mixture was filtered. Water (100 mL) was added to the reaction mixture and extracted with EtOAc (80 mL×3). The combined organic layers were washed with brine (100 mL), dried over Na₂SO₄, filtered and concentrated under reduced pressure to give a residue which was purified by flash silica gel chromatography (from PE/EtOAc=1/0 to 2/1, TLC: PE/EtOAc=0/1, R_{f}=0.60) to yield O-tert-butyl N-(1r,4r)[4-[[4-[3-(4-amino-2-fluorophenoxy)-2-pyridyl┐pyrimidin-2-yl┐amino┐cyclohexyl┐carbamate (700 mg, 1.13 mmol, 50.5% yield, 80.0% purity) as a yellow solid. ¹H NMR (400 MHz, DMSO-d₆) δ ppm 8.35 (d, J=4.89 Hz, 2H), 7.41 (dd, J=4.59, 8.29 Hz, 1H), 7.21-7.03 (m, 2H), 6.88 (d, J=5.01 Hz, 2H), 6.70 (s, 1H), 6.48 (dd, J=2.38, 13.23 Hz, 1H), 6.39 (dd, J=2.15, 8.70 Hz, 1H), 5.37 (s, 2H), 3.63 (brs, 1H), 3.17 (brs, 1H), 1.94-1.89 (m, 2H), 1.74 (brs, 2H), 1.38 (s, 9H), 1.34-1.20 (m, 4H); ES-LCMS m/z 495.3 [M+H]⁺.

### Step 6: O-tert-Butyl N-(1r,4r)[4-[[4-[3-[4-[(2-chlorophenyl)sulfonylamino]-2-fluorophenoxy]-2-pyridyl]pyrimidin-2-yl]amino]cyclohexyl] carbamate

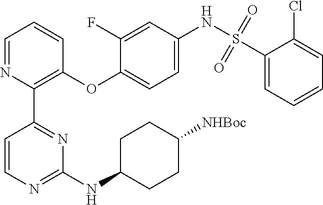 [SMILES: CC(C)(C)OC(=O)N[C@H]1CC[C@H](Nc2nccc(-c3ncccc3Oc3ccc(NS(=O)(=O)c4ccccc4Cl)cc3F)n2)CC1]

To a stirred solution of tert-butyl N-(1r,4r)[4-[[4-[3-(4-amino-2-fluorophenoxy)-2-pyridyl]pyrimidin-2-yl]amino]cyclohexyl] carbamate (300 mg, 533.81 μmol, 1 eq) in THF (8 mL) were added pyridine (422.24 mg, 5.34 mmol, 430.86 μL, 10 eq) and 2-chlorobenzenesulfonyl chloride (225.34 mg, 1.07 mmol, 145.57 μL, 2 eq). The reaction mixture was stirred at 25° C. for 5 h. To the mixture was added water (80 mL) and extracted with EtOAc (50 mL×3). The combined organic layers were washed with brine (50 mL), dried over Na₂SO₄, filtered and concentrated under reduced pressure to give a residue which was purified by preparative TLC (PE/EtOAc=3/1, TLC: PE/EtOAc=0/1, R_{f}=0.75) to yield O-tert-butyl N-(1r,4r)[4-[[4-[3-[4-[(2-chlorophenyl)sulfonylamino]-2-fluoro-phenoxy]-2-pyridyl┘pyrimidin-2-yl┘amino┘cyclohexyl┘ carbamate (350 mg, 491.66 μmol, 92.1% yield, 94.0% purity) as a white solid. ¹H NMR (400 MHz, DMSO-d₆) δ ppm 10.84 (brs, 1H), 8.42 (s, 1H), 8.30 (d, J=5.0 Hz, 1H), 8.04 (d, J=7.8 Hz, 1H), 7.65 (d, J=3.8 Hz, 2H), 7.56-7.50 (m, 1H), 7.44 (dd, J=4.5, 8.0 Hz, 1H), 7.31 (s, 1H), 7.06-6.94 (m, 3H), 6.90-6.78 (m, 2H), 6.60 (s, 1H), 3.46-3.41 (m, 1H), 3.16 (s, 1H), 1.89-1.62 (m, 4H), 1.39 (s, 9H), 1.35-1.18 (m, 4H); ES-LCMS m/z 669.2 [M+H]⁺.

### Step 7: N-[4-(1r,4r)[[2-[2-[(4-Aminocyclohexyl)amino]pyrimidin-4-yl]-3-pyridyl]oxy]-3-fluoro-phenyl] 2-chlorobenzenesulfonamide

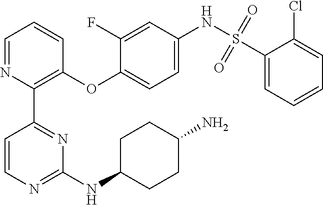 [SMILES: N[C@H]1CC[C@H](Nc2nccc(-c3ncccc3Oc3ccc(NS(=O)(=O)c4ccccc4Cl)cc3F)n2)CC1]

To a stirred solution of O-tert-butyl N-(1r,4r)4-[[4-[3-[4-[(2-chlorophenyl)sulfonylamino]-2-fluoro-phenoxy]-2-pyridyl]pyrimidin-2-yl]amino]cyclohexyl] carbamate (100 mg, 140.47 μmol, 1 eq) in DCM (3 mL) was added TFA (1.44 g, 12.65 mmol, 940.00 μL, 90.08 eq). The reaction mixture was stirred at 25° C. for 0.5 h. The reaction mixture was concentrated to yield a residue which was purified by preparative HPLC (column: Boston Green ODS 150*30 mm*5 μm; mobile phase: [water (FA)-ACN]; B %: 8%-38%, 12 min), followed by lyophilization to yield N-[4-(1r,4r)[[2-[2-[(4-aminocyclohexyl)amino]pyrimidin-4-yl]-3-pyridyl]oxy]-3-fluoro-phenyl] 2-chlorobenzenesulfonamide (42.8 mg, 69.59 μmol, 49.5% yield, 100.0% purity, 0.70 FA) as a white solid. ¹H NMR (400 MHz, DMSO-d₆, 75° C.) δ ppm 8.41 (dd, J=4.5, 1.4 Hz, 1H), 8.34 (d, J=5.0 Hz, 1H), 8.28 (s, 0.70 H (HCO₂H)), 7.99 (dd, J=7.7, 1.7 Hz, 1H), 7.52-7.32 (m, 5H), 6.92 (d, J=5.0 Hz, 1H), 6.85 (dd, J=13.9, 2.5 Hz, 1H), 6.78 (t, J=9.3 Hz, 1H)), 6.71-6.64 (m, 2H), 3.31-3.26 (m, 1H), 2.89 (brs, 1H), 1.93-1.80 (m, 4H), 1.35-1.22 (m, 4H); ES-LCMS m/z 569.3 [M+H]⁺.

### Example 12. Biological Data

### Surface Plasmon Resonance (SPR) Method Description

Compound interaction was profiled using a Biacore 8K instrument (Cytiva). Biotinylated N-terminal avi-tagged Cdk2(2-298) or Cdk1(1-297) was immobilized on a series S sensor Chip SA at a capture level between 2500 RU and 3000 RU (Cdk2) or 7000 RU (Cdk1) in buffer (25 mM HEPES, 150 mM NaCl, 0.05% Tween20, pH 7.5). A compound dilution plate (8 concentrations, 3-fold dilution) in DMSO was prepared (100× assay concentration). From this plate, compound DMSO stock solution was transferred into buffer, yielding a final DMSO concentration of 1% in assay. The remaining buffer was supplemented with 1% DMSO. Compounds were injected in a series of increasing concentrations in multi-cycle kinetics (MCK, “M”) or single-cycle kinetics (SCK, “S”) mode at a flow rate of 30 μl/min for 120 sec and dissociation was profiled for 180 sec (MCK) or 1800 sec (SCK). Data were analysed using Biacore Insight Evaluation Software (Cytiva). Results for exemplary compounds disclosed herein are summarized in Table 2.

In Table 2 below, “A” indicates a K_{D} of 10 nM or less; “B” indicates a K_{D} of 11-100 nM; “C” indicates a K_{D} of 100-1000 nM; “D” indicates a K_{D} of 1000-5000 nM; “E” indicates a K_{D} of >5000 nM.

TABLE 2

| Compound Number | Activity rating* | M/S (CK) |
|---|---|---|
| 1 | B | S |
| 2 | D | S |
| 3 | B | S |
| 4 | C | S |
| 5 | A | S |
| 6 | C | S |
| 7 | B | S |
| 8 | B | S |
| 9 | B | S |
| 10 | B | S |
| 11 | B | S |
| 16 | B | S |
| 17 | B | S |
| 18 | E | S |
| 19 | B | S |
| 20 | B | S |
| 21 | B | S |
| 22 | B | S |
| 23 | A | S |
| 24 | A | S |
| 25 | B | S |
| 26 | A | S |
| 27 | B | S |
| 28 | C | S |
| 29 | B | S |
| 30 | C | S |
| 31 | B | S |
| 32 | C | S |
| 33 | A | S |
| 34 | A | S |
| 35 | B | S |
| 36 | E | S |
| 37 | B | S |
| 38 | C | S |
| 39 | D | S |
| 40 | D | S |
| 41 | C | S |
| 42 | C | S |
| 43 | B | S |
| 44 | D | S |
| 45 | E | S |
| 46 | C | S |
| 47 | A | S |
| 48 | B | S |
| 49 | C | S |
| 50 | E | S |
| 51 | D | S |
| 52 | B | S |
| 53 | A | S |
| 54 | B | S |
| 55 | C | S |
| 56 | C | S |
| 57 | C | S |
| 58 | B | S |
| 59 | B | S |
| 60 | D | S |
| 61 | C | S |
| 62 | C | S |
| 63 | D | S |
| 64 | A | S |
| 65 | A | S |
| 66 | C | S |
| 67 | A | S |
| 68 | C | S |
| 69 | B | S |
| 70 | A | S |
| 71 | A | S |
| 72 | B | S |
| 73 | B | S |
| 74 | E | S |
| 75 | C | S |
| 76 | C | S |
| 77 | B | S |
| 78 | B | S |
| 79 | A | S |
| 81 | C | S |
| 82 | C | S |
| 83 | C | S |
| 84 | C | S |
| 85 | C | S |
| 86 | C | S |
| 87 | C | S |
| 88 | A | S |
| 89 | B | S |
| 90 | C | S |
| 91 | C | S |
| 92 | A | S |
| 93 | C | S |
| 94 | C | S |
| 95 | B | S |
| 96 | C | S |
| 97 | C | S |
| 98 | B | S |
| 99 | C | S |
| 100 | A | S |
| 101 | B | S |
| 102 | B | S |
| 103 | C | S |
| 104 | C | S |
| 105 | B | S |
| 106 | C | S |
| 107 | C | S |
| 108 | A | S |
| 109 | A | S |
| 110 | B | S |
| 111 | B | S |
| 112 | B | M |
| 113 | C | S |
| 116 | A | S |
| 117 | B | S |
| 118 | C | S |
| 127 | B | S |
| 128 | A | S |
| 129 | C | S |
| 130 | D | S |
| 131 | B | S |
| 132 | C | S |

1. A compound of Formula I: 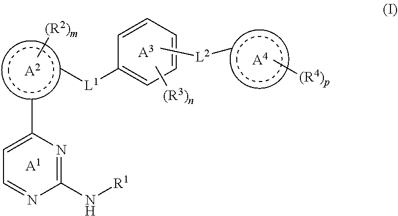
or a pharmaceutically acceptable salt thereof, wherein:
A² is phenyl, 2,3-naphthyl, or 5 or 6-membered heteroaryl;
A⁴ is aryl, heteroaryl, C₃₋₁₂ carbocyclyl, or 4-12 membered heterocyclyl;
L¹ is —O—, —S—, —NH—, —S(═O)₁₋₂—, —S(═O)(═NR⁵)—, —C(R⁶)₂—, or —C(═O)—;
L² is —NR⁷SO₂—, —C(R⁸)₂SO₂—, —NR⁷SO₂—C(R⁸)₂—, —C(R⁸)₂C(═O)—, —C(═O)C(R⁸)₂—, —C(═O)O—, —OC(═O)—, —C(R⁸)₂O—, —OC(R⁸)₂—, —C(═O)NR⁷—, —NR⁷C(═O)—, —C(R⁸)₂NR⁷—, —NR⁷C(R⁸)₂—, —C(R⁸)₂S(═O)₀₋₂—, —S(═O)₀₋₂C(R⁸)₂—, —C(R⁸)₂S(═O)NR⁷—, S(═O)NR⁷C(R⁸)₂—, or —C(R⁸)₂C(R⁸)₂—;
R¹ is C₃₋₁₂ carbocyclyl, 4-12 membered heterocyclyl, phenyl, 5-12 membered heteroaryl, —(C₇₋₆ alkylene)-(C₃₋₁₀ carbocyclyl), —(C₁₋₆ alkylene)-(4-12 membered heterocyclyl), —(C₁₋₆alkylene)-(phenyl), —(C₁₋₆alkylene)-(5-12 membered heteroaryl), —(C₁₋₆alkylene)-OR⁹, or —(C₁₋₆alkylene)-NR⁷R¹⁰, wherein R¹ is optionally substituted by 0-5 R¹¹;
R² is each independently H, halogen, cyano, nitro, hydroxy, —NR⁷R¹⁰, —NHCOR⁹, C₁₋₆ alkyl, C₂₋₆ alkenyl, C₂₋₆ alkynyl, C₁₋₆ alkoxy, C₂₋₆ alkenoxy, C₂₋₆ alkynoxy, COR⁹, CON(R⁹)₂, CF₃, CF₂H, CF₃CF₂, CF₃CH₂, C₁₋₆ haloalkyl, CF₃O, CF₂HO, CF₃CF₂O, CF₃CH₂O, C₁₋₆ haloalkoxy, C₃₋₈ cycloalkyl, C₃₋₈ cycloalkenyl, C₁₋₆ alkylthio, C₂₋₆ alkenylthio, C₁₋₆ alkylsulfonyl, aryl, or heteroaryl, wherein R² is each independently optionally substituted by 0-4 R¹¹;
R³ is each independently H, halogen, cyano, nitro, hydroxy, —NR⁷R¹⁰, —NHCOR⁹, C₁₋₆ alkyl, C₂₋₆ alkenyl, C₂₋₆ alkynyl, C₁₋₆ alkoxy, C₂₋₆ alkenoxy, C₂₋₆ alkynoxy, COR⁹, CON(R⁹)₂, CF₃, CF₂H, CF₃CF₂, CF₃CH₂, C₁₋₆ haloalkyl, CF₃O, CF₂HO, CF₃CF₂O, CF₃CH₂O, C₁₋₆ haloalkoxy, C₃₋₈ cycloalkyl, C₃₋₈ cycloalkenyl, C₁₋₆ alkylthio, C₂₋₆ alkenylthio, or C₁₋₆ alkylsulfonyl, wherein R³ is each independently optionally substituted by 0-4 R¹¹;
R⁴ is each independently H, halogen, cyano, nitro, hydroxy, NR⁷R¹⁰, NHCOR⁹, NR⁷CONR⁷, C₁₋₆ alkyl, C₂₋₆ alkenyl, C₂₋₆ alkynyl, COR⁹, CO₂R⁹, CONR⁷R¹⁰, C₁₋₆ alkoxy, C₂₋₆ alkenoxy, C₂₋₆ alkynoxy, CF₃, CF₂H, CF₃CF₂, CF₃CH₂, C₁₋₆ haloalkyl, CF₃O, CF₂HO, CF₃CF₂O, CF₃CH₂O, C₁₋₆ haloalkoxy, C₃₋₈ cycloalkyl, C₃₋₈ cycloalkenyl, C₁₋₆ alkylthio, C₂₋₆ alkenylthio, C₁₋₆ alkylsulfonyl, SO₂N(R⁷)₂, or phenyl, or any two occurrences of R⁴ on contiguous atoms, taken together with the contiguous atoms to which the two occurrences of R⁴ are attached, form a saturated or unsaturated 5-7 membered ring comprising 0-2 heteroatoms each independently selected from O, S(═O)₀₋₂, P(═O)R¹², N, and NR¹⁰, wherein R⁴ is each independently optionally substituted by 0-4 R¹¹;
R⁵ is each independently H or C₁₋₄ alkyl;
R⁶ is each independently H, F, OH, cyano, or C₁₋₆ alkyl;
R⁷ is each independently H, C₁₋₆ alkyl, C₁₋₆ haloalkyl, C₂₋₆ alkenyl, C₂₋₆ alkynyl, C₃₋₆ cycloalkyl, C₄₋₆ heterocycloalkyl, aryl, 5-10 membered heteroaryl, C₁₋₃ alkylene-C₃₋₆ cycloalkyl, or C₁₋₃ alkylene-C₄₋₆ heterocycloalkyl, or two R⁷s along with the N atom to which they are attached form a 4-7 membered ring comprising an additional 0-1 heteroatoms selected from NR⁵, O and S(═O)₀₋₂;
R⁸ is each independently H, halogen, cyano, C₁₋₆ alkyl, C₁₋₆ haloalkyl, C₂₋₆ alkenyl, C₂₋₆ alkynyl, C₂₋₆ cycloalkyl, C₄₋₆heterocycloalkyl, C₁₋₃ alkylene-C₃₋₆ cycloalkyl, C₁₋₃ alkylene-C₄₋₆ heterocycloalkyl, or two R⁸s taken together with the C atom to which they are attached form a 4-7 membered ring comprising 0-1 heteroatom selected from O, S(═O)₀₋₂, P(═O)R¹², and NR⁵, wherein R⁸ is optionally substituted by 1-3 R¹¹;
each R⁹ is H, C₁₋₆ alkyl, C₁₋₆ haloalkyl, C₂₋₆ alkenyl, C₂₋₆ alkynyl, C₃₋₁₀ cycloalkyl, C₄₋₁₀ heterocycloalkyl, C₁₋₃ alkylene-C₃₋₆ cycloalkyl, C₁₋₃ alkylene-C₄₋₆ heterocycloalkyl, C₁₋₆ acyl, phenyl, or 5-12 membered heteroaryl, wherein R⁹ is optionally substituted by 1-3 R¹¹;
each R¹⁰ is independently H, C₁₋₆ alkyl, C₁₋₆ haloalkyl, C₂₋₆ alkenyl, C₂₋₆ alkynyl, C₃₋₁₀ cycloalkyl, C₄₋₁₀ heterocycloalkyl, C₁₋₃ alkylene-C₃₋₆ cycloalkyl, C₁₋₃ alkylene-C₄₋₆heterocycloalkyl, C₁₋₆ acyl, C₁₋₆ alkylsulfonyl, phenyl, or 5-12 membered heteroaryl, wherein R¹⁰ is optionally substituted by 1-3 R¹¹;
or R⁷ and R¹⁰ on the same N atom may be taken together with the N atom to which they are attached form a 4-7 membered ring comprising an additional 0-1 heteroatoms selected from NR⁵, O, S(═O)₀₋₂, P(═O)R¹², and NR¹³;
R¹¹ is each independently R⁶, oxo, nitro, C₂₋₆ alkenyl, C₂₋₆ alkynyl, C₁₋₆ alkoxy, C₁₋₆ alkoxyalkyl, C₁₋₆hydroxyalkyl, C₁₋₆ haloalkyl, C₁₋₆ haloalkoxy, C₃₋₆ cycloalkyl, 4-6 membered heterocycloalkyl, C₁₋₃ alkylene-C₃₋₆ cycloalkyl, C₁₋₃ alkylene-C₄₋₆ heterocycloalkyl, C₆₋₁₀ aryl, C₅₋₁₀ heteroaryl, SO₂R⁷, S(═O)NR⁵, SO₂N(R⁷)₂, CON(R⁷)₂, or N(R⁷)₂;
R¹² is each independently H, OH, C₁₋₆ alkyl, C₁₋₆ alkoxy, or —N(R⁷)₂;
R¹³ is each independently H, C₁₋₆ alkyl, C₁₋₆ haloalkyl, C₂₋₆ alkenyl, C₂₋₆ alkynyl, C₂₋₆ cycloalkyl, C₄₋₆ heterocycloalkyl, C₁₋₃ alkylene-C₃₋₆ cycloalkyl, or C₁₋₃ alkylene-C₄₋₆ heterocycloalkyl, C₁₋₆ acyl, aroyl, or heteroaroyl;
m is an integer 0, 1 or 2;
n is an integer 0, 1, 2, or 3; and
p is an integer 0, 1, 2, or 3,
provided that
the A¹ ring and the L¹ linker are on contiguous C atoms of A², whereas L¹ and L² are not situated on contiguous atoms of the A³ ring;
and when A² is 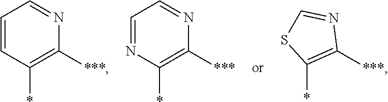
L¹ is not —O—, wherein * is the site covalently linked to the A¹ ring, and *** is the site covalently linked to L¹.

2. The compound of claim 1, wherein A⁴ is phenyl, naphthyl, 5-10 membered heteroaryl, C₃₋₁₀ carbocyclyl, or 5-10 membered heterocyclyl.

3. The compound of claim 1, wherein A⁴ is phenyl, naphthyl, thiophenyl, cyclopropyl, cyclobutyl, cyclopentyl, cyclohexyl,

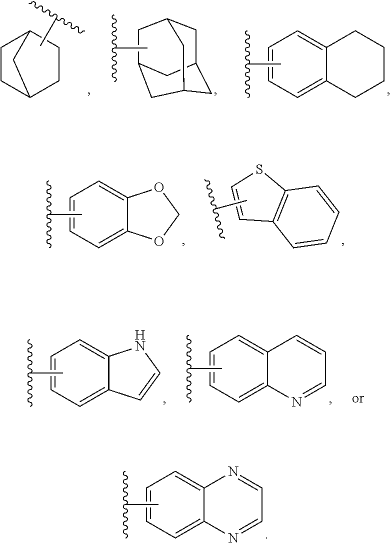

4. The compound of claim 1, wherein A⁴ is phenyl.

5. The compound of any one of claims 1-4, wherein A² is phenyl, 2,3-naphthyl, pyridinyl, or pyrimidinyl.

6. The compound of claim 5, wherein A² is 1,2-linked phenylene, 2,3-linked-naphthylene, 2-L¹-3-biaryl linked pyridylene, 4-L¹-3-biaryl linked pyridylene or 4-L¹-5-biaryl linked pyrimidylene.

7. The compound of claim 5, wherein A² is 1,2-linked phenylene.

8. The compound of any one of claims 1-7, wherein R¹ is C₆₋₁₀carbocyclyl, 4-8 membered heterocyclyl, phenyl, 5-6 membered heteroaryl, —(C₁₋₆alkylene)-(C₃₋₁₀carbocyclyl), —(C₁₋₆alkylene)-(4-8 membered heterocyclyl), —(C₁₋₆alkylene)-(5-6 membered heteroaryl), —(C₁₋₆alkylene)-OR⁹, or —(C₁₋₆alkylene)-NR⁷R¹⁰, wherein R¹ is optionally substituted by 1-2 R¹¹.

9. The compound of claim 8, wherein R¹ is C₆₋₁₀carbocyclyl, 4-8 membered heterocyclyl, phenyl, 5-6 membered heteroaryl, —(C₁₋₆alkylene)-(C₃₋₁₀carbocyclyl), —(C₁₋₆alkylene)-(4-8 membered heterocyclyl), —(C₁₋₆alkylene)-(5-6 membered heteroaryl), —(C₁₋₆alkylene)-OR⁹, or —(C₁₋₆alkylene)-NR⁷R¹⁰, wherein R¹ is optionally substituted by 1 R¹¹.

10. The compound of claim 8, wherein R¹ is cyclohexyl optionally substituted with 1 R¹¹.

11. The compound of claim 8, wherein R¹ is piperidinyl, oxetanyl, azetidinyl, pyrrolidinyl, or azaspiroheptanyl, optionally substituted with 1 R¹¹.

12. The compound of claim 8, wherein R¹ is —(C₁₋₃alkylene)-OR⁹ optionally substituted with 1 R¹¹.

13. The compound of claim 8, wherein R¹ is —(C₁₋₃alkylene)-NR⁷R¹⁰ optionally substituted with 1 R¹¹.

14. The compound of claim 8, wherein R¹ is —(C₁₋₃alkylene)-(4-6 membered heterocyclyl containing 1-2 heteroatoms selected from the group consisting of O, S, and N) optionally substituted by 1 R¹¹.

15. The compound of claim 8, wherein R¹ is —(C₁₋₃alkylene)-(5-6 membered heteroaryl containing 1-2 heteroatoms selected from the group consisting of O, S, and N) optionally substituted by 1 R¹¹.

16. The compound of any one of claims 1-15, wherein L¹ is —O—, —S—, —NH—, —S(═O)₁₋₂, —S(═O)(═NR⁵)—, —CHR⁶—, or —CF₂—.

17. The compound of claim 16, wherein L¹ is —O— or —S—.

18. The compound of claim 17, wherein L¹ is —O—.

19. The compound of claim 17, wherein L¹ is —S—.

20. The compound of any one of claims 1-19, wherein L² —NHSO₂—, —NHSO₂—CH₂—, —C(═O)NH—, or —NHC(═O)—.

21. The compound of any one of claims 1-19, wherein L² is —NR⁷SO₂—.

22. The compound of claim 21, wherein L² is —NHSO₂—.

23. The compound of any one of claims 1-22, wherein R⁶ is each independently H, methyl, ethyl, fluoro, chloro, cyano, CF₃, CHF₂, or CH₂F.

24. The compound of any one of claims 1-23, wherein R⁸ is each independently H, methyl, ethyl, fluoro, chloro, cyano, CF₃, CHF₂, CH₂F, methoxy, or ethoxy.

25. The compound of any one of claims 1-24, wherein m is 0.

26. The compound of any one of claims 1-24, wherein m is 1.

27. The compound of any one of claims 1-24 and 26, wherein R² is each independently H, halogen, cyano, C₁₋₆ alkyl, C₂₋₆alkenyl, C₂₋₆alkynyl, C₁₋₆haloalkyl, C₃₋₆cycloalkyl, aryl or heteroaryl.

28. The compound of any one of claims 1-24 and 26, wherein R² is each independently H, halogen, cyano, C₁₋₆ alkyl, C₂₋₄alkenyl, C₂₋₄alkynyl, C₁₋₄haloalkyl, or C₃₋₆cycloalkyl.

29. The compound of any one of claims 1-28, wherein n is 0, 1, or 2.

30. The compound of claim 29, wherein n is 1 or 2.

31. The compound of any one of claims 1-30, wherein R³ is each independently H, halogen, cyano, C₁₋₄alkyl, or C₁₋₄haloalkyl.

32. The compound of any one of claims 1-30, wherein R³ is each independently H, halogen, cyano, or C₁₋₂haloalkyl.

33. The compound of any one of claims 1-32, wherein p is 0, 1, or 2.

34. The compound of claim 33, wherein p is 1 or 2.

35. The compound of any one of claims 1-34, wherein R⁴ is each H, halogen, cyano, nitro, hydroxy, NR⁷R¹⁰, NHCOR⁹, NR⁷CONR⁷, C₁₋₆alkyl, C₂₋₆alkenyl, C₂₋₆alkynyl, COR⁹, CO₂R⁹, CONR⁷R¹⁰, C₁₋₆alkoxy, C₁₋₆haloalkyl, C₁₋₆haloalkoxy, or C₃₋₈cycloalkyl, wherein R⁴ is each independently optionally substituted by 0-1 R¹¹.

36. The compound of claim 35, wherein R⁴ is each independently H, halogen, cyano, nitro, C₁₋₆ alkyl optionally substituted with R¹¹, C₂₋₆alkenyl, C₂₋₆alkynyl, C₁₋₆haloalkyl, C₁₋₆alkoxy, C₃₋₆cycloalkyl, or CONR⁷R¹⁰.

37. The compound of any one of claims 1-36, wherein R⁹ is H, C₃₋₁₀ cycloalkyl, C₄₋₁₀ heterocycloalkyl, phenyl, or 5-12 membered heteroaryl, wherein R⁹ is optionally substituted by 1-2 R¹¹.

38. The compound of any one of claims 1-36, wherein R⁹ is C₁₋₆alkyl optionally substituted by 1 R¹¹.

39. The compound of any one of claims 1-38, wherein R⁷ is H or C₁₋₆alkyl.

40. The compound of any one of claims 1-39, wherein R¹⁰ is H or C₁₋₆alkyl, wherein the R¹⁰ is optionally substituted by 1-2 R¹¹.

41. The compound of any one of claims 1-40, wherein R¹¹ is each independently R⁶, nitro, C₂₋₆ alkenyl, C₂₋₆ alkynyl, C₁₋₆ alkoxy, C₁₋₆haloalkyl, C₁₋₆ haloalkoxy, C₃₋₆ cycloalkyl, 4-6 membered heterocycloalkyl, C₁₋₃ alkylene-C₃₋₆ cycloalkyl, C₁₋₃ alkylene-C₄₋₆ heterocycloalkyl, C₆₋₁₀ aryl, C₅₋₆ heteroaryl, SO₂R⁷, S(═O)NR⁵, SO₂N(R⁷)₂, CON(R⁷)₂, or N(R⁷)₂.

42. The compound of claim 31, wherein R¹¹ is each independently R⁶, C₁₋₆ alkoxy, C₁₋₆ haloalkyl, C₃₋₆ cycloalkyl, 4-6 membered heterocycloalkyl, C₆₋₁₀ aryl, C₅₋₆ heteroaryl, SO₂R⁷, S(═O)NR⁵, SO₂N(R⁷)₂, CON(R⁷)₂, or N(R⁷)₂.

43. The compound of claim 41, wherein R¹¹ is each independently N(R⁷)₂.

44. A compound of Formula III: 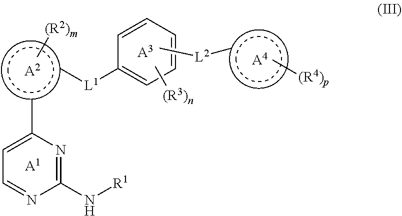
or a pharmaceutically acceptable salt thereof, wherein:
A² is 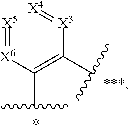
wherein * is the site covalently linked to the A¹ ring, and *** is the site covalently linked to L¹;
A⁴ is aryl, heteroaryl, C₃₋₁₂ carbocyclyl, or 4-12 membered heterocyclyl;
L¹ is —O—, —S—, —NH—, —S(═O)₁₋₂—, —S(═O)(═NR⁵)—, —C(R⁶)₂—, or —C(═O)—;
L² is —NR⁷SO₂—, —C(R⁸)₂SO₂—, —NR⁷SO₂—C(R⁸)₂—, —C(R⁸)₂C(═O)—, —C(═O)C(R⁸)₂—, —C(═O)O—, —OC(═O)—, —C(R⁸)₂O—, —OC(R⁸)₂—, —C(═O)NR⁷—, —NR⁷C(═O)—, —C(R⁸)₂NR⁷—, —NR⁷C(R⁸)₂—, —C(R⁸)₂S(═O)₀₋₂—, —S(═O)₀₋₂C(R⁸)₂—, —C(R⁸)₂S(═O)NR⁷—, S(═O)NR⁷C(R⁸)₂—, or —C(R⁸)₂C(R⁸)₂—;
R¹ is C₃₋₁₂ carbocyclyl, 4-12 membered heterocyclyl, phenyl, 5-12 membered heteroaryl, —(C₁₋₆ alkylene)-(C₃₋₁₀ carbocyclyl), —(C₁₋₆ alkylene)-(4-12 membered heterocyclyl), —(C₁₋₆alkylene)-(phenyl), —(C₁₋₆alkylene)-(5-12 membered heteroaryl), —(C₁₋₆alkylene)-OR⁹, or —(C₁₋₆alkylene)-NR⁷R¹⁰, wherein R¹ is optionally substituted by 0-5 R¹¹;
R² is each independently H, halogen, cyano, nitro, hydroxy, —NR⁷R¹⁰, —NHCOR⁹, C₁₋₆ alkyl, C₂₋₆ alkenyl, C₂₋₆ alkynyl, C₁₋₆ alkoxy, C₂₋₆ alkenoxy, C₂₋₆ alkynoxy, COR⁹, CON(R⁹)₂, CF₃, CF₂H, CF₃CF₂, CF₃CH₂, C₁₋₆ haloalkyl, CF₃O, CF₂HO, CF₃CF₂O, CF₃CH₂O, C₁₋₆ haloalkoxy, C₃₋₈ cycloalkyl, C₃₋₈ cycloalkenyl, C₁₋₆ alkylthio, C₂₋₆ alkenylthio, C₁₋₆ alkylsulfonyl, aryl or heteroaryl, wherein R² is each independently optionally substituted by 0-4 R¹¹;
R³ is each independently H, halogen, cyano, nitro, hydroxy, —NR⁷R¹⁰, —NHCOR⁹, C₁₋₆ alkyl, C₂₋₆ alkenyl, C₂₋₆ alkynyl, C₁₋₆ alkoxy, C₂₋₆ alkenoxy, C₂₋₆ alkynoxy, COR⁹, CON(R⁹)₂, CF₃, CF₂H, CF₃CF₂, CF₃CH₂, C₁₋₆haloalkyl, CF₃O, CF₂HO, CF₃CF₂O, CF₃CH₂O, C₁₋₆haloalkoxy, C₃₋₈ cycloalkyl, C₃₋₈ cycloalkenyl, C₁₋₆ alkylthio, C₂₋₆ alkenylthio, or C₁₋₆ alkylsulfonyl, wherein R³ is each independently optionally substituted by 0-4 R¹¹;
R⁴ is each independently H, halogen, cyano, nitro, hydroxy, NR⁷R¹⁰, NHCOR⁹, NR⁷CONR⁷, C₁₋₆ alkyl, C₂₋₆ alkenyl, C₂₋₆ alkynyl, COR⁹, CO₂R⁹, CONR⁷R¹⁰, C₁₋₆ alkoxy, C₂₋₆ alkenoxy, C₂₋₆ alkynoxy, CF₃, CF₂H, CF₃CF₂, CF₃CH₂, C₁₋₆ haloalkyl, CF₃O, CF₂HO, CF₃CF₂O, CF₃CH₂O, C₁₋₆ haloalkoxy, C₃₋₈ cycloalkyl, C₃₋₈ cycloalkenyl, C₁₋₆ alkylthio, C₂₋₆ alkenylthio, C₁₋₆ alkylsulfonyl, SO₂N(R⁷)₂, or phenyl, or any two occurrences of R⁴ on contiguous atoms, taken together with the contiguous atoms to which the two occurrences of R⁴ are attached, form a saturated or unsaturated 5-7 membered ring comprising 0-2 heteroatoms each independently selected from O, S(═O)₀₋₂, P(═O)R¹², N, and NR¹⁰, wherein R⁴ is each independently optionally substituted by 0-4 R¹¹;
R⁵ is each independently H or C₁₋₄ alkyl;
R⁶ is each independently H, F, OH, cyano, or C₁₋₆ alkyl;
R⁷ is each independently H, C₁₋₆ alkyl, C₁₋₆ haloalkyl, C₂₋₆ alkenyl, C₂₋₆ alkynyl, C₃₋₆ cycloalkyl, C₄₋₆ heterocycloalkyl, aryl, 5-10 membered heteroaryl, C₁₋₃ alkylene-C₃₋₆ cycloalkyl, or C₁₋₃ alkylene-C₄₋₆ heterocycloalkyl, or two R⁷s along with the N atom to which they are attached form a 4-7 membered ring comprising an additional 0-1 heteroatoms selected from NR⁵, O and S(═O)₀₋₂;
R⁸ is each independently H, halogen, cyano, C₁₋₆ alkyl, C₁₋₆ haloalkyl, C₂₋₆ alkenyl, C₂₋₆ alkynyl, C₂₋₆ cycloalkyl, C₄₋₆heterocycloalkyl, C₁₋₃ alkylene-C₃₋₆ cycloalkyl, C₁₋₃ alkylene-C₄₋₆ heterocycloalkyl, or two R⁸s taken together with the C atom to which they are attached form a 4-7 membered ring comprising 0-1 heteroatom selected from O, S(═O)₀₋₂, P(═O)R¹², and NR⁵, wherein R⁸ is optionally substituted by 1-3 R¹¹;
each R⁹ is H, C₁₋₆ alkyl, C₁₋₆ haloalkyl, C₂₋₆ alkenyl, C₂₋₆ alkynyl, C₃₋₁₀ cycloalkyl, C₄₋₁₀ heterocycloalkyl, C₁₋₃ alkylene-C₃₋₆ cycloalkyl, C₁₋₃ alkylene-C₄₋₆ heterocycloalkyl, C₁₋₆ acyl, phenyl, or 5-12 membered heteroaryl, wherein R⁹ is optionally substituted by 1-3 R¹¹;
each R¹⁰ is independently H, C₁₋₆ alkyl, C₁₋₆ haloalkyl, C₂₋₆ alkenyl, C₂₋₆ alkynyl, C₃₋₁₀ cycloalkyl, C₄₋₁₀ heterocycloalkyl, C₁₋₃ alkylene-C₃₋₆ cycloalkyl, C₁₋₃ alkylene-C₄₋₆ heterocycloalkyl, C₁₋₆ acyl, C₁₋₆ alkylsulfonyl, phenyl, or 5-12 membered heteroaryl, wherein R¹⁰ is optionally substituted by 1-3 R¹¹,
or R⁷ and R¹⁰ on the same N atom may be taken together with the N atom to which they are attached form a 4-7 membered ring comprising an additional 0-1 heteroatoms selected from NR⁵, O, S(═O)₀₋₂, P(═O)R¹², and NR¹³;
R¹¹ is each independently R⁶, oxo, nitro, C₂₋₆ alkenyl, C₂₋₆ alkynyl, C₁₋₆ alkoxy, C₁₋₆ alkoxyalkyl, C₁₋₆hydroxyalkyl, C₁₋₆ haloalkyl, C₁₋₆ haloalkoxy, C₃₋₆ cycloalkyl, 4-6 membered heterocycloalkyl, C₁₋₃ alkylene-C₃₋₆ cycloalkyl, C₁₋₃ alkylene-C₄₋₆ heterocycloalkyl, C₆₋₁₀ aryl, C₅₋₁₀ heteroaryl, SO₂R⁷, S(═O)NR⁵, SO₂N(R⁷)₂, CON(R⁷)₂, or N(R⁷)₂;
R¹² is each independently H, OH, C₁₋₆ alkyl, C₁₋₆ alkoxy, or —N(R⁷)₂;
R¹³ is each independently H, C₁₋₆ alkyl, C₁₋₆ haloalkyl, C₂₋₆ alkenyl, C₂₋₆ alkynyl, C₂₋₆ cycloalkyl, C₄₋₆ heterocycloalkyl, C₁₋₃ alkylene-C₃₋₆ cycloalkyl, or C₁₋₃ alkylene-C₄₋₆ heterocycloalkyl, C₁₋₆ acyl, aroyl, or heteroaroyl;
m is an integer 0, 1 or 2;
n is an integer 0, 1, 2, or 3; and
p is an integer 0, 1, 2, or 3,
provided that
L¹ and L² are not situated on contiguous atoms of the A³ ring;
and when A² is 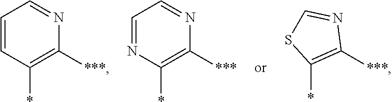
L¹ is not —O—, wherein * is the site covalently linked to the A¹ ring, and *** is the site covalently linked to L¹.

45. The compound of claim 44, wherein A⁴ is phenyl, naphthyl, 5-10 membered heteroaryl, C₃₋₁₀ carbocyclyl, or 5-10 membered heterocyclyl.

46. The compound of claim 44, wherein A⁴ is phenyl, naphthyl, thiophenyl, cyclopropyl, cyclobutyl, cyclopentyl, cyclohexyl,

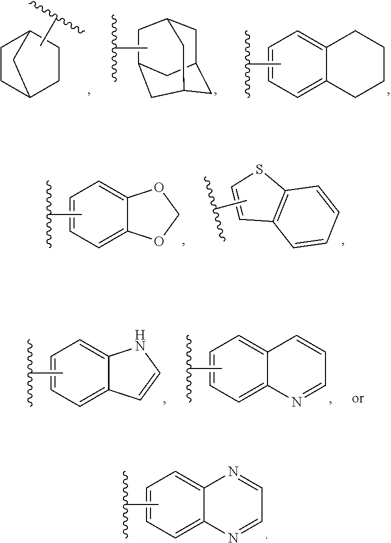

47. The compound of claim 44, wherein A⁴ is phenyl.

48. The compound of any one of claims 44-47, wherein A² is phenyl,

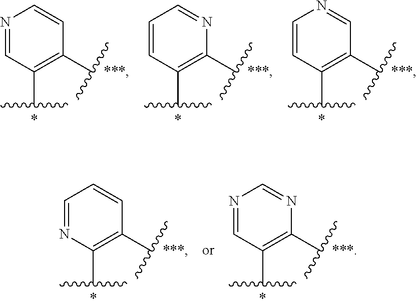 [SMILES: *c1ccncc1* ; *c1cccnc1* ; *c1ccncc1* ; *c1cccnc1* ; *c1cncnc1*]

49. The compound of any one of claims 44-48, wherein R¹ is cyclohexyl optionally substituted with 1 R¹¹.

50. The compound of any one of claims 44-48, wherein R¹ is piperidinyl, oxetanyl, azetidinyl, pyrrolidinyl, or azaspiroheptanyl, optionally substituted with 1 R¹¹.

51. The compound of any one of claims 44-50, wherein L¹ is —O— or —S—.

52. The compound of any one of claims 44-51, wherein L² is —NHSO₂—.

53. The compound of any one of claims 44-52, wherein m is 0.

54. The compound of any one of claims 44-52, wherein m is 1.

55. The compound of any one of claims 44-52 and 54, wherein R² is each independently H, halogen, cyano, C₁₋₆ alkyl, C₂₋₆alkenyl, C₂₋₆alkynyl, C₁₋₆haloalkyl, C₃₋₆cycloalkyl, aryl, or heteroaryl.

56. The compound of any one of claims 44-55, wherein n is 0, 1, or 2.

57. The compound of claim 56, wherein n is 1 or 2.

58. The compound of any one of claims 44-57, wherein R³ is each independently H, halogen, cyano, C₁₋₄alkyl, or C₁₋₄haloalkyl.

59. The compound of claim 58, wherein R³ is each independently halogen.

60. The compound of any one of claims 44-59, wherein p is 0, 1, or 2.

61. The compound of claim 60, wherein p is 1 or 2.

62. The compound of any one of claims 44-61, wherein R⁴ is each H, halogen, cyano, nitro, hydroxy, NR⁷R¹⁰, NHCOR⁹, NR⁷CONR⁷, C₁₋₆alkyl, C₂₋₆alkenyl, C₂₋₆alkynyl, COR⁹, CO₂R⁹, CONR⁷R¹⁰, C₁₋₆alkoxy, C₁₋₆haloalkyl, C₁₋₆haloalkoxy, or C₃₋₈cycloalkyl, wherein R⁴ is each independently optionally substituted by 0-1 R¹¹.

63. The compound of claim 62, wherein R⁴ is each independently H, halogen, cyano, nitro, C₁₋₆ alkyl optionally substituted with R¹¹, C₂₋₆alkenyl, C₂₋₆alkynyl, C₁₋₆haloalkyl, C₁₋₆alkoxy, C₃₋₆cycloalkyl, or CONR⁷R¹⁰.

64. The compound of claim 63, wherein R⁴ is each independently halogen.

65. A compound selected from the group consisting of: 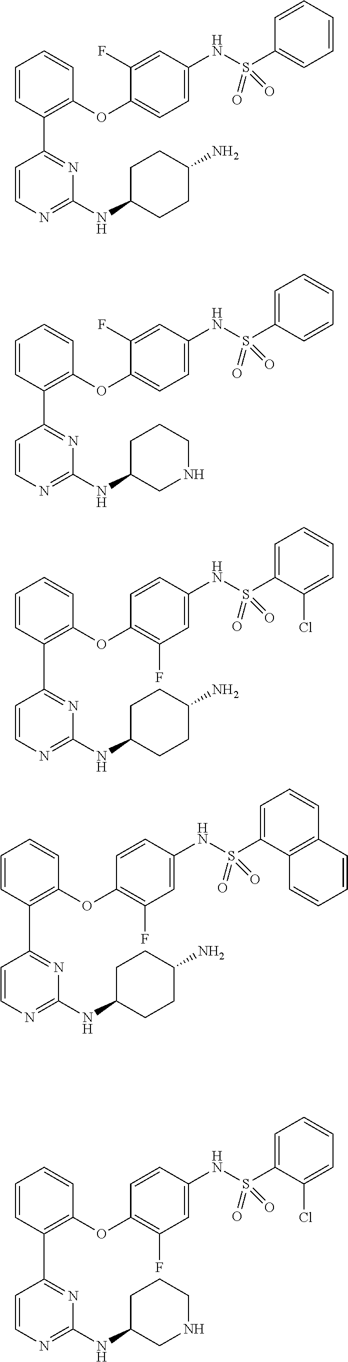 [SMILES: N[C@H]1CC[C@H](Nc2nccc(-c3ccccc3Oc3ccc(NS(=O)(=O)c4ccccc4)cc3F)n2)CC1 ; O=S(=O)(Nc1ccc(Oc2ccccc2-c2ccnc(N[C@H]3CCCNC3)n2)c(F)c1)c1ccccc1 ; N[C@H]1CC[C@H](Nc2nccc(-c3ccccc3Oc3ccc(NS(=O)(=O)c4ccccc4Cl)cc3F)n2)CC1 ; N[C@H]1CC[C@H](Nc2nccc(-c3ccccc3Oc3ccc(NS(=O)(=O)c4cccc5ccccc45)cc3F)n2)CC1 ; O=S(=O)(Nc1ccc(Oc2ccccc2-c2ccnc(N[C@H]3CCCNC3)n2)c(F)c1)c1ccccc1Cl] 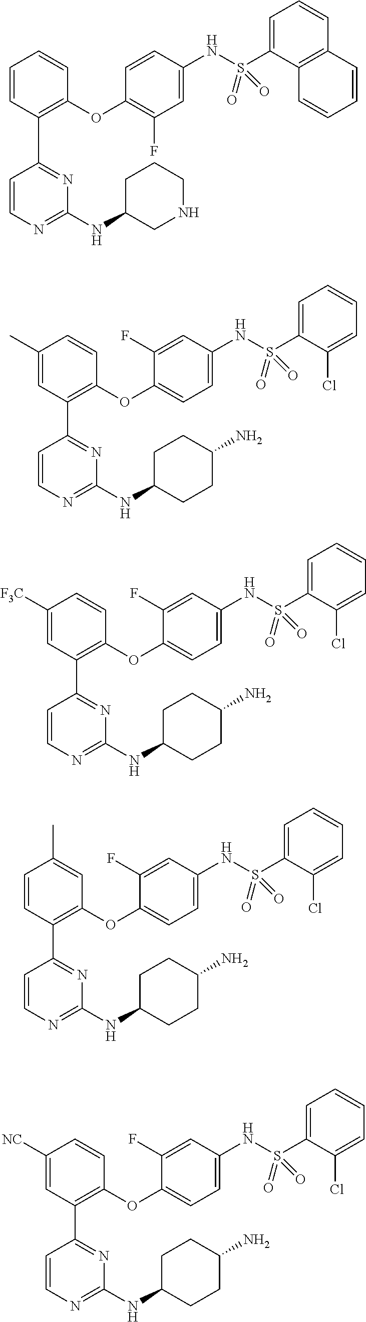 [SMILES: O=S(=O)(Nc1ccc(Oc2ccccc2-c2ccnc(N[C@H]3CCCNC3)n2)c(F)c1)c1cccc2ccccc12 ; Cc1ccc(Oc2ccc(NS(=O)(=O)c3ccccc3Cl)cc2F)c(-c2ccnc(N[C@H]3CC[C@H](N)CC3)n2)c1 ; N[C@H]1CC[C@H](Nc2nccc(-c3cc(C(F)(F)F)ccc3Oc3ccc(NS(=O)(=O)c4ccccc4Cl)cc3F)n2)CC1 ; Cc1ccc(-c2ccnc(N[C@H]3CC[C@H](N)CC3)n2)c(Oc2ccc(NS(=O)(=O)c3ccccc3Cl)cc2F)c1 ; N#Cc1ccc(Oc2ccc(NS(=O)(=O)c3ccccc3Cl)cc2F)c(-c2ccnc(N[C@H]3CC[C@H](N)CC3)n2)c1] 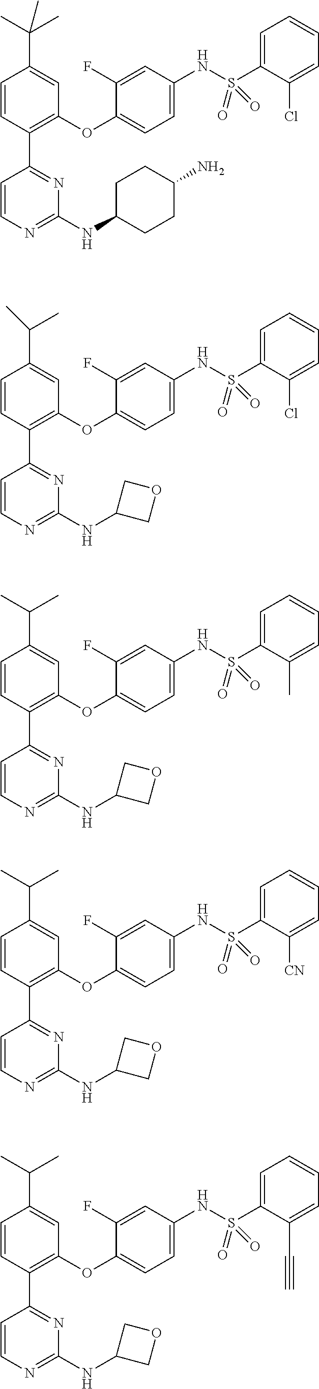 [SMILES: CC(C)(C)c1ccc(-c2ccnc(N[C@H]3CC[C@H](N)CC3)n2)c(Oc2ccc(NS(=O)(=O)c3ccccc3Cl)cc2F)c1 ; CC(C)c1ccc(-c2ccnc(NC3COC3)n2)c(Oc2ccc(NS(=O)(=O)c3ccccc3Cl)cc2F)c1 ; Cc1ccccc1S(=O)(=O)Nc1ccc(Oc2cc(C(C)C)ccc2-c2ccnc(NC3COC3)n2)c(F)c1 ; CC(C)c1ccc(-c2ccnc(NC3COC3)n2)c(Oc2ccc(NS(=O)(=O)c3ccccc3C#N)cc2F)c1 ; C#Cc1ccccc1S(=O)(=O)Nc1ccc(Oc2cc(C(C)C)ccc2-c2ccnc(NC3COC3)n2)c(F)c1] 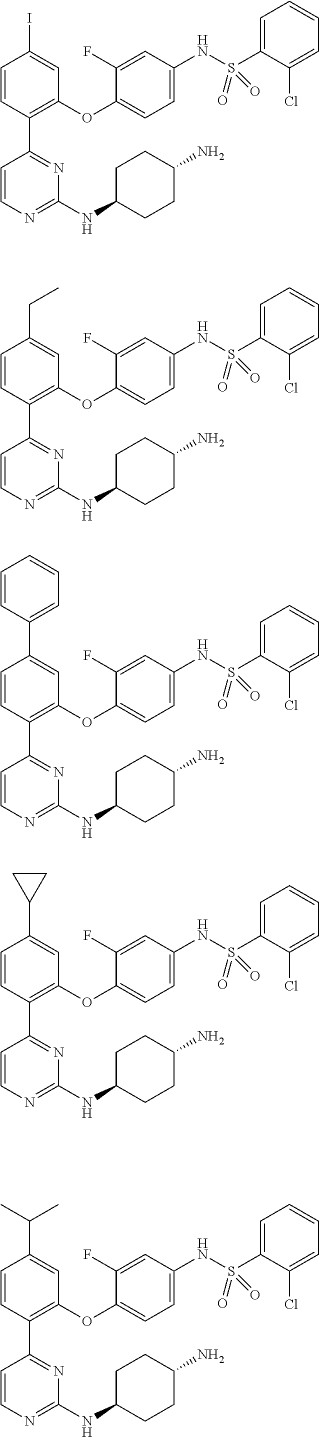 [SMILES: N[C@H]1CC[C@H](Nc2nccc(-c3ccc(I)cc3Oc3ccc(NS(=O)(=O)c4ccccc4Cl)cc3F)n2)CC1 ; CCc1ccc(-c2ccnc(N[C@H]3CC[C@H](N)CC3)n2)c(Oc2ccc(NS(=O)(=O)c3ccccc3Cl)cc2F)c1 ; N[C@H]1CC[C@H](Nc2nccc(-c3ccc(-c4ccccc4)cc3Oc3ccc(NS(=O)(=O)c4ccccc4Cl)cc3F)n2)CC1 ; N[C@H]1CC[C@H](Nc2nccc(-c3ccc(C4CC4)cc3Oc3ccc(NS(=O)(=O)c4ccccc4Cl)cc3F)n2)CC1 ; CC(C)c1ccc(-c2ccnc(N[C@H]3CC[C@H](N)CC3)n2)c(Oc2ccc(NS(=O)(=O)c3ccccc3Cl)cc2F)c1] 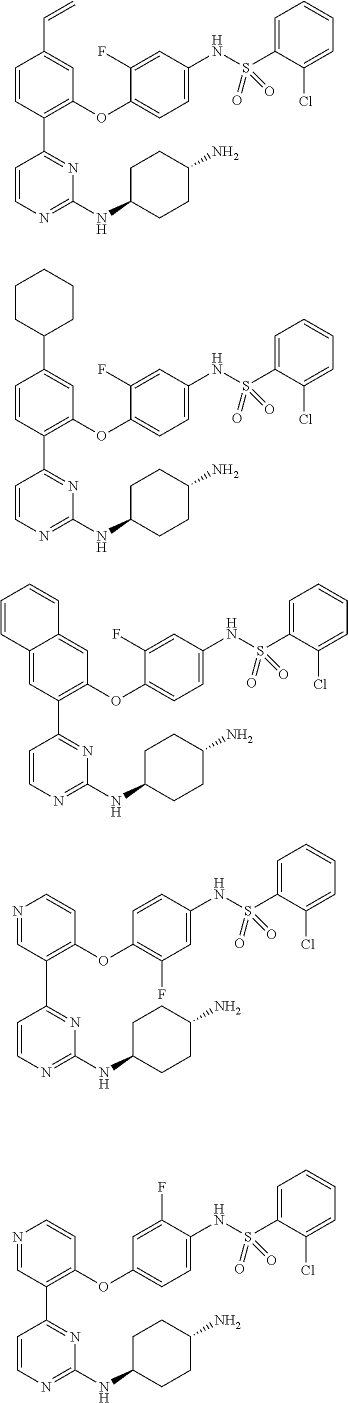 [SMILES: C=Cc1ccc(-c2ccnc(N[C@H]3CC[C@H](N)CC3)n2)c(Oc2ccc(NS(=O)(=O)c3ccccc3Cl)cc2F)c1 ; N[C@H]1CC[C@H](Nc2nccc(-c3ccc(C4CCCCC4)cc3Oc3ccc(NS(=O)(=O)c4ccccc4Cl)cc3F)n2)CC1 ; N[C@H]1CC[C@H](Nc2nccc(-c3cc4ccccc4cc3Oc3ccc(NS(=O)(=O)c4ccccc4Cl)cc3F)n2)CC1 ; N[C@H]1CC[C@H](Nc2nccc(-c3cnccc3Oc3ccc(NS(=O)(=O)c4ccccc4Cl)cc3F)n2)CC1 ; N[C@H]1CC[C@H](Nc2nccc(-c3cnccc3Oc3ccc(NS(=O)(=O)c4ccccc4Cl)c(F)c3)n2)CC1] 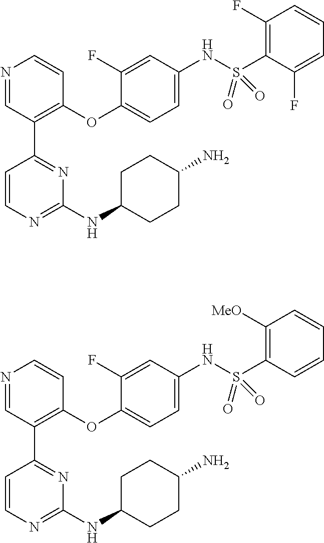 [SMILES: N[C@H]1CC[C@H](Nc2nccc(-c3cnccc3Oc3ccc(NS(=O)(=O)c4c(F)cccc4F)cc3F)n2)CC1 ; COc1ccccc1S(=O)(=O)Nc1ccc(Oc2ccncc2-c2ccnc(N[C@H]3CC[C@H](N)CC3)n2)c(F)c1] 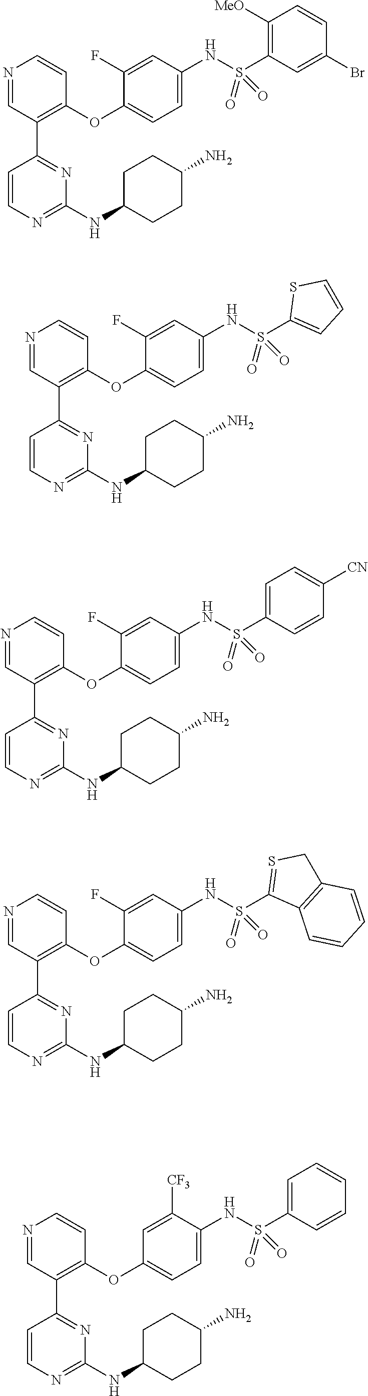 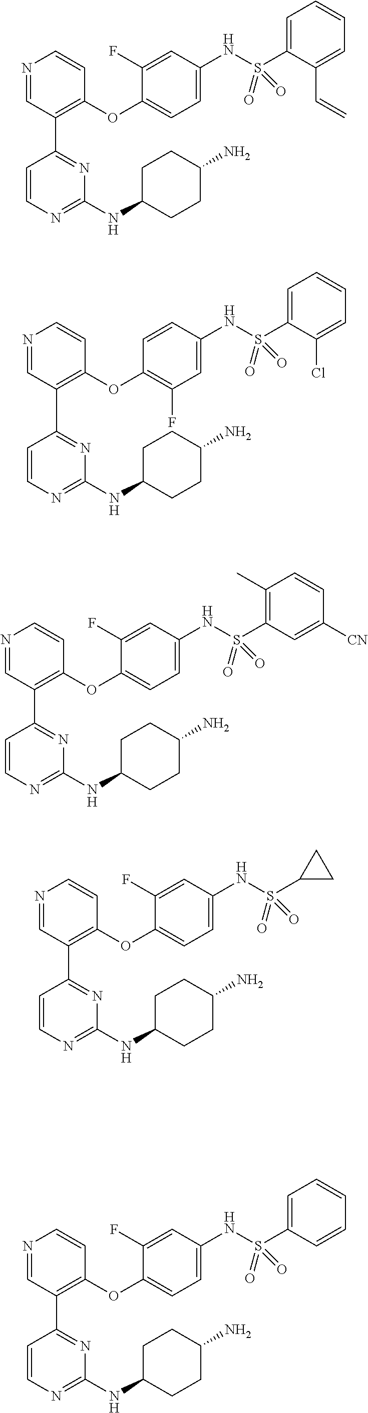 [SMILES: C=Cc1ccccc1S(=O)(=O)Nc1ccc(Oc2ccncc2-c2ccnc(N[C@H]3CC[C@H](N)CC3)n2)c(F)c1 ; N[C@H]1CC[C@H](Nc2nccc(-c3cnccc3Oc3ccc(NS(=O)(=O)c4ccccc4Cl)cc3F)n2)CC1 ; Cc1ccc(C#N)cc1S(=O)(=O)Nc1ccc(Oc2ccncc2-c2ccnc(N[C@H]3CC[C@H](N)CC3)n2)c(F)c1 ; N[C@H]1CC[C@H](Nc2nccc(-c3cnccc3Oc3ccc(NS(=O)(=O)C4CC4)cc3F)n2)CC1 ; N[C@H]1CC[C@H](Nc2nccc(-c3cnccc3Oc3ccc(NS(=O)(=O)c4ccccc4)cc3F)n2)CC1] 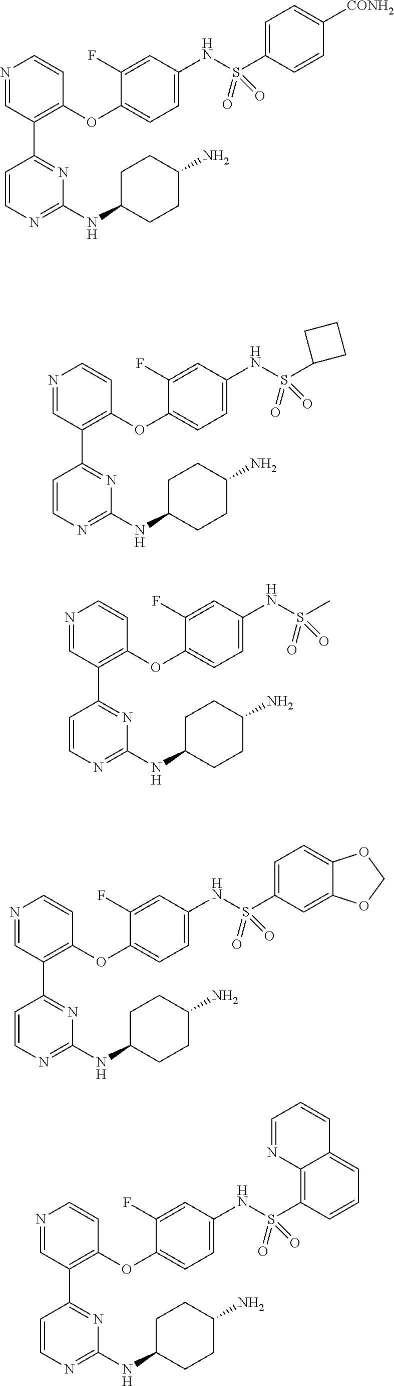 [SMILES: NC(=O)c1ccc(S(=O)(=O)Nc2ccc(Oc3ccncc3-c3ccnc(N[C@H]4CC[C@H](N)CC4)n3)c(F)c2)cc1 ; N[C@H]1CC[C@H](Nc2nccc(-c3cnccc3Oc3ccc(NS(=O)(=O)C4CCC4)cc3F)n2)CC1 ; CS(=O)(=O)Nc1ccc(Oc2ccncc2-c2ccnc(N[C@H]3CC[C@H](N)CC3)n2)c(F)c1 ; N[C@H]1CC[C@H](Nc2nccc(-c3cnccc3Oc3ccc(NS(=O)(=O)c4ccc5c(c4)OCO5)cc3F)n2)CC1 ; N[C@H]1CC[C@H](Nc2nccc(-c3cnccc3Oc3ccc(NS(=O)(=O)c4cccc5cccnc45)cc3F)n2)CC1] 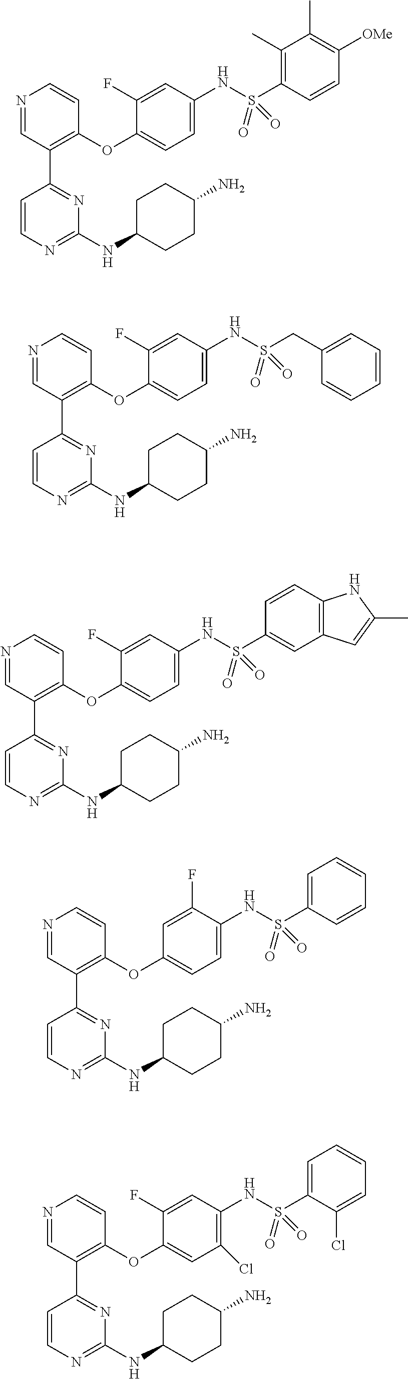 [SMILES: COc1ccc(S(=O)(=O)Nc2ccc(Oc3ccncc3-c3ccnc(N[C@H]4CC[C@H](N)CC4)n3)c(F)c2)c(C)c1C ; N[C@H]1CC[C@H](Nc2nccc(-c3cnccc3Oc3ccc(NS(=O)(=O)Cc4ccccc4)cc3F)n2)CC1 ; Cc1cc2cc(S(=O)(=O)Nc3ccc(Oc4ccncc4-c4ccnc(N[C@H]5CC[C@H](N)CC5)n4)c(F)c3)ccc2[nH]1 ; N[C@H]1CC[C@H](Nc2nccc(-c3cnccc3Oc3ccc(NS(=O)(=O)c4ccccc4)c(F)c3)n2)CC1 ; N[C@H]1CC[C@H](Nc2nccc(-c3cnccc3Oc3cc(Cl)c(NS(=O)(=O)c4ccccc4Cl)cc3F)n2)CC1] 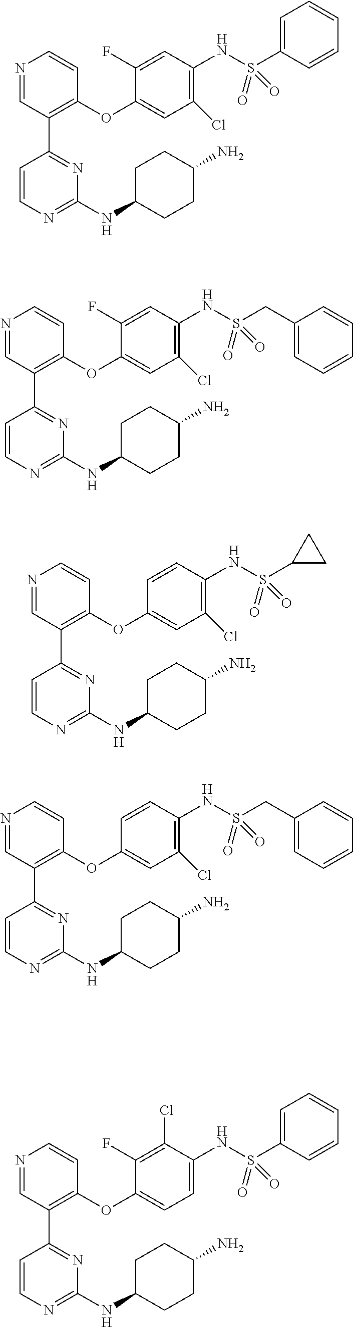 [SMILES: N[C@H]1CC[C@H](Nc2nccc(-c3cnccc3Oc3cc(Cl)c(NS(=O)(=O)c4ccccc4)cc3F)n2)CC1 ; N[C@H]1CC[C@H](Nc2nccc(-c3cnccc3Oc3cc(Cl)c(NS(=O)(=O)Cc4ccccc4)cc3F)n2)CC1 ; N[C@H]1CC[C@H](Nc2nccc(-c3cnccc3Oc3ccc(NS(=O)(=O)C4CC4)c(Cl)c3)n2)CC1 ; N[C@H]1CC[C@H](Nc2nccc(-c3cnccc3Oc3ccc(NS(=O)(=O)Cc4ccccc4)c(Cl)c3)n2)CC1 ; N[C@H]1CC[C@H](Nc2nccc(-c3cnccc3Oc3ccc(NS(=O)(=O)c4ccccc4)c(Cl)c3F)n2)CC1] 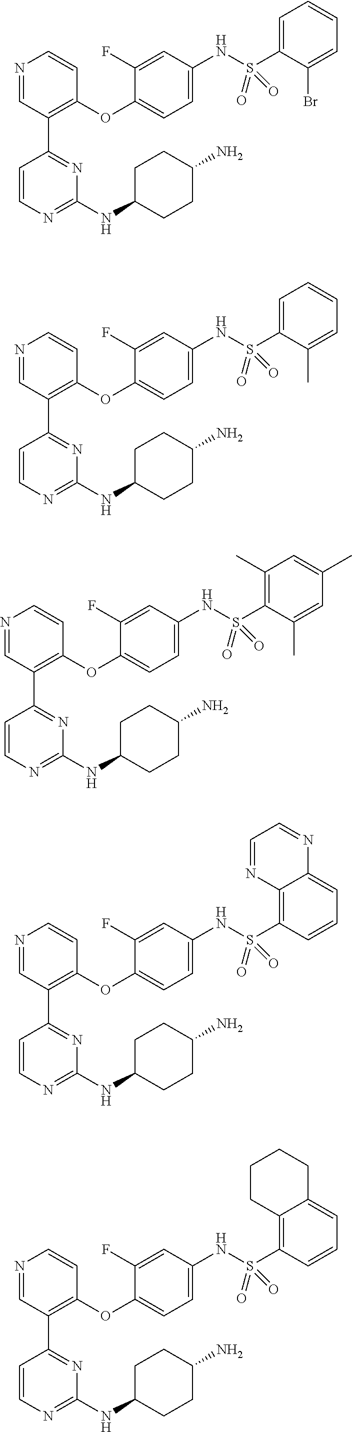 [SMILES: N[C@H]1CC[C@H](Nc2nccc(-c3cnccc3Oc3ccc(NS(=O)(=O)c4ccccc4Br)cc3F)n2)CC1 ; Cc1ccccc1S(=O)(=O)Nc1ccc(Oc2ccncc2-c2ccnc(N[C@H]3CC[C@H](N)CC3)n2)c(F)c1 ; Cc1cc(C)c(S(=O)(=O)Nc2ccc(Oc3ccncc3-c3ccnc(N[C@H]4CC[C@H](N)CC4)n3)c(F)c2)c(C)c1 ; N[C@H]1CC[C@H](Nc2nccc(-c3cnccc3Oc3ccc(NS(=O)(=O)c4cccc5nccnc45)cc3F)n2)CC1 ; N[C@H]1CC[C@H](Nc2nccc(-c3cnccc3Oc3ccc(NS(=O)(=O)c4cccc5c4CCCC5)cc3F)n2)CC1] 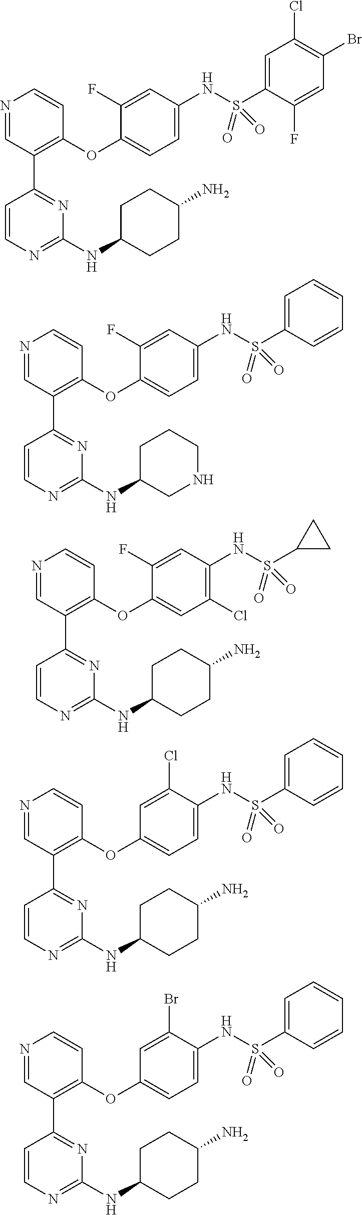 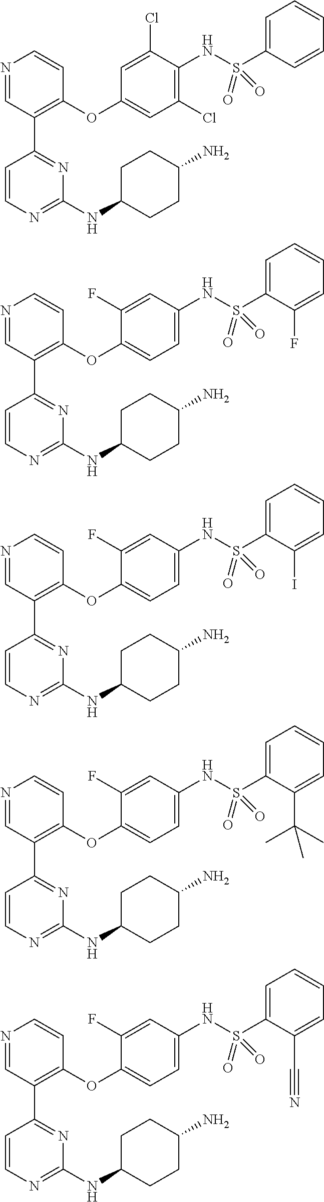 [SMILES: N[C@H]1CC[C@H](Nc2nccc(-c3cnccc3Oc3cc(Cl)c(NS(=O)(=O)c4ccccc4)c(Cl)c3)n2)CC1 ; N[C@H]1CC[C@H](Nc2nccc(-c3cnccc3Oc3ccc(NS(=O)(=O)c4ccccc4F)cc3F)n2)CC1 ; N[C@H]1CC[C@H](Nc2nccc(-c3cnccc3Oc3ccc(NS(=O)(=O)c4ccccc4I)cc3F)n2)CC1 ; CC(C)(C)c1ccccc1S(=O)(=O)Nc1ccc(Oc2ccncc2-c2ccnc(N[C@H]3CC[C@H](N)CC3)n2)c(F)c1 ; N#Cc1ccccc1S(=O)(=O)Nc1ccc(Oc2ccncc2-c2ccnc(N[C@H]3CC[C@H](N)CC3)n2)c(F)c1] 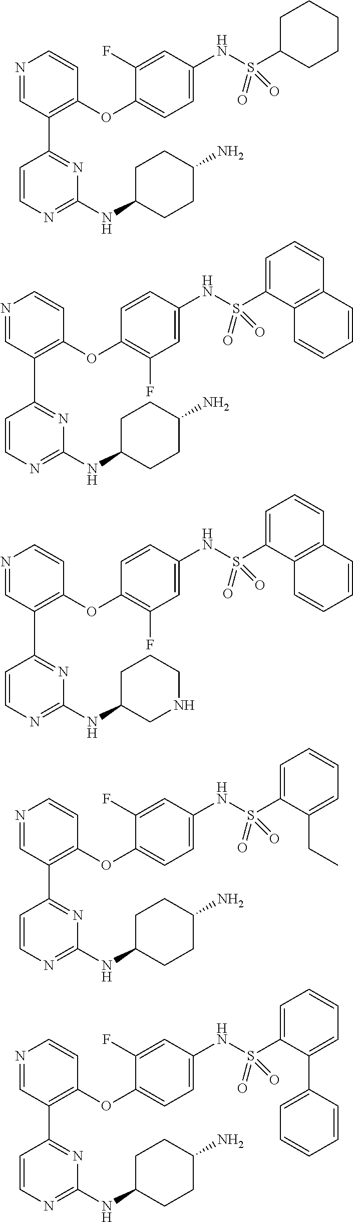 [SMILES: N[C@H]1CC[C@H](Nc2nccc(-c3cnccc3Oc3ccc(NS(=O)(=O)C4CCCCC4)cc3F)n2)CC1 ; N[C@H]1CC[C@H](Nc2nccc(-c3cnccc3Oc3ccc(NS(=O)(=O)c4cccc5ccccc45)cc3F)n2)CC1 ; O=S(=O)(Nc1ccc(Oc2ccncc2-c2ccnc(N[C@H]3CCCNC3)n2)c(F)c1)c1cccc2ccccc12 ; CCc1ccccc1S(=O)(=O)Nc1ccc(Oc2ccncc2-c2ccnc(N[C@H]3CC[C@H](N)CC3)n2)c(F)c1 ; N[C@H]1CC[C@H](Nc2nccc(-c3cnccc3Oc3ccc(NS(=O)(=O)c4ccccc4-c4ccccc4)cc3F)n2)CC1] 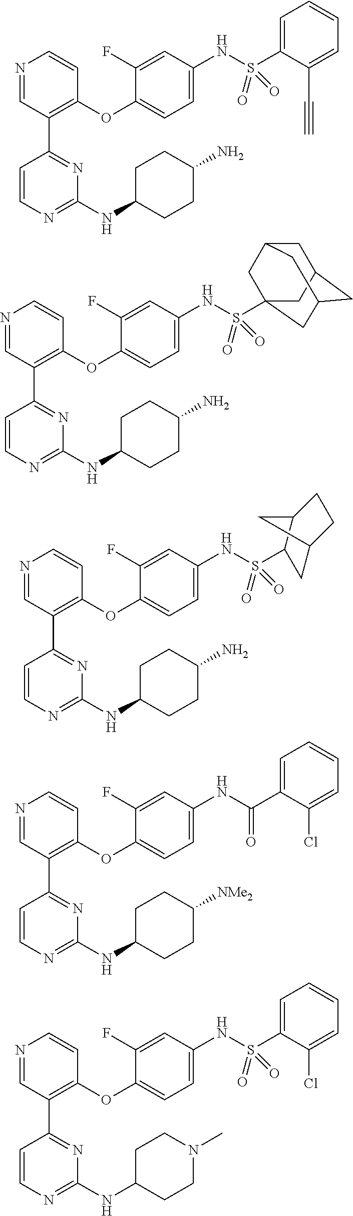 [SMILES: C#Cc1ccccc1S(=O)(=O)Nc1ccc(Oc2ccncc2-c2ccnc(N[C@H]3CC[C@H](N)CC3)n2)c(F)c1 ; N[C@H]1CC[C@H](Nc2nccc(-c3cnccc3Oc3ccc(NS(=O)(=O)C45CC6CC(CC(C6)C4)C5)cc3F)n2)CC1 ; N[C@H]1CC[C@H](Nc2nccc(-c3cnccc3Oc3ccc(NS(=O)(=O)C4CC5CCC4C5)cc3F)n2)CC1 ; CN(C)[C@H]1CC[C@H](Nc2nccc(-c3cnccc3Oc3ccc(NC(=O)c4ccccc4Cl)cc3F)n2)CC1 ; CN1CCC(Nc2nccc(-c3cnccc3Oc3ccc(NS(=O)(=O)c4ccccc4Cl)cc3F)n2)CC1] 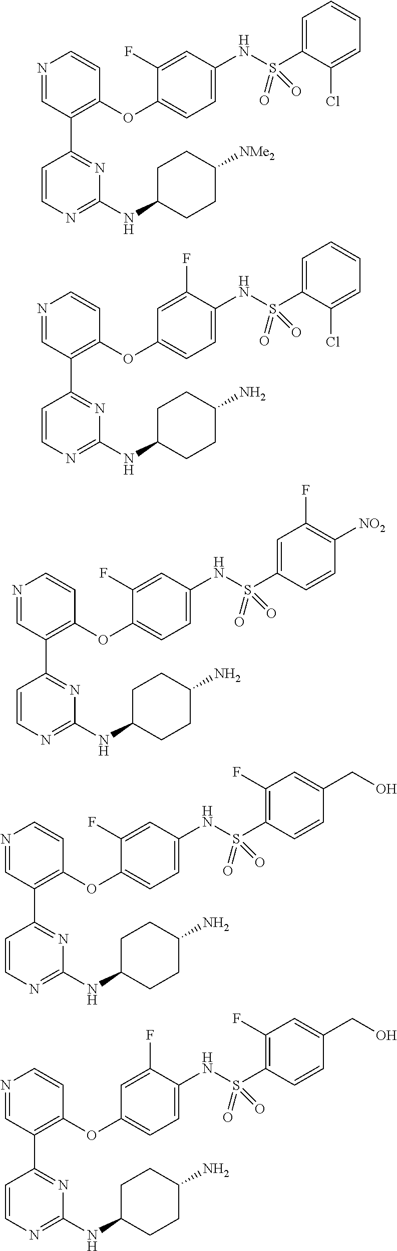 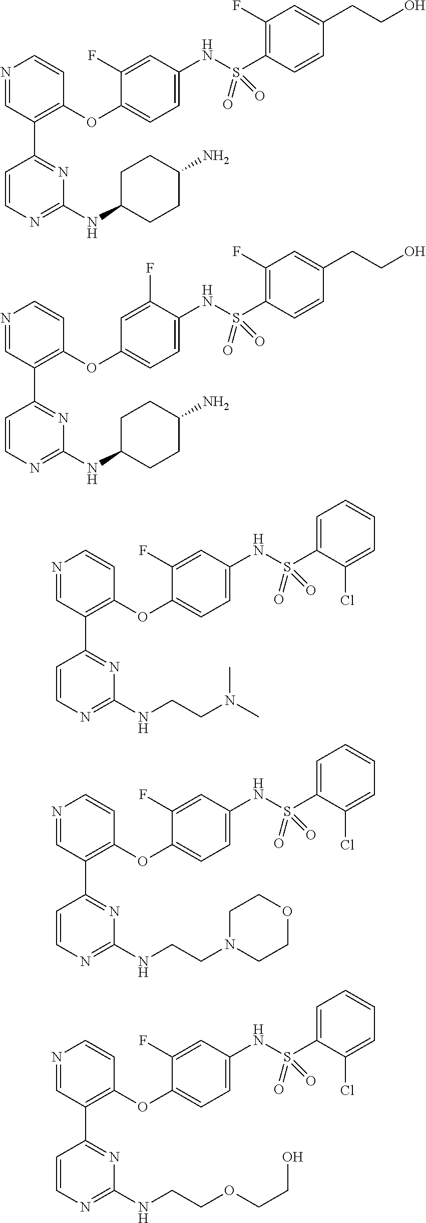 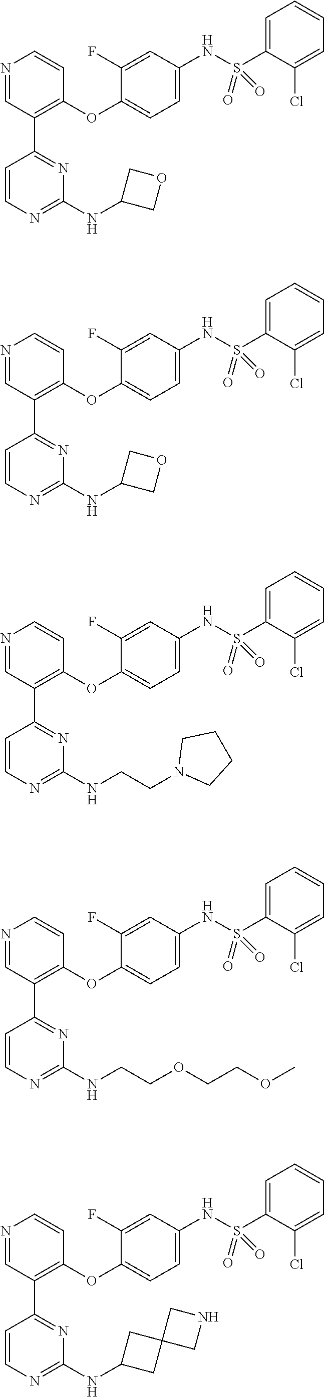 [SMILES: O=S(=O)(Nc1ccc(Oc2ccncc2-c2ccnc(NC3COC3)n2)c(F)c1)c1ccccc1Cl ; O=S(=O)(Nc1ccc(Oc2ccncc2-c2ccnc(NC3COC3)n2)c(F)c1)c1ccccc1Cl ; O=S(=O)(Nc1ccc(Oc2ccncc2-c2ccnc(NCCN3CCCC3)n2)c(F)c1)c1ccccc1Cl ; COCCOCCNc1nccc(-c2cnccc2Oc2ccc(NS(=O)(=O)c3ccccc3Cl)cc2F)n1 ; O=S(=O)(Nc1ccc(Oc2ccncc2-c2ccnc(NC3CC4(CNC4)C3)n2)c(F)c1)c1ccccc1Cl] 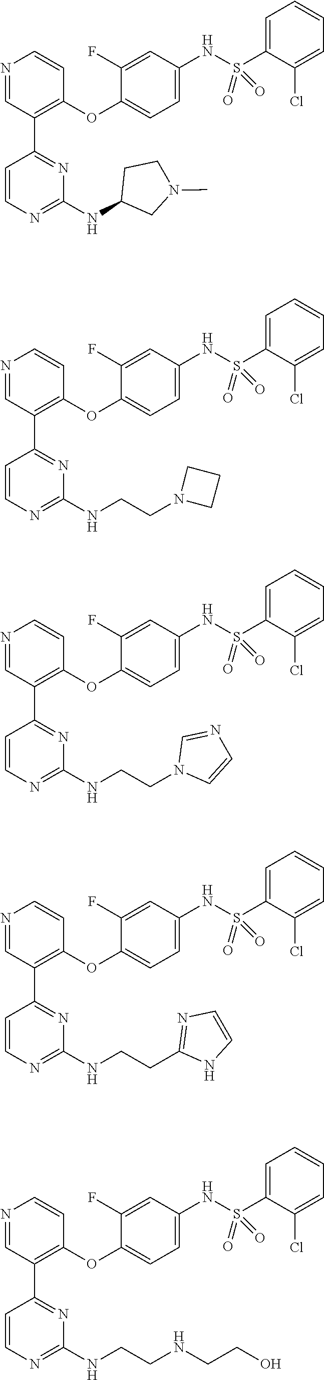 [SMILES: CN1CC[C@H](Nc2nccc(-c3cnccc3Oc3ccc(NS(=O)(=O)c4ccccc4Cl)cc3F)n2)C1 ; O=S(=O)(Nc1ccc(Oc2ccncc2-c2ccnc(NCCN3CCC3)n2)c(F)c1)c1ccccc1Cl ; O=S(=O)(Nc1ccc(Oc2ccncc2-c2ccnc(NCCn3ccnc3)n2)c(F)c1)c1ccccc1Cl ; O=S(=O)(Nc1ccc(Oc2ccncc2-c2ccnc(NCCc3ncc[nH]3)n2)c(F)c1)c1ccccc1Cl ; O=S(=O)(Nc1ccc(Oc2ccncc2-c2ccnc(NCCNCCO)n2)c(F)c1)c1ccccc1Cl] 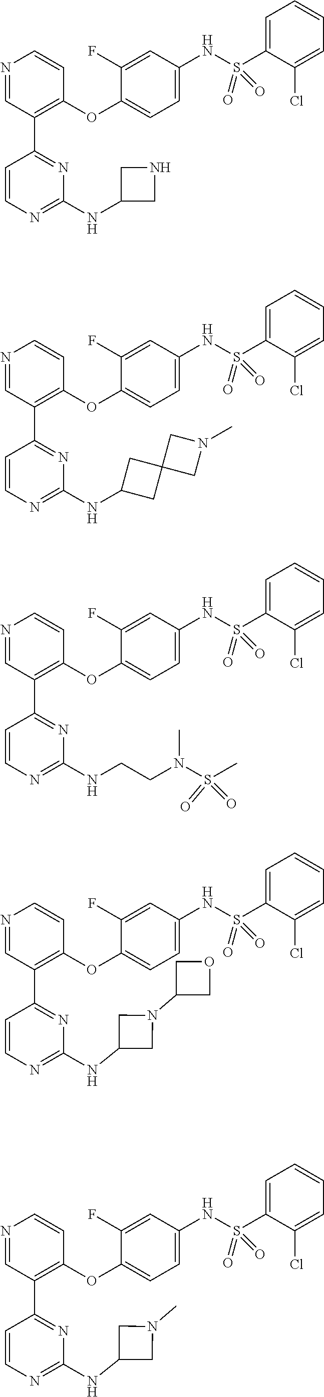 [SMILES: O=S(=O)(Nc1ccc(Oc2ccncc2-c2ccnc(NC3CNC3)n2)c(F)c1)c1ccccc1Cl ; CN1CC2(CC(Nc3nccc(-c4cnccc4Oc4ccc(NS(=O)(=O)c5ccccc5Cl)cc4F)n3)C2)C1 ; CN(CCNc1nccc(-c2cnccc2Oc2ccc(NS(=O)(=O)c3ccccc3Cl)cc2F)n1)S(C)(=O)=O ; O=S(=O)(Nc1ccc(Oc2ccncc2-c2ccnc(NC3CN(C4COC4)C3)n2)c(F)c1)c1ccccc1Cl ; CN1CC(Nc2nccc(-c3cnccc3Oc3ccc(NS(=O)(=O)c4ccccc4Cl)cc3F)n2)C1] 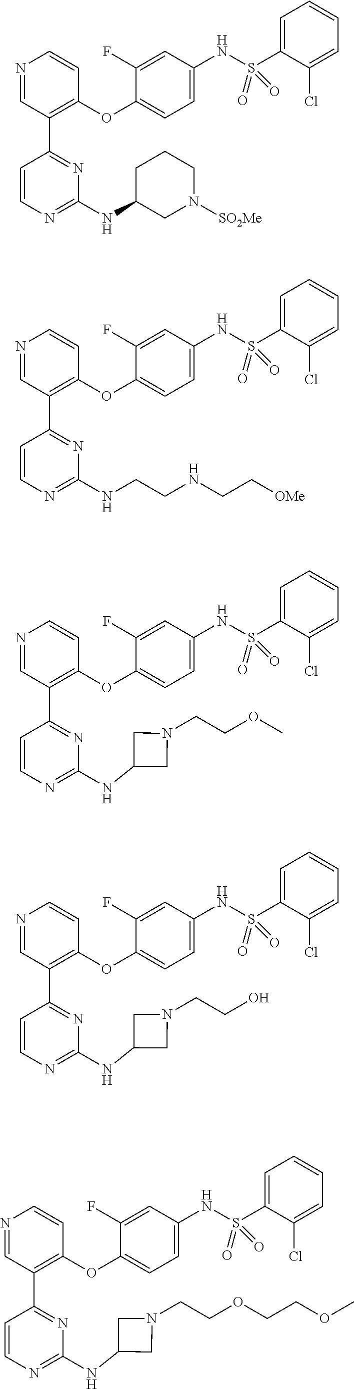 [SMILES: CS(=O)(=O)N1CCC[C@H](Nc2nccc(-c3cnccc3Oc3ccc(NS(=O)(=O)c4ccccc4Cl)cc3F)n2)C1 ; COCCNCCNc1nccc(-c2cnccc2Oc2ccc(NS(=O)(=O)c3ccccc3Cl)cc2F)n1 ; COCCN1CC(Nc2nccc(-c3cnccc3Oc3ccc(NS(=O)(=O)c4ccccc4Cl)cc3F)n2)C1 ; O=S(=O)(Nc1ccc(Oc2ccncc2-c2ccnc(NC3CN(CCO)C3)n2)c(F)c1)c1ccccc1Cl ; COCCOCCN1CC(Nc2nccc(-c3cnccc3Oc3ccc(NS(=O)(=O)c4ccccc4Cl)cc3F)n2)C1] 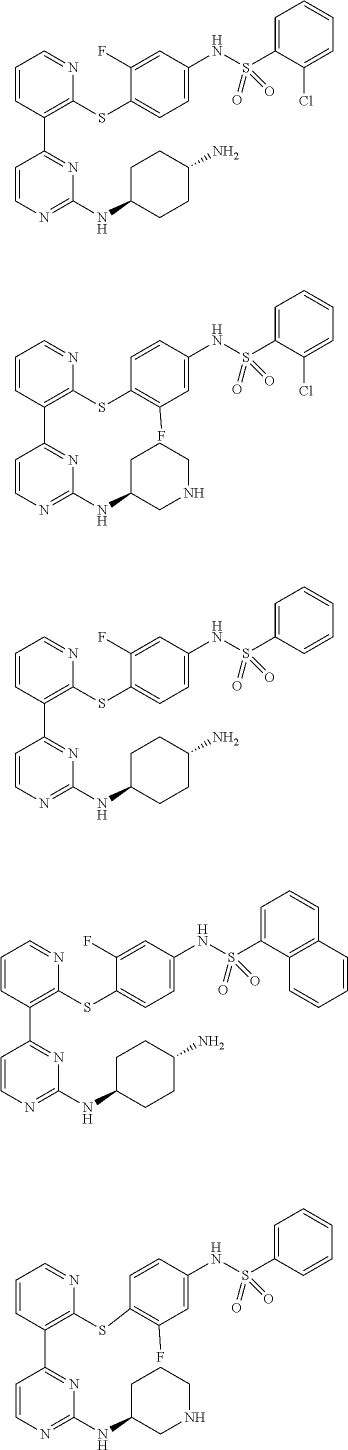 [SMILES: N[C@H]1CC[C@H](Nc2nccc(-c3cccnc3Sc3ccc(NS(=O)(=O)c4ccccc4Cl)cc3F)n2)CC1 ; O=S(=O)(Nc1ccc(Sc2ncccc2-c2ccnc(N[C@H]3CCCNC3)n2)c(F)c1)c1ccccc1Cl ; N[C@H]1CC[C@H](Nc2nccc(-c3cccnc3Sc3ccc(NS(=O)(=O)c4ccccc4)cc3F)n2)CC1 ; N[C@H]1CC[C@H](Nc2nccc(-c3cccnc3Sc3ccc(NS(=O)(=O)c4cccc5ccccc45)cc3F)n2)CC1 ; O=S(=O)(Nc1ccc(Sc2ncccc2-c2ccnc(N[C@H]3CCCNC3)n2)c(F)c1)c1ccccc1] 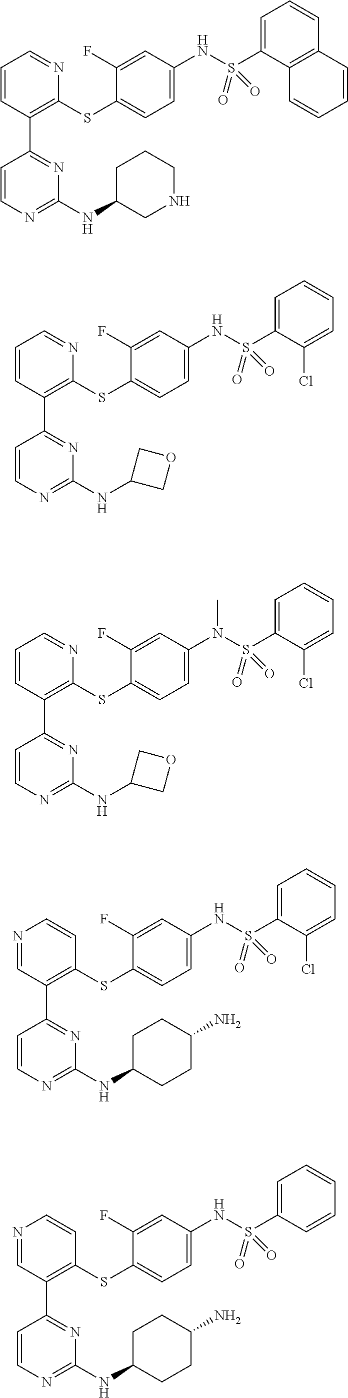 [SMILES: O=S(=O)(Nc1ccc(Sc2ncccc2-c2ccnc(N[C@H]3CCCNC3)n2)c(F)c1)c1cccc2ccccc12 ; O=S(=O)(Nc1ccc(Sc2ncccc2-c2ccnc(NC3COC3)n2)c(F)c1)c1ccccc1Cl ; CN(c1ccc(Sc2ncccc2-c2ccnc(NC3COC3)n2)c(F)c1)S(=O)(=O)c1ccccc1Cl ; N[C@H]1CC[C@H](Nc2nccc(-c3cnccc3Sc3ccc(NS(=O)(=O)c4ccccc4Cl)cc3F)n2)CC1 ; N[C@H]1CC[C@H](Nc2nccc(-c3cnccc3Sc3ccc(NS(=O)(=O)c4ccccc4)cc3F)n2)CC1] 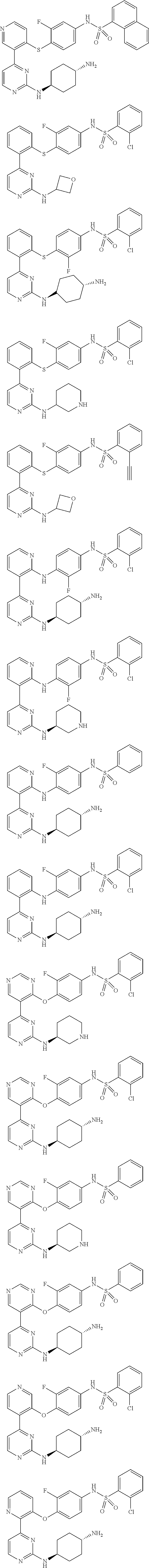
or pharmaceutically acceptable salts thereof.

66. A pharmaceutical composition, comprising:
a) a compound of any one of claims 1-65, or a pharmaceutically acceptable salt thereof; and
b) a pharmaceutically acceptable excipient.

67. A compound of any one of claims 1-65, or a pharmaceutically acceptable salt thereof, or a pharmaceutical composition of claim 66 for use in inhibiting CDK2 in a subject in need thereof.

68. A compound of any one of claims 1-65, or a pharmaceutically acceptable salt thereof, or a pharmaceutical composition of claim 66 for use in treating a disease or disorder characterized by overexpression or amplification of cyclin E in a subject in need thereof.

69. The compound or composition for use of claim 68, wherein the cyclin E is CCNE1 or CCNE2.

70. A compound of any one of claims 1-65, or a pharmaceutically acceptable salt thereof, or a pharmaceutical composition of claim 66 for use in of treating a disease or disorder characterized by overexpression or amplification of cyclin A in a subject in need thereof.

71. A compound of any one of claims 1-65, or a pharmaceutically acceptable salt thereof, or a pharmaceutical composition of claim 66 for use in of treating cancer in a subject in need thereof.

72. The compound or composition for use of claim 71, wherein the cancer is associated with amplification or overexpression of cyclin E or cyclin A.

73. The compound or composition for use of claim 71 or claim 72, wherein the cancer is breast cancer, ovarian cancer, uterine cancer, endometrial cancer, cervical cancer, melanoma, lung cancer, pancreatic cancer, stomach cancer, esophageal cancer, bladder cancer, colon cancer, rectal cancer, testicular cancer, prostate cancer, renal cancer, hepatic cancer, head and neck cancer, thyroid cancer, glioblastoma, sarcoma, lymphoma, or leukemia.

74. A compound of any one of claims 1-65, or a pharmaceutically acceptable salt thereof, or a pharmaceutical composition of claim 66 for use in of treating an autoimmune disorder or disease in a subject in need thereof.

75. The compound or composition for use of claim 74, wherein the autoimmune disorder or disease is associated with amplification or overexpression of cyclin E or cyclin A.

76. The compound or composition for use of claim 74 or 75, wherein the autoimmune disorder or disease is rheumatoid arthritis, Lupus, Crohn's Disease, Addison disease, celiac disease, dermatomyositis, Graves' disease, thyroiditis, multiple sclerosis, pernicious anemia, reactive arthritis, or pemphigus vulgaris.

77. A compound of any one of claims 1-65, or a pharmaceutically acceptable salt thereof, or a pharmaceutical composition of claim 66 for use in of treating an inflammatory disease or disorder in a subject in need thereof.

78. The compound or composition for use of claim 77, wherein the inflammatory disease or disorder is associated with amplification or overexpression of cyclin E or cyclin A.

79. The compound or composition for use of claim 77 or 78, wherein the inflammatory disease or disorder is asthma, chronic peptic ulcers, psoriasis, inflammatory bowel disease, tuberculosis, rheumatoid arthritis, periodontitis, ulcerative colitis, or hepatitis.

80. A compound of any one of claims 1-65, or a pharmaceutically acceptable salt thereof, or a pharmaceutical composition of claim 66 for use in of treating a neurodegenerative disease or disorder in a subject in need thereof.

81. The compound or composition for use of claim 80, wherein the neurodegenerative disease or disorder is associated with amplification or overexpression of cyclin E or cyclin A.

82. The compound or composition for use of claim 80 or 81, wherein the neurodegenerative disease or disorder is Alzheimer's disease, AIDS-related dementia, Parkinson's disease, amyotrophic lateral sclerosis, retinitis pigmentosa, spinal muscular atrophy, or cerebellar degeneration.

83. A conjugate, comprising a compound of any one of claims 1-65, or a pharmaceutically acceptable salt thereof, bound to CDK2.
